(19) United States
(12) Patent Application Publication
(10) Pub. No.: US 2025/0169358 A1
(43) Pub. Date: May 22, 2025

(54) Organic Compound, And Organic Light Emitting Diode And Organic Light Emitting Device Including The Same

(71) Applicants: LG Display Co., Ltd., Seoul (KR); LT Materials Co., Ltd, Yongin-si (KR)

(72) Inventors: Yeong-Su Lim, Paju-si (KR); Young-Jun Yu, Paju-si (KR); Seon-Keun Yoo, Paju-si (KR); Sang-Beom Kim, Paju-si (KR); Woo-Sam Kim, Yongin-si (KR); Hyung-Keun Jang, Yongin-si (KR); Dae-Hyuk Choi, Yongin-si (KR); Dong-Jun Kim, Yongin-si (KR); Young-Seok No, Yongin-si (KR); Hyun-Joo Lee, Yongin-si (KR); Geon-Yu Park, Yongin-si (KR); Sang-Hun Ahn, Yongin-si (KR)

(21) Appl. No.: 18/920,683

(22) Filed: Oct. 18, 2024

(30) Foreign Application Priority Data
Nov. 6, 2023 (KR) 10-2023-0151867

(51) Int. Cl. H10K 85/60; C07D 413/14; C09K 11/02; C09K 11/06; H10K 50/12; H10K 50/13; H10K 50/16; H10K 85/30

(52) U.S. Cl. CPC H10K 85/6572; C07D 413/14; C09K 11/02; C09K 11/06; H10K 85/342; H10K 85/615; H10K 85/622; H10K 85/626; H10K 85/654; H10K 85/6574; H10K 85/6576; C09K 2211/1007; C09K 2211/1029; C09K 2211/185; H10K 50/12; H10K 50/13; H10K 50/16

(57) ABSTRACT

An organic compound, and an organic light emitting diode and an organic light emitting device including the same are disclosed. For example, an organic compound is represented by the following chemical formula. The organic light emitting diode and the organic light emitting device each includes the organic compound.

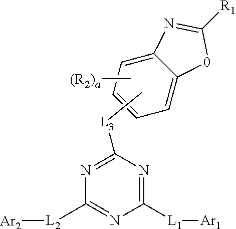

### CROSS-REFERENCE TO RELATED APPLICATIONS

The present application claims the benefit of and the priority to Republic of Korea Patent Application No. 10-2023-0151867 filed in the Republic of Korea on Nov. 6, 2023.

### TECHNICAL FIELD

The present disclosure relates to an organic compound, and more specifically, to an organic compound than can improve the driving voltage, the emitting efficiency and the lifespan and an organic light emitting device including the same.

### BACKGROUND

Recently, as demand for a flat panel display device having a small area have increased, an organic light emitting display device including an organic light emitting diode (OLED) has been the subject of recent research and development.

The OLED emits light by injecting electrons from a cathode as an electron injection electrode and holes from an anode as a hole injection electrode into an emitting material layer (EML), combining the electrons with the holes, generating an exciton, and transforming the exciton from an excited state to a ground state. A flexible substrate, for example, a plastic substrate, can be used as a base substrate where elements are formed. In addition, the organic light emitting display device can be operated at a voltage (e.g., 10 V or below) lower than a voltage to operate other display devices. Moreover, the organic light emitting display device has improved power consumption and colour.

The OLED includes a first electrode as an anode on a substrate, a second electrode as a cathode being spaced apart from and facing the first electrode and an organic light emitting layer between the first and second electrodes.

Although there have been many studies and developments on the materials of the organic light emitting layer, the OLED still has a limitation in the driving voltage, the emitting efficiency and the lifespan.

### SUMMARY

The present disclosure is directed to an organic compound, an organic light emitting diode and an organic light emitting device that substantially obviate one or more of the problems associated with the limitations and disadvantages of the related art.

Additional features and advantages of the present disclosure are set forth in the description which follows, and will be apparent from the description, or evident by practice of the present disclosure. The objectives and other advantages of the present disclosure are realized and attained by the features described herein as well as in the appended drawings.

To achieve these and other advantages in accordance with the purpose of the embodiments of the present disclosure, as described herein, an aspect of the present disclosure is an organic compound represented by Formula 1: [Formula 1]

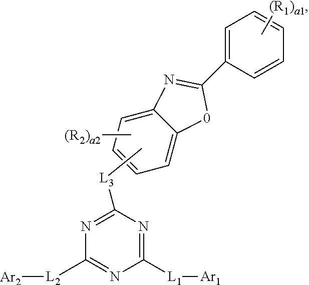

wherein a1 is an integer of 0 to 5, a2 is an integer of 0 to 3, wherein each R₁ is selected from the group consisting of a substituted or unsubstituted C1 to C10 alkyl group, wherein each R₂ is selected from the group consisting of a substituted or unsubstituted C1 to C10 alkyl group, a substituted or unsubstituted C6 to C60 aryl group and a substituted or unsubstituted C3 to C60 heteroaryl group, wherein when a1 is 2 or more, 2 or more R₁ groups are the same or different, wherein when a2 is 2 or more, 2 or more R₂ groups are the same or different, wherein each L₁, L₂ and L₃ is selected from the group consisting of a single bond, a substituted or unsubstituted C6 to C60 arylene group and a substituted or unsubstituted C3 to C60 heteroarylene group, wherein Ar₁ is selected from Formula 1a-1 to Formula 1a-5,

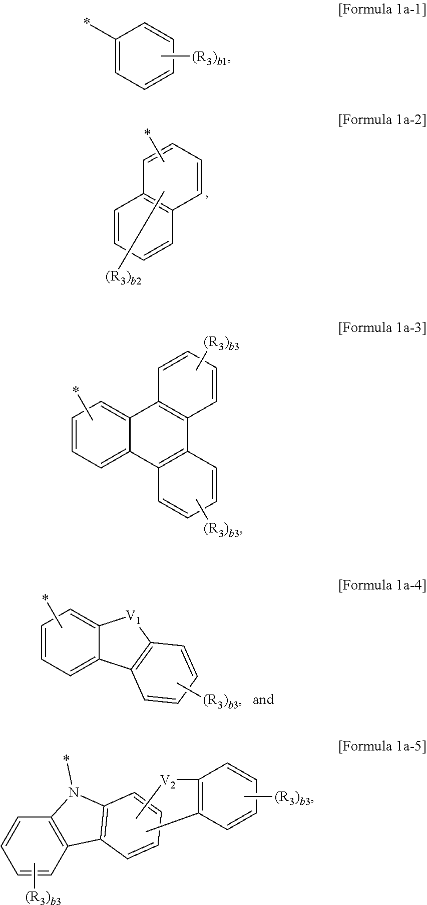

wherein in Formula 1a-1, b1 is an integer of 0 to 5, wherein in Formula 1a-2, b2 is an integer of 0 to 7, wherein in each of Formulas 1a-3 to 1a-5, each b3 is independently an integer of 0 to 4, wherein when each b1, b2 and b3 is 2 or more, 2 or more R₃ groups are the same or different, wherein in Formula 1a-4, V₁ is selected from O, S and C(R₃)₂, wherein in Formula 1a-5, V₂ is selected from O and S, wherein in each of Formulas 1a-1 to 1a-5, R₃ is selected from the group consisting of deuterium, a substituted or unsubstituted C1 to C10 alkyl group, a substituted or unsubstituted C6 to C60 aryl group and a substituted or unsubstituted C3 to C60 heteroaryl group, wherein Ar₂ is selected from Formula 1b-1 and Formula 1b-2,

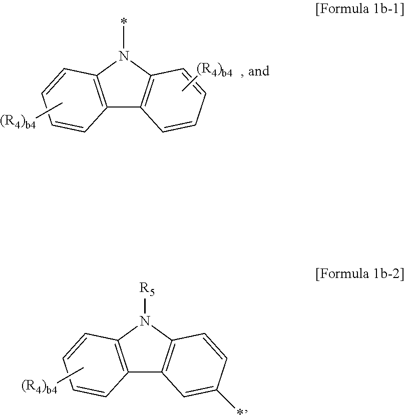

wherein in each of Formulas 1b-1 and 1b-2, b4 is an integer of 0 to 4, wherein each R₄ is independently selected from the group consisting of deuterium, a substituted or unsubstituted C1 to C10 alkyl group, a substituted or unsubstituted C6 to C60 aryl group and a substituted or unsubstituted C3 to C60 heteroaryl group, wherein when b4 is 2 or more, 2 or more R₄ groups are the same or different, and
wherein in Formula 1b-2, R₅ is selected from the group consisting of hydrogen, deuterium, a substituted or unsubstituted C1 to C10 alkyl group and a substituted or unsubstituted C6 to C60 aryl group.

In preferred embodiments, Ar1 and Ar2 are different.

Another aspect of the present disclosure is an organic light emitting device comprising a substrate; and an organic light emitting diode positioned on the substrate and including a first electrode; a second electrode facing the first electrode; and a first emitting part between the first and second electrodes, the first emitting part including a first emitting material layer, wherein the first emitting material layer includes a first compound that is the above-described organic compound.

It is to be understood that both the foregoing general description and the following detailed description are exemplary and explanatory and are intended to further explain the present disclosure as claimed.

### BRIEF DESCRIPTION OF THE DRAWINGS

The accompanying drawings, which are included to provide a further understanding of the present disclosure and are incorporated in and constitute a part of this specification, illustrate embodiments of the present disclosure and together with the description serve to explain the principles of the present disclosure.

FIG. 1 illustrates a schematic circuit diagram for an organic light emitting display device according to an example embodiment of the present disclosure.

FIG. 2 illustrates a schematic cross-sectional view of an organic light emitting display device according to a first embodiment of the present disclosure.

FIG. 3 illustrates a schematic cross-sectional view of an OLED according to a second embodiment of the present disclosure.

FIG. 4 illustrates a schematic cross-sectional view of an OLED according to a third embodiment of the present disclosure.

FIG. 5 illustrates a schematic cross-sectional view of an organic light emitting display device according to a fourth embodiment of the present disclosure.

FIG. 6 illustrates a schematic cross-sectional view of an OLED according to a fifth embodiment of the present disclosure.

FIG. 7 illustrates a schematic cross-sectional view of an OLED according to a sixth embodiment of the present disclosure.

FIG. 8 illustrates a schematic cross-sectional view of an OLED according to a seventh embodiment of the present disclosure.

FIG. 9 illustrates a schematic cross-sectional view of an OLED according to an eighth embodiment of the present disclosure.

FIG. 10 illustrates a schematic cross-sectional view of an OLED according to a ninth embodiment of the present disclosure.

### DETAILED DESCRIPTION

Reference will now be made in detail to some of the examples and embodiments of the disclosure illustrated in the accompanying drawings. Wherever possible, the same reference numbers will be used throughout the drawings to refer to the same or like parts.

Advantages and features of the present disclosure, and implementation methods thereof will be clarified through the following example embodiments described with reference to the accompanying drawings. The present disclosure may, however, be embodied in different forms and should not be construed as limited to the example embodiments set forth herein. Rather, these example embodiments are provided so that this disclosure may be sufficiently thorough and complete to assist those skilled in the art to fully understand the scope of the present disclosure. Further, the protected scope of the present disclosure is defined by claims and their equivalents.

The shapes, sizes, ratios, angles, numbers, and the like, which are illustrated in the drawings to describe various example embodiments of the present disclosure, are merely given by way of example. Therefore, the present disclosure is not limited to the illustrations in the drawings. The same or similar elements are designated by the same reference numerals throughout the specification unless otherwise specified.

In the following description, where the detailed description of the relevant known function or configuration may unnecessarily obscure an important point of the present disclosure, a detailed description of such known function of configuration may be omitted.

In the present specification, where the terms “comprise,” “have,” “include,” and the like are used, one or more other elements may be added unless the term, such as “only,” is used. An element described in the singular form is intended to include a plurality of elements, and vice versa, unless the context clearly indicates otherwise.

In construing an element, the element is to be construed as including an error or tolerance range even where no explicit description of such an error or tolerance range is provided.

In the description of the various embodiments of the present disclosure, where positional relationships are described, for example, where the positional relationship between two parts is described using “on,” “over,” “under,” “above,” “below,” “beside,” “next,” or the like, one or more other parts may be located between the two parts unless a more limiting term, such as “immediate(ly),” “direct(ly),” or “close(ly)” is used. For example, where an element or layer is disposed “on” another element or layer, a third layer or element may be interposed therebetween.

In describing a temporal relationship, when the temporal order is described as, for example, “after,” “subsequent,” “next,” or “before,” a case which is not continuous may be included unless a more limiting term, such as “just,” “immediate(ly),” or “direct(ly),” is used.

Although the terms “first,” “second,” and the like may be used herein to describe various elements, the elements should not be limited by these terms. These terms are used only to identify one element from another. For example, a first element could be termed a second element, and similarly, a second element could be termed a first element, without departing from the scope of the present disclosure.

Although the terms “first,” “second,” A, B, (a), (b), and the like may be used herein to describe various elements, the elements should not be interpreted to be limited by these terms as they are not used to define a particular order, precedence, or number of the corresponding elements. These terms are used only to identify one element from another.

The expression that an element or layer is “connected” to another element or layer means the element or layer can not only be directly connected to another element or layer, but also be indirectly connected or adhered to another element or layer with one or more intervening elements or layers “disposed,” or “interposed” between the elements or layers, unless otherwise specified.

The term “at least one” should be understood as including any and all combinations of one or more of the associated listed items. For example, the meaning of “at least one of a first element, a second element, and a third element” encompasses the combination of all three listed elements, combinations of any two of the three elements, as well as each individual element, the first element, the second element, and the third element.

Features of various embodiments of the present disclosure may be partially or overall coupled to or combined with each other, and may be variously inter-operated with each other and driven technically as those skilled in the art can sufficiently understand. Embodiments of the present disclosure may be carried out independently from each other, or may be carried out together in a co-dependent relationship.

Hereinafter, example embodiments of the present disclosure will be described in detail with reference to the accompanying drawings. In adding reference numerals to elements of each of the drawings, although the same elements are illustrated in other drawings, like reference numerals may refer to like elements. Also, for convenience of description, a scale in which each of elements is illustrated in the accompanying drawings may differ from an actual scale. Thus, the illustrated elements are not limited to the specific scale in which they are illustrated in the drawings.

The OLED of the present disclosure may include an example of an organic compound of the present disclosure. The OLED may be included in an organic light emitting display device or an organic light emitting lighting device. The explanation below is focused on an example of an organic light emitting display device including the OLED of the present disclosure.

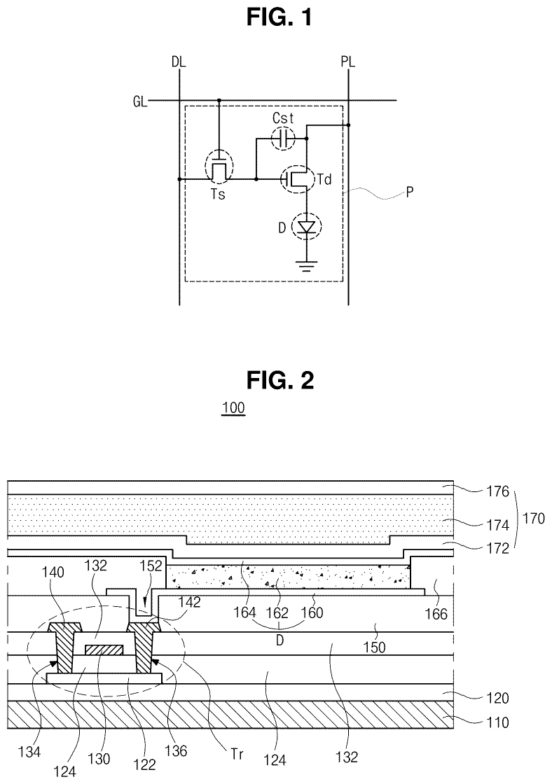

FIG. 1 illustrates a schematic circuit diagram for an organic light emitting display device according to an example embodiment of the present disclosure.

As illustrated in FIG. 1, a gate line GL and a data line DL, which may cross each other to define a pixel region P, and a power line PL may be formed in an organic light display device. A switching thin film transistor (TFT) Ts, a driving thin film transistor (TFT) Td, a storage capacitor Cst, and an OLED D may be formed in the pixel region P. The pixel region P may include a red pixel region, a green pixel region, and a blue pixel region. In addition, the pixel region P may further include a white pixel region.

The switching thin film transistor Ts may be connected to the gate line GL and the data line DL, and the driving thin film transistor Td and the storage capacitor Cst may be connected between the switching thin film transistor Ts and the power line PL. The OLED D may be connected to the driving thin film transistor Td. When the switching thin film transistor Ts is turned on by the gate signal applied through the gate line GL, the data signal applied through the data line DL may be applied to a gate electrode of the driving thin film transistor Td and one electrode of the storage capacitor Cst through the switching thin film transistor Ts.

The driving thin film transistor Td may be turned on by the data signal applied to the gate electrode so that a current proportional to the data signal may be supplied from the power line PL to the OLED D through the driving thin film transistor Td. The OLED D may emit light having a luminance proportional to the current flowing through the driving thin film transistor Td. In this case, the storage capacitor Cst may be charged with a voltage proportional to the data signal so that the voltage of the gate electrode in the driving thin film transistor Td may be kept constant or similar during one frame. Therefore, the organic light emitting display device can display a desired image.

FIG. 2 illustrates a schematic cross-sectional view of an organic light emitting display device according to a first embodiment of the present disclosure.

As illustrated in FIG. 2, the organic light emitting display device 100 may include a substrate 110, a TFT Tr, and an OLED D connected to the TFT Tr. For example, the organic light emitting display device 100 may include a red pixel region, a green pixel region, and a blue pixel region, and the OLED D may be disposed in each of the red, green and blue pixel regions. The organic light emitting display device 100 may further include a yellow-green pixel region, and the OLED D may be disposed in the yellow-green pixel region. For example, the OLEDs D emitting red light, green light, blue light, yellow-green light may be provided in the red, green, blue and yellow-green pixel regions, respectively.

The substrate 110 may be a glass substrate or a flexible substrate. For example, the flexible substrate may be a polyimide (PI) substrate, a polyethersulfone (PES) substrate, a polyethylenenaphthalate (PEN) substrate, a polyethylene terephthalate (PET) substrate or a polycarbonate (PC) substrate.

A buffer layer 120 may be formed on the substrate, and the TFT Tr may be formed on the buffer layer 120. The buffer layer 120 may be formed of an inorganic insulating material, e.g., silicon oxide or silicon nitride. The buffer layer 120 may have a multi-layered structure including a first layer of silicon oxide and a second layer of silicon nitride. The buffer layer 120 may be omitted, and the TFT Tr may be disposed on the substrate 110.

A semiconductor layer 122 may be formed on the buffer layer 120. The semiconductor layer 122 may include an oxide semiconductor material or polycrystalline silicon.

When the semiconductor layer 122 includes the oxide semiconductor material, a light-shielding pattern (not shown) may be formed under the semiconductor layer 122. The light to the semiconductor layer 122 may be shielded or blocked by the light-shielding pattern such that thermal degradation of the semiconductor layer 122 can be prevented or reduced. On the other hand, when the semiconductor layer 122 includes polycrystalline silicon, impurities may be doped into both sides of the semiconductor layer 122.

A gate insulating layer 124 may be formed on the semiconductor layer 122. The gate insulating layer 124 may be formed of an inorganic insulating material such as silicon oxide or silicon nitride.

A gate electrode 130, which may be formed of a conductive material, e.g., metal, may be formed on the gate insulating layer 124 to correspond to a center of the semiconductor layer 122.

In FIG. 2, the gate insulating layer 124 may be formed on an entire surface of the substrate 110. Alternatively, the gate insulating layer 124 may be patterned to have the same shape as the gate electrode 130. However, embodiments of the present disclosure are not limited to such examples.

An interlayer insulating layer 132, which may be formed of an insulating material, may be formed on the gate electrode 130. The interlayer insulating layer 132 may be formed of an inorganic insulating material, e.g., silicon oxide or silicon nitride, or an organic insulating material, e.g., benzocyclobutene or photo-acryl.

The interlayer insulating layer 132 may include first and second contact holes 134 and 136 exposing both sides of the semiconductor layer 122. The first and second contact holes 134 and 136 may not cover a portion of the surface of the semiconductor layer 122 that is nearer to the opposing ends than to a center of the semiconductor layer 122. The first and second contact holes 134 and 136 may be positioned at both sides of the gate electrode 130 to be spaced apart from the gate electrode 130.

The first and second contact holes 134 and 136 may be formed through the interlayer insulating layer 132 and the gate insulating layer 124. Alternatively, when the gate insulating layer 124 is patterned to have the same shape as the gate electrode 130, the first and second contact holes 134 and 136 may be formed only through the interlayer insulating layer 132. However, embodiments of the present disclosure are not limited to such examples.

A source electrode 140 and a drain electrode 142, which may be formed of a conductive material, e.g., metal, may be formed on the interlayer insulating layer 132.

The source electrode 140 and the drain electrode 142 may be spaced apart from each other with respect to the gate electrode 130 and may contact both sides of the semiconductor layer 122 through the first and second contact holes 134 and 136, respectively.

The semiconductor layer 122, the gate electrode 130, the source electrode 140, and the drain electrode 142 may constitute the TFT Tr. The TFT Tr may serve as a driving element. For example, the TFT Tr may correspond to the driving TFT Td (of FIG. 1).

In the TFT Tr, the gate electrode 130, the source electrode 140, and the drain electrode 142 may be positioned on the semiconductor layer 122. For example, the TFT Tr may have a coplanar structure.

Alternatively, in the TFT Tr, the gate electrode may be positioned under the semiconductor layer, and the source and drain electrodes may be positioned on the semiconductor layer such that the TFT Tr may have an inverted staggered structure. In this instance, the semiconductor layer may include amorphous silicon. However, embodiments of the present disclosure are not limited to such examples.

Although not shown, the gate line and the data line may cross each other to define the pixel region, and the switching TFT may be formed to be connected to the gate and data lines. The switching TFT may be connected to the TFT Tr as the driving element.

In addition, the power line, which may be formed to be parallel to and spaced apart from one of the gate and data lines, and the storage capacitor for maintaining the voltage of the gate electrode of the TFT Tr in one frame may be further formed.

A planarization layer (or a passivation layer) 150, which may include a drain contact hole 152 exposing the drain electrode 142 of the TFT Tr, may be formed to cover the TFT Tr. The drain contact hole 152 may not cover the drain electrode 142.

A first electrode 160, which may be connected to the drain electrode 142 of the TFT Tr through the drain contact hole 152, may be separately formed in each pixel region and on the planarization layer 150.

The first electrode 160 may be an anode and may include a transparent conductive oxide material layer formed of a conductive material, e.g., a transparent conductive oxide (TCO), having a relatively high work function. For example, the transparent conductive oxide material layer of the first electrode 160 may include at least one of indium-tin-oxide (ITO) indium-zinc-oxide (IZO), indium-tin-zinc oxide; ITZO), tin oxide (SnO), zinc oxide (ZnO), indium-copper-oxide (ICO) and Al:ZnO (AZO).

When the organic light emitting display device 100 is operated as a bottom-emission type, the first electrode 160 may have a single-layered structure of the transparent conductive oxide material layer.

Alternatively, when the organic light emitting display device 100 is operated as a top-emission type, the first electrode 160 may further include a reflection layer to have a double-layered structure or a triple-layered structure. For example, the reflection layer may be formed of silver (Ag) or aluminium-palladium-copper (APC) alloy. In the top-emission type organic light emitting display device 100, the first electrode 160 may have a double-layered structure of Ag/ITO or APC/ITO or a triple-layered structure of ITO/Ag/ITO or ITO/APC/ITO. However, embodiments of the present disclosure are not limited to such examples.

A bank layer 166 may be formed on the planarization layer 150 to cover an edge of the first electrode 160. For example, the bank layer 166 may be positioned at a boundary of the pixel region and exposes a center of the first electrode 160 in the pixel region.

An organic light emitting layer 162 may be formed on the first electrode 160. The organic light emitting layer 162 may include one emitting part including an emitting material layer (EML). Alternatively, the organic light emitting layer 162 may include a plurality of emitting parts and each emitting part may include the EML. In addition, the organic light emitting layer 162 may further include a charge generation layer between adjacent emitting parts. Embodiments of the present disclosure are not limited to such examples.

The emitting part or each of the emitting parts may have a multi-layered structure including at least one of a hole injection layer (HIL), a hole transporting layer (HTL), an electron blocking layer (EBL), a hole blocking layer (HBL), an electron transporting layer (ETL), and an electron injection layer (EIL).

The organic light emitting layer 162 may be separated in each of the red, green, and blue pixel regions. As described herein, in the OLED D in the green pixel region according to an example embodiment in the present disclosure, the organic light emitting layer 162 may include an example of an organic compound of the present disclosure. As a result, in the OLED D and the organic light emitting display device 100 including the organic compound, the driving voltage may be decreased, and the emitting efficiency and the lifespan may be increased. For example, the EML of the organic light emitting layer 162 may include the organic compound of the present disclosure.

A second electrode 164 may be formed over the substrate 110 where the organic light emitting layer 162 is formed. The second electrode 164 may cover an entire surface of the display area and may be formed of a conductive material having a relatively low work function to serve as a cathode. For example, the second electrode 164 may be formed of aluminium (Al), magnesium (Mg), calcium (Ca), silver (Ag) or their alloy or a combination thereof. In the top-emission type organic light emitting display device 100, the second electrode 164 may have a thin profile (small thickness) to provide a light transmittance property (or a semi-transmittance property).

The first electrode 160, the organic light emitting layer 162, and the second electrode 164 may constitute the OLED D.

An encapsulation layer (e.g., an encapsulation film) 170 may be formed on the second electrode 164 to prevent penetration of moisture into the OLED D. The encapsulation layer 170 may include a first inorganic insulating layer 172, an organic insulating layer 174, and a second inorganic insulating layer 176 sequentially stacked. However, embodiments of the present disclosure are not limited to such examples. The encapsulation layer 170 may be omitted.

In the bottom-emission type organic light emitting display device 100, a metal plate may be further disposed on the encapsulation layer 170.

The organic light emitting display device 100 may further include a colour filter layer corresponding to the red, green and blue pixel regions. The colour filter layer may include red, green and blue colour filter patterns respectively corresponding to the red, green and blue pixel regions. When the organic light emitting display device 100 includes the colour filter layer, a colour purity of the organic light emitting display device 100 may be improved.

In the bottom-emission type organic light emitting display device 100, the colour filter layer may be positioned between the OLED D and the substrate 100, e.g., between the interlayer insulating layer 132 and the planarization layer 150. Alternatively, in the top-emission type organic light emitting display device 100, the colour filter layer may be positioned over the OLED D, e.g., over the second electrode 164 or the encapsulation layer 170.

The organic light emitting display device 100 may further include a polarization plate (not shown) for reducing an ambient light reflection. For example, the polarization plate may be a circular polarization plate. In the bottom-emission type organic light emitting display device 100, the polarization plate may be disposed under the substrate 110. In the top-emission type organic light emitting display device 100, the polarization plate may be disposed on or over the encapsulation layer 170.

In addition, in the top-emission type organic light emitting display device 100, a cover window (not shown) may be attached to the encapsulation layer 170 or the polarization plate. In this instance, the substrate 110 and the cover window may have a flexible property such that a flexible organic light emitting display device may be provided.

Moreover, the organic light emitting display device 100 may further include a touch layer or a touch panel. The touch layer or the touch panel may be disposed over the OLED D, e.g., between the OLED D and the cover window, or under the substrate 100.

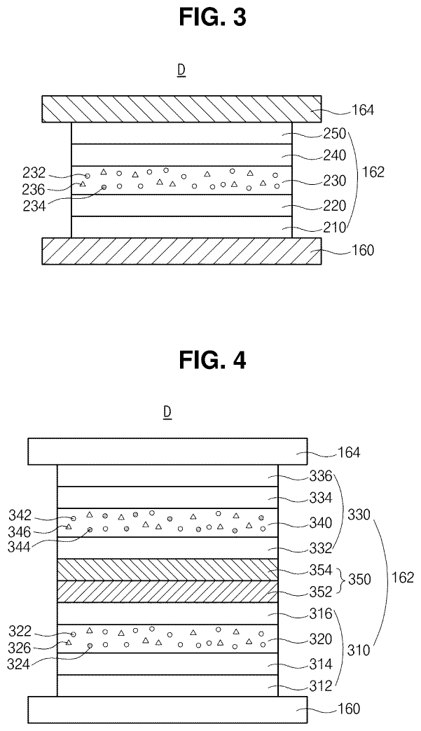

FIG. 3 illustrates a schematic cross-sectional view of an OLED according to a second embodiment of the present disclosure.

As illustrated in FIG. 3, an OLED D may include first and second electrodes 160 and 164, which may face each other, and an organic light emitting layer 162 therebetween. The organic light emitting layer 162 may include a EML 230 between the first and second electrodes 160 and 164. The EML 230 may be a green EML or a yellow-green EML.

The organic light emitting display device 100 (of FIG. 2) may include at least one of a red pixel region, a green pixel region, a blue pixel region and a yellow-green pixel region. The OLED D may be positioned in at least one of the green pixel region and the yellow-green pixel region. The OLED D in the red pixel region may include a red EML, and the OLED D in the blue pixel region may include a blue EML.

The first electrode 160 may be an anode injecting a hole, and the second electrode 164 may be a cathode injecting an electron. In addition, one of the first and second electrodes 160 and 164 may be a reflective electrode, and the other one of the first and second electrodes 160 and 164 may be a transparent (or a semi-transparent) electrode.

For example, the first electrode 160 may include a transparent conductive material layer formed of ITO or IZO. The second electrode 164 may be formed of one of Al, Mg, Ag, AlMg, and MgAg.

The EML 230 may include an organic compound of the present disclosure as a first compound 232. The first compound 232 may be represented by Formula 1.

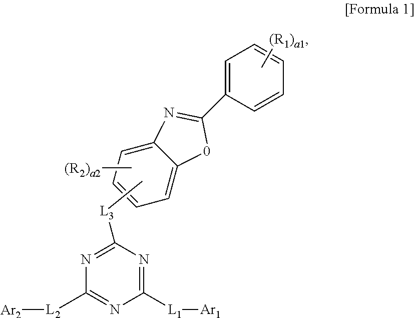

In Formula 1, a1 is an integer of 0 to 5, a2 is an integer of 0 to 3,
each R₁ is independently selected from the group consisting of a substituted or unsubstituted C1 to C10 alkyl group,
each R₂ is independently selected from the group consisting of a substituted or unsubstituted C1 to C10 alkyl group, a substituted or unsubstituted C6 to C60 aryl group and a substituted or unsubstituted C3 to C60 heteroaryl group,
when a1 is 2 or more, 2 or more R₁ groups are the same or different,
when a2 is 2 or more, 2 or more R₂ groups are the same or different,
each L₁, L₂ and L₃ is independently selected from the group consisting of a single bond (e.g., a direct bond), a substituted or unsubstituted C6 to C60 arylene group and a substituted or unsubstituted C3 to C60 heteroarylene group,
Ar₁ is selected from Formula 1a-1 to Formula 1a-5,

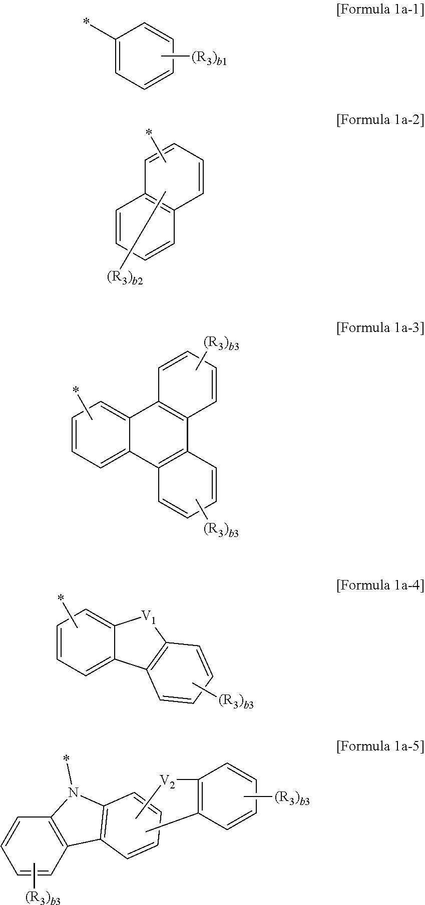

in Formula 1a-1, b1 is an integer of 0 to 5,
in Formula 1a-2, b2 is an integer of 0 to 7,
in each of Formulas 1a-3 to 1a-5, each b3 is independently an integer of 0 to 4,
when each b1, b2 and b3 is 2 or more, 2 or more R₃ groups are the same or different,
in Formula 1a-4, V₁ is selected from O, S and C(R₃)₂,
in Formula 1a-5, V₂ is selected from O and S,
in each of Formulas 1a-1 to 1a-5, each R₃ is independently selected from the group consisting of deuterium, a substituted or unsubstituted C1 to C10 alkyl group, a substituted or unsubstituted C6 to C60 aryl group and a substituted or unsubstituted C3 to C60 heteroaryl group,
Ar₂ is selected from Formula 1b-1 and Formula 1b-2,

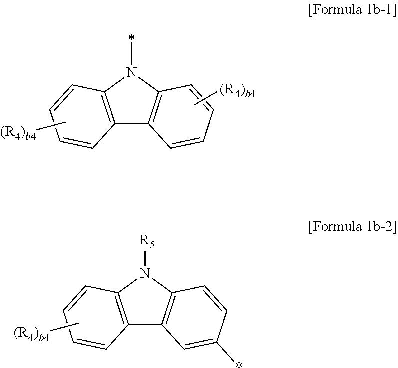

in each of Formulas 1b-1 and 1b-2, b4 is an integer of 0 to 4,
each R₄ is independently selected from the group consisting of deuterium, a substituted or unsubstituted C1 to C10 alkyl group, a substituted or unsubstituted C6 to C60 aryl group and a substituted or unsubstituted C3 to C60 heteroaryl group,
wherein when b4 is 2 or more, 2 or more R₄ groups are the same or different, and
in Formula 1b-2, R₅ is selected from the group consisting of hydrogen, deuterium, a substituted or unsubstituted C1 to C10 alkyl group and a substituted or unsubstituted C6 to C60 aryl group.

In each of Formulas 1a-1 to 1a-5, 1b-1 and 1b-2, the mark “*” denotes the bonding site.

It will be appreciated that R-groups represent substitution as valency allows. Where said groups represent no-substitution, hydrogen atoms are present as required to satisfy the valency requirements of the compound.

In the present disclosure, without specific definition, a substituent of an alkyl group, a cycloalkyl group, an alkoxy group, an aryl group, a heteroaryl group, an arylene group, and a heteroarylene group may be selected from the group consisting of deuterium (D), halogen, cyano, a hydroxyl group, a C1 to C10 alkyl group, a C1 to C10 alkoxy group, a C3 to C30 cycloalkyl group, a C1 to C10 alkylsilyl group, a C1 to C10 alkylamino group, a C6 to C30 arylsilyl group, a C6 to C30 arylamino group, a C6 to C30 aryl group and a C3 to C30 heteroaryl group. For example, the substituent may be at least one selected from the group consisting of D, F, Br, CN, hydroxyl, methyl, ethyl, propyl, butyl (e.g., tert-butyl), methoxy, ethoxy, propoxy, butoxy (e.g., tert-butoxy), cyclopropyl, cyclobutyl, cryclopentyl, cyclohexyl, trimethylsilyl, trimethylamino, triphenylsiliyl, triphenylamino, phenyl, biphenyl, naphthyl, anthracenyl, pyridyl, carbazolyl, dibenzofuranyl and dibenzothiophenyl.

In the present disclosure, without specific definition, the term “alkyl” means a substituted or unsubstituted, saturated, linear or branched hydrocarbon chain radical. For example, the C1 to C10 alkyl group may be selected from the group consisting of methyl, ethyl, propyl and butyl, e.g., tert-butyl.

In the present disclosure, without specific definition, the term “aryl” means a monovalent monocyclic or polycyclic conjugated ring structure. For example, the C6 to C60 aryl group may be selected from the group consisting of phenyl, biphenyl, terphenyl, naphthyl, anthracenyl, pentanenyl, indenyl, indenoindenyl, heptalenyl, biphenylenyl, indacenyl, phenanthrenyl, benzophenanthrenyl, dibenzophenanthrenyl, azulenyl, pyrenyl, fluoranthenyl, triphenylenyl, chrysenyl, tetraphenyl, tetrasenyl, picenyl, pentaphenyl, pentacenyl, fluorenyl, indenofluorenyl, and spiro-fluorenyl.

In the present disclosure, without specific definition, the term “arylene” means a divalent monocyclic or polycyclic conjugated ring structure. For example, the C6 to C60 arylene group may be selected from the group consisting of phenylene, biphenylene, terphenylene, naphthylene, anthracenylene, pentanenylene, indenylene, indenoindenylene, heptalenylene, biphenylenylene, indacenylene, phenanthrenylene, benzophenanthrenylene, dibenzophenanthrenylene, azulenylene, pyrenylene, fluoranthenylene, triphenylenylene, chrysenylene, tetraphenylene, tetrasenylene, picenylene, pentaphenylene, pentacenylene, fluorenylene, indenofluorenylene, and spiro-fluorenylene.

In the present disclosure, without specific definition, the term “heteroaryl” refers to a 5- to 7-membered aromatic ring which includes 1, 2, 3 or 4 hetero atoms such as nitrogen, oxygen or sulfur and such rings fused to an aryl, cycloalkyl, heteroaryl or heterocycloalkyl ring. For example, the C3 to C60 heteroaryl group may be selected from the group consisting of pyrrolyl, pyridinyl, pyrimidinyl, pyrazinyl, pyridazinyl, triazinyl, tetrazinyl, imidazolyl, pyrazolyl, indolyl, isoindolyl, indazolyl, indolizinyl, pyrrolizinyl, carbazolyl, benzocarbazolyl, dibenzocarbazolyl, indolocarbazolyl, indenocarbazolyl, benzofurocarbazolyl, benzothienocarbazolyl, quinolinyl, isoquinolinyl, phthalazinyl, quinoxalinyl, cinnolinyl, quinazolinyl, quinozolinyl, purinyl, benzoquinolinyl, benzoisoquinolinyl, benzoquinazolinyl, benzoquinoxalinyl, acridinyl, phenanthrolinyl, perimidinyl, phenanthridinyl, pteridinyl, cinnolinyl, naphtharidinyl, furanyl, oxazinyl, oxazolyl, oxadiazolyl, triazolyl, dioxynyl, benzofuranyl, dibenzofuranyl, thiopyranyl, xanthenyl, chromanyl, isochromanyl, thioazinyl, thiophenyl, benzothiophenyl, dibenzothiophenyl, difuropyrazinyl, benzofurodibenzofuranyl, benzothienobenzothiophenyl, benzothienodibenzothiophenyl, benzothienobenzofuranyl, and benzothienodibenzofuranyl.

In the present disclosure, without specific definition, the term “heteroarylene” refers to the divalent counterpart of a heteroaryl group, as defined above. For example, the C3 to C60 heteroarylene group may be selected from the group consisting of pyrrolylene, pyridinylene, pyrimidinylene, pyrazinylene, pyridazinylene, triazinylene, tetrazinylene, imidazolylene, pyrazolylene, indolylene, isoindolylene, indazolylene, indolizinylene, pyrrolizinylene, carbazolylene, benzocarbazolylene, dibenzocarbazolylene, indolocarbazolylene, indenocarbazolylene, benzofurocarbazolylene, benzothienocarbazolylene, quinolinylene, isoquinolinylene, phthalazinylene, quinoxalinylene, cinnolinylene, quinazolinylene, quinozolinylene, purinylene, benzoquinolinylene, benzoisoquinolinylene, benzoquinazolinylene, benzoquinoxalinylene, acridinylene, phenanthrolinylene, perimidinylene, phenanthridinylene, pteridinylene, cinnolinylene, naphtharidinylene, furanylene, oxazinylene, oxazolylene, oxadiazolylene, triazolylene, dioxynylene, benzofuranylene, dibenzofuranylene, thiopyranylene, xanthenylene, chromanylene, isochromanylene, thioazinylene, thiophenylene, benzothiophenylene, dibenzothiophenylene, difuropyrazinylene, benzofurodibenzofuranylene, benzothienobenzothiophenylene, benzothienodibenzothiophenylene, benzothienobenzofuranylene, and benzothienodibenzofuranylene.

In an aspect of the present disclosure, R₁ may be a C6 to C60 aryl group, e.g., phenyl, unsubstituted or substituted with a C1 to C10 alkyl group, e.g., methyl.

In an aspect of the present disclosure, a1 may be 0.

In an aspect of the present disclosure, each L₁, L₂ and L₃ may independently be a C6 to C60 arylene group, e.g., phenylene.

In an aspect of the present disclosure, each b1 to b3 may be independently an integer of 0 to 2.

In an aspect of the present disclosure, each R₃ may be a substituted or unsubstituted C1 to C10 alkyl group, e.g., methyl or tert-butyl, or a substituted or unsubstituted C6 to C60 aryl group, e.g., phenyl.

In an aspect of the present disclosure, b4 may be 0 or 1.

In an aspect of the present disclosure, each R₄ and R₅ may independently be a substituted or unsubstituted C6 to C60 aryl group, e.g., phenyl.

As shown in Formula 1, the organic compound of the present disclosure has a structure, where a first moiety of benzoxazole, a second moiety of carbazole, and a third moiety, which is selected from Formulas 1a-1 to 1a-5, are connected (joined, bonded, or coupled) to a triazine core directly or through a linker. Namely, the organic compound of the present disclosure, a triazine core, a first moiety of benzoxazole, a second moiety of carbazole, and a third moiety being different from the first and second moieties. As a result, the OLED D and the organic light emitting display device 100 using the organic compound have an advantage in that at least one of the driving voltage, the emitting efficiency and the lifespan is improved.

In Formula 1, a linking position of L₃ to the benzoxazole moiety is specified. For example, the organic compound of the present disclosure may be represented by one of Formulas 1-1 to 1-4.

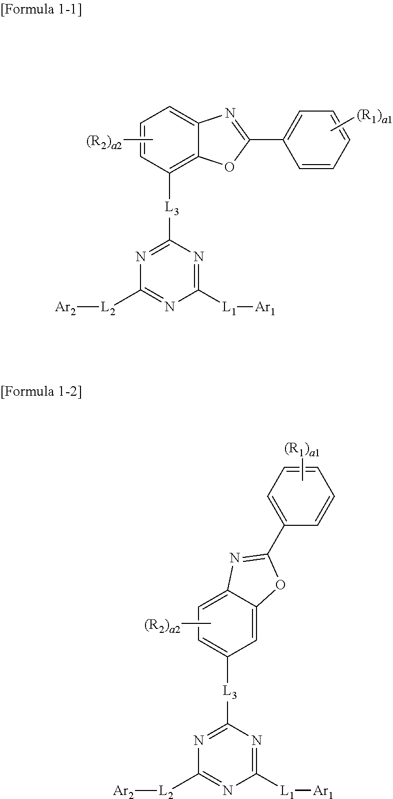

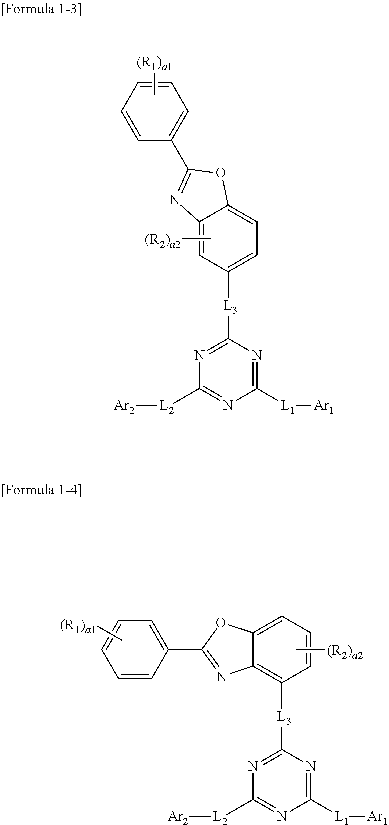

In each of Formulas 1-1 to 1-4, the definition of a1, a2, R₁, R₂, L₁, L₂, L₃, Ar₁, and Ar₂ may be the same as in Formula 1.

In an aspect of the present disclosure, Ar₂ may be represented by Formula 1b-1, L₂ may be phenylene, and a linking position of Ar₂ may be specified. For example, the organic compound may be represented by Formula 1-5.

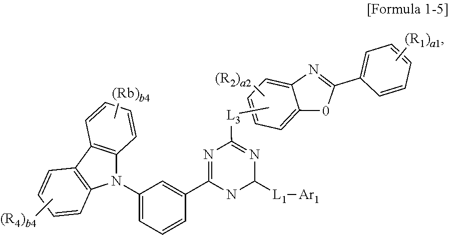

In Formula 1-5, the definition of a1, a2, R₁, R₂, L₁, L₃ and Ar₁ is the same as in Formula 1, and the definition of R₄ and b4 is the same as in Formula 1b-1.

In an aspect of the present disclosure, Ar₂ may be represented by Formula 1b-2, and L₂ may be a single bond. For example, the organic compound may be represented by Formula 1-6.

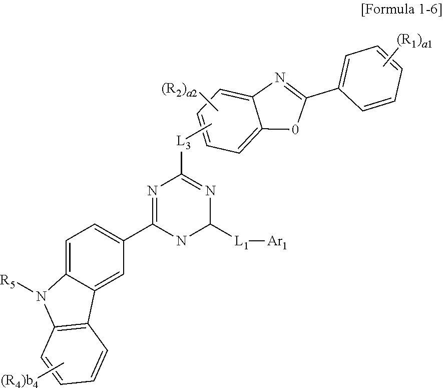

In Formula 1-6, the definition of a1, a2, R₁, R₂, L₁, L₃ and Ar₁ is the same as in Formula 1, and the definition of R₄, R₅ and b4 is the same as in Formula 1b-1.

The first compound 232 being the organic compound of the present disclosure may be one of the compounds in Formula 2.

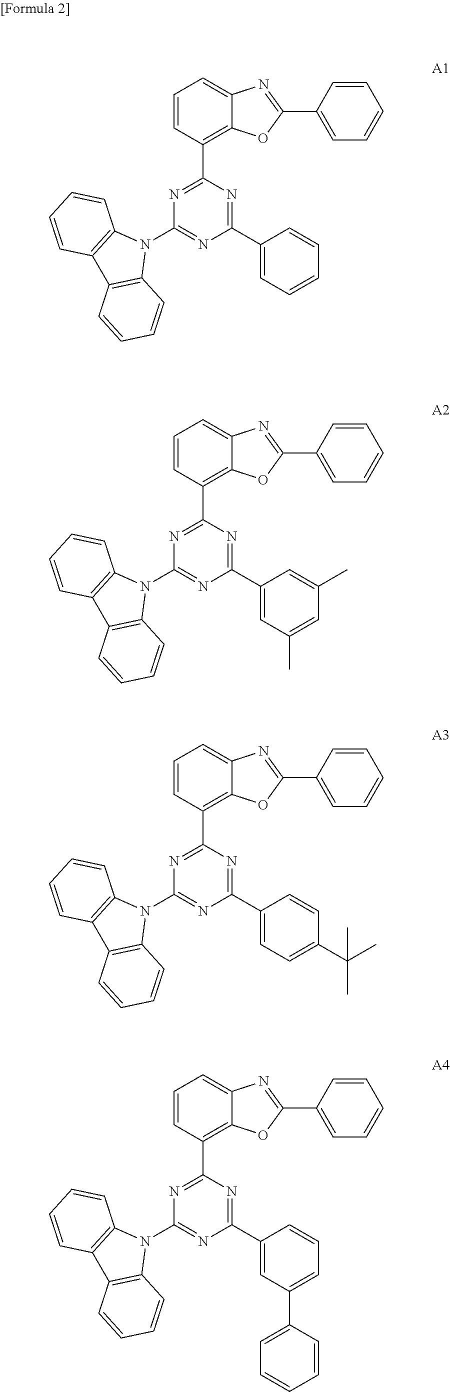 [SMILES: c1ccc(-c2nc(-c3cccc4nc(-c5ccccc5)oc34)nc(-n3c4ccccc4c4ccccc43)n2)cc1 ; Cc1cc(C)cc(-c2nc(-c3cccc4nc(-c5ccccc5)oc34)nc(-n3c4ccccc4c4ccccc43)n2)c1 ; CC(C)(C)c1ccc(-c2nc(-c3cccc4nc(-c5ccccc5)oc34)nc(-n3c4ccccc4c4ccccc43)n2)cc1 ; c1ccc(-c2cccc(-c3nc(-c4cccc5nc(-c6ccccc6)oc45)nc(-n4c5ccccc5c5ccccc54)n3)c2)cc1]

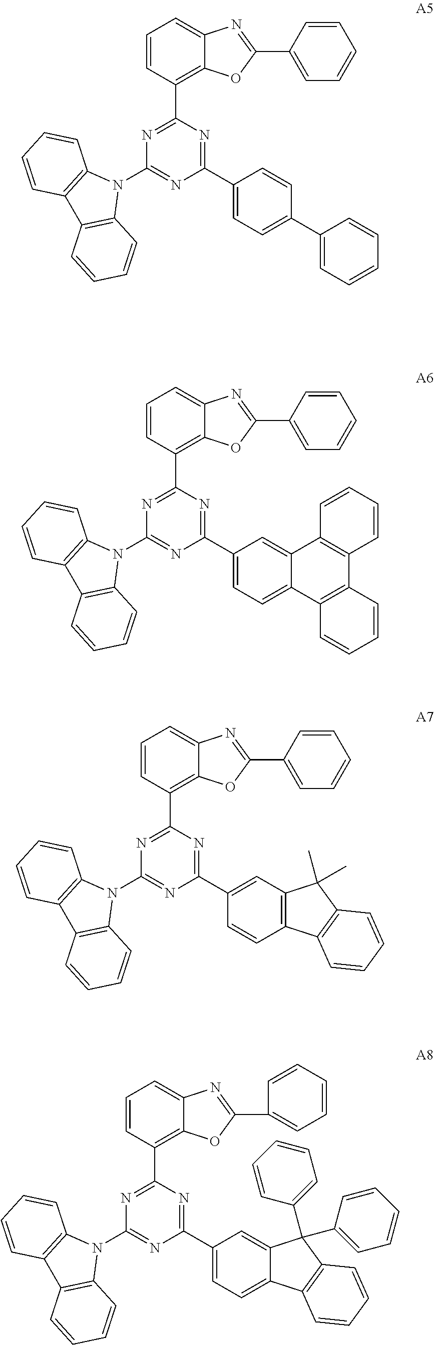 [SMILES: c1ccc(-c2ccc(-c3nc(-c4cccc5nc(-c6ccccc6)oc45)nc(-n4c5ccccc5c5ccccc54)n3)cc2)cc1 ; c1ccc(-c2nc3cccc(-c4nc(-c5ccc6c7ccccc7c7ccccc7c6c5)nc(-n5c6ccccc6c6ccccc65)n4)c3o2)cc1 ; CC1(C)c2ccccc2-c2ccc(-c3nc(-c4cccc5nc(-c6ccccc6)oc45)nc(-n4c5ccccc5c5ccccc54)n3)cc21 ; c1ccc(-c2nc3cccc(-c4nc(-c5ccc6c(c5)C(c5ccccc5)(c5ccccc5)c5ccccc5-6)nc(-n5c6ccccc6c6ccccc65)n4)c3o2)cc1]

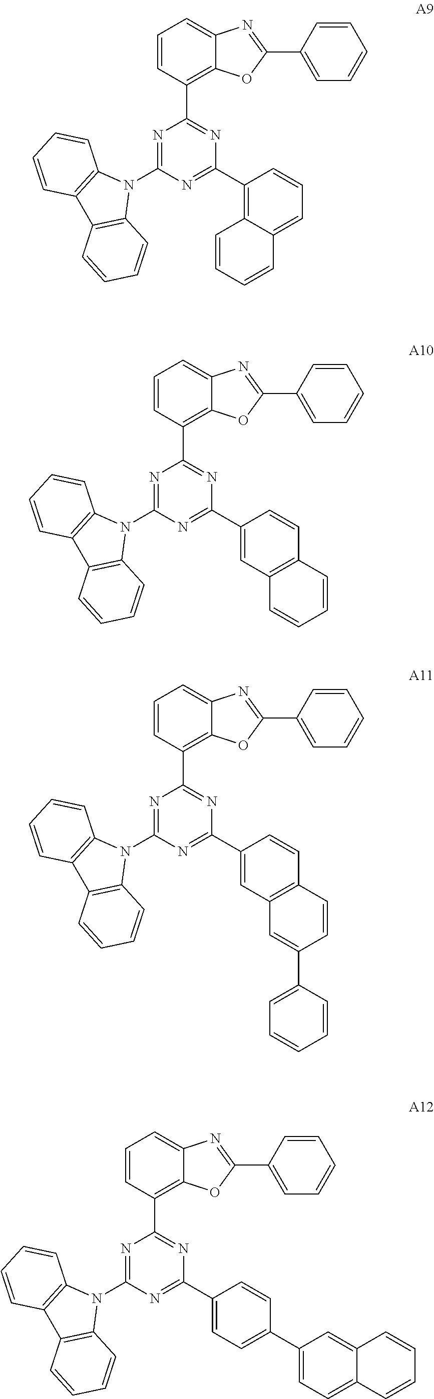 [SMILES: c1ccc(-c2nc3cccc(-c4nc(-c5cccc6ccccc56)nc(-n5c6ccccc6c6ccccc65)n4)c3o2)cc1 ; c1ccc(-c2nc3cccc(-c4nc(-c5ccc6ccccc6c5)nc(-n5c6ccccc6c6ccccc65)n4)c3o2)cc1 ; c1ccc(-c2ccc3ccc(-c4nc(-c5cccc6nc(-c7ccccc7)oc56)nc(-n5c6ccccc6c6ccccc65)n4)cc3c2)cc1 ; c1ccc(-c2nc3cccc(-c4nc(-c5ccc(-c6ccc7ccccc7c6)cc5)nc(-n5c6ccccc6c6ccccc65)n4)c3o2)cc1]

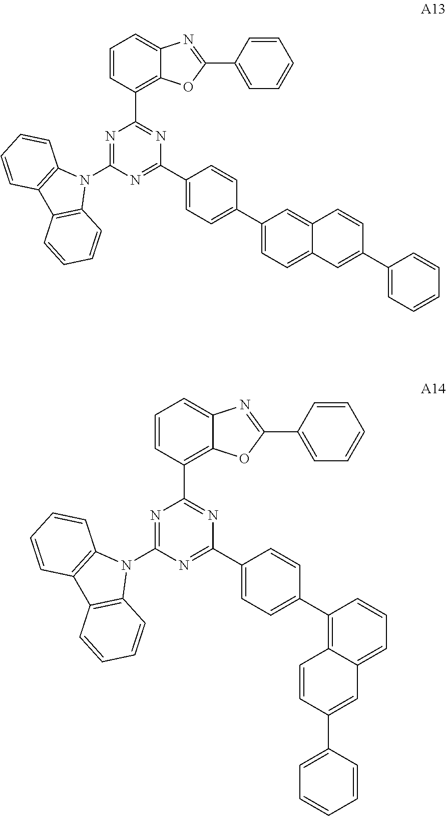 [SMILES: c1ccc(-c2ccc3cc(-c4ccc(-c5nc(-c6cccc7nc(-c8ccccc8)oc67)nc(-n6c7ccccc7c7ccccc76)n5)cc4)ccc3c2)cc1 ; c1ccc(-c2ccc3c(-c4ccc(-c5nc(-c6cccc7nc(-c8ccccc8)oc67)nc(-n6c7ccccc7c7ccccc76)n5)cc4)cccc3c2)cc1]

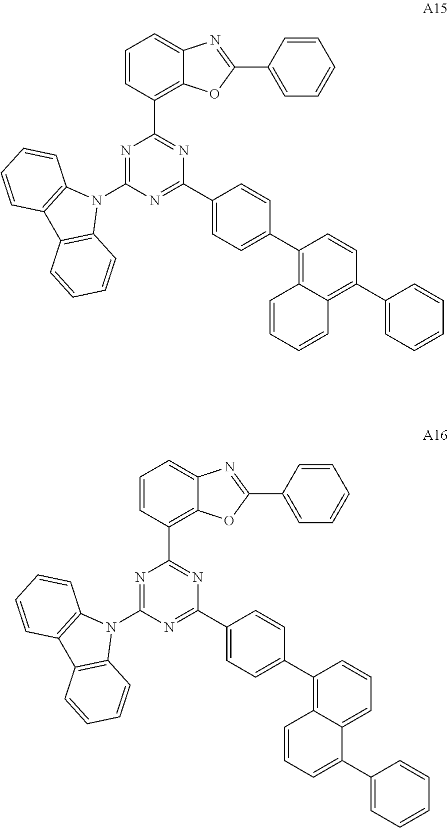 [SMILES: c1ccc(-c2nc3cccc(-c4nc(-c5ccc(-c6ccc(-c7ccccc7)c7ccccc67)cc5)nc(-n5c6ccccc6c6ccccc65)n4)c3o2)cc1 ; c1ccc(-c2nc3cccc(-c4nc(-c5ccc(-c6cccc7c(-c8ccccc8)cccc67)cc5)nc(-n5c6ccccc6c6ccccc65)n4)c3o2)cc1]

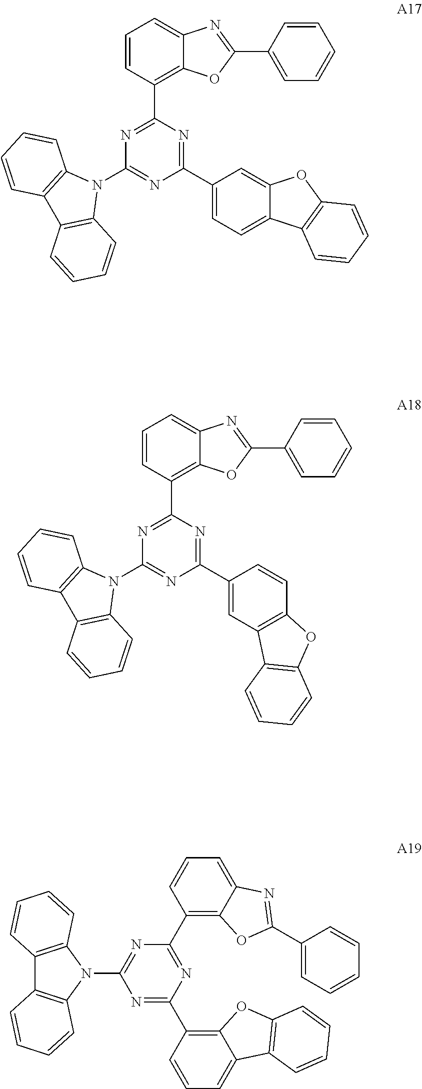 [SMILES: c1ccc(-c2nc3cccc(-c4nc(-c5ccc6c(c5)oc5ccccc56)nc(-n5c6ccccc6c6ccccc65)n4)c3o2)cc1 ; c1ccc(-c2nc3cccc(-c4nc(-c5ccc6oc7ccccc7c6c5)nc(-n5c6ccccc6c6ccccc65)n4)c3o2)cc1 ; c1ccc(-c2nc3cccc(-c4nc(-c5cccc6c5oc5ccccc56)nc(-n5c6ccccc6c6ccccc65)n4)c3o2)cc1]

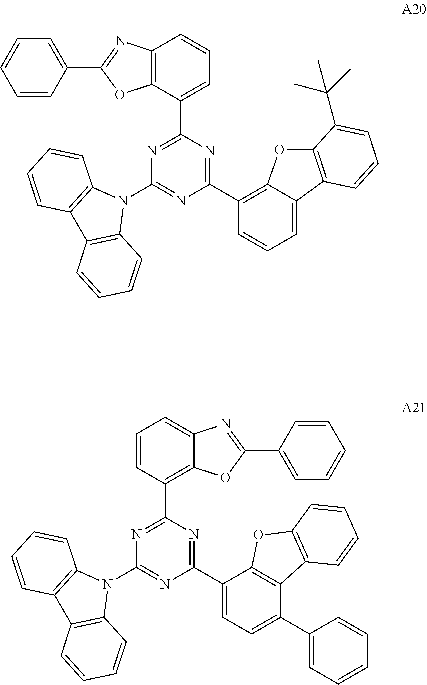 [SMILES: CC(C)(C)c1cccc2c1oc1c(-c3nc(-c4cccc5nc(-c6ccccc6)oc45)nc(-n4c5ccccc5c5ccccc54)n3)cccc12 ; c1ccc(-c2nc3cccc(-c4nc(-c5ccc(-c6ccccc6)c6c5oc5ccccc56)nc(-n5c6ccccc6c6ccccc65)n4)c3o2)cc1]

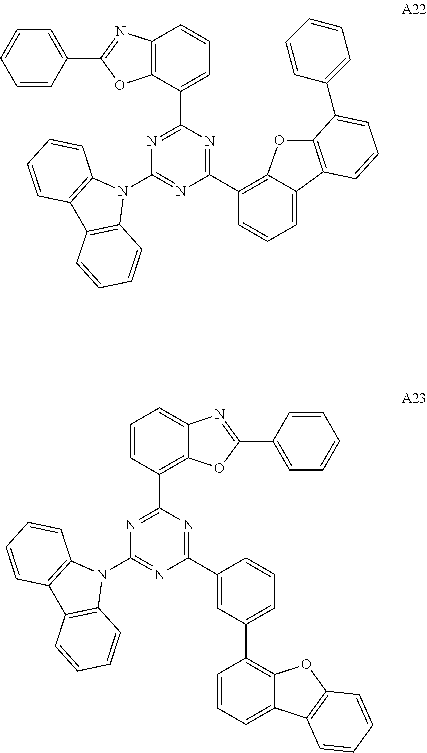 [SMILES: c1ccc(-c2nc3cccc(-c4nc(-c5cccc6c5oc5c(-c7ccccc7)cccc56)nc(-n5c6ccccc6c6ccccc65)n4)c3o2)cc1 ; c1ccc(-c2nc3cccc(-c4nc(-c5cccc(-c6cccc7c6oc6ccccc67)c5)nc(-n5c6ccccc6c6ccccc65)n4)c3o2)cc1]

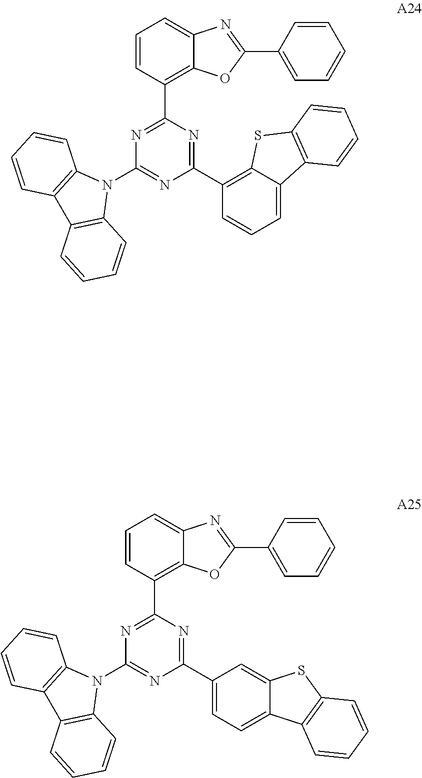 [SMILES: c1ccc(-c2nc3cccc(-c4nc(-c5cccc6c5sc5ccccc56)nc(-n5c6ccccc6c6ccccc65)n4)c3o2)cc1 ; c1ccc(-c2nc3cccc(-c4nc(-c5ccc6c(c5)sc5ccccc56)nc(-n5c6ccccc6c6ccccc65)n4)c3o2)cc1]

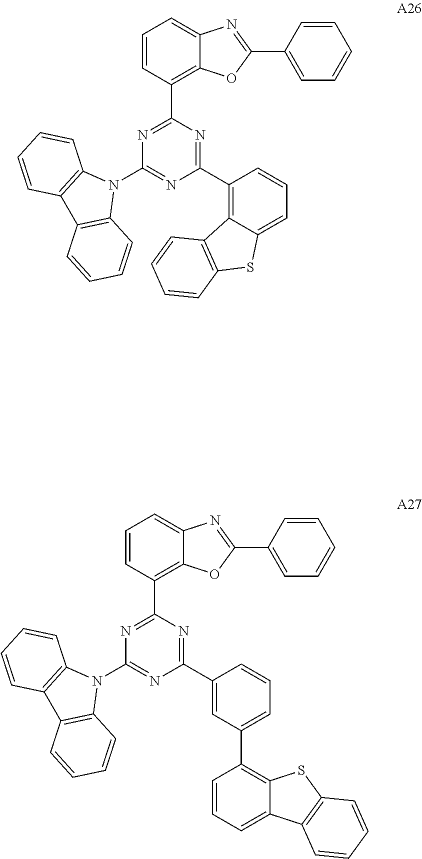 [SMILES: c1ccc(-c2nc3cccc(-c4nc(-c5cccc6sc7ccccc7c56)nc(-n5c6ccccc6c6ccccc65)n4)c3o2)cc1 ; c1ccc(-c2nc3cccc(-c4nc(-c5cccc(-c6cccc7c6sc6ccccc67)c5)nc(-n5c6ccccc6c6ccccc65)n4)c3o2)cc1]

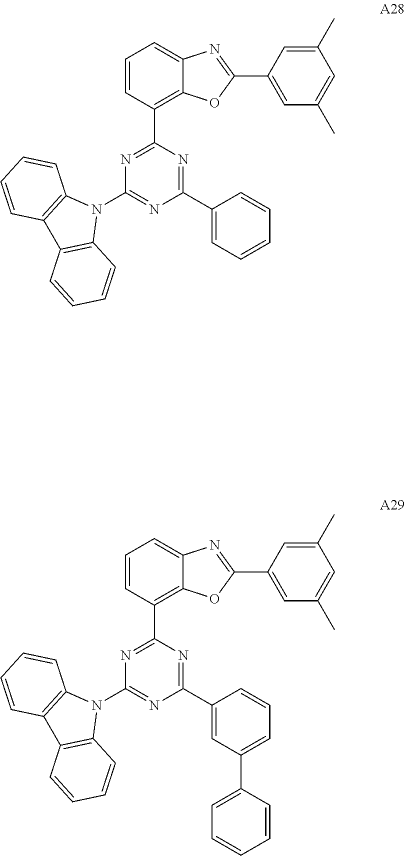 [SMILES: Cc1cc(C)cc(-c2nc3cccc(-c4nc(-c5ccccc5)nc(-n5c6ccccc6c6ccccc65)n4)c3o2)c1 ; Cc1cc(C)cc(-c2nc3cccc(-c4nc(-c5cccc(-c6ccccc6)c5)nc(-n5c6ccccc6c6ccccc65)n4)c3o2)c1]

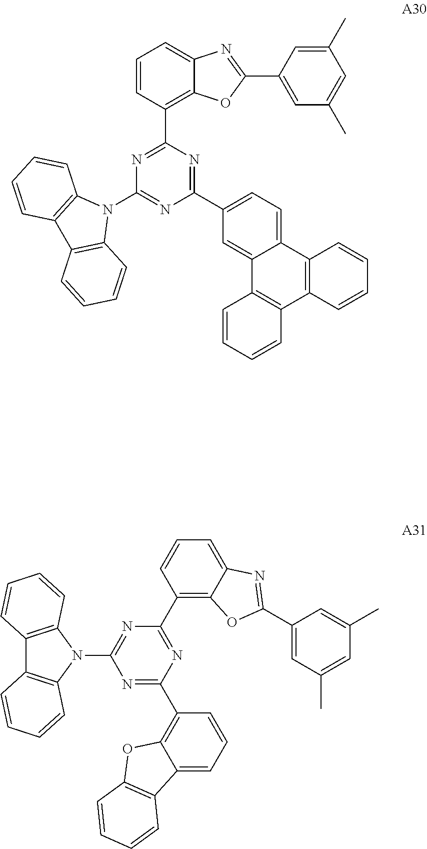 [SMILES: Cc1cc(C)cc(-c2nc3cccc(-c4nc(-c5ccc6c7ccccc7c7ccccc7c6c5)nc(-n5c6ccccc6c6ccccc65)n4)c3o2)c1 ; Cc1cc(C)cc(-c2nc3cccc(-c4nc(-c5cccc6c5oc5ccccc56)nc(-n5c6ccccc6c6ccccc65)n4)c3o2)c1]

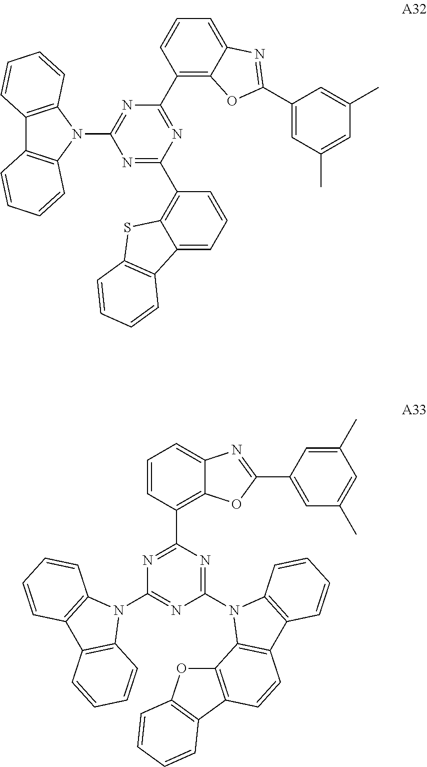 [SMILES: Cc1cc(C)cc(-c2nc3cccc(-c4nc(-c5cccc6c5sc5ccccc56)nc(-n5c6ccccc6c6ccccc65)n4)c3o2)c1 ; Cc1cc(C)cc(-c2nc3cccc(-c4nc(-n5c6ccccc6c6ccccc65)nc(-n5c6ccccc6c6ccc7c8ccccc8oc7c65)n4)c3o2)c1]

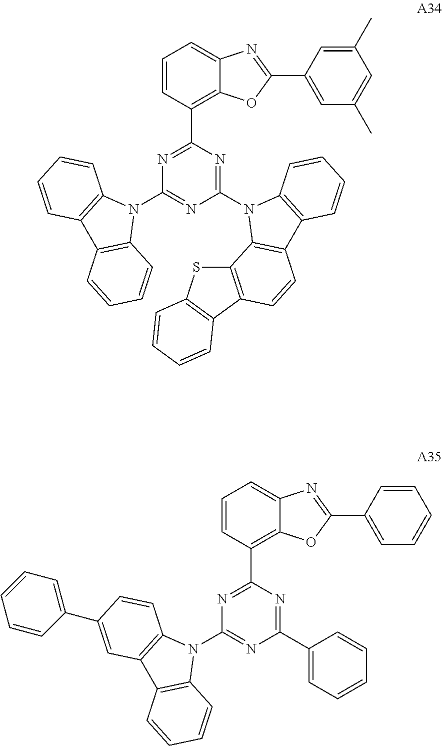 [SMILES: Cc1cc(C)cc(-c2nc3cccc(-c4nc(-n5c6ccccc6c6ccccc65)nc(-n5c6ccccc6c6ccc7c8ccccc8sc7c65)n4)c3o2)c1 ; c1ccc(-c2ccc3c(c2)c2ccccc2n3-c2nc(-c3ccccc3)nc(-c3cccc4nc(-c5ccccc5)oc34)n2)cc1]

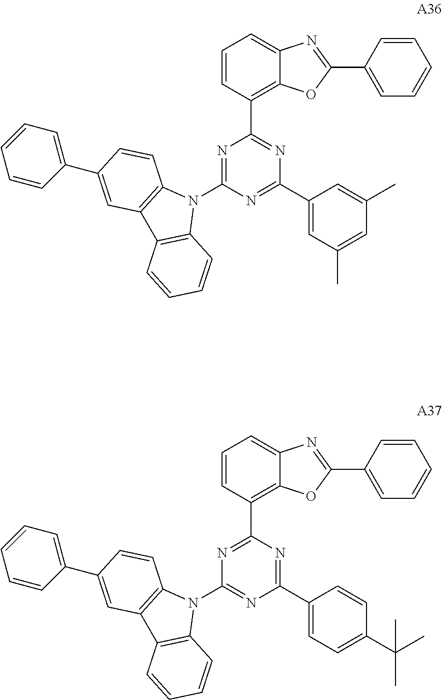 [SMILES: Cc1cc(C)cc(-c2nc(-c3cccc4nc(-c5ccccc5)oc34)nc(-n3c4ccccc4c4cc(-c5ccccc5)ccc43)n2)c1 ; CC(C)(C)c1ccc(-c2nc(-c3cccc4nc(-c5ccccc5)oc34)nc(-n3c4ccccc4c4cc(-c5ccccc5)ccc43)n2)cc1]

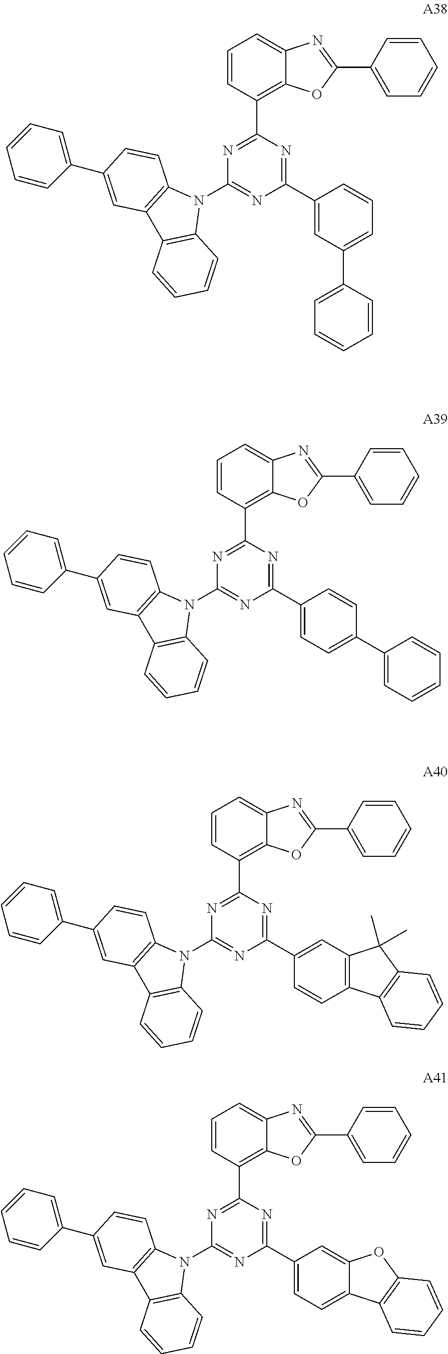 [SMILES: c1ccc(-c2cccc(-c3nc(-c4cccc5nc(-c6ccccc6)oc45)nc(-n4c5ccccc5c5cc(-c6ccccc6)ccc54)n3)c2)cc1 ; c1ccc(-c2ccc(-c3nc(-c4cccc5nc(-c6ccccc6)oc45)nc(-n4c5ccccc5c5cc(-c6ccccc6)ccc54)n3)cc2)cc1 ; CC1(C)c2ccccc2-c2ccc(-c3nc(-c4cccc5nc(-c6ccccc6)oc45)nc(-n4c5ccccc5c5cc(-c6ccccc6)ccc54)n3)cc21 ; c1ccc(-c2ccc3c(c2)c2ccccc2n3-c2nc(-c3ccc4c(c3)oc3ccccc34)nc(-c3cccc4nc(-c5ccccc5)oc34)n2)cc1]

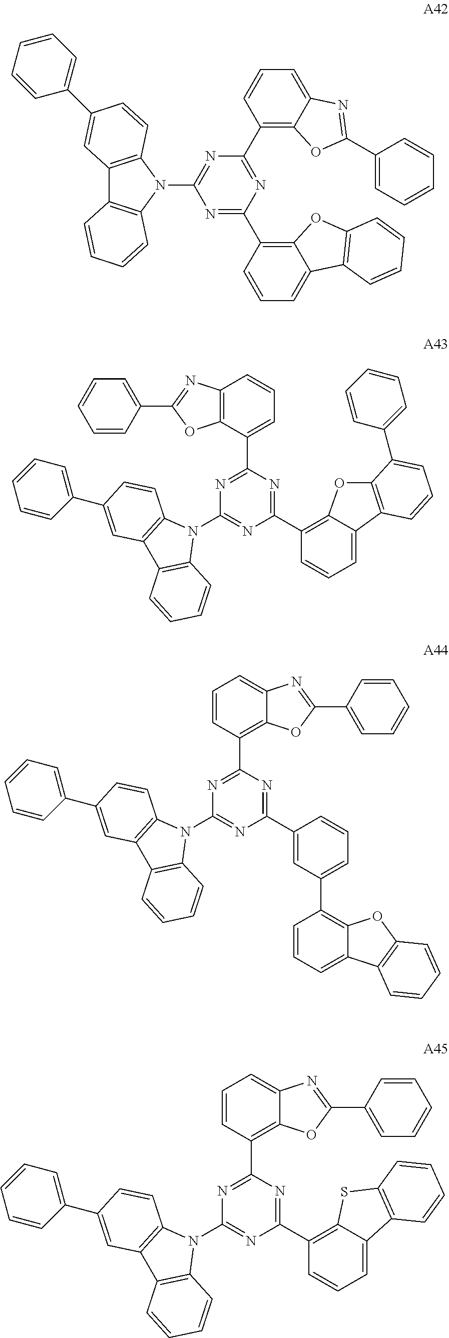 [SMILES: c1ccc(-c2ccc3c(c2)c2ccccc2n3-c2nc(-c3cccc4nc(-c5ccccc5)oc34)nc(-c3cccc4c3oc3ccccc34)n2)cc1 ; c1ccc(-c2ccc3c(c2)c2ccccc2n3-c2nc(-c3cccc4nc(-c5ccccc5)oc34)nc(-c3cccc4c3oc3c(-c5ccccc5)cccc34)n2)cc1 ; c1ccc(-c2ccc3c(c2)c2ccccc2n3-c2nc(-c3cccc(-c4cccc5c4oc4ccccc45)c3)nc(-c3cccc4nc(-c5ccccc5)oc34)n2)cc1 ; c1ccc(-c2ccc3c(c2)c2ccccc2n3-c2nc(-c3cccc4nc(-c5ccccc5)oc34)nc(-c3cccc4c3sc3ccccc34)n2)cc1]

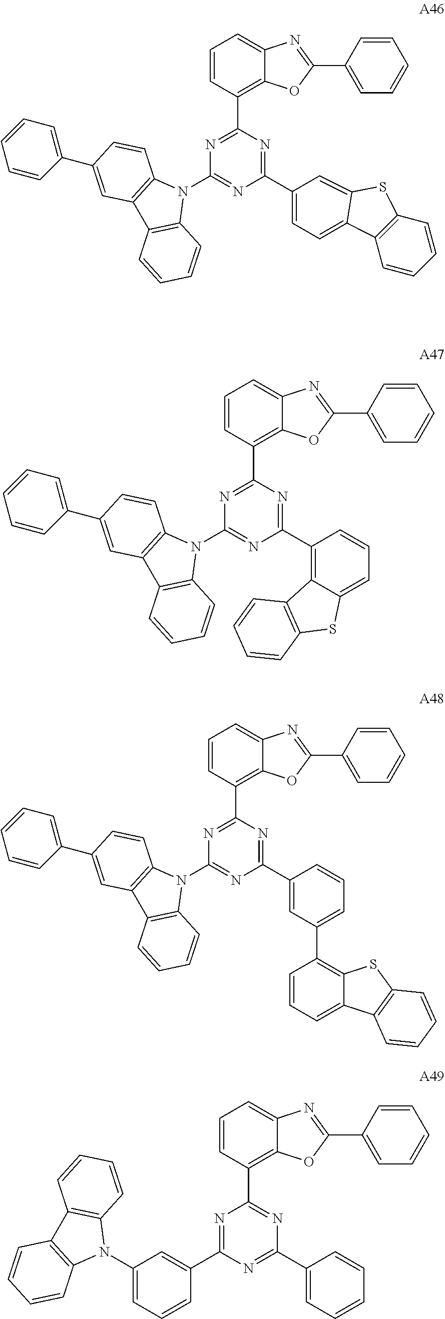 [SMILES: c1ccc(-c2ccc3c(c2)c2ccccc2n3-c2nc(-c3ccc4c(c3)sc3ccccc34)nc(-c3cccc4nc(-c5ccccc5)oc34)n2)cc1 ; c1ccc(-c2ccc3c(c2)c2ccccc2n3-c2nc(-c3cccc4nc(-c5ccccc5)oc34)nc(-c3cccc4sc5ccccc5c34)n2)cc1 ; c1ccc(-c2ccc3c(c2)c2ccccc2n3-c2nc(-c3cccc(-c4cccc5c4sc4ccccc45)c3)nc(-c3cccc4nc(-c5ccccc5)oc34)n2)cc1 ; c1ccc(-c2nc(-c3cccc(-n4c5ccccc5c5ccccc54)c3)nc(-c3cccc4nc(-c5ccccc5)oc34)n2)cc1]

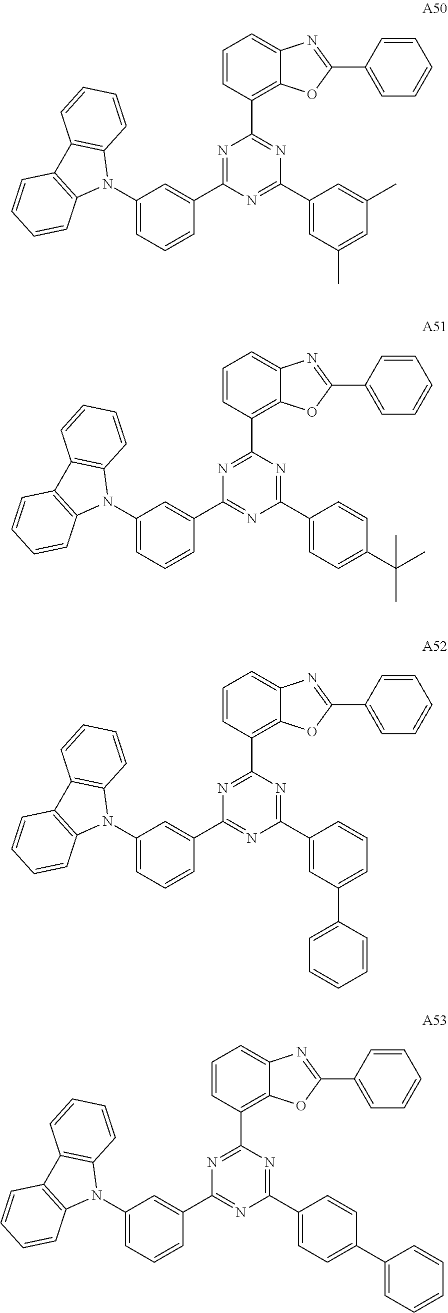 [SMILES: Cc1cc(C)cc(-c2nc(-c3cccc(-n4c5ccccc5c5ccccc54)c3)nc(-c3cccc4nc(-c5ccccc5)oc34)n2)c1 ; CC(C)(C)c1ccc(-c2nc(-c3cccc(-n4c5ccccc5c5ccccc54)c3)nc(-c3cccc4nc(-c5ccccc5)oc34)n2)cc1 ; c1ccc(-c2cccc(-c3nc(-c4cccc(-n5c6ccccc6c6ccccc65)c4)nc(-c4cccc5nc(-c6ccccc6)oc45)n3)c2)cc1 ; c1ccc(-c2ccc(-c3nc(-c4cccc(-n5c6ccccc6c6ccccc65)c4)nc(-c4cccc5nc(-c6ccccc6)oc45)n3)cc2)cc1]

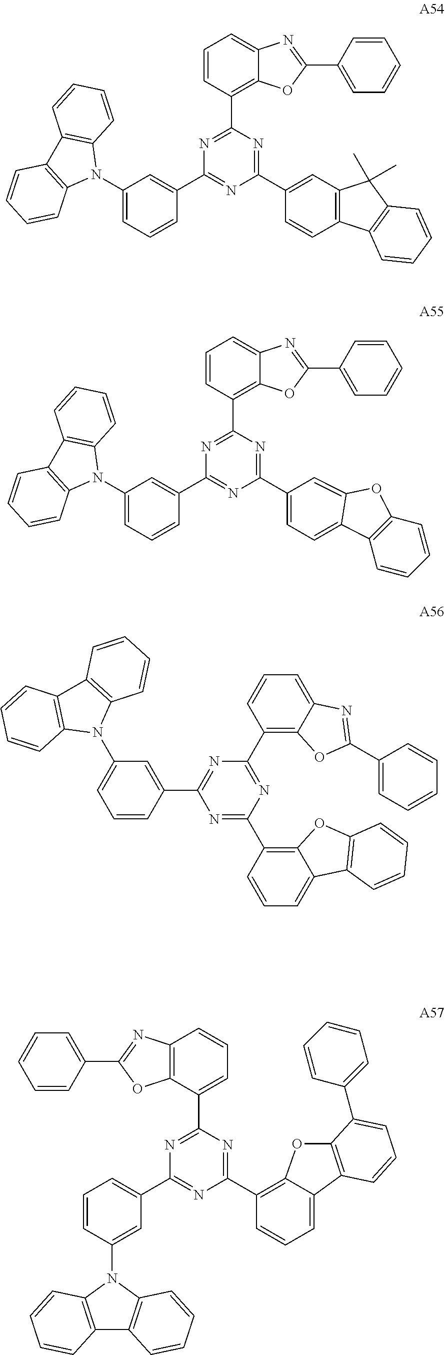 [SMILES: CC1(C)c2ccccc2-c2ccc(-c3nc(-c4cccc(-n5c6ccccc6c6ccccc65)c4)nc(-c4cccc5nc(-c6ccccc6)oc45)n3)cc21 ; c1ccc(-c2nc3cccc(-c4nc(-c5cccc(-n6c7ccccc7c7ccccc76)c5)nc(-c5ccc6c(c5)oc5ccccc56)n4)c3o2)cc1 ; c1ccc(-c2nc3cccc(-c4nc(-c5cccc(-n6c7ccccc7c7ccccc76)c5)nc(-c5cccc6c5oc5ccccc56)n4)c3o2)cc1 ; c1ccc(-c2nc3cccc(-c4nc(-c5cccc(-n6c7ccccc7c7ccccc76)c5)nc(-c5cccc6c5oc5c(-c7ccccc7)cccc56)n4)c3o2)cc1]

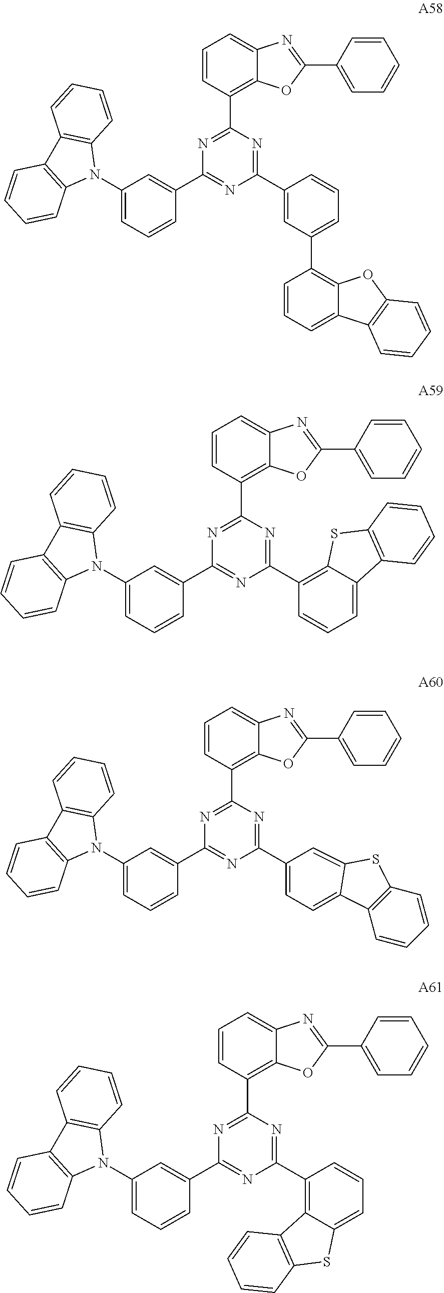 [SMILES: c1ccc(-c2nc3cccc(-c4nc(-c5cccc(-c6cccc7c6oc6ccccc67)c5)nc(-c5cccc(-n6c7ccccc7c7ccccc76)c5)n4)c3o2)cc1 ; c1ccc(-c2nc3cccc(-c4nc(-c5cccc(-n6c7ccccc7c7ccccc76)c5)nc(-c5cccc6c5sc5ccccc56)n4)c3o2)cc1 ; c1ccc(-c2nc3cccc(-c4nc(-c5cccc(-n6c7ccccc7c7ccccc76)c5)nc(-c5ccc6c(c5)sc5ccccc56)n4)c3o2)cc1 ; c1ccc(-c2nc3cccc(-c4nc(-c5cccc(-n6c7ccccc7c7ccccc76)c5)nc(-c5cccc6sc7ccccc7c56)n4)c3o2)cc1]

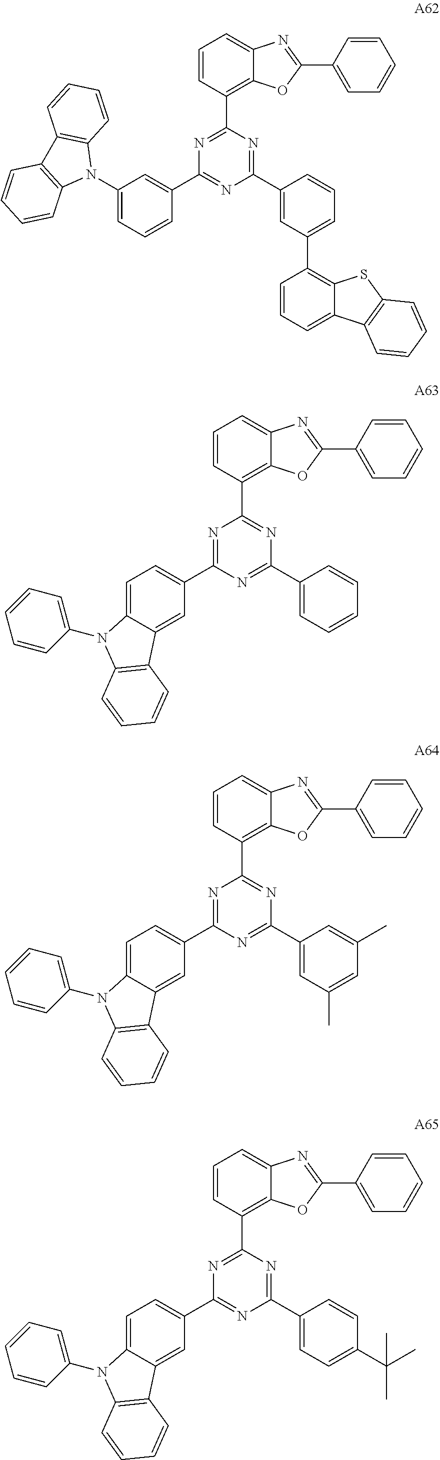 [SMILES: c1ccc(-c2nc3cccc(-c4nc(-c5cccc(-c6cccc7c6sc6ccccc67)c5)nc(-c5cccc(-n6c7ccccc7c7ccccc76)c5)n4)c3o2)cc1 ; c1ccc(-c2nc(-c3ccc4c(c3)c3ccccc3n4-c3ccccc3)nc(-c3cccc4nc(-c5ccccc5)oc34)n2)cc1 ; Cc1cc(C)cc(-c2nc(-c3ccc4c(c3)c3ccccc3n4-c3ccccc3)nc(-c3cccc4nc(-c5ccccc5)oc34)n2)c1 ; CC(C)(C)c1ccc(-c2nc(-c3ccc4c(c3)c3ccccc3n4-c3ccccc3)nc(-c3cccc4nc(-c5ccccc5)oc34)n2)cc1]

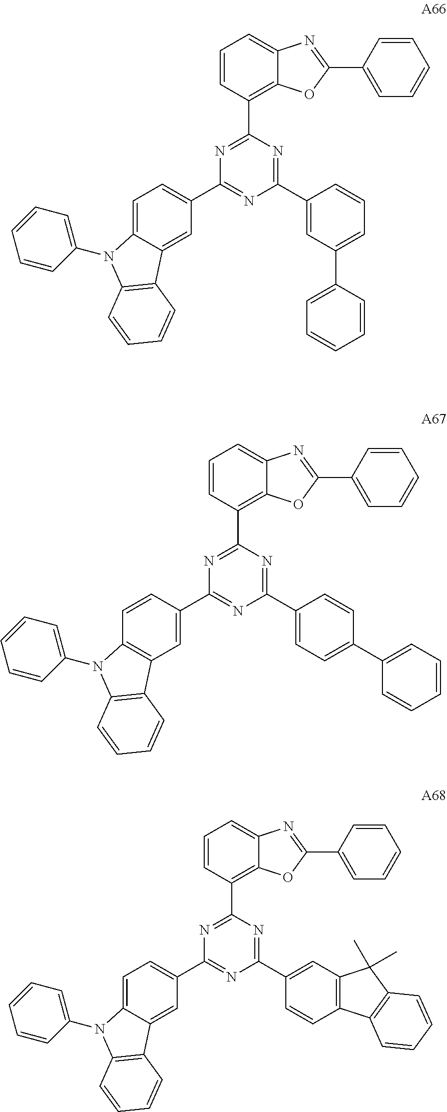 [SMILES: c1ccc(-c2cccc(-c3nc(-c4ccc5c(c4)c4ccccc4n5-c4ccccc4)nc(-c4cccc5nc(-c6ccccc6)oc45)n3)c2)cc1 ; c1ccc(-c2ccc(-c3nc(-c4ccc5c(c4)c4ccccc4n5-c4ccccc4)nc(-c4cccc5nc(-c6ccccc6)oc45)n3)cc2)cc1 ; CC1(C)c2ccccc2-c2ccc(-c3nc(-c4ccc5c(c4)c4ccccc4n5-c4ccccc4)nc(-c4cccc5nc(-c6ccccc6)oc45)n3)cc21]

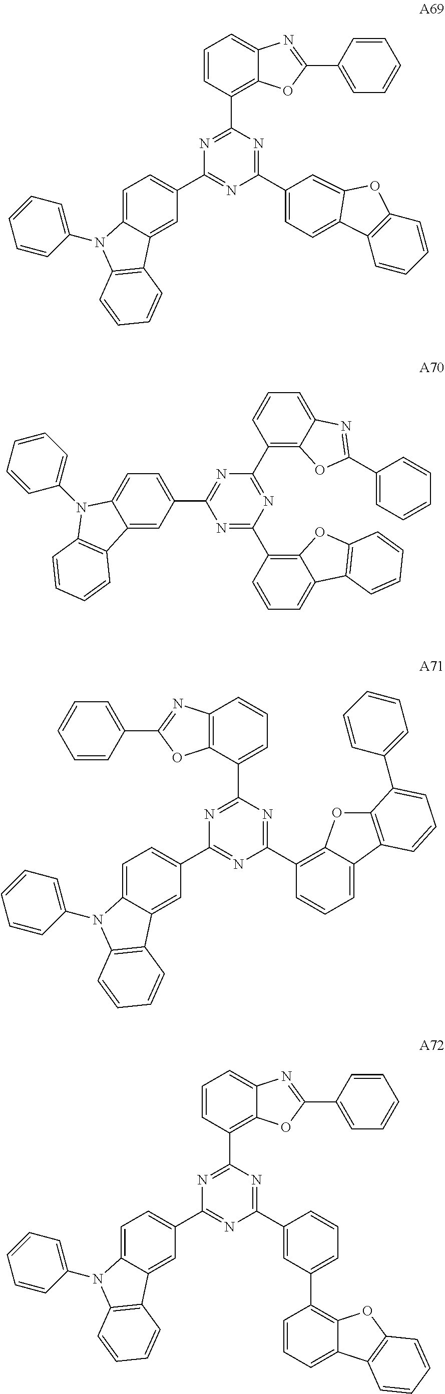 [SMILES: c1ccc(-c2nc3cccc(-c4nc(-c5ccc6c(c5)oc5ccccc56)nc(-c5ccc6c(c5)c5ccccc5n6-c5ccccc5)n4)c3o2)cc1 ; c1ccc(-c2nc3cccc(-c4nc(-c5ccc6c(c5)c5ccccc5n6-c5ccccc5)nc(-c5cccc6c5oc5ccccc56)n4)c3o2)cc1 ; c1ccc(-c2nc3cccc(-c4nc(-c5ccc6c(c5)c5ccccc5n6-c5ccccc5)nc(-c5cccc6c5oc5c(-c7ccccc7)cccc56)n4)c3o2)cc1 ; c1ccc(-c2nc3cccc(-c4nc(-c5cccc(-c6cccc7c6oc6ccccc67)c5)nc(-c5ccc6c(c5)c5ccccc5n6-c5ccccc5)n4)c3o2)cc1]

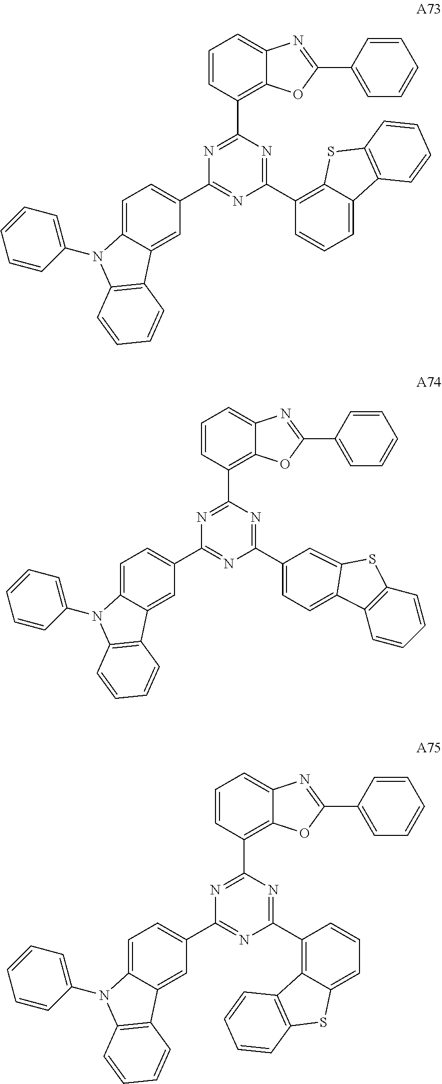 [SMILES: c1ccc(-c2nc3cccc(-c4nc(-c5ccc6c(c5)c5ccccc5n6-c5ccccc5)nc(-c5cccc6c5sc5ccccc56)n4)c3o2)cc1 ; c1ccc(-c2nc3cccc(-c4nc(-c5ccc6c(c5)sc5ccccc56)nc(-c5ccc6c(c5)c5ccccc5n6-c5ccccc5)n4)c3o2)cc1 ; c1ccc(-c2nc3cccc(-c4nc(-c5ccc6c(c5)c5ccccc5n6-c5ccccc5)nc(-c5cccc6sc7ccccc7c56)n4)c3o2)cc1]

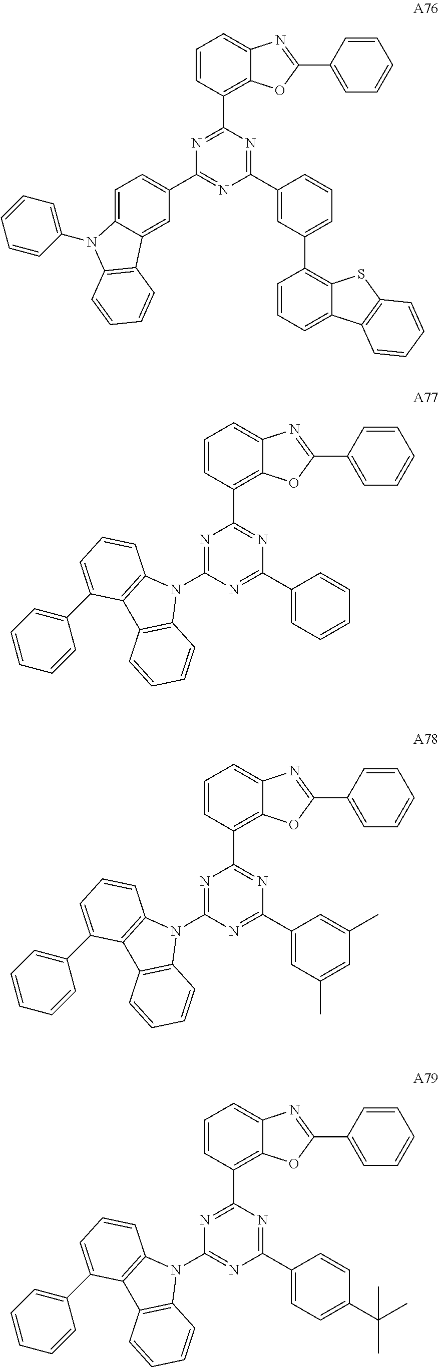 [SMILES: c1ccc(-c2nc3cccc(-c4nc(-c5cccc(-c6cccc7c6sc6ccccc67)c5)nc(-c5ccc6c(c5)c5ccccc5n6-c5ccccc5)n4)c3o2)cc1 ; c1ccc(-c2nc(-c3cccc4nc(-c5ccccc5)oc34)nc(-n3c4ccccc4c4c(-c5ccccc5)cccc43)n2)cc1 ; Cc1cc(C)cc(-c2nc(-c3cccc4nc(-c5ccccc5)oc34)nc(-n3c4ccccc4c4c(-c5ccccc5)cccc43)n2)c1 ; CC(C)(C)c1ccc(-c2nc(-c3cccc4nc(-c5ccccc5)oc34)nc(-n3c4ccccc4c4c(-c5ccccc5)cccc43)n2)cc1]

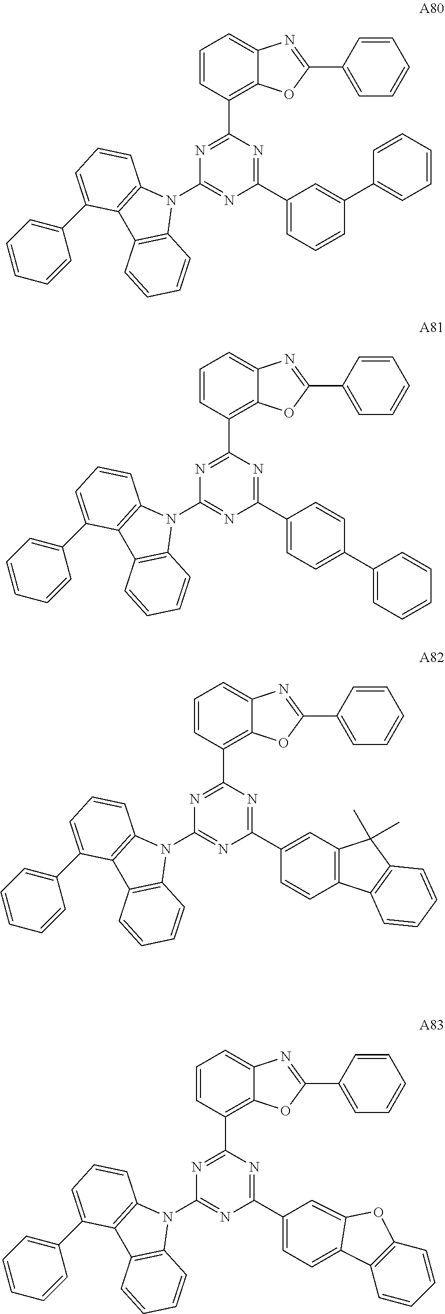 [SMILES: c1ccc(-c2cccc(-c3nc(-c4cccc5nc(-c6ccccc6)oc45)nc(-n4c5ccccc5c5c(-c6ccccc6)cccc54)n3)c2)cc1 ; c1ccc(-c2ccc(-c3nc(-c4cccc5nc(-c6ccccc6)oc45)nc(-n4c5ccccc5c5c(-c6ccccc6)cccc54)n3)cc2)cc1 ; CC1(C)c2ccccc2-c2ccc(-c3nc(-c4cccc5nc(-c6ccccc6)oc45)nc(-n4c5ccccc5c5c(-c6ccccc6)cccc54)n3)cc21 ; c1ccc(-c2nc3cccc(-c4nc(-c5ccc6c(c5)oc5ccccc56)nc(-n5c6ccccc6c6c(-c7ccccc7)cccc65)n4)c3o2)cc1]

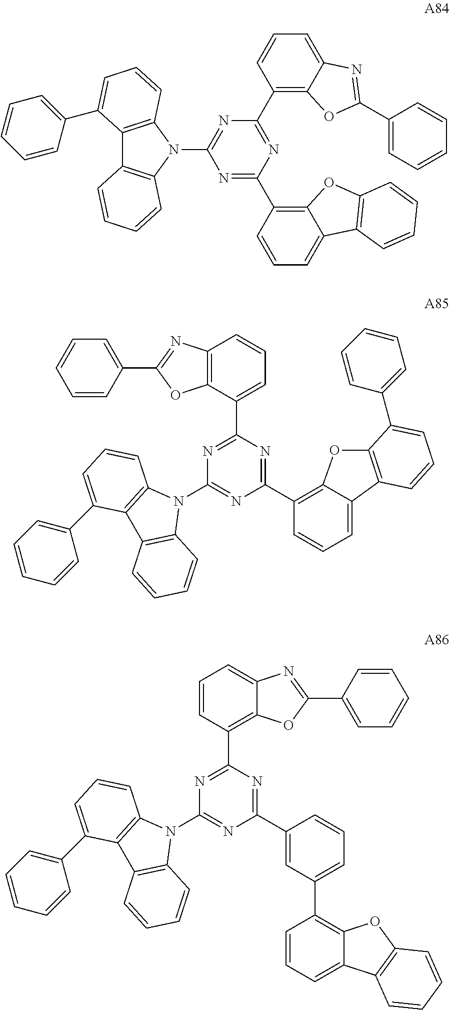 [SMILES: c1ccc(-c2nc3cccc(-c4nc(-c5cccc6c5oc5ccccc56)nc(-n5c6ccccc6c6c(-c7ccccc7)cccc65)n4)c3o2)cc1 ; c1ccc(-c2nc3cccc(-c4nc(-c5cccc6c5oc5c(-c7ccccc7)cccc56)nc(-n5c6ccccc6c6c(-c7ccccc7)cccc65)n4)c3o2)cc1 ; c1ccc(-c2nc3cccc(-c4nc(-c5cccc(-c6cccc7c6oc6ccccc67)c5)nc(-n5c6ccccc6c6c(-c7ccccc7)cccc65)n4)c3o2)cc1]

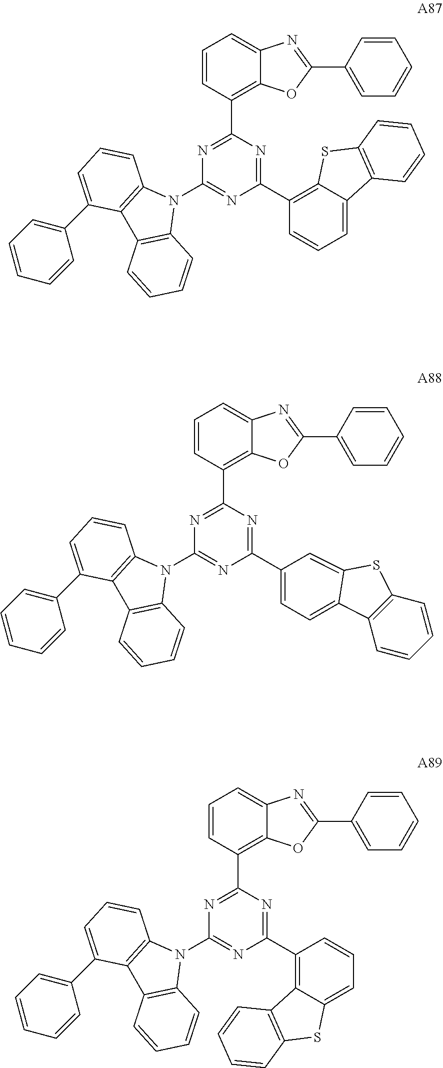 [SMILES: c1ccc(-c2nc3cccc(-c4nc(-c5cccc6c5sc5ccccc56)nc(-n5c6ccccc6c6c(-c7ccccc7)cccc65)n4)c3o2)cc1 ; c1ccc(-c2nc3cccc(-c4nc(-c5ccc6c(c5)sc5ccccc56)nc(-n5c6ccccc6c6c(-c7ccccc7)cccc65)n4)c3o2)cc1 ; c1ccc(-c2nc3cccc(-c4nc(-c5cccc6sc7ccccc7c56)nc(-n5c6ccccc6c6c(-c7ccccc7)cccc65)n4)c3o2)cc1]

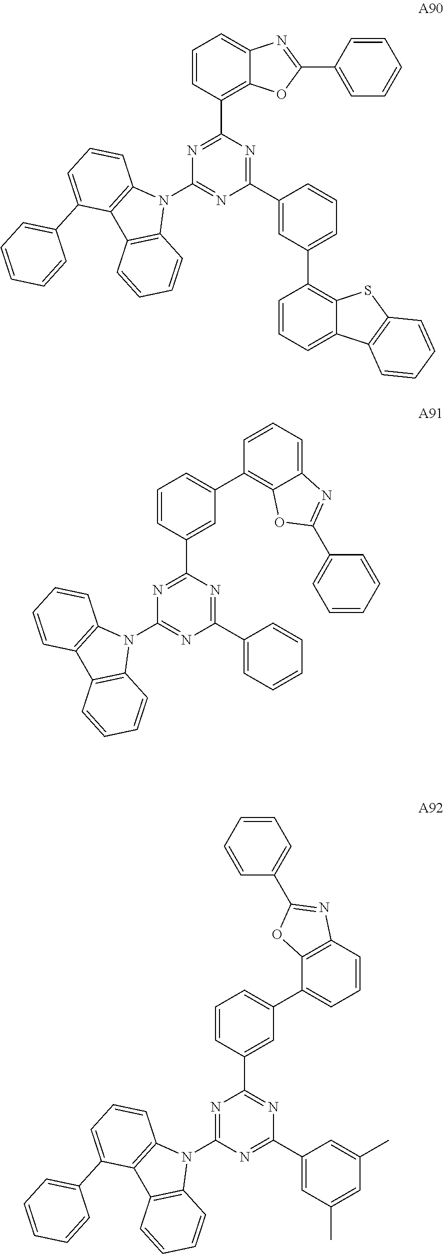 [SMILES: c1ccc(-c2nc3cccc(-c4nc(-c5cccc(-c6cccc7c6sc6ccccc67)c5)nc(-n5c6ccccc6c6c(-c7ccccc7)cccc65)n4)c3o2)cc1 ; c1ccc(-c2nc(-c3cccc(-c4cccc5nc(-c6ccccc6)oc45)c3)nc(-n3c4ccccc4c4ccccc43)n2)cc1 ; Cc1cc(C)cc(-c2nc(-c3cccc(-c4cccc5nc(-c6ccccc6)oc45)c3)nc(-n3c4ccccc4c4c(-c5ccccc5)cccc43)n2)c1]

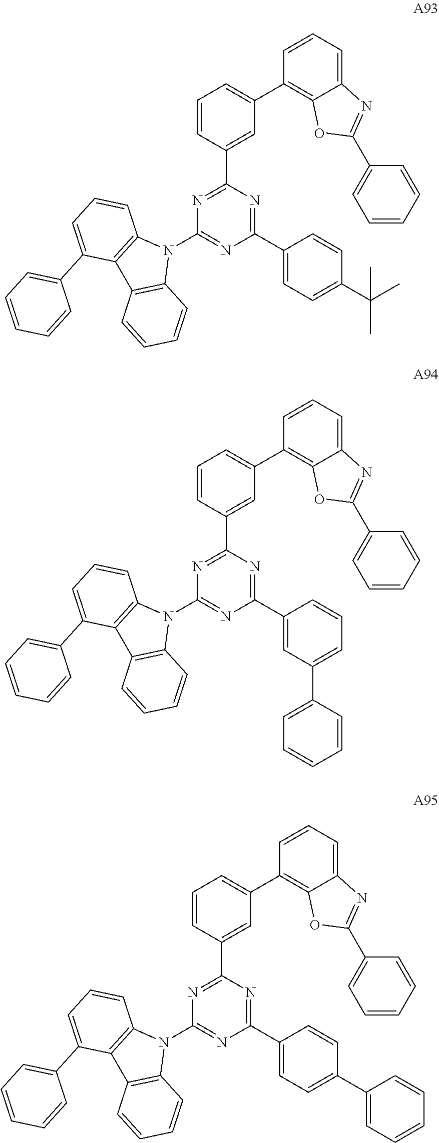 [SMILES: CC(C)(C)c1ccc(-c2nc(-c3cccc(-c4cccc5nc(-c6ccccc6)oc45)c3)nc(-n3c4ccccc4c4c(-c5ccccc5)cccc43)n2)cc1 ; c1ccc(-c2cccc(-c3nc(-c4cccc(-c5cccc6nc(-c7ccccc7)oc56)c4)nc(-n4c5ccccc5c5c(-c6ccccc6)cccc54)n3)c2)cc1 ; c1ccc(-c2ccc(-c3nc(-c4cccc(-c5cccc6nc(-c7ccccc7)oc56)c4)nc(-n4c5ccccc5c5c(-c6ccccc6)cccc54)n3)cc2)cc1]

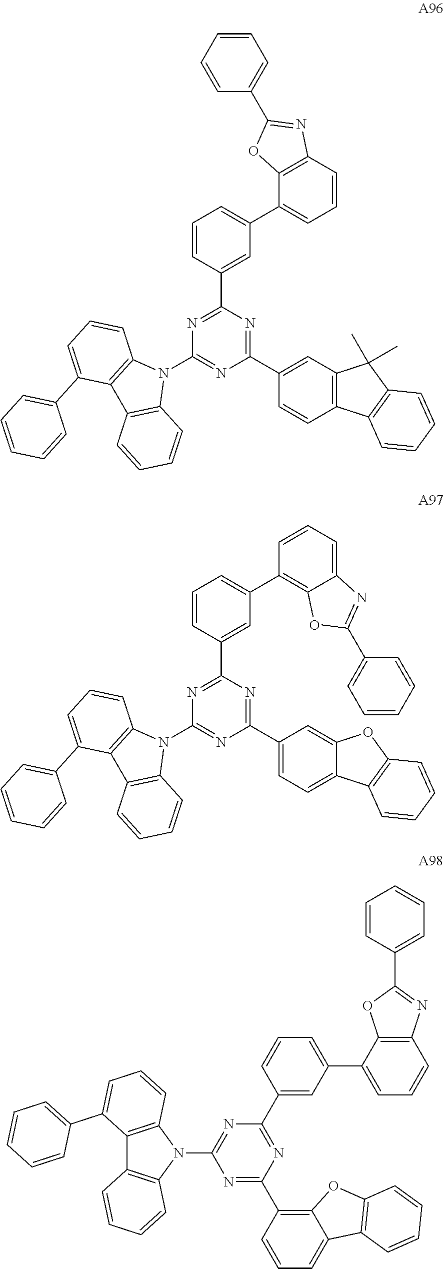 [SMILES: CC1(C)c2ccccc2-c2ccc(-c3nc(-c4cccc(-c5cccc6nc(-c7ccccc7)oc56)c4)nc(-n4c5ccccc5c5c(-c6ccccc6)cccc54)n3)cc21 ; c1ccc(-c2nc3cccc(-c4cccc(-c5nc(-c6ccc7c(c6)oc6ccccc67)nc(-n6c7ccccc7c7c(-c8ccccc8)cccc76)n5)c4)c3o2)cc1 ; c1ccc(-c2nc3cccc(-c4cccc(-c5nc(-c6cccc7c6oc6ccccc67)nc(-n6c7ccccc7c7c(-c8ccccc8)cccc76)n5)c4)c3o2)cc1]

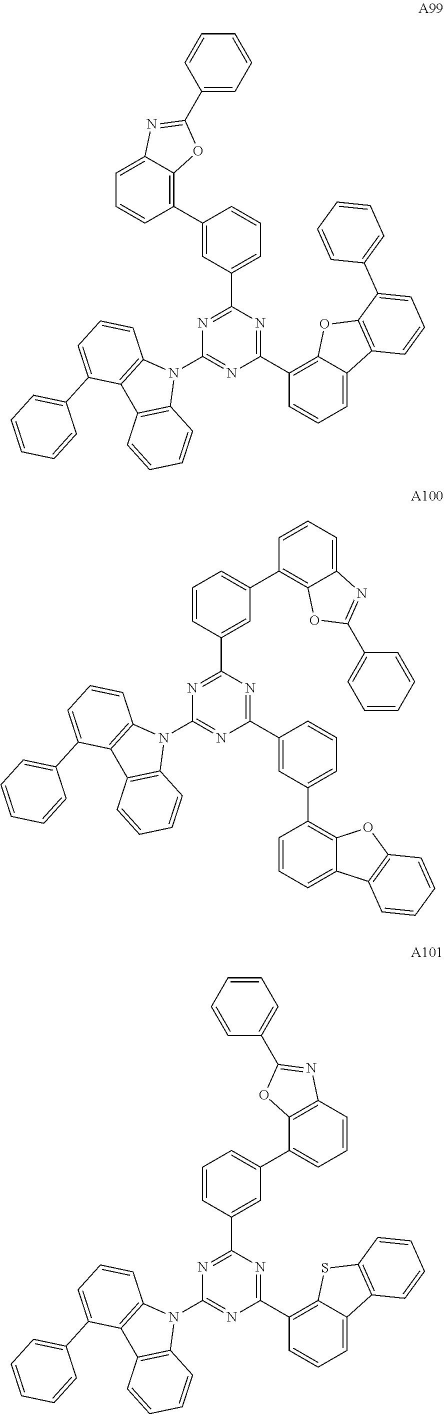 [SMILES: c1ccc(-c2nc3cccc(-c4cccc(-c5nc(-c6cccc7c6oc6c(-c8ccccc8)cccc67)nc(-n6c7ccccc7c7c(-c8ccccc8)cccc76)n5)c4)c3o2)cc1 ; c1ccc(-c2nc3cccc(-c4cccc(-c5nc(-c6cccc(-c7cccc8c7oc7ccccc78)c6)nc(-n6c7ccccc7c7c(-c8ccccc8)cccc76)n5)c4)c3o2)cc1 ; c1ccc(-c2nc3cccc(-c4cccc(-c5nc(-c6cccc7c6sc6ccccc67)nc(-n6c7ccccc7c7c(-c8ccccc8)cccc76)n5)c4)c3o2)cc1]

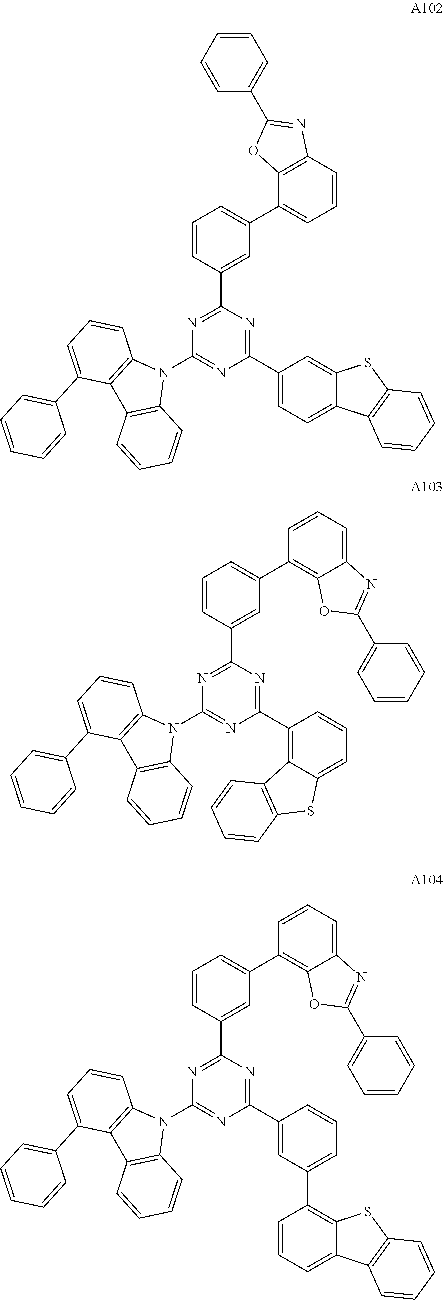 [SMILES: c1ccc(-c2nc3cccc(-c4cccc(-c5nc(-c6ccc7c(c6)sc6ccccc67)nc(-n6c7ccccc7c7c(-c8ccccc8)cccc76)n5)c4)c3o2)cc1 ; c1ccc(-c2nc3cccc(-c4cccc(-c5nc(-c6cccc7sc8ccccc8c67)nc(-n6c7ccccc7c7c(-c8ccccc8)cccc76)n5)c4)c3o2)cc1 ; c1ccc(-c2nc3cccc(-c4cccc(-c5nc(-c6cccc(-c7cccc8c7sc7ccccc78)c6)nc(-n6c7ccccc7c7c(-c8ccccc8)cccc76)n5)c4)c3o2)cc1]

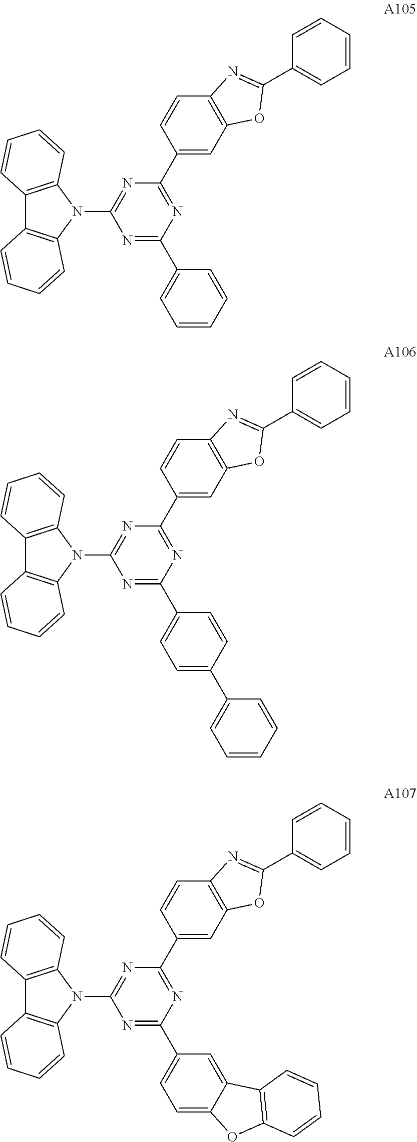 [SMILES: c1ccc(-c2nc(-c3ccc4nc(-c5ccccc5)oc4c3)nc(-n3c4ccccc4c4ccccc43)n2)cc1 ; c1ccc(-c2ccc(-c3nc(-c4ccc5nc(-c6ccccc6)oc5c4)nc(-n4c5ccccc5c5ccccc54)n3)cc2)cc1 ; c1ccc(-c2nc3ccc(-c4nc(-c5ccc6oc7ccccc7c6c5)nc(-n5c6ccccc6c6ccccc65)n4)cc3o2)cc1]

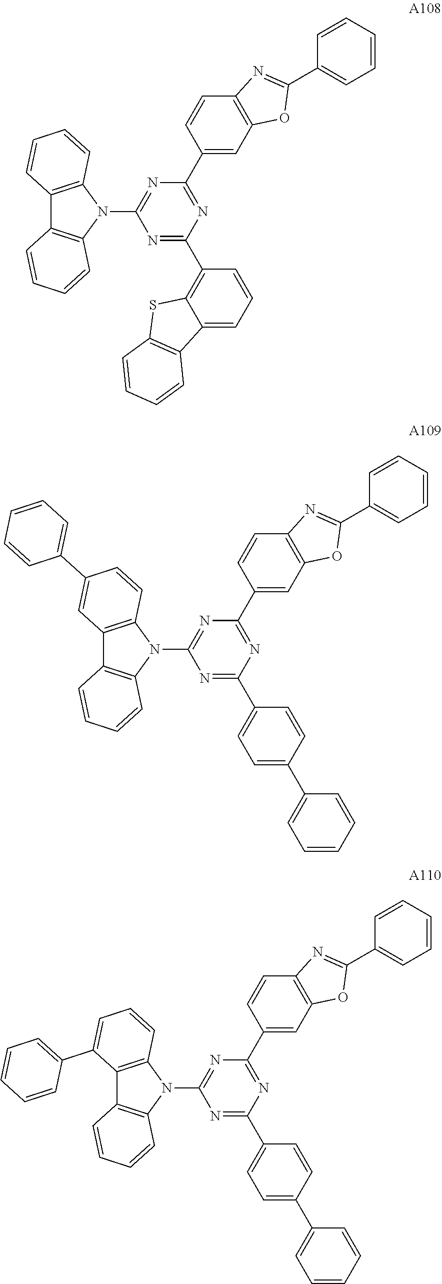 [SMILES: c1ccc(-c2nc3ccc(-c4nc(-c5cccc6c5sc5ccccc56)nc(-n5c6ccccc6c6ccccc65)n4)cc3o2)cc1 ; c1ccc(-c2ccc(-c3nc(-c4ccc5nc(-c6ccccc6)oc5c4)nc(-n4c5ccccc5c5cc(-c6ccccc6)ccc54)n3)cc2)cc1 ; c1ccc(-c2ccc(-c3nc(-c4ccc5nc(-c6ccccc6)oc5c4)nc(-n4c5ccccc5c5c(-c6ccccc6)cccc54)n3)cc2)cc1]

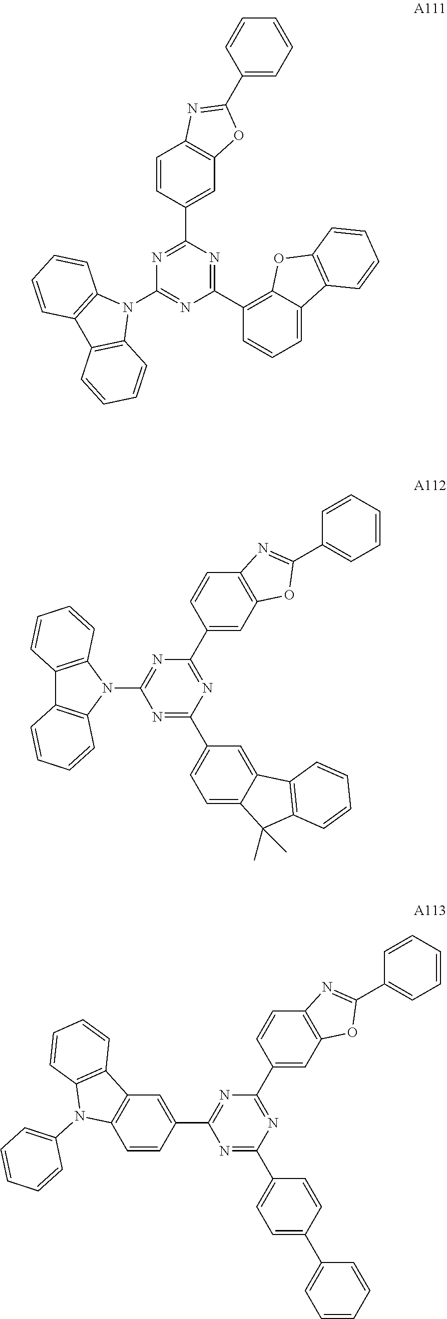 [SMILES: c1ccc(-c2nc3ccc(-c4nc(-c5cccc6c5oc5ccccc56)nc(-n5c6ccccc6c6ccccc65)n4)cc3o2)cc1 ; CC1(C)c2ccccc2-c2cc(-c3nc(-c4ccc5nc(-c6ccccc6)oc5c4)nc(-n4c5ccccc5c5ccccc54)n3)ccc21 ; c1ccc(-c2ccc(-c3nc(-c4ccc5nc(-c6ccccc6)oc5c4)nc(-c4ccc5c(c4)c4ccccc4n5-c4ccccc4)n3)cc2)cc1]

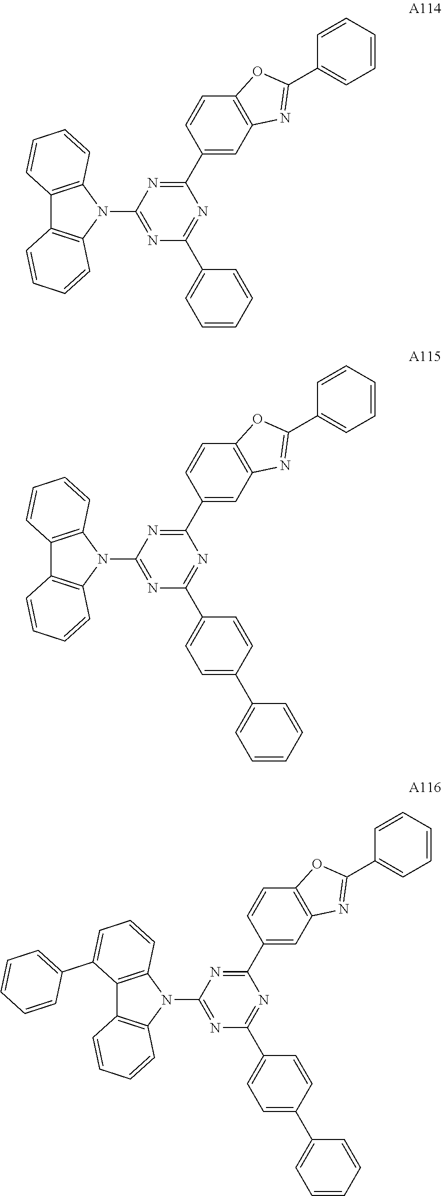 [SMILES: c1ccc(-c2nc(-c3ccc4oc(-c5ccccc5)nc4c3)nc(-n3c4ccccc4c4ccccc43)n2)cc1 ; c1ccc(-c2ccc(-c3nc(-c4ccc5oc(-c6ccccc6)nc5c4)nc(-n4c5ccccc5c5ccccc54)n3)cc2)cc1 ; c1ccc(-c2ccc(-c3nc(-c4ccc5oc(-c6ccccc6)nc5c4)nc(-n4c5ccccc5c5c(-c6ccccc6)cccc54)n3)cc2)cc1]

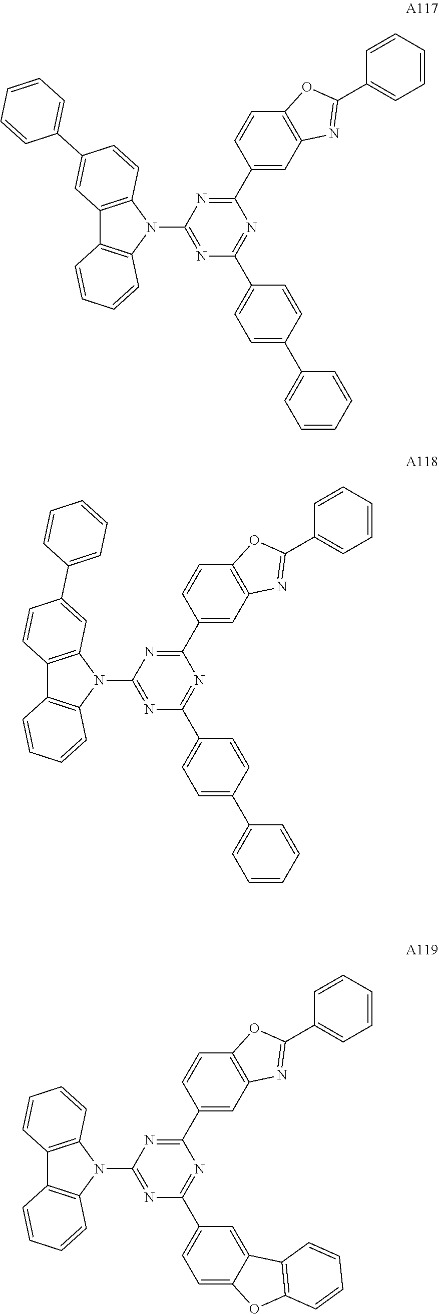 [SMILES: c1ccc(-c2ccc(-c3nc(-c4ccc5oc(-c6ccccc6)nc5c4)nc(-n4c5ccccc5c5cc(-c6ccccc6)ccc54)n3)cc2)cc1 ; c1ccc(-c2ccc(-c3nc(-c4ccc5oc(-c6ccccc6)nc5c4)nc(-n4c5ccccc5c5ccc(-c6ccccc6)cc54)n3)cc2)cc1 ; c1ccc(-c2nc3cc(-c4nc(-c5ccc6oc7ccccc7c6c5)nc(-n5c6ccccc6c6ccccc65)n4)ccc3o2)cc1]

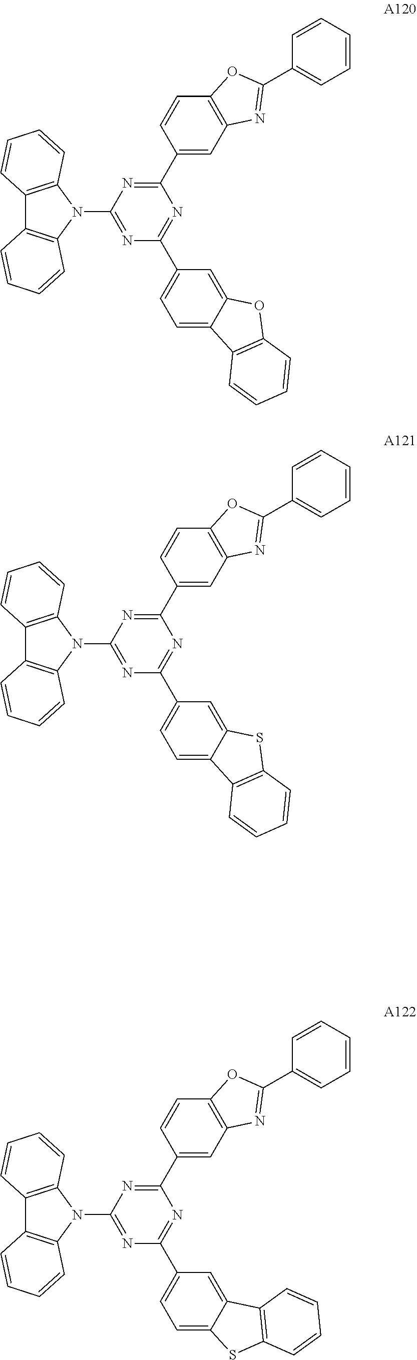 [SMILES: c1ccc(-c2nc3cc(-c4nc(-c5ccc6c(c5)oc5ccccc56)nc(-n5c6ccccc6c6ccccc65)n4)ccc3o2)cc1 ; c1ccc(-c2nc3cc(-c4nc(-c5ccc6c(c5)sc5ccccc56)nc(-n5c6ccccc6c6ccccc65)n4)ccc3o2)cc1 ; c1ccc(-c2nc3cc(-c4nc(-c5ccc6sc7ccccc7c6c5)nc(-n5c6ccccc6c6ccccc65)n4)ccc3o2)cc1]

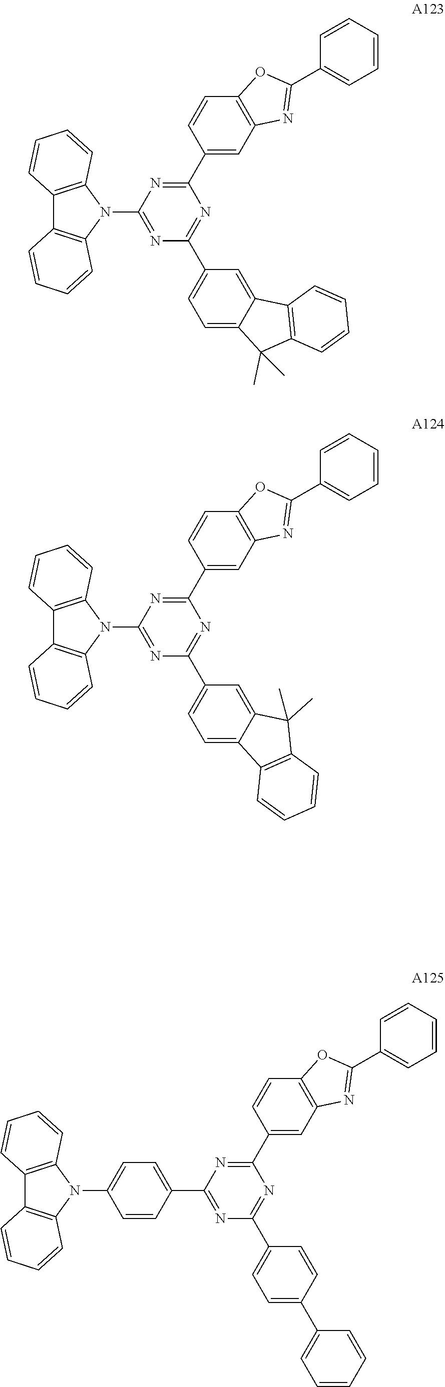 [SMILES: CC1(C)c2ccccc2-c2cc(-c3nc(-c4ccc5oc(-c6ccccc6)nc5c4)nc(-n4c5ccccc5c5ccccc54)n3)ccc21 ; CC1(C)c2ccccc2-c2ccc(-c3nc(-c4ccc5oc(-c6ccccc6)nc5c4)nc(-n4c5ccccc5c5ccccc54)n3)cc21 ; c1ccc(-c2ccc(-c3nc(-c4ccc(-n5c6ccccc6c6ccccc65)cc4)nc(-c4ccc5oc(-c6ccccc6)nc5c4)n3)cc2)cc1]

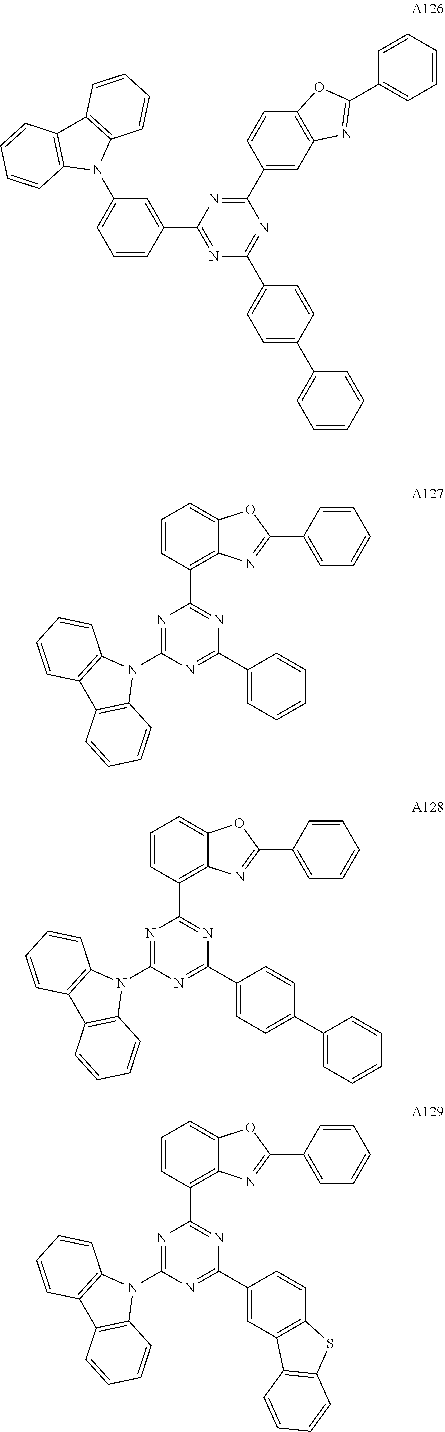

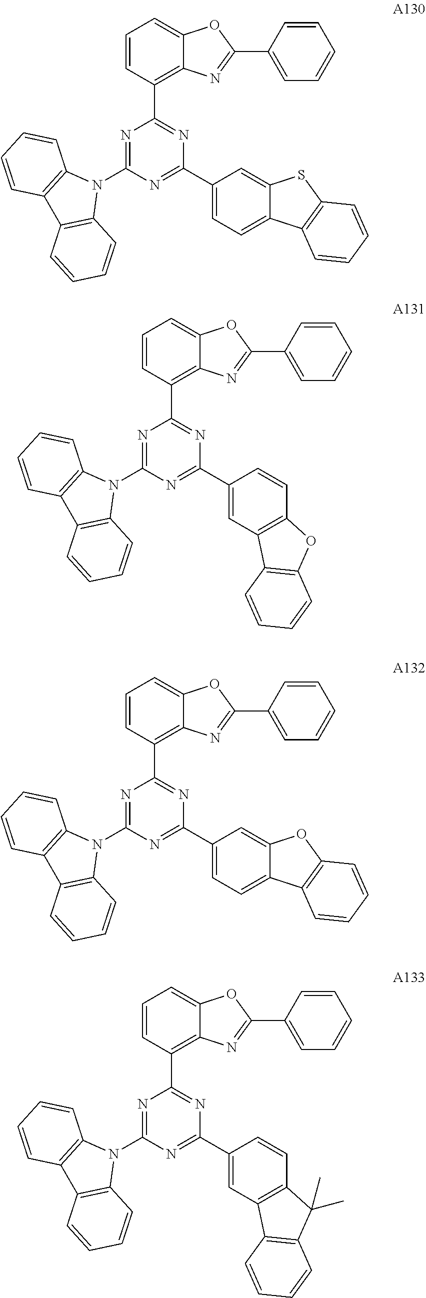 [SMILES: c1ccc(-c2nc3c(-c4nc(-c5ccc6c(c5)sc5ccccc56)nc(-n5c6ccccc6c6ccccc65)n4)cccc3o2)cc1 ; c1ccc(-c2nc3c(-c4nc(-c5ccc6oc7ccccc7c6c5)nc(-n5c6ccccc6c6ccccc65)n4)cccc3o2)cc1 ; c1ccc(-c2nc3c(-c4nc(-c5ccc6c(c5)oc5ccccc56)nc(-n5c6ccccc6c6ccccc65)n4)cccc3o2)cc1 ; CC1(C)c2ccccc2-c2cc(-c3nc(-c4cccc5oc(-c6ccccc6)nc45)nc(-n4c5ccccc5c5ccccc54)n3)ccc21]

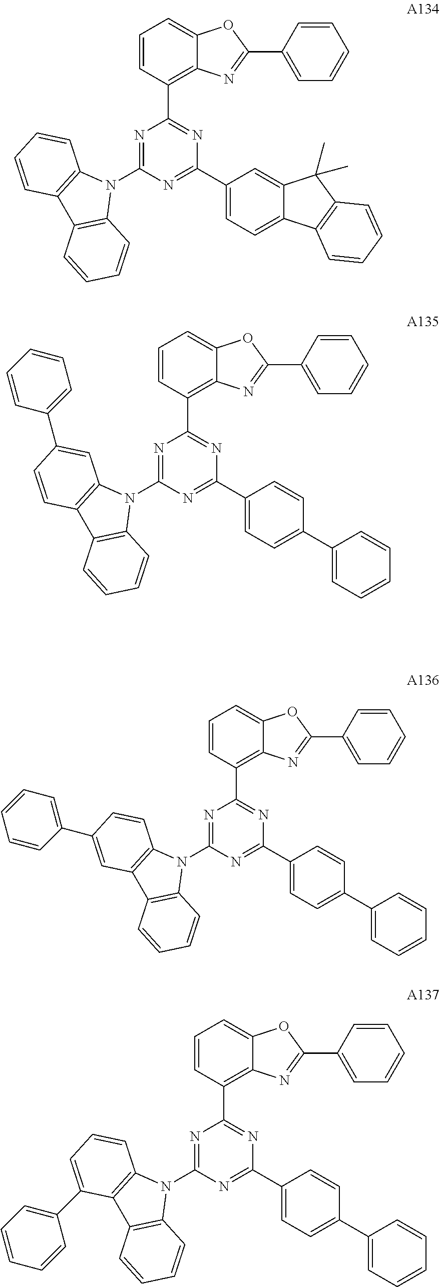 [SMILES: CC1(C)c2ccccc2-c2ccc(-c3nc(-c4cccc5oc(-c6ccccc6)nc45)nc(-n4c5ccccc5c5ccccc54)n3)cc21 ; c1ccc(-c2ccc(-c3nc(-c4cccc5oc(-c6ccccc6)nc45)nc(-n4c5ccccc5c5ccc(-c6ccccc6)cc54)n3)cc2)cc1 ; c1ccc(-c2ccc(-c3nc(-c4cccc5oc(-c6ccccc6)nc45)nc(-n4c5ccccc5c5cc(-c6ccccc6)ccc54)n3)cc2)cc1 ; c1ccc(-c2ccc(-c3nc(-c4cccc5oc(-c6ccccc6)nc45)nc(-n4c5ccccc5c5c(-c6ccccc6)cccc54)n3)cc2)cc1]

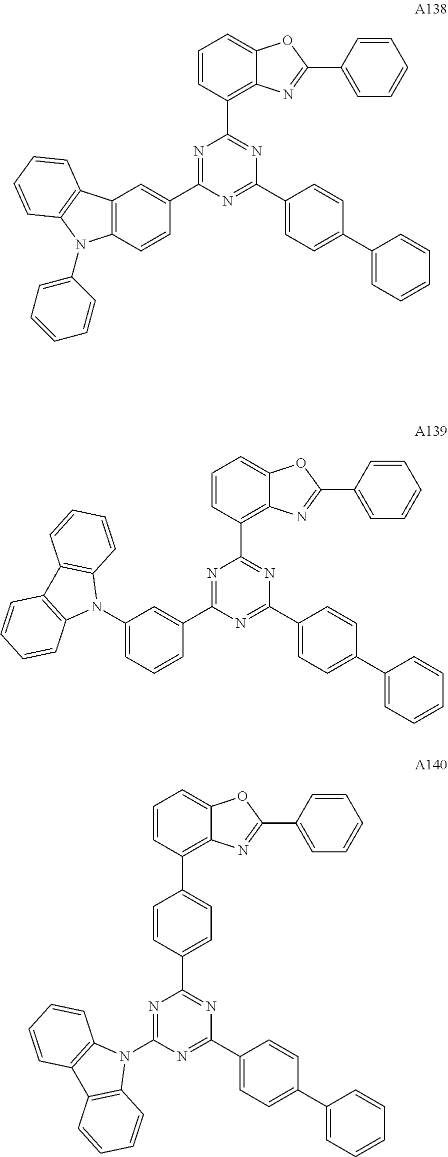 [SMILES: c1ccc(-c2ccc(-c3nc(-c4ccc5c(c4)c4ccccc4n5-c4ccccc4)nc(-c4cccc5oc(-c6ccccc6)nc45)n3)cc2)cc1 ; c1ccc(-c2ccc(-c3nc(-c4cccc(-n5c6ccccc6c6ccccc65)c4)nc(-c4cccc5oc(-c6ccccc6)nc45)n3)cc2)cc1 ; c1ccc(-c2ccc(-c3nc(-c4ccc(-c5cccc6oc(-c7ccccc7)nc56)cc4)nc(-n4c5ccccc5c5ccccc54)n3)cc2)cc1]

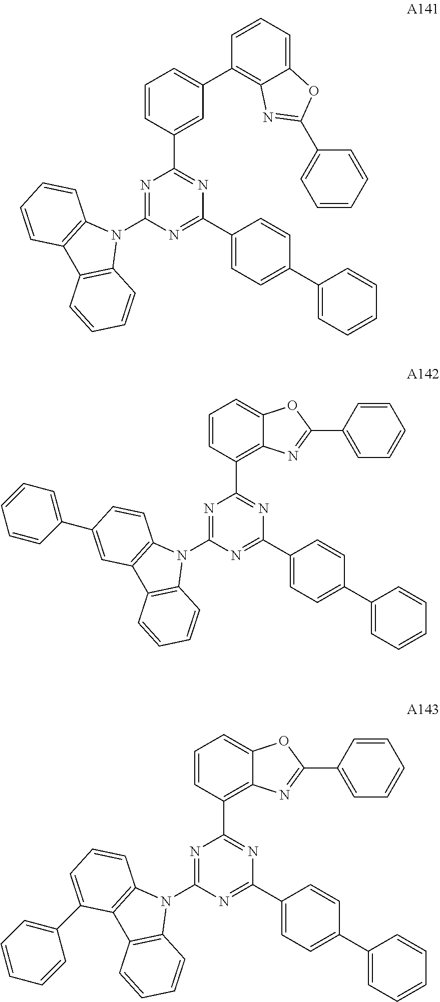 [SMILES: c1ccc(-c2ccc(-c3nc(-c4cccc(-c5cccc6oc(-c7ccccc7)nc56)c4)nc(-n4c5ccccc5c5ccccc54)n3)cc2)cc1 ; c1ccc(-c2ccc(-c3nc(-c4cccc5oc(-c6ccccc6)nc45)nc(-n4c5ccccc5c5cc(-c6ccccc6)ccc54)n3)cc2)cc1 ; c1ccc(-c2ccc(-c3nc(-c4cccc5oc(-c6ccccc6)nc45)nc(-n4c5ccccc5c5c(-c6ccccc6)cccc54)n3)cc2)cc1]

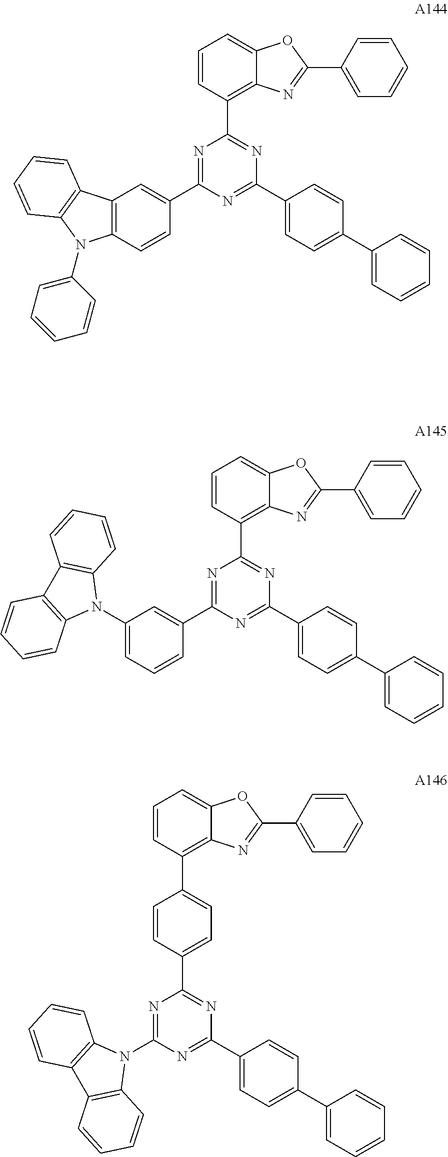 [SMILES: c1ccc(-c2ccc(-c3nc(-c4ccc5c(c4)c4ccccc4n5-c4ccccc4)nc(-c4cccc5oc(-c6ccccc6)nc45)n3)cc2)cc1 ; c1ccc(-c2ccc(-c3nc(-c4cccc(-n5c6ccccc6c6ccccc65)c4)nc(-c4cccc5oc(-c6ccccc6)nc45)n3)cc2)cc1 ; c1ccc(-c2ccc(-c3nc(-c4ccc(-c5cccc6oc(-c7ccccc7)nc56)cc4)nc(-n4c5ccccc5c5ccccc54)n3)cc2)cc1]

### [Synthesis]

### 1. Synthesis of the Compound A1

### (1) Compound A

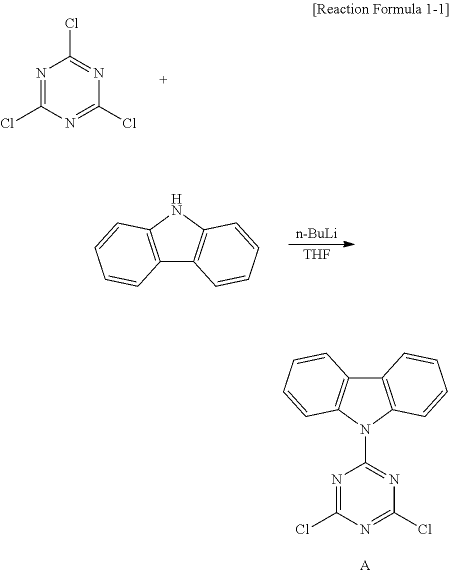

In the one-neck round-bottom flask, 2,4,6-trichloro-1,3,5-triazine (10 g, 0.054 mol) was dissolved in 100 ml of THF and cooled to −78° C. under nitrogen condition. 2.5 M n-BuLi solution (19 ml, 0.048 mol) was slowly added, and 30 minutes later, carbazole (8.1 g, 0.048 mol) was additionally added. After raising the temperature to room temperature, the mixture was reacted for 3 hours. After the reaction was completed, the mixture was completely precipitated using methanol, filtered, and then dissolved again in methylene chloride (MC). The mixture was columned with hexane and methylene chloride (MC), and the obtained material was concentrated. Impurities were removed using acetone slurry and filtered to obtain the compound A. (12.7 g, 83%)

### (2) Compound C-1

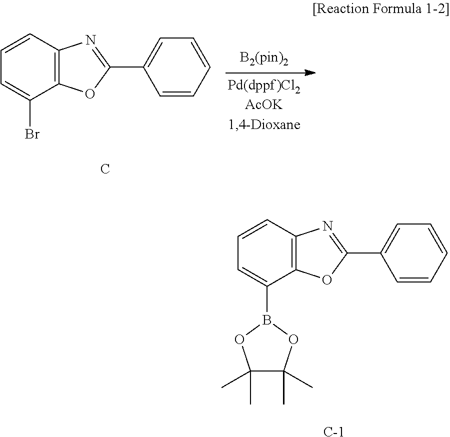

In a one-neck round-bottom flask, 7-bromo-2-phenyl-1,3-benzthiazole (20 g, 0.072 mol), bis(diphenylphosphino)ferrocene]dichloropalladium(II) (Pd(dppf)Cl₂, 2.6 g, 0.0004 mol), potassium acetate (14.3 g, 0.14 mol), and bis(pinacolato)diboron (B₂(pin)₂, 27.7 g, 0.11 mol) were dissolved in 250 ml of 1,4-dioxane and refluxed for 3 hours. After the reaction solution was sufficiently cooled to room temperature, the mixture was filtered. The mixture was washed with MC and concentrated. The concentrated material was dissolved in MC and then filtered using MC and silicagel. After concentrating the filtered solution, impurities were removed using MeOH slurry and filtered to obtain the compound C-1. (21 g, 90%)

### (3) Compound A1-i

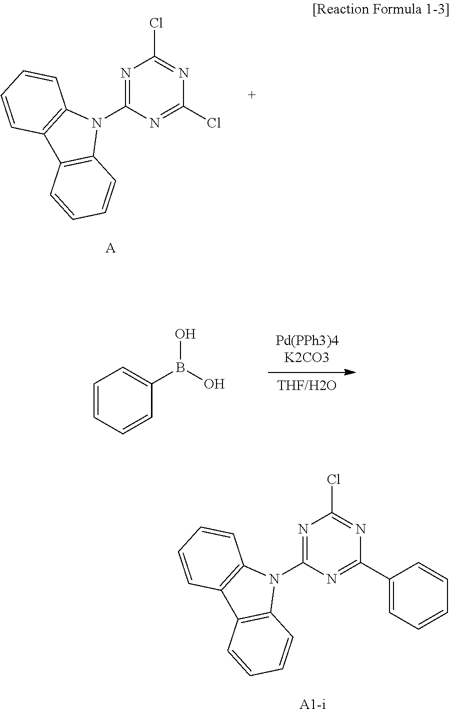

In a one-neck round-bottom flask, 9-(4,6-dichloro-1,3,5-triazin-2-yl)carbazole (10 g, 0.032 mol), phenyl boronic acid (3.5 g, 0.029 mol), palladium-tetrakis(triphenylphosphine) (Pd(PPh₃)₄, 1.8 g, 0.0018 mol) and K₂CO₃ (8.77 g, 0.063 mol) were dissolved in a mixture of tetrahydrofuran (100 mL) and water (20 mL) and refluxed at 70° C. for 2 hours. After the reaction solution was cooled to room temperature, an excess amount of methanol was added to completely precipitate the material and then filtered. The precipitated material was completely dissolved by heating with DCB (1,2-dichlrobenzene) and then filtered using chloroform and silica gel. The filtered solution was concentrated, impurities were removed using methanol slurry, and then filtered to obtain the compound A1-i. (6.2 g, 54%)

### (4) Compound A1

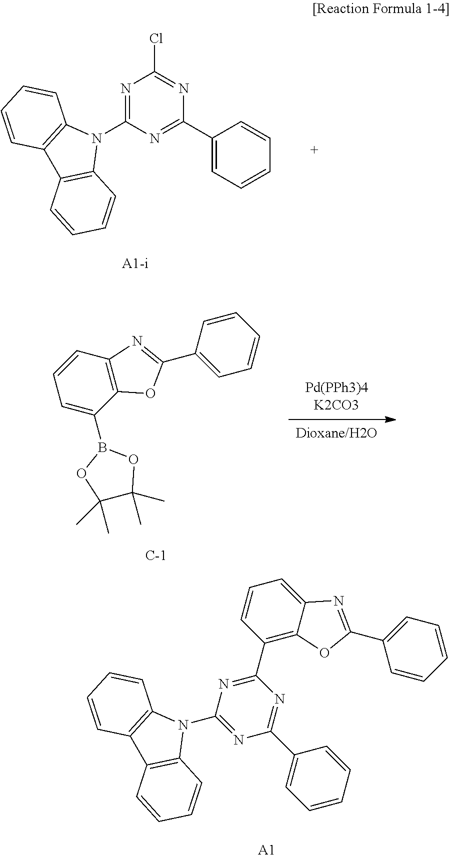

In a one-neck round-bottom flask, the compound A1-i (6.2 g, 0.017 mol), the compound C-1 (6.04 g, 0.018 mol), palladium-tetrakis(triphenylphosphine) (Pd(PPh₃)₄, 1 g, 0.0009 mol), and K₂CO₃ (4.7 g, 0.034 mol) were dissolved in a mixture of 1,4-dioxane (100 mL) and water (20 mL) and reacted under reflux for 4 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A1. (9.2 g, 80.6%)

### 2. Synthesis of the Compound A5

### (1) Compound A5-i

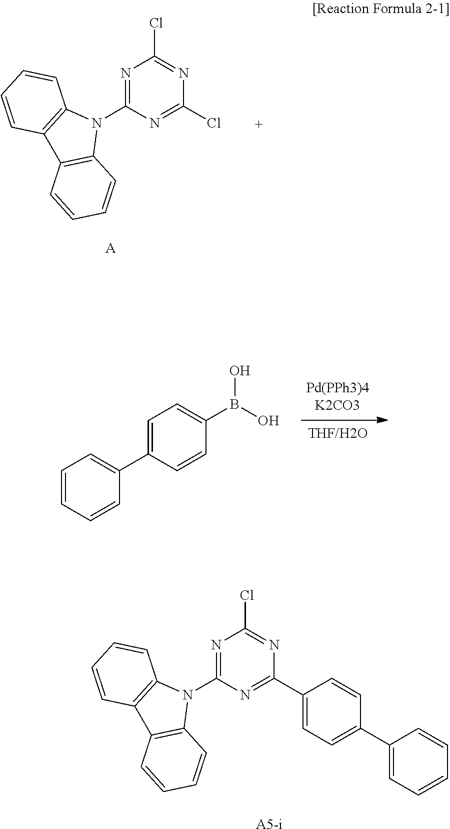

In a one-neck round-bottom flask, 9-(4,6-dichloro-1,3,5-triazin-2-yl)carbazole (10 g, 0.032 mol), [1,1′-biphenyl]-4-ylboronic acid (5.6 g, 0.029 mol), palladium-tetrakis(triphenylphosphine) (Pd(PPh₃)₄, 1.8 g, 0.0018 mol) and K₂CO₃ (8.77 g, 0.063 mol) were dissolved in a mixture of tetrahydrofuran (100 mL) and water (20 mL) and refluxed at 70° C. for 2 hours. After the reaction solution was cooled to room temperature, an excess amount of methanol was added to completely precipitate the material and then filtered. The precipitated material was completely dissolved by heating with DCB and then filtered using chloroform and silica gel. The filtered solution was concentrated, impurities were removed using methanol slurry, and then filtered to obtain the compound A5-i. (8.1 g, 59%)

### (2) Compound A5

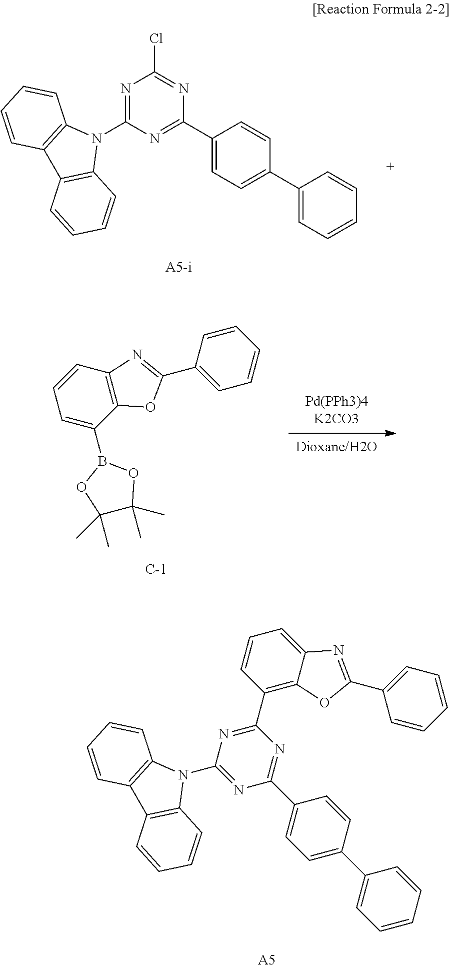

In a one-neck round-bottom flask, the compound A5-i (8.1 g, 0.019 mol), the compound C-1 (6.6 g, 0.021 mol), palladium-tetrakis(triphenylphosphine) (Pd(PPh₃)₄, 1.1 g, 0.0010 mol), and K₂CO₃ (5.17 g, 0.037 mol) were dissolved in a mixture of 1,4-dioxane (100 mL) and water (20 mL) and reacted under reflux for 4 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A5. (8.5 g, 76.8%)

### 3. Synthesis of the Compound A6

### (1) Compound A6-i

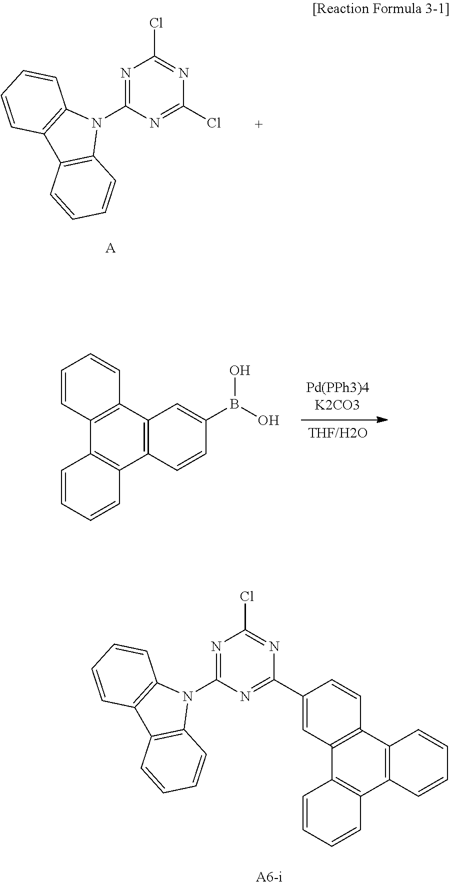

In a one-neck round-bottom flask, 9-(4,6-dichloro-1,3,5-triazin-2-yl)carbazole (10 g, 0.032 mol), triphenylen-2-ylboronic acid (7.7 g, 0.029 mol), palladium-tetrakis(triphenylphosphine) (Pd(PPh₃)₄, 1.8 g, 0.0018 mol), and K₂CO₃ (8.77 g, 0.063 mol) were dissolved in a mixture of tetrahydrofuran (100 mL) and water (20 mL) and refluxed at 70° C. for 2 hours. After the reaction solution was cooled to room temperature, an excess amount of methanol was added to completely precipitate the material and then filtered. The precipitated material was completely dissolved by heating with DCB (1,2-dichlrobenzene) and then filtered using chloroform and silica gel. The filtered solution was concentrated, impurities were removed using methanol slurry, and then filtered to obtain the compound A6-i. (9.2 g, 57%)

### (2) Compound A6

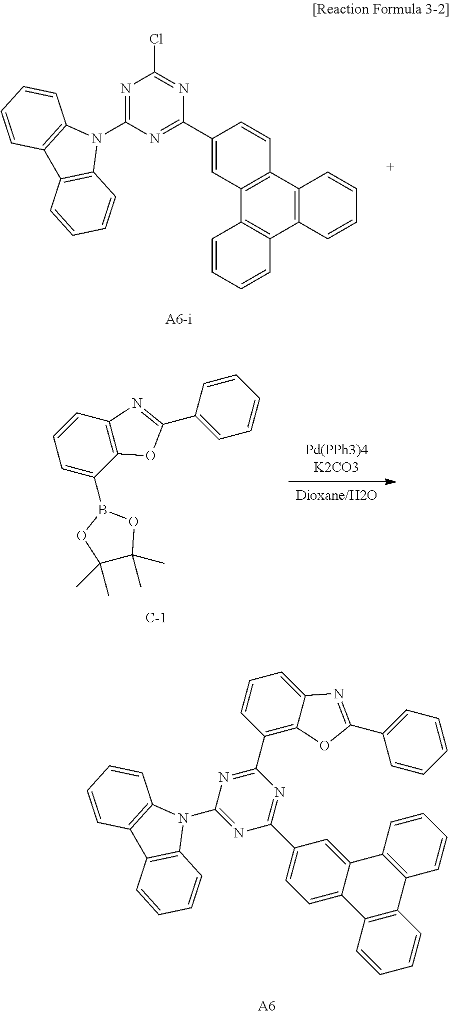

In a one-neck round-bottom flask, the compound A6-i (9.2 g, 0.018 mol), the compound C-1 (6.4 g, 0.020 mol), palladium-tetrakis(triphenylphosphine) (Pd(PPh₃)₄, 1.0 g, 0.0009 mol), and K₂CO₃ (5.00 g, 0.036 mol) were dissolved in a mixture of 1,4-dioxane (100 mL) and water (20 mL) and reacted under reflux for 2 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A6. (10.2 g, 83.8%)

### 4. Synthesis of the Compound A7

### (1) Compound A7-i

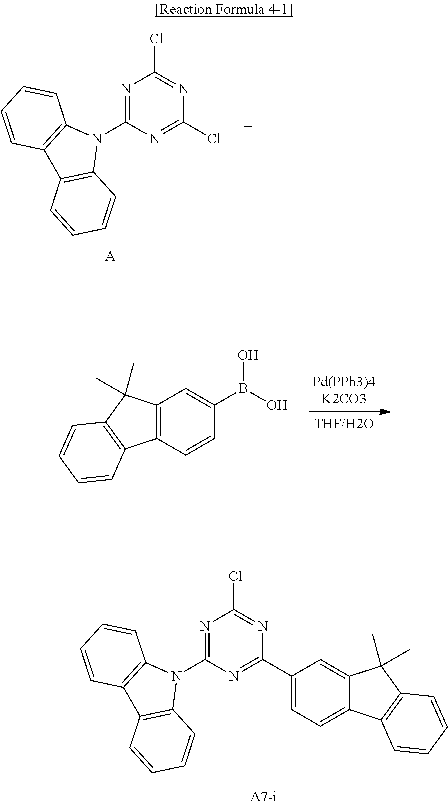

In a one-neck round-bottom flask, 9-(4,6-dichloro-1,3,5-triazin-2-yl)carbazole (10 g, 0.032 mol), (9,9-dimethyl-9H-fluoren-2-yl)boronic acid (6.8 g, 0.029 mol), palladium-tetrakis(triphenylphosphine) (Pd(PPh₃)₄, 1.8 g, 0.0018 mol) and K₂CO₃ (8.77 g, 0.063 mol) were dissolved in a mixture of tetrahydrofuran (100 mL) and water (20 mL) and refluxed at 70° C. for 2 hours. After the reaction solution was cooled to room temperature, an excess amount of methanol was added to completely precipitate the material and then filtered. The precipitated material was completely dissolved by heating with DCB and then filtered using chloroform and silica gel. The filtered solution was concentrated, impurities were removed using methanol slurry, and then filtered to obtain the compound A7-i. (8.9 g, 59%)

### (2) Compound A7

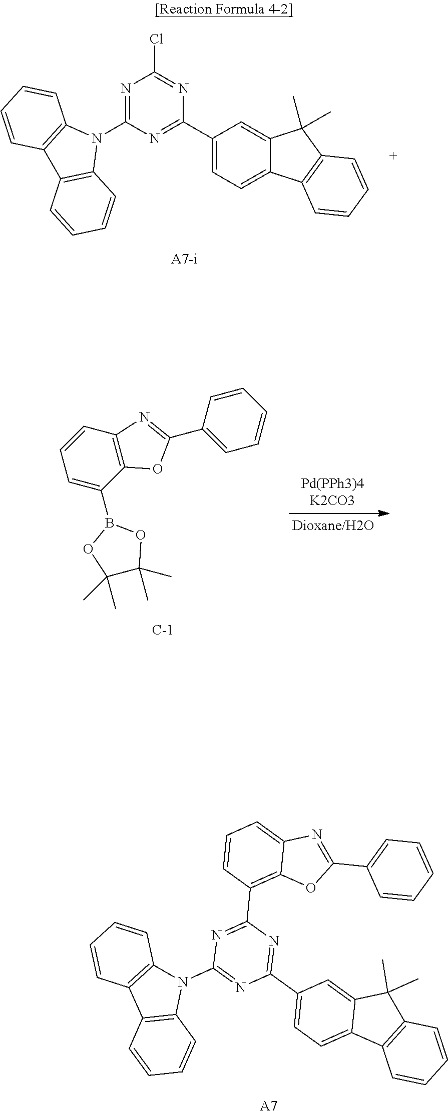

In a one-neck round-bottom flask, the compound A7-i (8.9 g, 0.019 mol), the compound C-1 (6.6 g, 0.021 mol), palladium-tetrakis(triphenylphosphine) (Pd(PPh₃)₄, 1.1 g, 0.0010 mol), and K₂CO₃ (5.17 g, 0.037 mol) were dissolved in a mixture of 1,4-dioxane (100 mL) and water (20 mL) and reacted under reflux for 4 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A7. (7.5 g, 63.1%)

### 5. Synthesis of the Compound A8

### (1) Compound A8-i

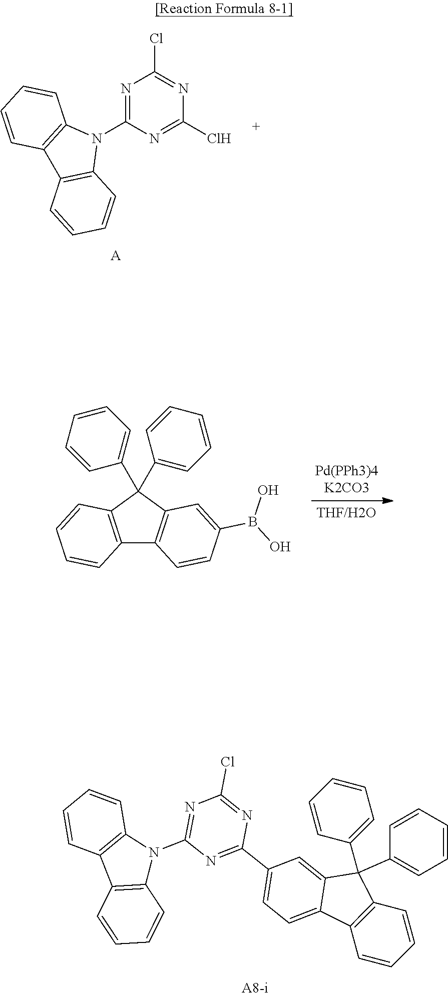

In a one-neck round-bottom flask, 9-(4,6-dichloro-1,3,5-triazin-2-yl)carbazole (10 g, 0.032 mol), (9,9-diphenyl-fluoren-2-yl)boronic acid (10.3, 0.029 mol), palladium-tetrakis(triphenylphosphine) (Pd(PPh₃)₄, 1.8 g, 0.0018 mol) and K₂CO₃ (8.77 g, 0.063 mol) were dissolved in a mixture of tetrahydrofuran (100 mL) and water (20 mL) and refluxed at 70° C. for 2 hours. After the reaction solution was cooled to room temperature, an excess amount of methanol was added to completely precipitate the material and then filtered. The precipitated material was completely dissolved by heating with DCB and then filtered using chloroform and silica gel. The filtered solution was concentrated, impurities were removed using methanol slurry, and then filtered to obtain the compound A8-i. (12.7 g, 67%)

### (2) Compound A8

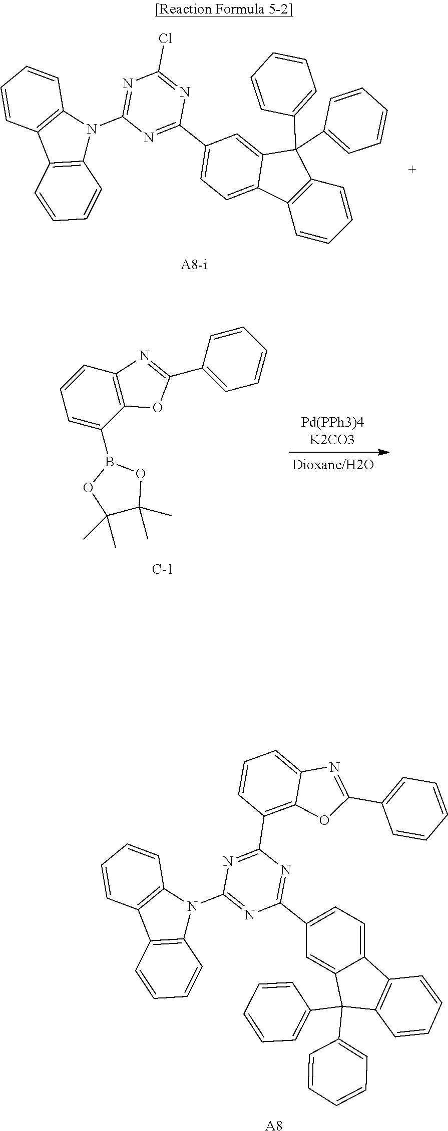

In a one-neck round-bottom flask, the compound A8-i (12.7, 0.021 mol), the compound C-1 (7.5 g, 0.023 mol), palladium-tetrakis(triphenylphosphine) (Pd(PPh₃)₄, 1.2 g, 0.0011 mol), and K₂CO₃ (5.9 g, 0.043 mol) were dissolved in a mixture of 1,4-dioxane (100 mL) and water (20 mL) and reacted under reflux for 4 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A8. (13.0 g, 81.0%)

### 6. Synthesis of the Compound A9

### (1) Compound A9-i

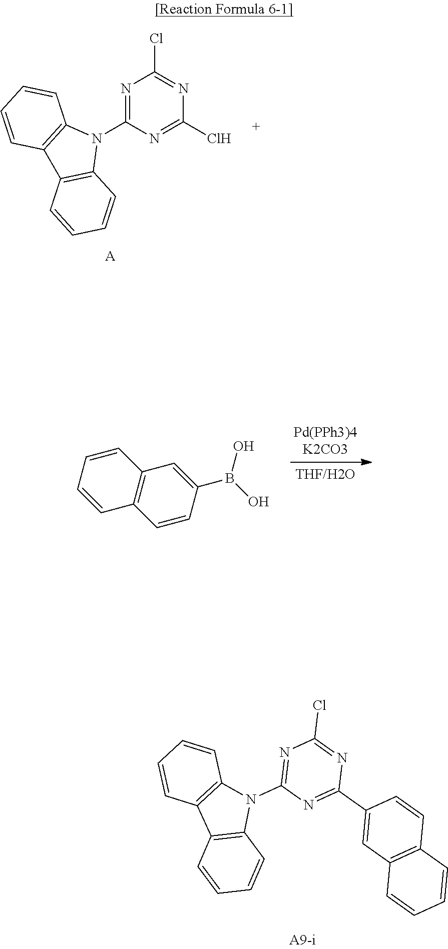

In a one-neck round-bottom flask, 9-(4,6-dichloro-1,3,5-triazin-2-yl)carbazole (10.1 g, 0.032 mol), naphthalen-2-ylboronic acid (5.0 g, 0.029 mol), palladium-tetrakis(triphenylphosphine) (Pd(PPh₃)₄, 1.8 g, 0.0018 mol), and K₂CO₃ (8.76 g, 0.063 mol) were dissolved in a mixture of tetrahydrofuran (100 mL) and water (20 mL) and refluxed at 70° C. for 2 hours. After the reaction solution was cooled to room temperature, an excess amount of methanol was added to completely precipitate the material and then filtered. The precipitated material was completely dissolved by heating with DCB and then filtered using chloroform and silica gel. The filtered solution was concentrated, impurities were removed using methanol slurry, and then filtered to obtain the compound A9-i. (7.8 g, 66%)

### (2) Compound A9

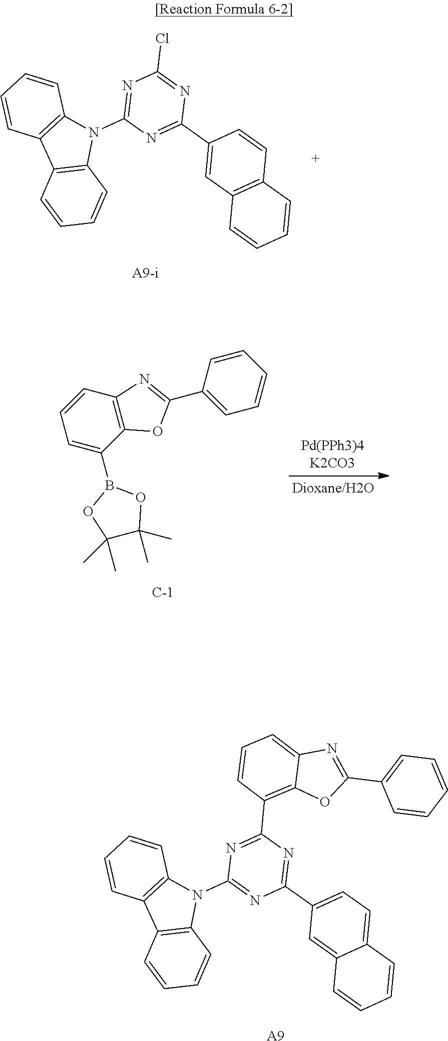

In a one-neck round-bottom flask, the compound A9-i (7.8 g, 0.021 mol), the compound C-1 (7.5 g, 0.023 mol), palladium-tetrakis(triphenylphosphine) (Pd(PPh₃)₄, 1.2 g, 0.0011 mol), and K₂CO₃ (6.0 g, 0.044 mol) were dissolved in a mixture of 1,4-dioxane (100 mL) and water (20 mL) and reacted under reflux for 4 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A9. (9.7 g, 81.0%)

### 7. Synthesis of the Compound A19

### (1) Compound A19-i

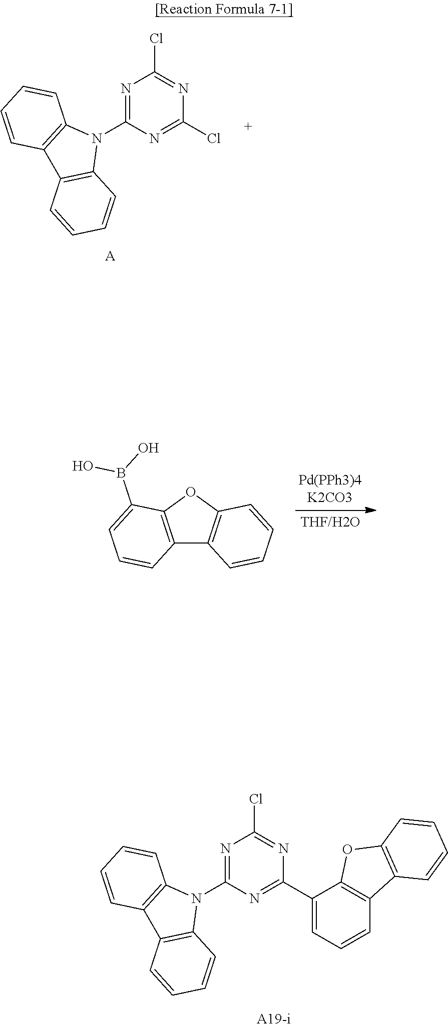

In a one-neck round-bottom flask, 9-(4,6-dichloro-1,3,5-triazin-2-yl)carbazole (10 g, 0.032 mol), dibenzofuran-4-ylboronic acid (6.0 g, 0.029 mol), palladium-tetrakis(triphenylphosphine) (Pd(PPh₃)₄, 1.8 g, 0.0018 mol) and K₂CO₃ (8.77 g, 0.063 mol) were dissolved in a mixture of tetrahydrofuran (100 mL) and water (20 mL) and reflux at 70° C. for 2 hours. After the reaction solution was cooled to room temperature, an excess amount of methanol was added to completely precipitate the material and then filtered. The precipitated material was completely dissolved by heating with DCB and then filtered using chloroform and silica gel. The filtered solution was concentrated, impurities were removed using methanol slurry, and then filtered to obtain the compound A19-i. (8.5 g, 60%)

### (2) Compound A19

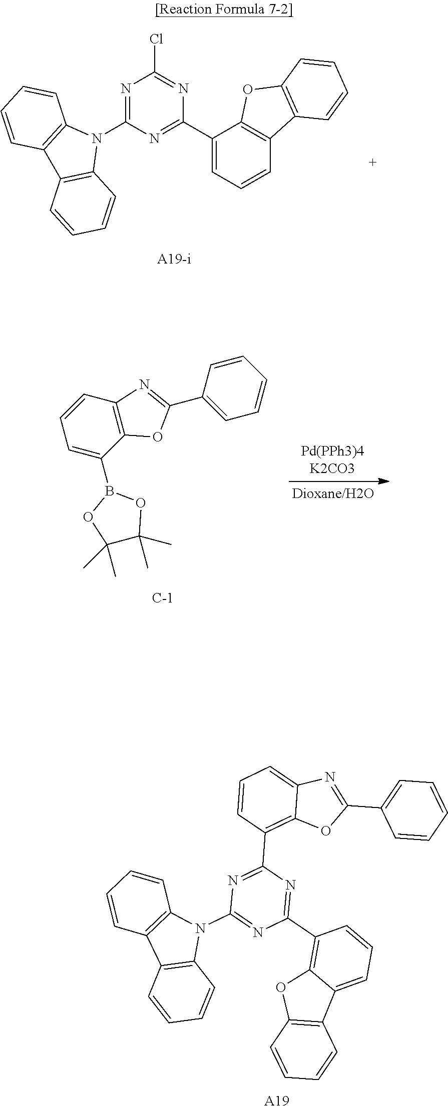

In a one-neck round-bottom flask, the compound A19-i (8.5 g, 0.019 mol), the compound C-1 (6.7 g, 0.021 mol), palladium-tetrakis(triphenylphosphine) (Pd(PPh₃)₄, 1.1 g, 0.0010 mol), and K₂CO₃ (5.26 g, 0.038 mol) were dissolved in a mixture of 1,4-dioxane (100 mL) and water (20 mL) and reacted under reflux for 4 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A19. (8.8 g, 76.4%)

### 8. Synthesis of the Compound A29

### (1) Compound C-2

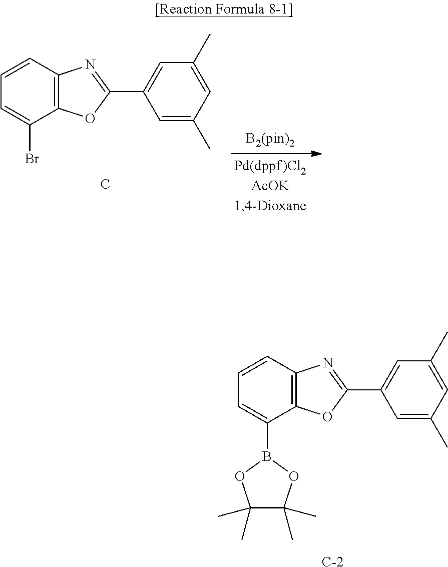

In a one-neck round-bottom flask, 7-bromo-2-phenyl-1,3-benzthiazole (20 g 0.072 mol), bis(diphenylphosphino)ferrocene]dichloropalladium(II) (Pd(dppf)Cl₂, 2.6 g, 0.0004 mol), potassium acetate (14.3 g, 0.14 mol), and bis(pinacolato)diboron (B₂(pin)₂, 27.7 g, 0.11 mol) were dissolved in 250 ml of 1,4-dioxane and refluxed for 3 hours. After the reaction solution was sufficiently cooled to room temperature, the mixture was filtered. The mixture was washed with MC and concentrated. The concentrated material was dissolved in MC and then filtered using MC and silica gel. After concentrating the filtered solution, impurities were removed using MeOH slurry and filtered to obtain the compound C-2. (21 g, 90%)

### (2) Compound A29-i

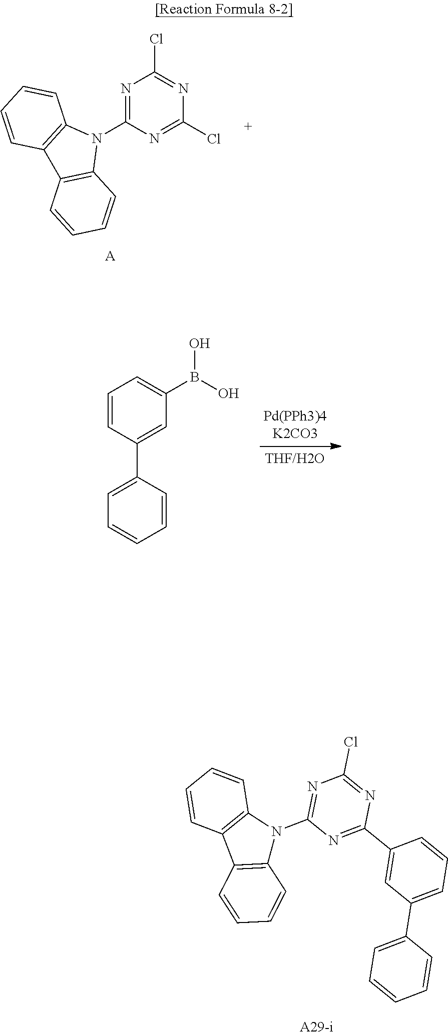

In a one-neck round-bottom flask, 9-(4,6-dichloro-1,3,5-triazin-2-yl)carbazole (10 g, 0.032 mol), [1,1′-biphenyl]-3-ylboronic acid (5.6 g, 0.029 mol), palladium-tetrakis(triphenylphosphine) (Pd(PPh₃)₄, 1.8 g, 0.0018 mol) and K₂CO₃ (8.77 g, 0.063 mol) were dissolved in a mixture of tetrahydrofuran (100 mL) and water (20 mL) and refluxed at 70° C. for 2 hours. After the reaction solution was cooled to room temperature, an excess amount of methanol was added to completely precipitate the material and then filtered. The precipitated material was completely dissolved by heating with DCB and then filtered using chloroform and silica gel. The filtered solution was concentrated, impurities were removed using methanol slurry, and then filtered to obtain the compound A29-i. (8.1 g, 65%)

### (3) Compound A29

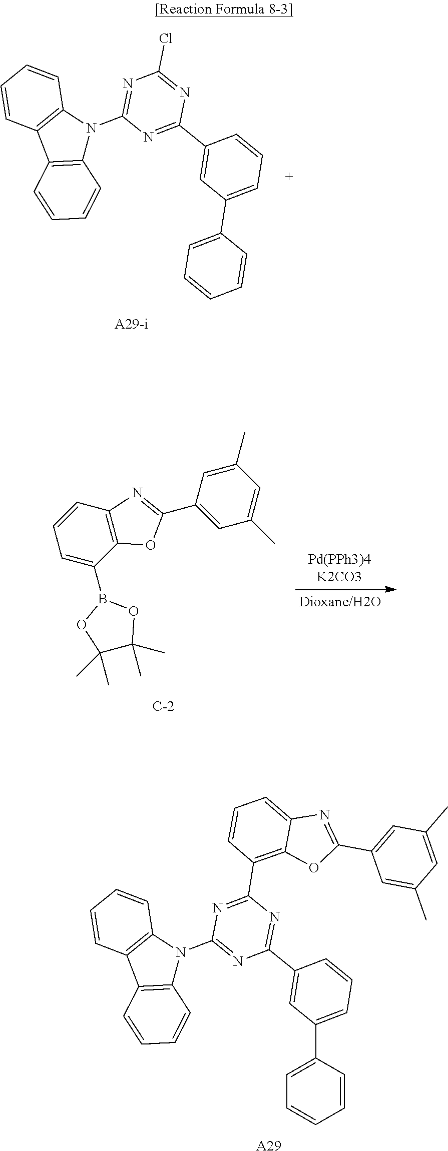

In a one-neck round-bottom flask, the compound A29-i (8.1 g, 0.019 mol), the compound C-2 (6.29 g, 0.018 mol), palladium-tetrakis(triphenylphosphine) (Pd(PPh₃)₄, 1 g, 0.0009 mol), and K₂CO₃ (4.7 g, 0.034 mol) were dissolved in a mixture of 1,4-dioxane (100 mL) and water (20 mL) and reacted under reflux for 4 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A29. (8.6 g, 80.6%)

### 9. Synthesis of the Compound A30

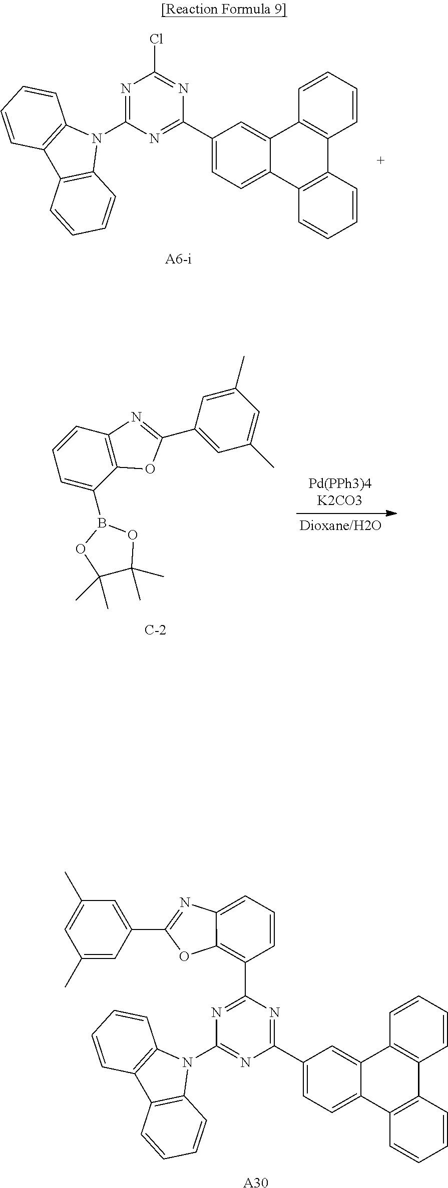

In a one-neck round-bottom flask, the compound A6-i (8.6 g, 0.017 mol), the compound C-2 (6.29 g, 0.018 mol), palladium-tetrakis(triphenylphosphine) (Pd(PPh₃)₄, 1 g, 0.0009 mol), and K₂CO₃ (4.7 g, 0.034 mol) were dissolved in a mixture of 1,4-dioxane (100 mL) and water (20 mL) and reacted under reflux for 4 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A30. (8.9 g, 79.0%)

### 10. Synthesis of the Compound A105

### (1) Compound C-3

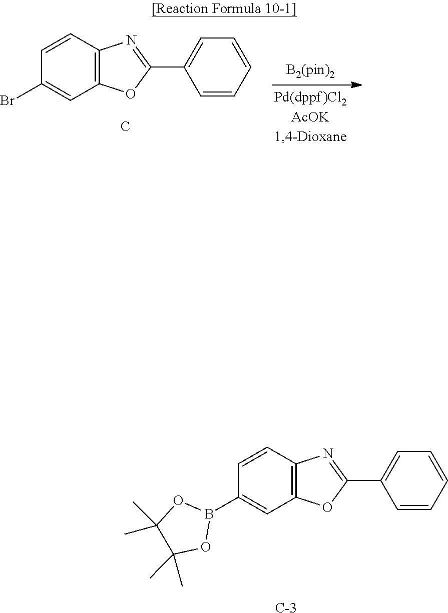

In a one-neck round-bottom flask, 6-bromo-2-phenyl-1,3-benzoxazole (20 g 0.072 mol), bis(diphenylphosphino)ferrocene]dichloropalladium(II) (Pd(dppf)Cl₂, 2.6 g, 0.0004 mol), potassium acetate (14.3 g, 0.14 mol), and bis(pinacolato)diboron (B₂(pin)₂, 27.7 g, 0.11 mol) were dissolved in 250 ml of 1,4-dioxane and refluxed for 3 hours. After the reaction solution was sufficiently cooled to room temperature, the mixture was filtered. The mixture was washed with MC and concentrated. The concentrated material was dissolved in MC and then filtered using MC and silica gel. After concentrating the filtered solution, impurities were removed using MeOH slurry and filtered to obtain the compound C-3. (22 g, 94%)

### (2) Compound A105

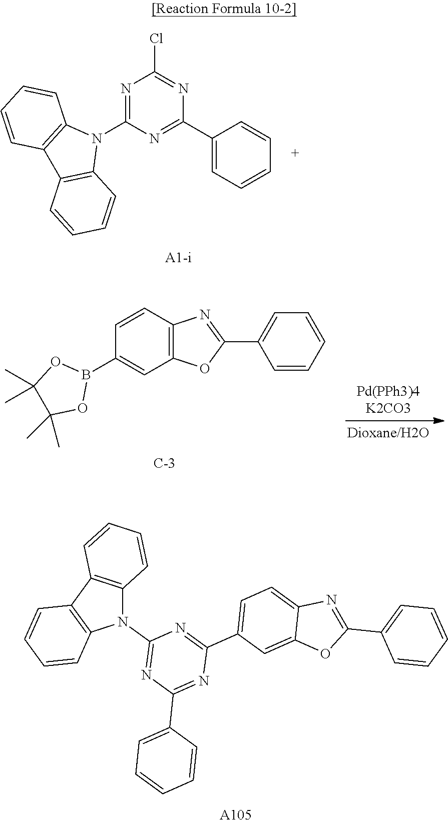

In a one-neck round-bottom flask, the compound A1-i (10.0 g, 0.028 mol), the compound C-3 (9.9 g, 0.031 mol), palladium-tetrakis(triphenylphosphine) (Pd(PPh₃)₄, 1.6 g, 0.0014 mol), and K₂CO₃ (7.8 g, 0.056 mol) were dissolved in a mixture of 1,4-dioxane (100 mL) and water (20 mL) and reacted under reflux for 4 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A105. (11.1 g, 76.8%)

### 11. Synthesis of the Compound A115

### (1) Compound C-4

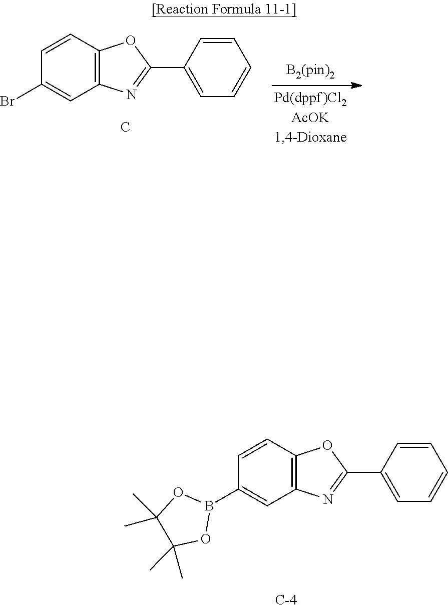

In a one-neck round-bottom flask, 5-bromo-2-phenyl-1,3-benzooxazole (20 g 0.072 mol), bis(diphenylphosphino)ferrocene]dichloropalladium(II) (Pd(dppf)Cl₂, 2.6 g, 0.0004 mol), potassium acetate (14.3 g, 0.14 mol), and bis(pinacolato)diboron (B₂(pin)₂, 27.7 g, 0.11 mol) were dissolved in 250 ml of 1,4-dioxane and refluxed for 3 hours. After the reaction solution was sufficiently cooled to room temperature, the mixture was filtered. The mixture was washed with MC and concentrated. The concentrated material was dissolved in MC and then filtered using MC and silica gel. After concentrating the filtered solution, impurities were removed using MeOH slurry and filtered to obtain the compound C-4. (22.2 g, 95%)

### (2) Compound A115

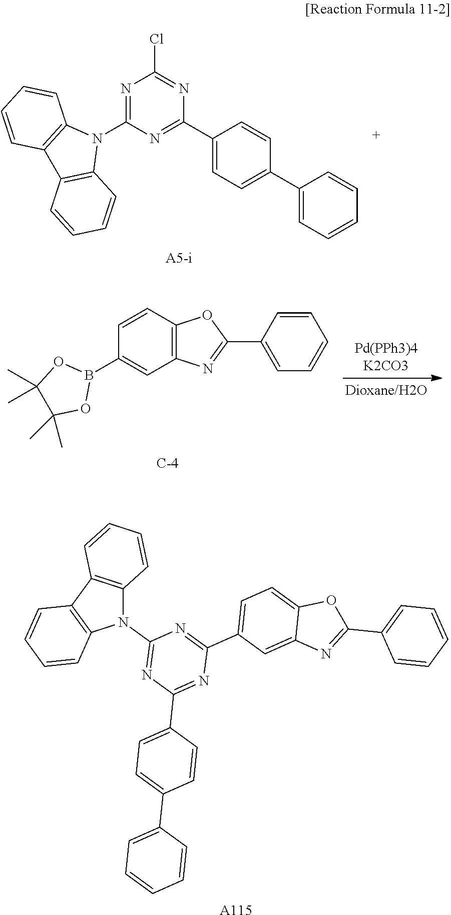

In a one-neck round-bottom flask, the compound A5-i (8.7 g, 0.019 mol), the compound C-4 (7.45 g, 0.023 mol), palladium-tetrakis(triphenylphosphine) (Pd(PPh₃)₄, 1.0 g, 0.0010 mol), and K₂CO₃ (5.3 g, 0.038 mol) were dissolved in a mixture of 1,4-dioxane (100 mL) and water (20 mL) and reacted under reflux for 2 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A115. (9.2 g, 72.6%)

### 12. Synthesis of the Compound A128

### (1) Compound C-5

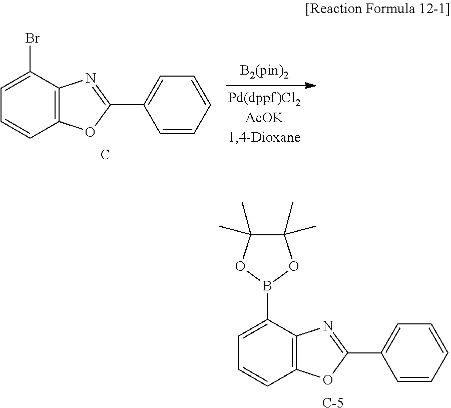

In a one-neck round-bottom flask, 6-bromo-2-phenyl-1,3-benzthiazole (20 g 0.072 mol), bis(diphenylphosphino)ferrocene]dichloropalladium(II) (Pd(dppf)Cl₂, 2.6 g, 0.0004 mol), potassium acetate (14.3 g, 0.14 mol), and bis(pinacolato)diboron (B₂(pin)₂, 27.7 g, 0.11 mol) were dissolved in 250 ml of 1,4-dioxane and refluxed for 3 hours. After the reaction solution was sufficiently cooled to room temperature, the mixture was filtered. The mixture was washed with MC and concentrated. The concentrated material were dissolved in MC and then filtered using MC and silica gel. After concentrating the filtered solution, impurities were removed using MeOH slurry and filtered to obtain the compound C-5. (21 g, 92%)

### (2) Compound A128

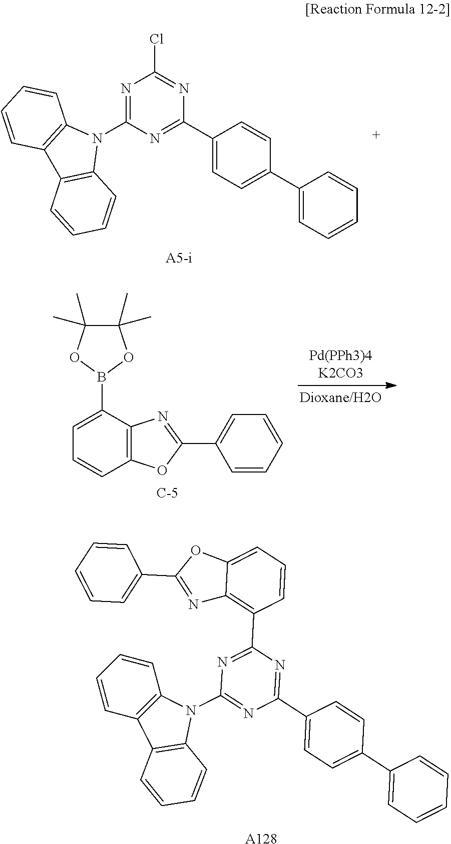

In a one-neck round-bottom flask, the compound A5-i (8.7 g, 0.019 mol), the compound C-5 (7.45 g, 0.023 mol), palladium-tetrakis(triphenylphosphine) (Pd(PPh₃)₄, 1.0 g, 0.0010 mol), and K₂CO₃ (5.3 g, 0.038 mol) were dissolved in a mixture of 1,4-dioxane (100 mL) and water (20 mL) and reacted under reflux for 5 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A128. (9.2 g, 72.6%)

### 13. Synthesis of the Compound A132

### (1) Compound 132-i

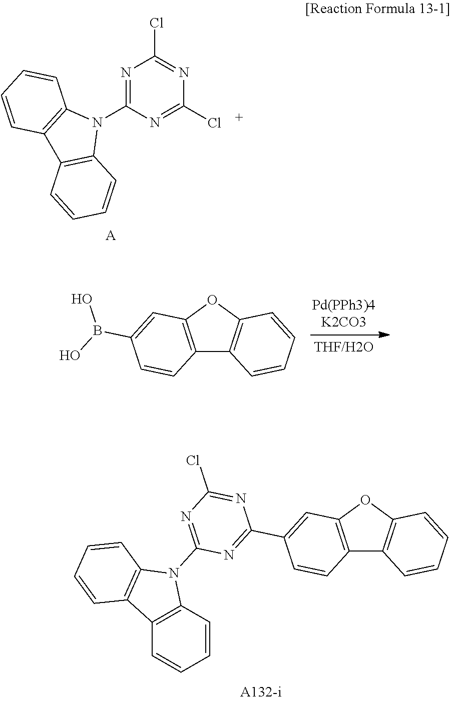

In a one-neck round-bottom flask, 9-(4,6-dichloro-1,3,5-triazin-2-yl)carbazole (10 g, 0.032 mol), dibenzofuran-3-ylboronic acid (5.4 g, 0.025 mol), palladium-tetrakis(triphenylphosphine) (Pd(PPh₃)₄, 1.8 g, 0.0016 mol) and K₂CO₃ (8.8 g, 0.064 mol) were dissolved in a mixture of tetrahydrofuran (100 mL) and water (20 mL) and refluxed at 70° C. for 2 hours. After the reaction solution was cooled to room temperature, an excess amount of methanol was added to completely precipitate the material and then filtered. The precipitated material was completely dissolved by heating with DCB and then filtered using chloroform and silica gel. The filtered solution was concentrated, impurities were removed using methanol slurry, and then filtered to obtain the compound A132-i. (9.9 g, 70%)

### (2) Compound A132

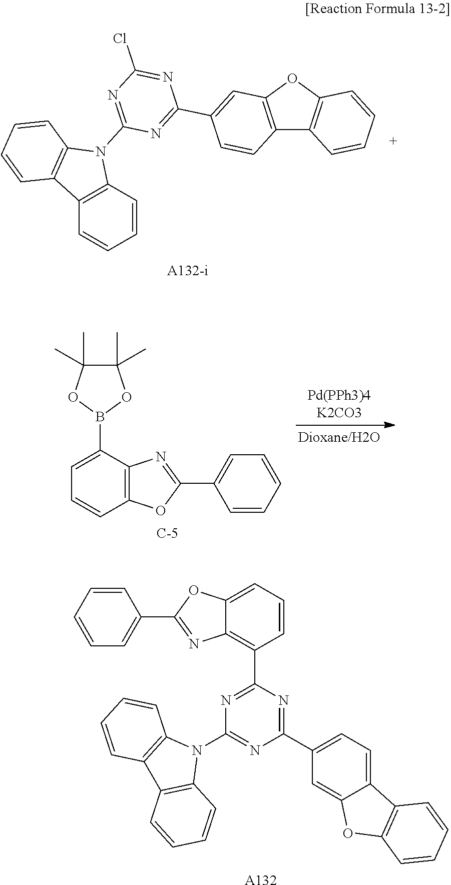

In a one-neck round-bottom flask, the compound A132-i (9.9 g, 0.022 mol), the compound C-5 (0.8 g, 0.024 mol), palladium-tetrakis(triphenylphosphine) (Pd(PPh₃)₄, 1.3 g, 0.0011 mol), and K₂CO₃ (6.1 g, 0.044 mol)) were dissolved in a mixture of 1,4-dioxane (100 mL) and water (20 mL) and reacted under reflux for 4 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A132. (9.2 g, 83.1%)

### 14. Synthesis of the Compound A83

### (1) Compound D-i

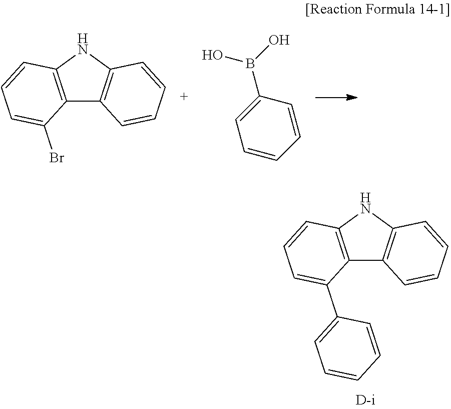 [SMILES: Brc1cccc2[nH]c3ccccc3c12 ; OB(O)c1ccccc1 ; c1ccc(-c2cccc3[nH]c4ccccc4c23)cc1]

In a one-neck round-bottom flask, 1-bromo carbazole (25 g, 0.1 mol), phenyl boronic acid (19 g, 0.153 mol), palladium-tetrakis(triphenylphosphine) (Pd(PPh₃)₄, 5.9 g, 0.0050 mol) and K₂CO₃ (28.1 g, 0.044 mol) were dissolved in a mixture of tetrahydrofuran (300 mL) and water (1000 mL) and refluxed at 110° C. for 12 hours. After the reaction solution was cooled to room temperature, the precipitated material was filtered and washed with water and methanol. The filtered material was completely dissolved with MC and filtered using silica gel. The obtained solution was concentrated, impurities were removed using methanol slurry, and then filtered to obtain the compound D-i. (21.4 g, 86.6%)

### (2) Compound D

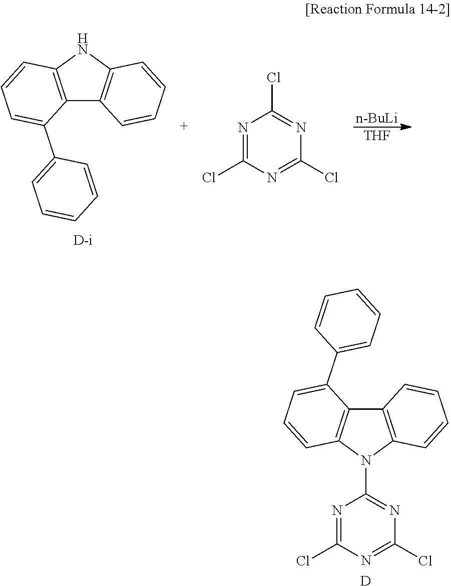

In a one-neck round-bottom flask, 2,4,6-trichloro-1,3,5-triazine (20 g, 0.11 mol) was dissolved in 200 ml of THF and cooled to −78° C. under nitrogen condition. 2.5M n-BuLi solution (38 ml, 0.096 mol) was slowly added, and 30 minutes later, 1-phenyl carbazole (21.4 g, 0.096 mol) was additionally added. After raising the temperature to room temperature, the mixture was reacted for 3 hours. After the reaction was completed, the mixture was completely precipitated using methanol, filtered, and then dissolved again in methylene chloride (MC). The mixture was columned with hexane and methylene chloride (MC), and the obtained material was concentrated. Impurities were removed using acetone slurry and filtered to obtain the compound D. (17.9 g, 54%)

### (3) Compound A83-i

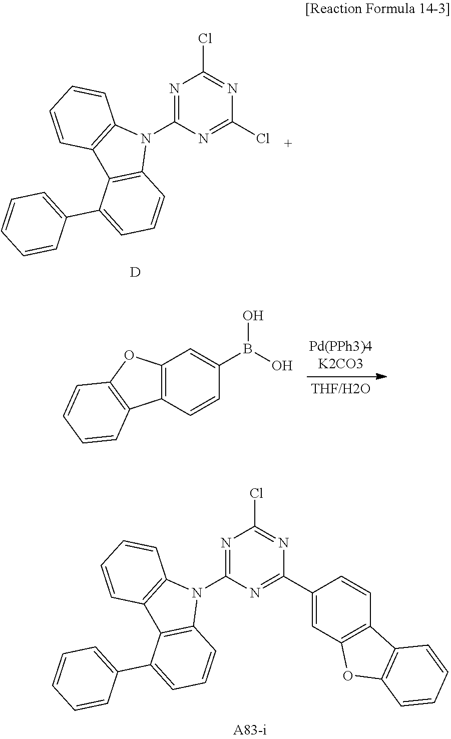

In a one-neck round-bottom flask, 9-(4,6-dichloro-1,3,5-triazin-2-yl)-4-phenyl-carbazole (10 g, 0.026 mol), dibenzofuran-3-ylboronic acid (4.8 g, 0.022 mol), palladium-tetrakis(triphenylphosphine) (Pd(PPh₃)₄, 1.5 g, 0.0013 mol) and K₂CO₃ (7.1 g, 0.051 mol) were dissolved in a mixture of tetrahydrofuran (100 mL) and water (20 mL) and refluxed at 70° C. for 2 hours. After the reaction solution was cooled to room temperature, an excess amount of methanol was added to completely precipitate the material and then filtered. The precipitated material was completely dissolved by heating with DCB (1,2-dichlrobenzene) and then filtered using chloroform and silica gel. The filtered solution was concentrated, impurities were removed using methanol slurry, and then filtered to obtain the compound A83-i. (9.9 g, 70%)

### (4) Compound A83

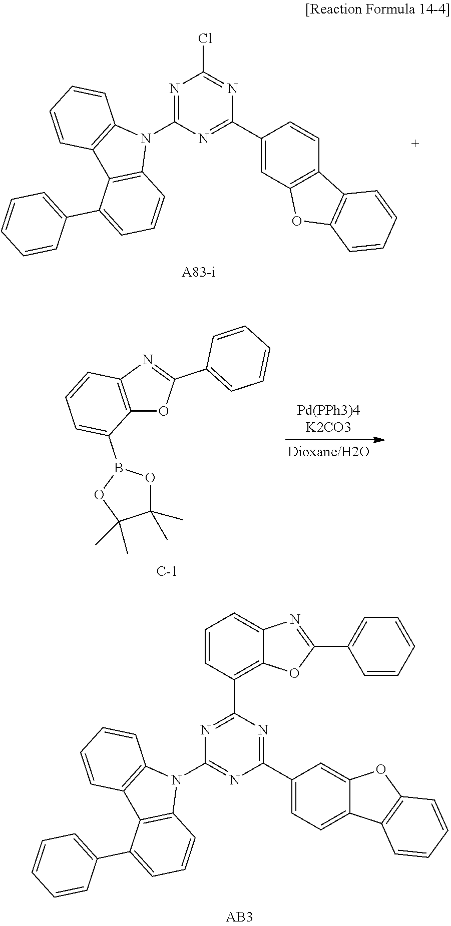

In a one-neck round-bottom flask, the compound A83-i (10.0 g, 0.017 mol), the compound C-1 (6.04 g, 0.018 mol), palladium-tetrakis(triphenylphosphine) (Pd(PPh₃)₄, 1 g, 0.0009 mol), and K₂CO₃ (4.7 g, 0.034 mol) were dissolved in a mixture of 1,4-dioxane (100 mL) and water (20 mL) and reacted under reflux for 4 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A83. (10.8 g, 82.9%)

### 15. Synthesis of the Compound A102

### (1) Compound 102-ii

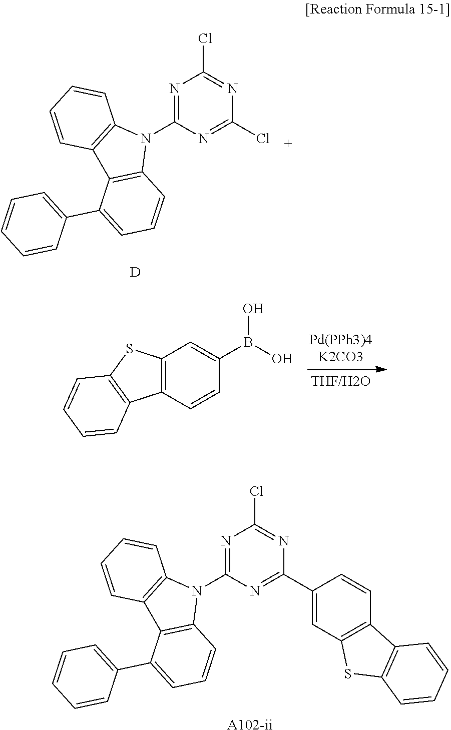

In a one-neck round-bottom flask, 9-(4,6-dichloro-1,3,5-triazin-2-yl)carbazole (10 g, 0.026 mol), dibnezothiophen-3-ylboronic acid (4.4 g, 0.020 mol), palladium-tetrakis(triphenylphosphine) (Pd(PPh₃)₄, 1.5 g, 0.0013 mol) and K₂CO₃ (7.07 g, 0.051 mol) were dissolved in a mixture of tetrahydrofuran (100 mL) and water (20 mL) and refluxed at 70° C. for 3 hours. After the reaction solution was cooled to room temperature, an excess amount of methanol was added to completely precipitate the material and then filtered. The precipitated material was completely dissolved by heating with DCB (1,2-dichlrobenzene) and then filtered using chloroform and silica gel. The filtered solution was concentrated, impurities were removed using methanol slurry, and then filtered to obtain the compound A102-i. (8.8 g, 64%)

### (2) Compound 102-i

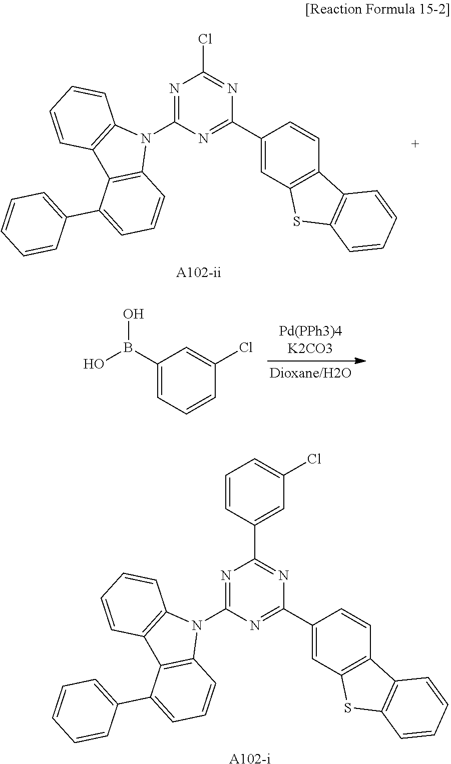 [SMILES: Clc1nc(-c2ccc3c(c2)sc2ccccc23)nc(-n2c3ccccc3c3c(-c4ccccc4)cccc32)n1 ; "A102-ii" ; OB(O)c1cccc(Cl)c1 ; Clc1cccc(-c2nc(-c3ccc4c(c3)sc3ccccc34)nc(-n3c4ccccc4c4c(-c5ccccc5)cccc43)n2)c1 ; "A102-i"]

In a one-neck round-bottom flask, the compound A102-ii (8.8 g, 0.016 mol), 3-chlorophenylboronic acid (3.1 g, 0.020 mol), palladium-tetrakis(triphenylphosphine) (Pd(PPh₃)₄, 0.9 g, 0.0008 mol) and K₂CO₃ (4.5 g, 0.032 mol) were dissolved in a mixture of tetrahydrofuran (100 mL) and water (20 mL) and refluxed at 110° C. for 4 hours. After the reaction solution was cooled to room temperature, the precipitated solid was filtered and washed with water and methanol. The filtered material was completely dissolved by heating with DCB and then filtered using chloroform and silica gel. The filtered solution was concentrated, impurities were removed using acetone slurry, and then filtered to obtain the compound A102-i. (8.1 g, 80.7%)

### (3) Compound 102

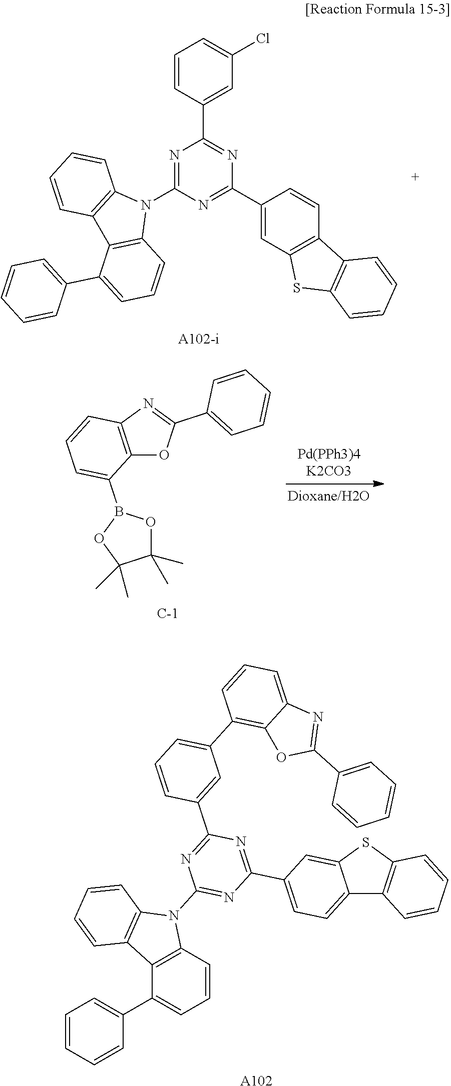

In a one-neck round-bottom flask, the compound A102-i (8.1 g, 0.013 mol), the compound C-1 (4.7 g, 0.015 mol), palladium-tetrakis(triphenylphosphine) (Pd(PPh₃)₄, 0.8 g, 0.0007 mol), and K₂CO₃ (3.6 g, 0.026 mol) were dissolved in a mixture of 1,4-dioxane (100 mL) and water (20 mL) and reacted under reflux for 8 hours at 110° C. After the reaction solution was sufficiently cooled to room temperature, the precipitated solid was filtered and thoroughly washed with water and methanol. The filtered material was completely dissolved by heating with DCB and then filtered using chloroform and silicagel. The obtained solution was concentrated, impurities were removed using acetone slurry, and then filtered to obtain the compound A102. (8.5 g, 83.4%)

### 16. Synthesis of the Compound 141

### (1) Compound 141-ii

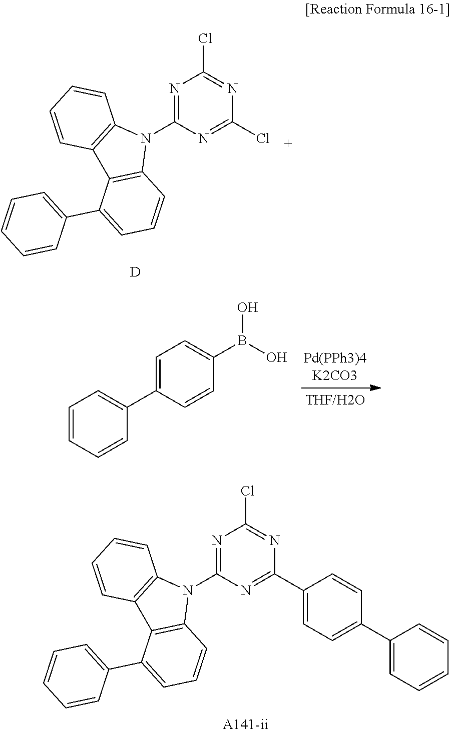

In a one-neck round-bottom flask, 9-(4,6-dichloro-1,3,5-triazin-2-yl)carbazole (10 g, 0.026 mol), [1,1′-biphenyl]-4-ylboronic acid (3.96 g, 0.020 mol), palladium-tetrakis(triphenylphosphine) (Pd(PPh₃)₄, 1.5 g, 0.0013 mol) and K₂CO₃ (7.07 g, 0.051 mol) were dissolved in a mixture of tetrahydrofuran (100 mL) and water (20 mL) and refluxed at 70° C. for 3 hours. After the reaction solution was cooled to room temperature, an excess amount of methanol was added to completely precipitate the material and then filtered. The precipitated material was completely dissolved by heating with DCB and then filtered using chloroform and silica gel. The filtered solution was concentrated, impurities were removed using methanol slurry, and then filtered to obtain the compound A141-i. (6.75 g, 67%)

### (2) Compound 141-i

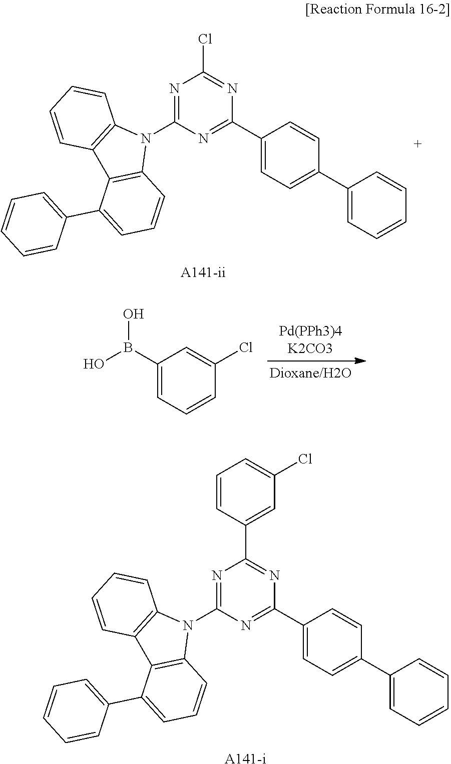 [SMILES: Clc1nc(-c2ccc(-c3ccccc3)cc2)nc(-n2c3ccccc3c3c(-c4ccccc4)cccc32)n1 ; "A141-ii" ; OB(O)c1cccc(Cl)c1 ; Clc1cccc(-c2nc(-c3ccc(-c4ccccc4)cc3)nc(-n3c4ccccc4c4c(-c5ccccc5)cccc43)n2)c1 ; "A141-i"]

In a one-neck round-bottom flask, the compound A141-ii (8.8 g, 0.016 mol), 3-chlorophenylboronic acid (2.5 g, 0.016 mol), palladium-tetrakis(triphenylphosphine) (Pd(PPh₃)₄, 0.9 g, 0.0008 mol) and K₂CO₃ (4.5 g, 0.032 mol) were dissolved in a mixture of tetrahydrofuran (100 mL) and water (20 mL) and refluxed at 110° C. for 4 hours. After the reaction solution was cooled to room temperature, the precipitated solid was filtered and washed with water and methanol. The filtered material was completely dissolved by heating with DCB and then filtered using chloroform and silica gel. The filtered solution was concentrated, impurities were removed using acetone slurry, and then filtered to obtain the compound A141-i. (6.2 g, 82.1%)

### (3) Compound 141

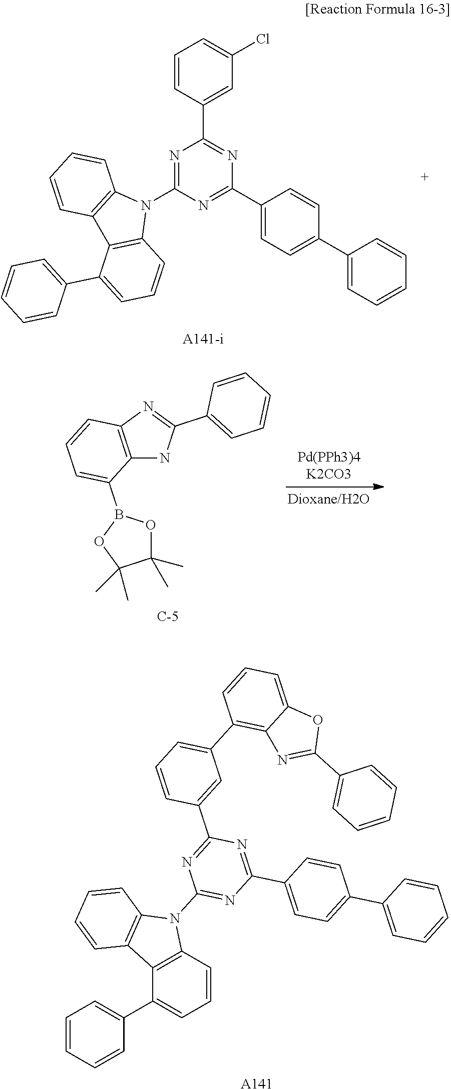

In the a-neck round-bottom flask, the compound A141-i (6.2 g, 0.0107 mol), the compound C-5 (4.7 g, 0.015 mol), palladium-tetrakis(triphenylphosphine) (Pd(PPh₃)₄, 0.8 g, 0.0007 mol), and K₂CO₃ (3.6 g, 0.026 mol)) were dissolved in a mixture of 1,4-dioxane (100 mL) and water (20 mL) and reacted under reflux for 8 hours at 110° C. After the reaction solution was sufficiently cooled to room temperature, the precipitated solid was filtered and thoroughly washed with water and methanol. The filtered material was completely dissolved by heating with DCB and then filtered using chloroform and silica gel. The obtained solution was concentrated, impurities were removed using acetone slurry, and then filtered to obtain the compound A141. (6.56 g, 82.9%)

The green EML 230 may further include a second compound 234 represented by Formula 3.

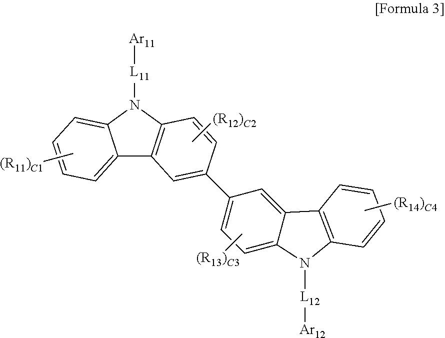

In Formula 3, each c1 and c4 is independently an integer of 0 to 4, each c2 and c3 is independently an integer of 0 to 3,
each R₁₁, R₁₂, R₁₃, and R₁₄ is independently selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each L₁₁ and L₁₂ is independently selected from the group consisting of a single bond, a substituted or unsubstituted C6 to C60 arylene group, and a substituted or unsubstituted C3 to C60 heteroarylene group, and
each Ar₁₁ and Ar₁₂ is independently selected from the group consisting of a substituted or unsubstituted C6 to C60 aryl group, and a substituted or unsubstituted C3 to C60 heteroaryl group.

When c1 is 2 or more, 2 or more Rn groups are the same or different. When c1 is 2 or more, 2 or more R₁₂ groups are the same or different. When c3 is 2 or more, 2 or more R₁₃ groups are the same or different. When c4 is 2 or more, 2 or more R₁₄ groups are the same or different.

In Formula 3, each L₁₁ and L₁₂ may be a single bond, and each Arii and Ar₁₂ may be independently selected from a substituted or unsubstituted phenyl. For example, Formula 3 may be represented by Formula 3a.

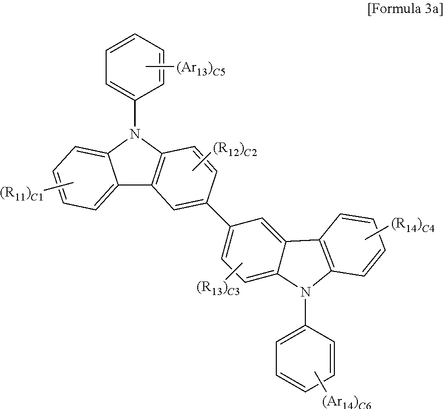

In Formula 3a, the definition of R₁₁, R₁₂, R₁₃, R₁₄, c1, c2, c3, and c4 is the same as in Formula 3,
each Ar₁₃ and Ar₁₄ is independently selected from a substituted or unsubstituted C6 to C30 aryl group, and
each c5 and c6 is independently an integer of 0 to 5.

For example, in Formula 3a, each Ar₁₃ and Ar₁₄ may be phenyl, and each c5 and c6 may be independently 0 or 1.

The second compound 234 may be one of the compounds in Formula 4.

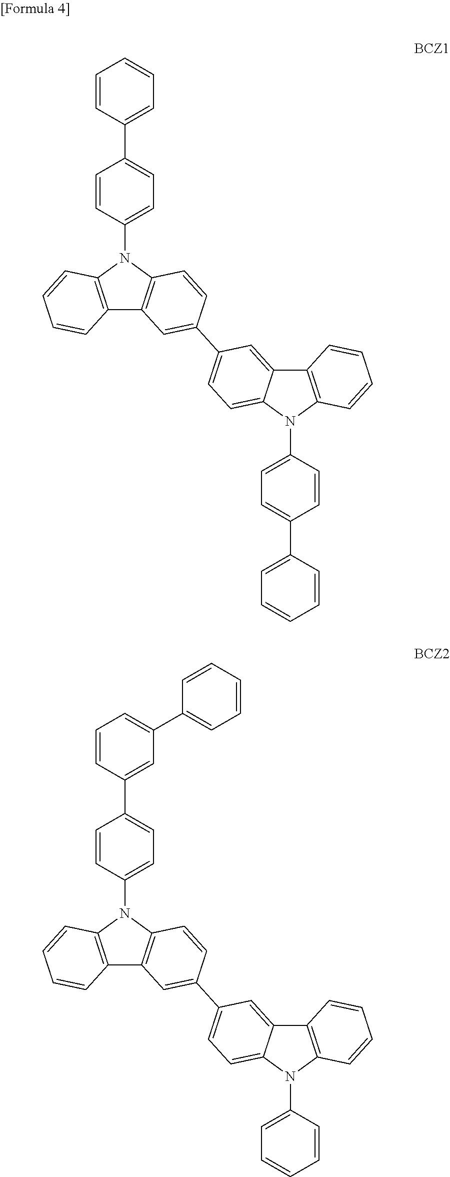 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

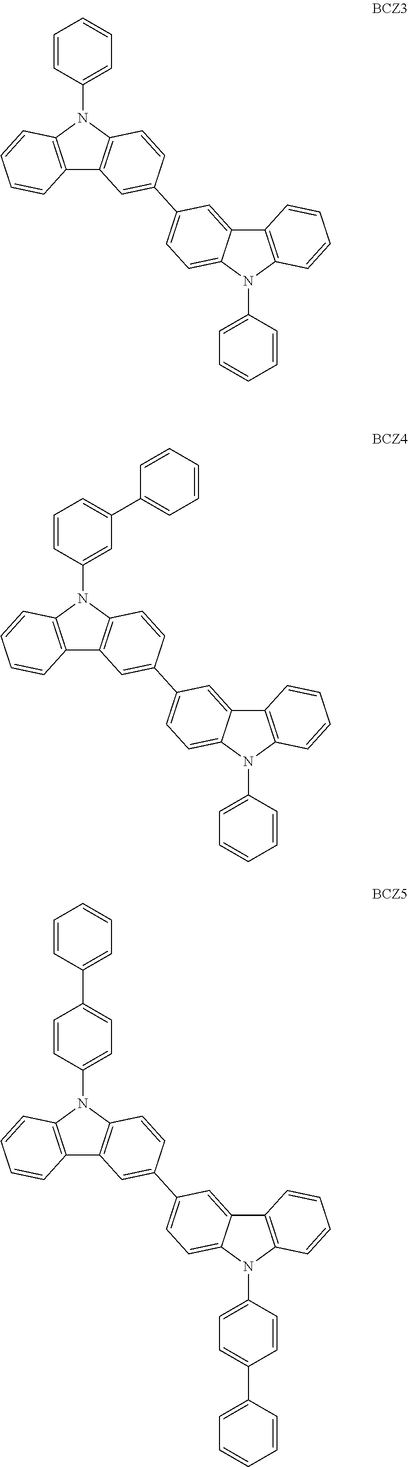 [SMILES: c1ccc(-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

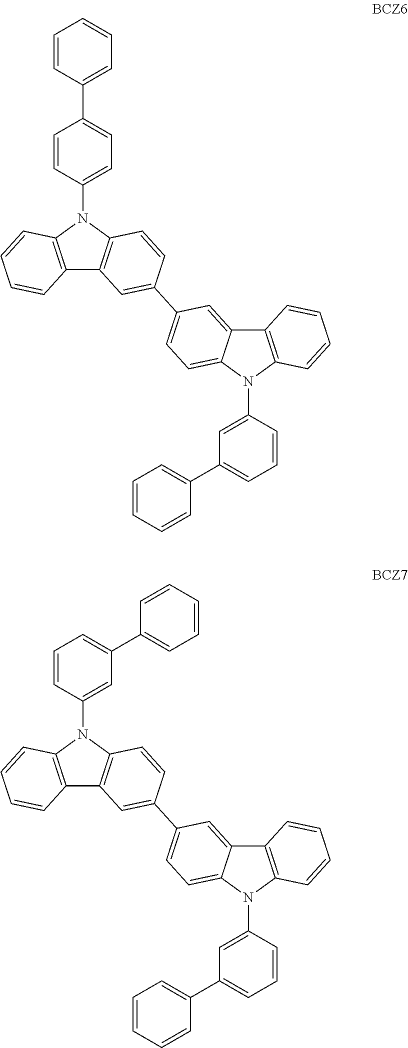 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

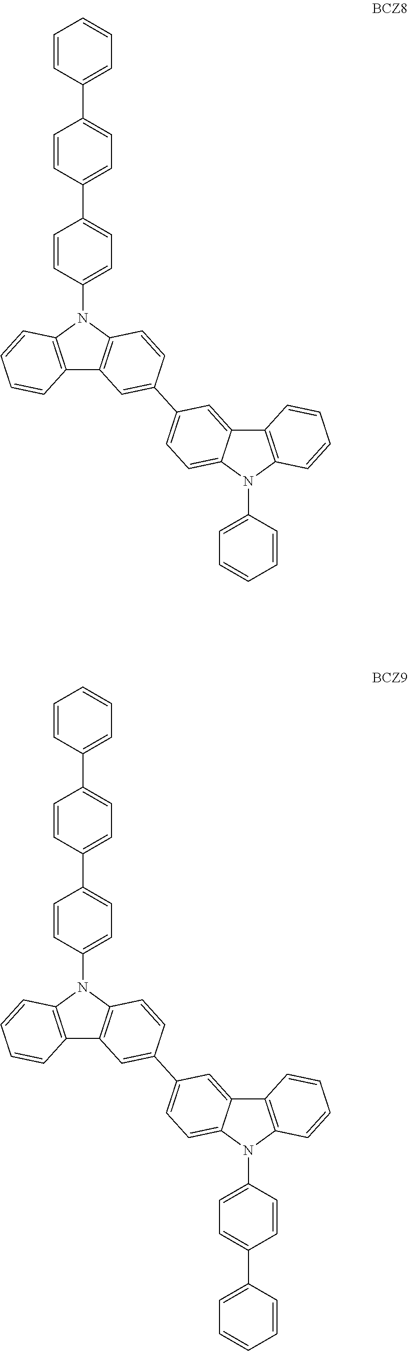 [SMILES: c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1]

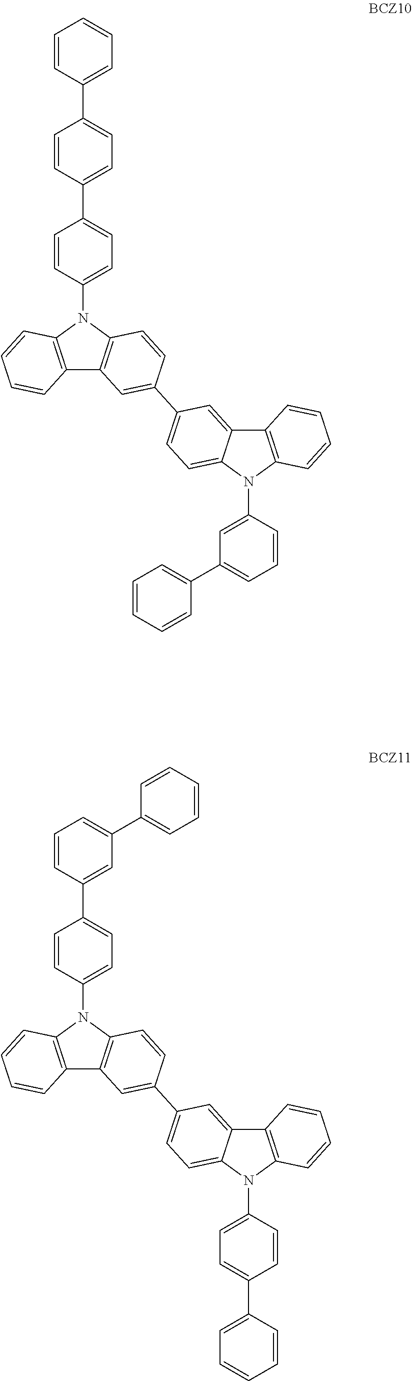 [SMILES: c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1]

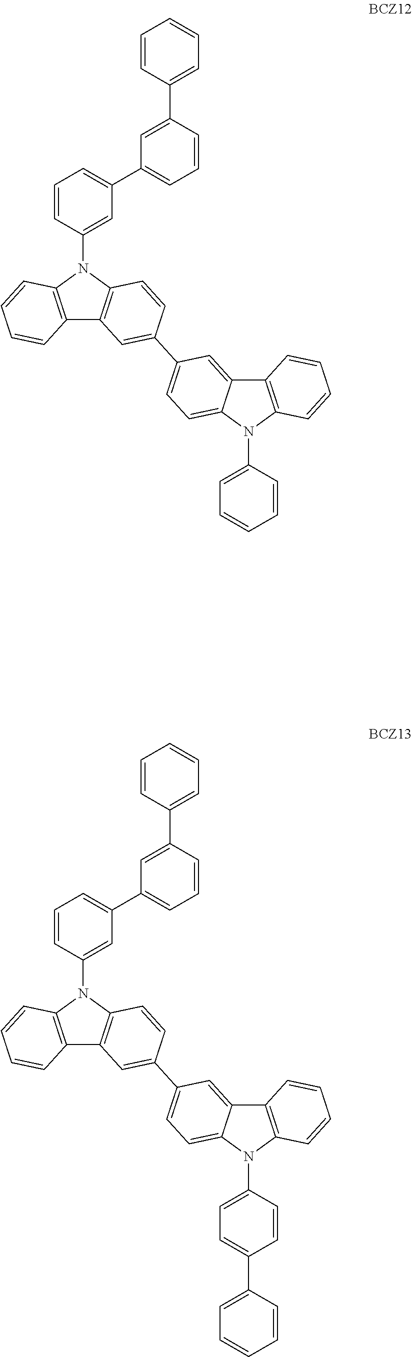 [SMILES: c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2ccc(-n3c4ccccc4c4cc(-c5ccc6c(c5)c5ccccc5n6-c5cccc(-c6cccc(-c7ccccc7)c6)c5)ccc43)cc2)cc1]

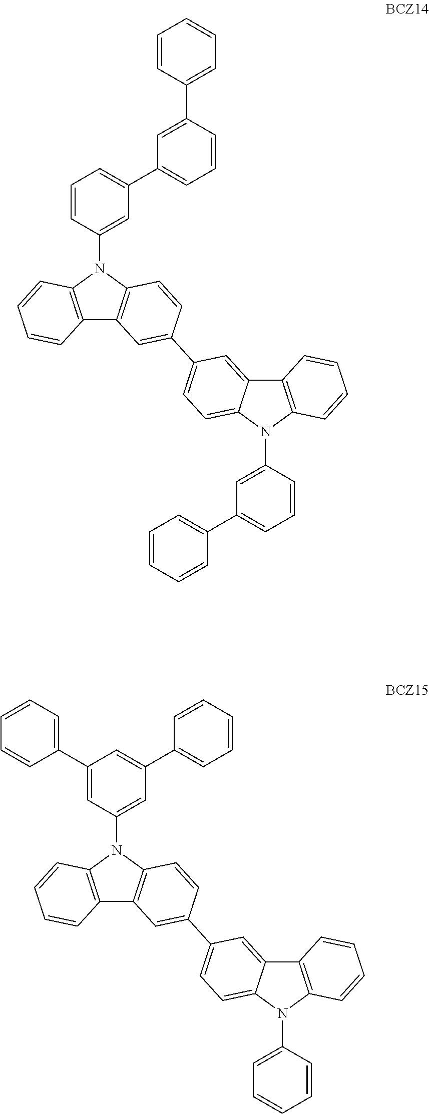 [SMILES: c1ccc(-c2cccc(-c3cccc(-n4c5ccccc5c5cc(-c6ccc7c(c6)c6ccccc6n7-c6cccc(-c7ccccc7)c6)ccc54)c3)c2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

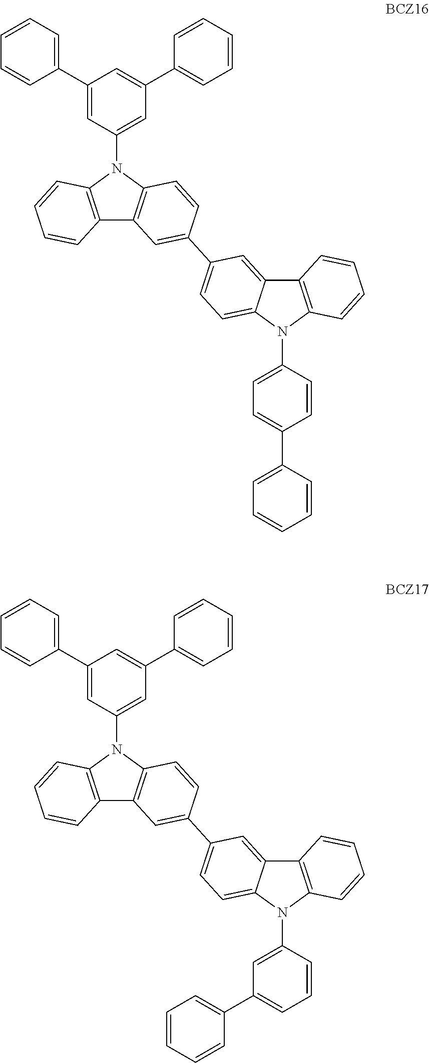 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

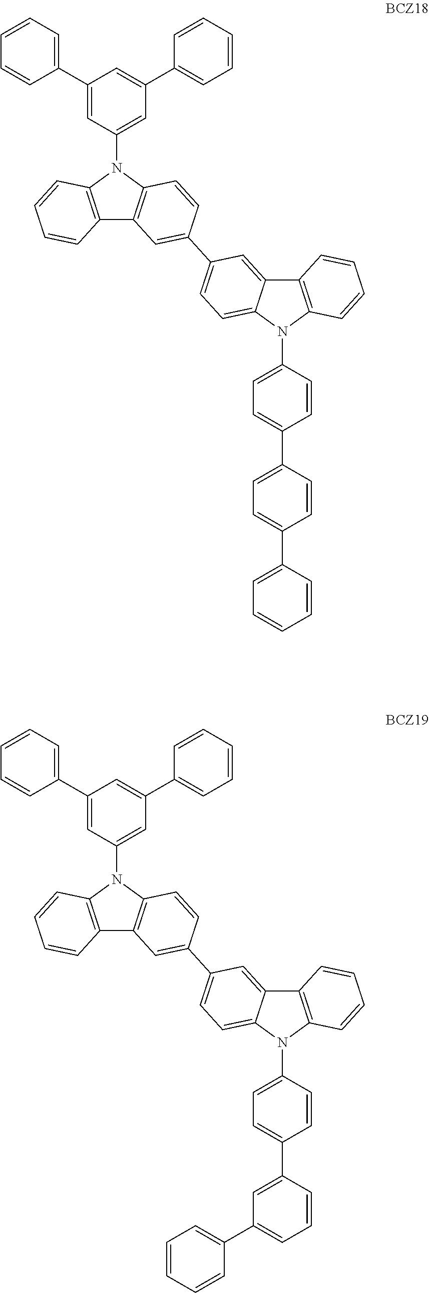 [SMILES: c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2cccc(-c3ccc(-n4c5ccccc5c5cc(-c6ccc7c(c6)c6ccccc6n7-c6cc(-c7ccccc7)cc(-c7ccccc7)c6)ccc54)cc3)c2)cc1]

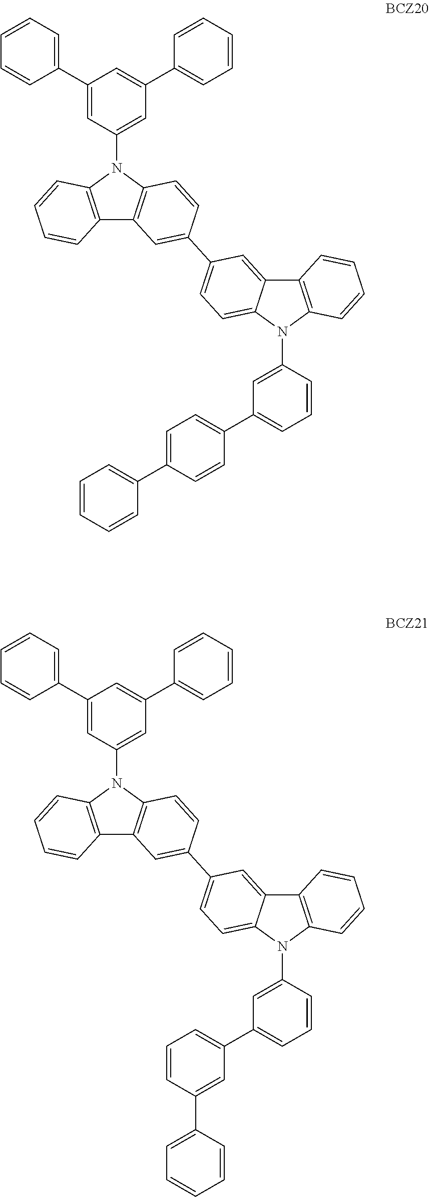 [SMILES: c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1 ; c1ccc(-c2cccc(-c3cccc(-n4c5ccccc5c5cc(-c6ccc7c(c6)c6ccccc6n7-c6cc(-c7ccccc7)cc(-c7ccccc7)c6)ccc54)c3)c2)cc1]

In the green pixel region, the EML 230 may include one of the compounds in Formula 5 as third compound 236.

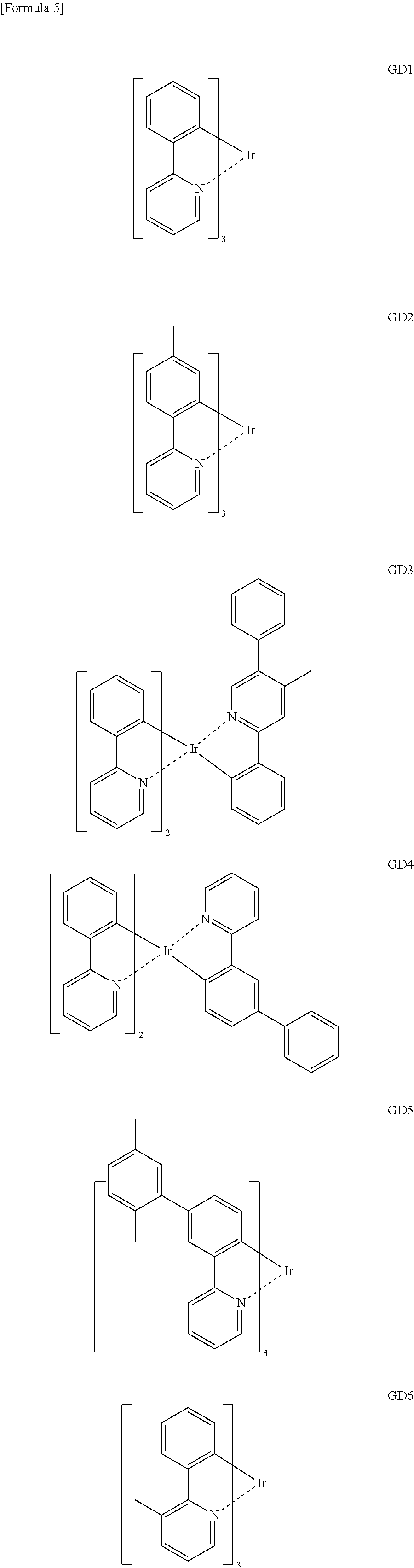

In the yellow-green pixel region, the EML 230 may include one of the compounds in Formula 6 as a third compound 236.

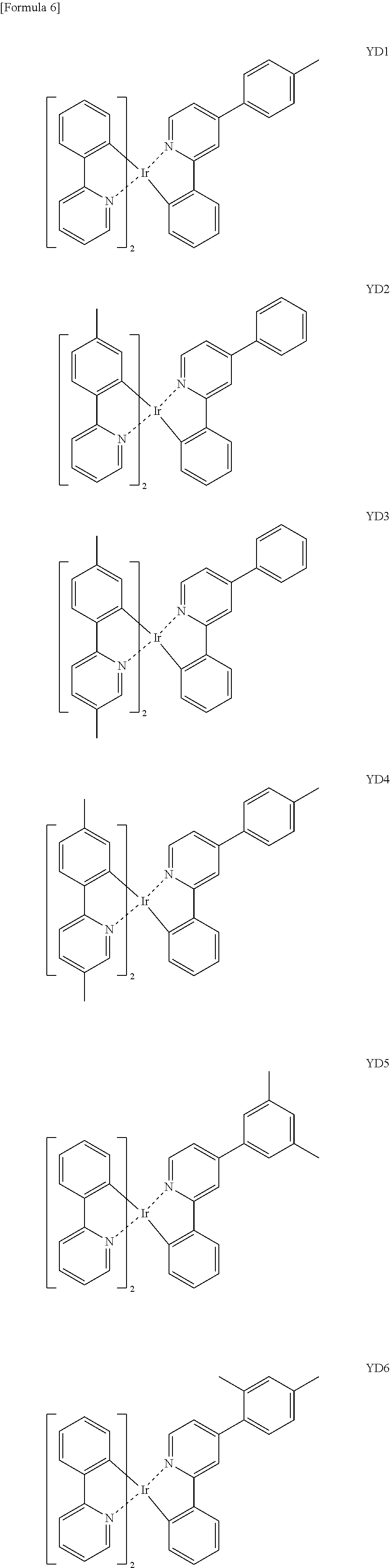

In the EML 230, the first compound 232 may be an n-type host (e.g., a first host), the second compound 234 may be a p-type host (e.g., a second host), and the third compound 236 may be an emitter (e.g., a dopant). The EML 230 may have a thickness of 50 to 600 Å, for example, 200 to 400 Å.

In the EML 230, a weight % of each of the first and second compounds 232 and 234 may be greater than that of the third compound 236. The weight % of the first compound 232 and the weight % of the second compound 234 may be the same or different. In the green EML 230, a weight % ratio of the first compound 232 to the second compound 234 may be 1:9 to 9:1, 2:8 to 8:2 or 7:3 to 3:7. In some embodiments, the weight % of the first compound 232 and the weight % of the second compound 234 may be same. For example, the first compound 232 and the second compound 234 may be present at the same weight %. The third compound 236 may be present in an amount of 5 to 25 weight % in the green EML 230, based on a total weight of the components in the green EML 230.

The organic light emitting layer 162 may further include an ETL 240 between the green EML 230 and the second electrode 164. For example, the ETL 240 may contact the green EML 230. The ETL 240 may have a thickness that may be substantially the same as the green EML 230. For example, the ETL 240 may have a thickness of 50 to 600 Å, for example, 200 to 400 Å.

The ETL 240 may include at least one of a compound (e.g., a first electron transporting material) represented by Formula 7, a compound (e.g., a second electron transporting material) represented by Formula 8 and a compound (e.g., a third electron transporting material) represented by Formula 9.

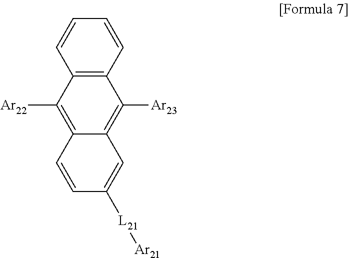

In Formula 7, L₂₁ is selected from the group consisting of a single bond, a substituted or unsubstituted C6 to C60 arylene group, and a substituted or unsubstituted C3 to C60 heteroarylene group,
Ar₂₁ is represented by Formula 7a or Formula 7b,
each Ar₂₂ and Ar₂₃ is independently selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,

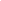

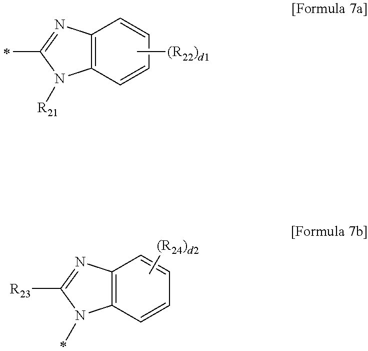

in Formula 7a, d1 is an integer of 0 to 4,
R₂₁ is selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each R₂₂ is independently selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
in Formula 7b, d2 is an integer of 0 to 4,
R₂₃ is selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each R₂₄ is independently selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group.

In each of Formulas 7a and b, the mark “*” denotes a bonding site.

In an aspect of the present disclosure, each Ar₂₂ and Ar₂₃ may be independently a C6 to C60 aryl group, e.g., phenyl or naphthyl, unsubstituted or substituted with a C1 to C10 alkyl group, e.g., tert-butyl.

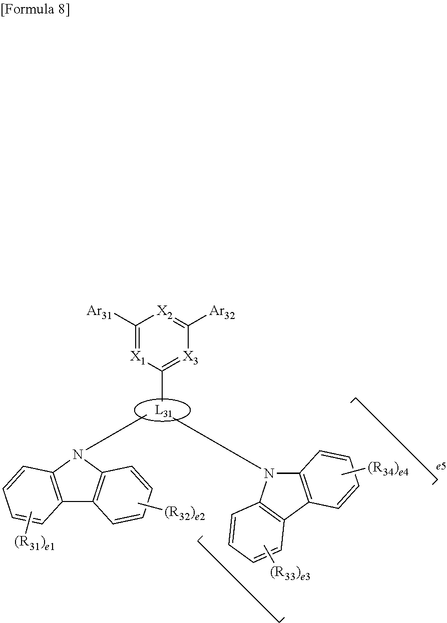

In Formula 8, each e1, e2, e3 and e4 is independently an integer of 0 to 4, and e5 is 0 or 1,
each R₃₁, R₃₂, R₃₃, and R₃₄ is independently selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each X₁, X₂ and X₃ is independently N or CR₃₅, wherein at least two of X₁, X₂ and X₃ are N,
each R₃₅ is independently selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each Ar₃₁ and Ar₃₂ is independently selected from the group consisting of a substituted or unsubstituted C6 to C60 aryl group, and a substituted or unsubstituted C3 to C60 heteroaryl group, and
L₃₁ is selected from the group consisting of a substituted or unsubstituted C6 to C60 arylene group, and a substituted or unsubstituted C3 to C60 heteroarylene group.

In an aspect of the present disclosure, each e1, e2, e3 and e4 may be 0 or 1.

In an aspect of the present disclosure, each R₃₁, R₃₂, R₃₃, and R₃₄ may be independently a substituted or unsubstituted C6 to C60 aryl group, e.g., phenyl.

In an aspect of the present disclosure, two of X₁, X₂ and X₃ may be N, the other one of X₁, X₂ and X₃ may be CR₃₅, and R₃₅ may be hydrogen.

In an aspect of the present disclosure, each Ar₃₁ and Ar₃₂ may be independently a substituted or unsubstituted C6 to C60 aryl group, e.g., phenyl or biphenyl.

In an aspect of the present disclosure, L₃₁ may be a substituted or unsubstituted C6 to C60 arylene group, e.g., phenylene.

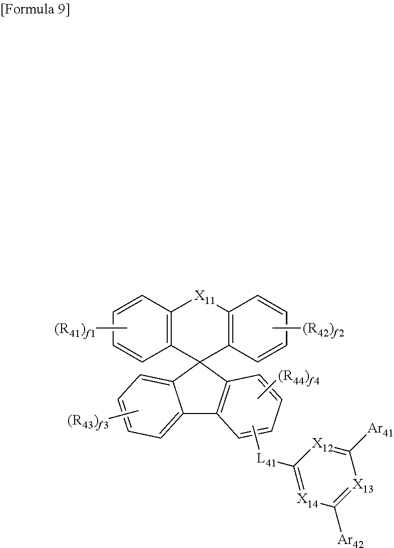

In Formula 9, each f1, f2 and f3 is independently an integer of 0 to 4, and f4 is an integer of 0 to 3,
each R₄₁, R₄₂, R₄₃, and R₄₄ is independently selected from the group consisting of a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
X₁₁ is O, S or NR₄₅,
X₄₅ is a substituted or unsubstituted C6 to C60 aryl group and form a ring with adjacent benzene ring,
each X₁₂, X₁₃ and X₁₄ is independently N or CR₄₆, wherein at least two of X₁₂, X₁₃ and X₁₄ are N,
each Ar₄₁ and Ar₄₂, R₄₆ is independently selected from the group consisting of hydrogen, a substituted or unsubstituted C6 to C60 aryl group, and a substituted or unsubstituted C3 to C60 heteroaryl group, and
L₄₁ is selected from the group consisting of a single bond, a substituted or unsubstituted C6 to C60 arylene group, and a substituted or unsubstituted C3 to C60 heteroarylene group.

In an aspect of the present disclosure, each f1, f2, f3 and f4 may independently be 0.

In an aspect of the present disclosure, R₄₅ may be phenyl and form carbazole with nitrogen atom and adjacent benzene ring.

In an aspect of the present disclosure, L₄₁ may be a substituted or unsubstituted C6 to C60 arylene group, e.g., phenylene.

In an aspect of the present disclosure, each Ar₄₁ and Ar₄₂ is independently a substituted or unsubstituted C6 to C60 aryl group, e.g., phenyl, naphthyl or naphthylphenyl.

In an aspect of the present disclosure, R₄₆ may be hydrogen.

The first electron transporting material of Formula 7 used as the electron transporting material 282 may be one of the compounds in Formula 10.

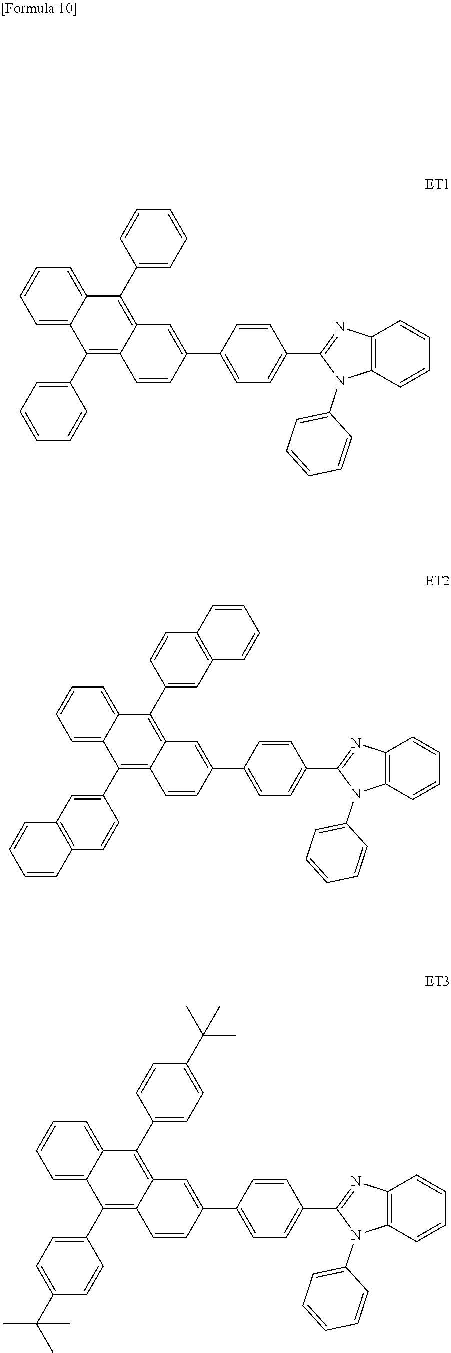 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CC(C)(C)c1ccc(-c2c3ccccc3c(-c3ccc(C(C)(C)C)cc3)c3cc(-c4ccc(-c5nc6ccccc6n5-c5ccccc5)cc4)ccc23)cc1]

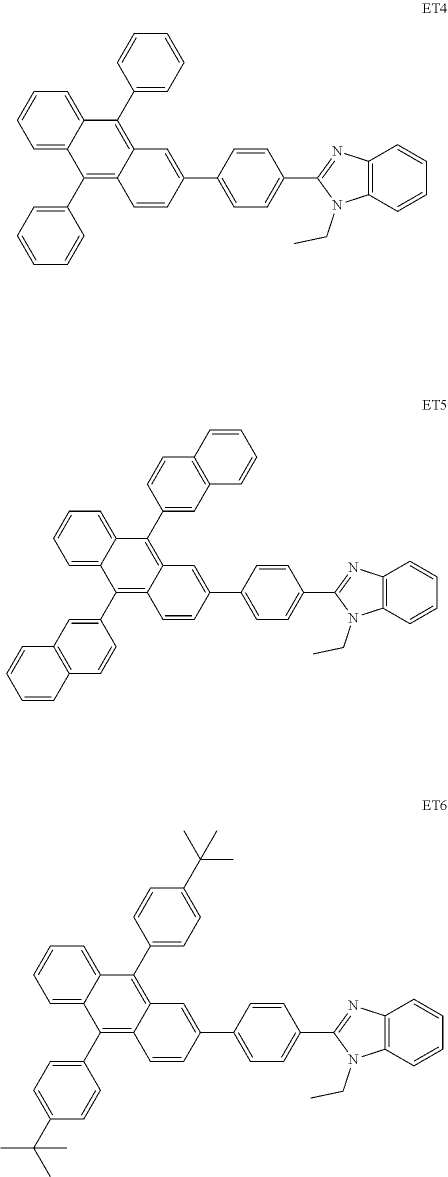 [SMILES: CCn1c(-c2ccc(-c3ccc4c(-c5ccccc5)c5ccccc5c(-c5ccccc5)c4c3)cc2)nc2ccccc21 ; CCn1c(-c2ccc(-c3ccc4c(-c5ccc6ccccc6c5)c5ccccc5c(-c5ccc6ccccc6c5)c4c3)cc2)nc2ccccc21 ; CCn1c(-c2ccc(-c3ccc4c(-c5ccc(C(C)(C)C)cc5)c5ccccc5c(-c5ccc(C(C)(C)C)cc5)c4c3)cc2)nc2ccccc21]

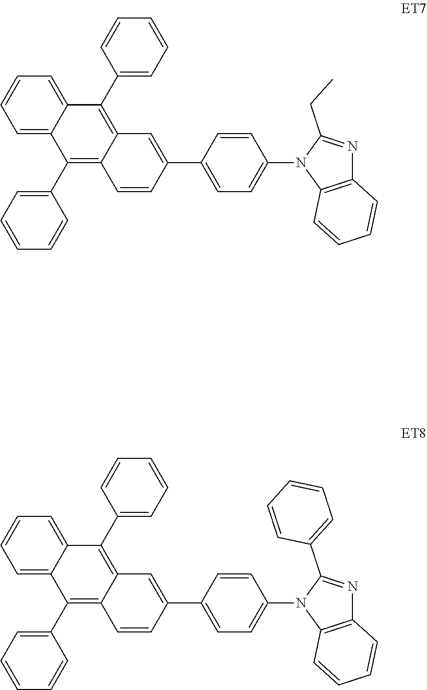 [SMILES: CCc1nc2ccccc2n1-c1ccc(-c2ccc3c(-c4ccccc4)c4ccccc4c(-c4ccccc4)c3c2)cc1 ; c1ccc(-c2c3ccccc3c(-c3ccccc3)c3cc(-c4ccc(-n5c(-c6ccccc6)nc6ccccc65)cc4)ccc23)cc1]

The second electron transporting material of Formula 8 used as the electron transporting material 282 may be one of the compounds in Formula 11.

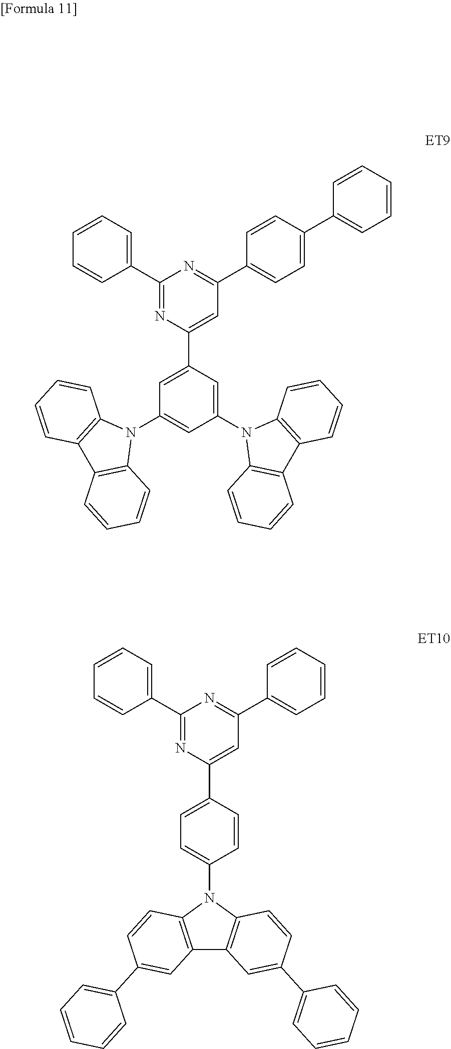 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2ccc3c(c2)c2cc(-c4ccccc4)ccc2n3-c2ccc(-c3cc(-c4ccccc4)nc(-c4ccccc4)n3)cc2)cc1]

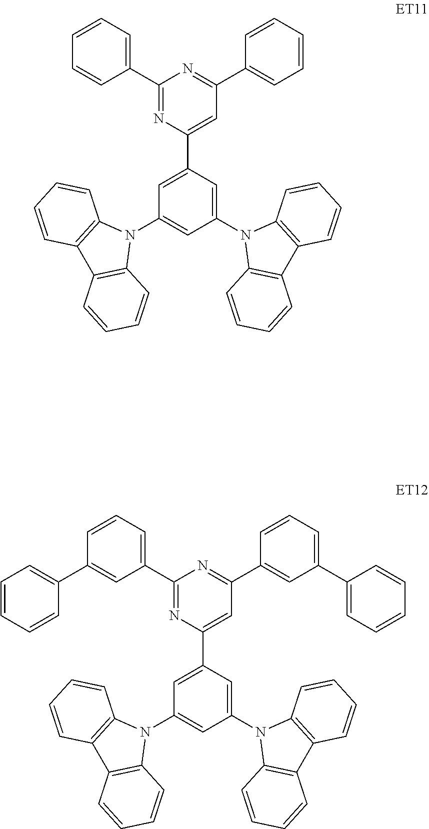 [SMILES: c1ccc(-c2cc(-c3cc(-n4c5ccccc5c5ccccc54)cc(-n4c5ccccc5c5ccccc54)c3)nc(-c3ccccc3)n2)cc1 ; c1ccc(-c2cccc(-c3cc(-c4cc(-n5c6ccccc6c6ccccc65)cc(-n5c6ccccc6c6ccccc65)c4)nc(-c4cccc(-c5ccccc5)c4)n3)c2)cc1]

The third electron transporting material of Formula 9 used as the electron transporting material 282 may be one of the compounds in Formula 12.

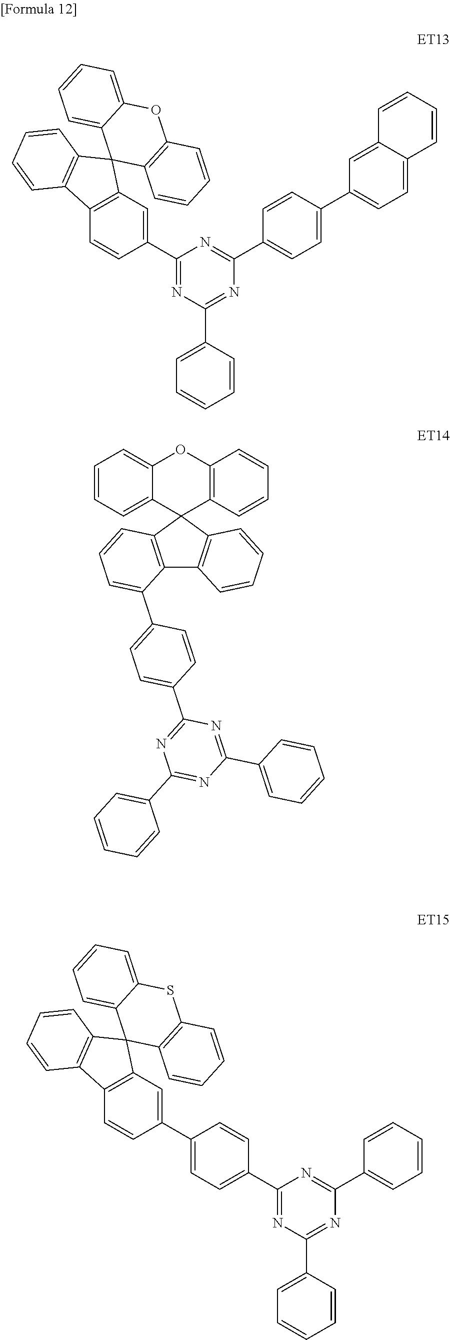 [SMILES: c1ccc(-c2nc(-c3ccc(-c4ccc5ccccc5c4)cc3)nc(-c3ccc4c(c3)C3(c5ccccc5Oc5ccccc53)c3ccccc3-4)n2)cc1 ; c1ccc(-c2nc(-c3ccccc3)nc(-c3ccc(-c4cccc5c4-c4ccccc4C54c5ccccc5Oc5ccccc54)cc3)n2)cc1 ; c1ccc(-c2nc(-c3ccccc3)nc(-c3ccc(-c4ccc5c(c4)C4(c6ccccc6Sc6ccccc64)c4ccccc4-5)cc3)n2)cc1]

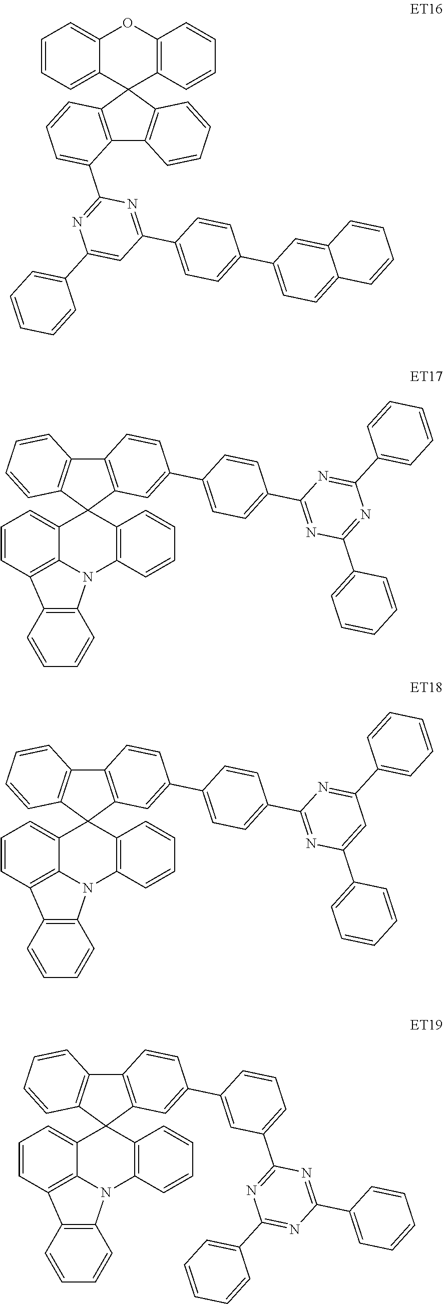 [SMILES: c1ccc(-c2cc(-c3ccc(-c4ccc5ccccc5c4)cc3)nc(-c3cccc4c3-c3ccccc3C43c4ccccc4Oc4ccccc43)n2)cc1 ; c1ccc(-c2nc(-c3ccccc3)nc(-c3ccc(-c4ccc5c(c4)C4(c6ccccc6-5)c5ccccc5-n5c6ccccc6c6cccc4c65)cc3)n2)cc1 ; c1ccc(-c2cc(-c3ccccc3)nc(-c3ccc(-c4ccc5c(c4)C4(c6ccccc6-5)c5ccccc5-n5c6ccccc6c6cccc4c65)cc3)n2)cc1 ; c1ccc(-c2nc(-c3ccccc3)nc(-c3cccc(-c4ccc5c(c4)C4(c6ccccc6-5)c5ccccc5-n5c6ccccc6c6cccc4c65)c3)n2)cc1]

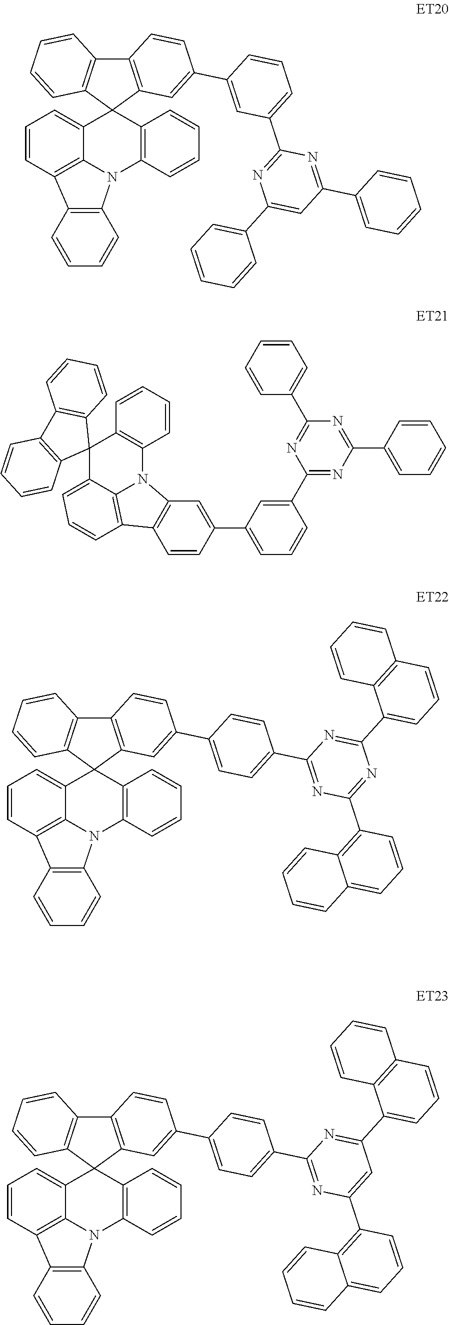 [SMILES: c1ccc(-c2cc(-c3ccccc3)nc(-c3cccc(-c4ccc5c(c4)C4(c6ccccc6-5)c5ccccc5-n5c6ccccc6c6cccc4c65)c3)n2)cc1 ; c1ccc(-c2nc(-c3ccccc3)nc(-c3cccc(-c4ccc5c6cccc7c6n(c5c4)-c4ccccc4C74c5ccccc5-c5ccccc54)c3)n2)cc1 ; c1ccc2c(c1)-c1ccc(-c3ccc(-c4nc(-c5cccc6ccccc56)nc(-c5cccc6ccccc56)n4)cc3)cc1C21c2ccccc2-n2c3ccccc3c3cccc1c32 ; c1ccc2c(c1)-c1ccc(-c3ccc(-c4nc(-c5cccc6ccccc56)cc(-c5cccc6ccccc56)n4)cc3)cc1C21c2ccccc2-n2c3ccccc3c3cccc1c32]

The organic light emitting layer 162 may further include an HTL 220 between the first electrode 160 and the green EML 230. A thickness of the HTL 220 may be greater than that of each of the green EML 230 and the ETL 240. For example, the HTL may have a thickness of 800 to 1200 Å, for example, 900 to 1100 Å.

In addition, the organic light emitting layer 162 may further include at least one of an HIL 210 between the first electrode 160 and the HTL 220 and an EIL 250 between the second electrode 164 and the ETL 240.

Although not shown, the organic light emitting layer 162 may further include at least one of an EBL between the HTL 220 and the green EML 230 and an HBL between the green EML 230 and the ETL 240.

The HIL 210 may include at least one compound selected from the group consisting of 4,4′,4″-tris(3-methylphenylamino)triphenylamine (MTDATA), 4,4′,4″-tris(N,N-diphenyl-amino)triphenylamine (NATA), 4,4′,4″-tris(N-(naphthalene-1-yl)-N-phenyl-amino)triphenylamine (1T-NATA), 4,4′,4″-tris(N-(naphthalene-2-yl)-N-phenyl-amino)triphenylamine (2T-NATA), copper phthalocyanine (CuPc), tris(4-carbazoyl-9-yl-phenyl)amine (TCTA), N,N′-diphenyl-N,N′-bis(1-naphthyl)-1,1′-biphenyl-4,4″-diamine (NPB or NPD), 1,4,5,8,9,11-hexaazatriphenylenehexacarbonitrile(dipyrazino[2,3-f:2′3′-h]quinoxaline-2,3,6,7,10,11-hexacarbonitrile (HAT-CN), 1,3,5-tris[4-(diphenylamino)phenyl]benzene (TDAPB), poly(3,4-ethylenedioxythiphene)polystyrene sulfonate (PEDOT/PSS), and N-(biphenyl-4-yl)-9,9-dimethyl-N-(4-(9-phenyl-9H-carbazol-3-yl)phenyl)-9H-fluoren-2-amine. The HIL 210 may have a thickness of 10 to 100 Å, for example, 30 to 70 Å.

The HTL 220 may include at least one compound selected from the group consisting of N,N′-diphenyl-N,N′-bis(3-methylphenyl)-1,1′-biphenyl-4,4′-diamine (TPD), NPB (or NPD), 4,4′-bis(N-carbazolyl)-1,1′-biphenyl (CBP), poly[N,N′-bis(4-butylphenyl)-N,N′-bis(phenyl)-benzidine](poly-TPD), (poly[(9,9-dioctylfluorenyl-2,7-diyl)-co-(4,4′-(N-(4-sec-butylphenyl)diphenylamine))](TFB), di-[4-(N,N-di-p-tolyl-amino)-phenyl]cyclohexane (TAPC), 3,5-di(9H-carbazol-9-yl)-N,N-diphenylaniline (DCDPA), N-(biphenyl-4-yl)-9,9-dimethyl-N-(4-(9-phenyl-9H-carbazol-3-yl)phenyl)-9H-fluoren-2-amine, and N-(biphenyl-4-yl)-N-(4-(9-phenyl-9H-carbazol-3-yl)phenyl)biphenyl-4-amine. Alternatively, the HTL 220 may include a compound of Formula 10 below. However, embodiments of the present disclosure are not limited to such examples.

The EIL 250 may include at least one of an alkali metal, such as L₁, an alkali halide compound, such as LiF, CsF, NaF, or BaF₂, and an organo-metallic compound, such as Liq, lithium benzoate, or sodium stearate. The EIL 250 may have a thickness of 10 to 100 Å, for example, 30 to 70 Å.

The EBL may include at least one compound selected from the group consisting of tris(4-carbazoyl-9-yl-phenyl)amine (TCTA), tris[4-(diethylamino)phenyl]amine, N-(biphenyl-4-yl)-9,9-dimethyl-N-(4-(9-phenyl-9H-carbazol-3-yl)phenyl)-9H-fluoren-2-amine, di-[4-(N,N-di-p-tolyl-amino)-phenyl]cyclohexane (TAPC), 4,4′,4″-tris(3-methylphenylamino)triphenylamine (MTDATA), 1,3-bis(carbazol-9-yl)benzene (mCP), 3,3′-bis(N-carbazolyl)-1,1′-biphenyl (mCBP), copper phthalocyanine (CuPc), N,N′-bis[4-[bis(3-methylphenyl)amino]phenyl]-N,N′-diphenyl-[1,1′-biphenyl]-4,4′-diamine (DNTPD), 1,3,5-tris[4-(diphenylamino)phenyl]benzene (TDAPB), 3,5-di(9H-carbazol-9-yl)-N,N-diphenylaniline (DCDPA) and 2,8-bis(9-phenyl-9H-carbazol-3-yl)dibenzo[b,d]thiophene).

The HBL may include at least one compound selected from the group consisting of 2,9-dimethyl-4,7-diphenyl-1,10-phenathroline (BCP), bis(2-methyl-8-quinolinolato-N1,O8)-(1,1′-biphenyl-4-olato)aluminium (BAlq), tris-(8-hydroxyquinoline aluminium (Alq₃), 2-biphenyl-4-yl-5-(4-t-butylphenyl)-1,3,4-oxadiazole (PBD), spiro-PBD, Liq, bis-4,6-(3,5-di-3-pyridylphenyl)-2-methylpyrimidine (B3PYMPM), bis[2-(diphenylphosphino)phenyl]ether oxide (DPEPO), 9-(6-9H-carbazol-9-yl)pyridine-3-yl)-9H-3,9′-bicarbazole, and diphenyl-4-triphenylsilyl-phenylphosphine oxide (TSPO1).

As illustrated above, in the OLED D in at least one of the green pixel region and the yellow-green pixel region, the EML 230 may include the first compound 232 that is the organic compound of the present disclosure and represented by Formula 1. As a result, in the OLED D, the driving voltage may be reduced, and the emitting efficiency and the lifespan may be improved.

In the OLED D positioned in at least one of the green pixel region and the yellow-green pixel region, the EML 230 may further include the second compound 234 as a second host being represented by Formula 3 with the first compound 232 as a first host so that the OLED D and the organic light emitting display device 100 have big improvements in driving voltage, emitting efficiency and lifespan.

In addition, in the OLED D of the green pixel region, the EML 230 may further include the third compound 236 as a dopant being one of the compounds in Formula 5 with the first compound 232 as a first host and the second compound 234 as a second host so that the OLED D and the organic light emitting display device 100 have big improvements in driving voltage, emitting efficiency and lifespan.

In addition, in the OLED D of the yellow-green pixel region, the EML 230 may further include the third compound 236 as a dopant being one of the compounds in Formula 6 with the first compound 232 as a first host and the second compound 234 as a second host so that the OLED D and the organic light emitting display device 100 have big improvements in driving voltage, emitting efficiency and lifespan.

The OLED D positioned in at least one of the green pixel region and the yellow-green pixel region may further include the ETL 250, which is positioned between the EML 240 and the second electrode 164 as a cathode and includes at least one of the first electron transporting material represented by Formula 7, the second electron transporting material represented by Formula 8 and the third electron transporting material represented by Formula 9 so that the OLED D and the organic light emitting display device 100 have big improvements in driving voltage, emitting efficiency and lifespan.

### [OLED]

An anode (ITO), an HIL (e.g., the compound in Formula 13, 50 Å), an HTL (e.g., the compound in Formula 14, 1000 Å), a green EML (e.g., a first host, a second host, and a dopant (the compound GD1 in Formula 5, 15 wt %), 300 Å), an ETL (e.g., the compound ET2 in Formula 10, 300 Å), an EIl (e.g., LiF, 50 Å) and a cathode (e.g., A1, 1000 Å) were sequentially deposited to form the OLED.

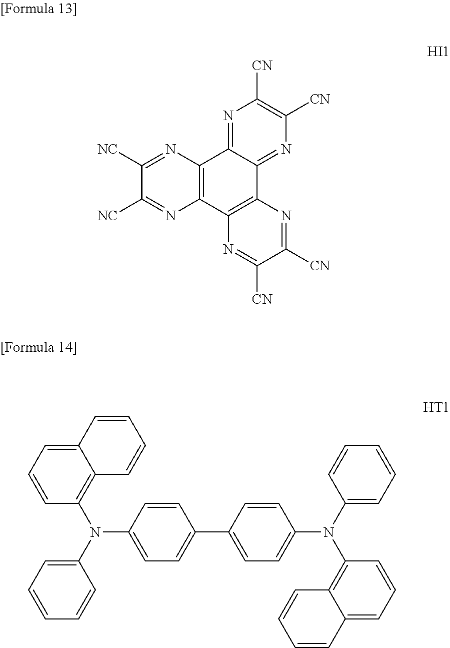 [SMILES: N#Cc1nc2c3nc(C#N)c(C#N)nc3c3nc(C#N)c(C#N)nc3c2nc1C#N ; c1ccc(N(c2ccc(-c3ccc(N(c4ccccc4)c4cccc5ccccc45)cc3)cc2)c2cccc3ccccc23)cc1]

### 1. Comparative Example

### (1) Comparative Example 1 (Ref1)

The compound CF1 in Formula 15 was used as the first host and the compound BCZ1 in Formula 4 was used as the second host to form the green EML. (a weight ratio of (the first host):(the second host)=1:1)

### (2) Comparative Example 2 (Ref2)

The compound CF2 in Formula 15 was used as the first host and the compound BCZ1 in Formula 4 was used as the second host to form the green EML. (a weight ratio of (the first host):(the second host)=1:1)

### (3) Comparative Example 3 (Ref3)

The compound CF3 in Formula 15 was used as the first host and the compound BCZ1 in Formula 4 was used as the second host to form the green EML. (a weight ratio of (the first host):(the second host)=1:1)

### (4) Comparative Example 4 (Ref4)

The compound CF4 in Formula 15 was used as the first host and the compound BCZ1 in Formula 4 was used as the second host to form the green EML. (a weight ratio of (the first host):(the second host)=1:1)

### (5) Comparative Example 5 (Ref5)

The compound CF5 in Formula 15 was used as the first host and the compound BCZ1 in Formula 4 was used as the second host to form the green EML. (a weight ratio of (the first host):(the second host)=1:1)

### (6) Comparative Example 6 (Ref6)

The compound CF6 in Formula 15 was used as the first host and the compound BCZ1 in Formula 4 was used as the second host to form the green EML. (a weight ratio of (the first host):(the second host)=1:1)

### (7) Comparative Example 7 (Ref7)

The compound CF7 in Formula 15 was used as the first host and the compound BCZ1 in Formula 4 was used as the second host to form the green EML. (a weight ratio of (the first host):(the second host)=1:1)

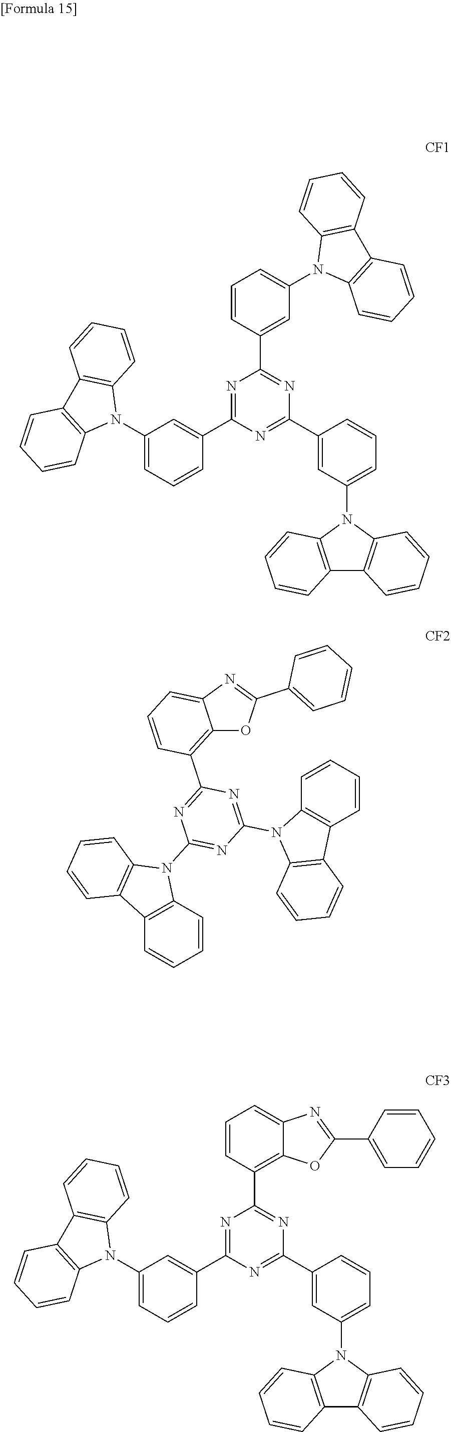 [SMILES: c1cc(-c2nc(-c3cccc(-n4c5ccccc5c5ccccc54)c3)nc(-c3cccc(-n4c5ccccc5c5ccccc54)c3)n2)cc(-n2c3ccccc3c3ccccc32)c1 ; c1ccc(-c2nc3cccc(-c4nc(-n5c6ccccc6c6ccccc65)nc(-n5c6ccccc6c6ccccc65)n4)c3o2)cc1 ; c1ccc(-c2nc3cccc(-c4nc(-c5cccc(-n6c7ccccc7c7ccccc76)c5)nc(-c5cccc(-n6c7ccccc7c7ccccc76)c5)n4)c3o2)cc1]

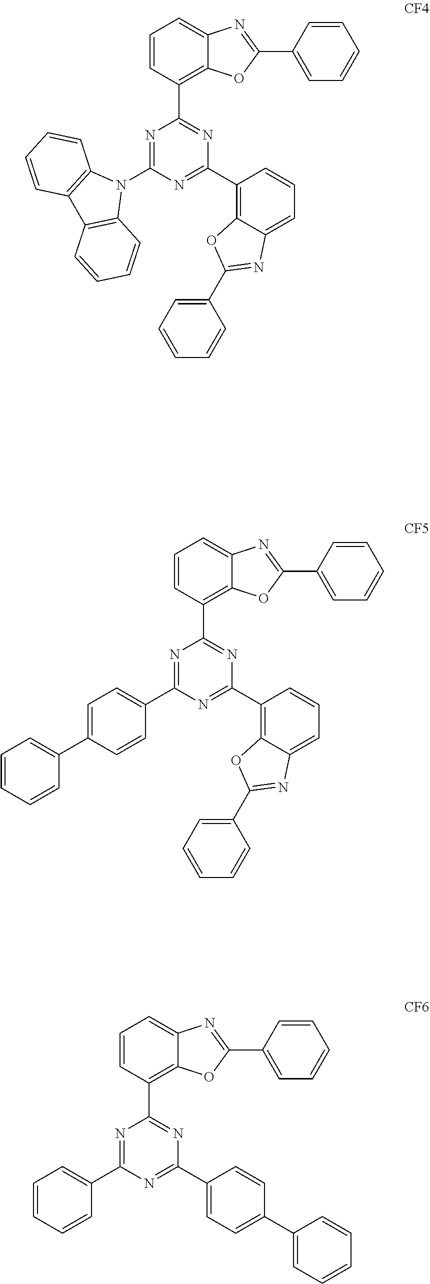 [SMILES: c1ccc(-c2nc3cccc(-c4nc(-c5cccc6nc(-c7ccccc7)oc56)nc(-n5c6ccccc6c6ccccc65)n4)c3o2)cc1 ; c1ccc(-c2ccc(-c3nc(-c4cccc5nc(-c6ccccc6)oc45)nc(-c4cccc5nc(-c6ccccc6)oc45)n3)cc2)cc1 ; c1ccc(-c2ccc(-c3nc(-c4ccccc4)nc(-c4cccc5nc(-c6ccccc6)oc45)n3)cc2)cc1]

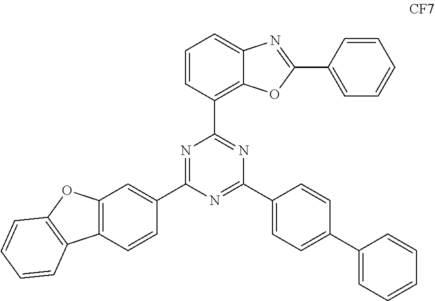 [SMILES: c1ccc(-c2ccc(-c3nc(-c4ccc5c(c4)oc4ccccc45)nc(-c4cccc5nc(-c6ccccc6)oc45)n3)cc2)cc1]

### 2. Example

### (1) Example 1 (Ex1)

The compound A1 in Formula 2 was used as the first host and the compound BCZ1 in Formula 4 was used as the second host to form the green EML. (a weight ratio of (the first host):(the second host)=1:1)

### (2) Example 2 (Ex2)

The compound A5 in Formula 2 was used as the first host and the compound BCZ1 in Formula 4 was used as the second host to form the green EML. (a weight ratio of (the first host):(the second host)=1:1)

### (3) Example 3 (Ex3)

The compound A6 in Formula 2 was used as the first host and the compound BCZ1 in Formula 4 was used as the second host to form the green EML. (a weight ratio of (the first host):(the second host)=1:1)

### (4) Example 4 (Ex4)

The compound A7 in Formula 2 was used as the first host and the compound BCZ1 in Formula 4 was used as the second host to form the green EML. (a weight ratio of (the first host):(the second host)=1:1)

### (5) Example 5 (Ex5)

The compound A8 in Formula 2 was used as the first host and the compound BCZ1 in Formula 4 was used as the second host to form the green EML. (a weight ratio of (the first host):(the second host)=1:1)

### (6) Example 6 (Ex6)

The compound A9 in Formula 2 was used as the first host and the compound BCZ1 in Formula 4 was used as the second host to form the green EML. (a weight ratio of (the first host):(the second host)=1:1)

### (7) Example 7 (Ex7)

The compound A19 in Formula 2 was used as the first host and the compound BCZ1 in Formula 4 was used as the second host to form the green EML. (a weight ratio of (the first host):(the second host)=1:1)

### (8) Example 8 (Ex8)

The compound A29 in Formula 2 was used as the first host and the compound BCZ1 in Formula 4 was used as the second host to form the green EML. (a weight ratio of (the first host):(the second host)=1:1)

### (9) Example 9 (Ex9)

The compound A30 in Formula 2 was used as the first host and the compound BCZ1 in Formula 4 was used as the second host to form the green EML. (a weight ratio of (the first host):(the second host)=1:1)

### (10) Example 10 (Ex10)

The compound A49 in Formula 2 was used as the first host and the compound BCZ1 in Formula 4 was used as the second host to form the green EML. (a weight ratio of (the first host):(the second host)=1:1)

### (11) Example 11 (Ex11)

The compound A63 in Formula 2 was used as the first host and the compound BCZ1 in Formula 4 was used as the second host to form the green EML. (a weight ratio of (the first host):(the second host)=1:1)

### (12) Example 12 (Ex12)

The compound A83 in Formula 2 was used as the first host and the compound BCZ1 in Formula 4 was used as the second host to form the green EML. (a weight ratio of (the first host):(the second host)=1:1)

### (13) Example 13 (Ex13)

The compound A102 in Formula 2 was used as the first host and the compound BCZ1 in Formula 4 was used as the second host to form the green EML. (a weight ratio of (the first host):(the second host)=1:1)

### (14) Example 14 (Ex14)

The compound A105 in Formula 2 was used as the first host and the compound BCZ1 in Formula 4 was used as the second host to form the green EML. (a weight ratio of (the first host):(the second host)=1:1)

### (15) Example 15 (Ex15)

The compound A116 in Formula 2 was used as the first host and the compound BCZ1 in Formula 4 was used as the second host to form the green EML. (a weight ratio of (the first host):(the second host)=1:1)

### (16) Example 16 (Ex16)

The compound A128 in Formula 2 was used as the first host and the compound BCZ1 in Formula 4 was used as the second host to form the green EML. (a weight ratio of (the first host):(the second host)=1:1)

### (17) Example 17 (Ex17)

The compound A132 in Formula 2 was used as the first host and the compound BCZ1 in Formula 4 was used as the second host to form the green EML. (a weight ratio of (the first host):(the second host)=1:1)

### (18) Example 18 (Ex18)

The compound A141 in Formula 2 was used as the first host and the compound BCZ1 in Formula 4 was used as the second host to form the green EML. (a weight ratio of (the first host):(the second host)=1:1)

### (19) Examples 19 to 36 (Ex19 to Ex36)

The compound BCZ2 in Formula 4 was used as the second host instead of the compound BCZ1 of Examples 1 to 18.

The properties, e.g., the driving voltage (AV) and the emitting efficiency, of the OLED in Comparative Examples 1 to 7 and Examples 1 to 36 were measured and listed in Tables 1 to 3.

TABLE 1

| | Host2 | Host1 | ΔV | Efficiency |
|---|---|---|---|---|
| Ref1 | BCZ1 | CF1 | 0.00 | 100% |
| Ref2 | BCZ1 | CF2 | −0.03 | 103% |
| Ref3 | BCZ1 | CF3 | −0.07 | 108% |
| Ref4 | BCZ1 | CF4 | −0.11 | 113% |
| Ref5 | BCZ1 | CF5 | 0.27 | 87% |
| Ref6 | BCZ1 | CF6 | 0.31 | 83% |
| Ref7 | BCZ1 | CF7 | 0.24 | 90% |

TABLE 2

| | Host2 | Host1 | ΔV | Efficiency |
|---|---|---|---|---|
| Ex1 | BCZ1 | A1 | −0.20 | 123% |
| Ex2 | BCZ1 | A5 | −0.22 | 126% |
| Ex3 | BCZ1 | A6 | −0.23 | 126% |
| Ex4 | BCZ1 | A7 | −0.23 | 126% |
| Ex5 | BCZ1 | A8 | −0.25 | 129% |
| Ex6 | BCZ1 | A9 | −0.21 | 124% |
| Ex7 | BCZ1 | A19 | −0.24 | 128% |
| Ex8 | BCZ1 | A29 | −0.25 | 129% |
| Ex9 | BCZ1 | A30 | −0.26 | 130% |
| Ex10 | BCZ1 | A49 | −0.17 | 120% |
| Ex11 | BCZ1 | A63 | −0.15 | 117% |
| Ex12 | BCZ1 | A83 | −0.19 | 122% |
| Ex13 | BCZ1 | A102 | −0.25 | 128% |
| Ex14 | BCZ1 | A105 | −0.23 | 127% |
| Ex15 | BCZ1 | A115 | −0.31 | 135% |
| Ex16 | BCZ1 | A128 | −0.25 | 129% |
| Ex17 | BCZ1 | A132 | −0.25 | 129% |
| Ex18 | BCZ1 | A141 | −0.27 | 132% |

TABLE 3

| | Host2 | Host1 | ΔV | Efficiency |
|---|---|---|---|---|
| Ex19 | BCZ2 | A1 | −0.19 | 121% |
| Ex20 | BCZ2 | A5 | −0.21 | 124% |
| Ex21 | BCZ2 | A6 | −0.21 | 125% |
| Ex22 | BCZ2 | A7 | −0.22 | 125% |
| Ex23 | BCZ2 | A8 | −0.24 | 128% |
| Ex24 | BCZ2 | A9 | −0.20 | 123% |
| Ex25 | BCZ2 | A19 | −0.23 | 126% |
| Ex26 | BCZ2 | A29 | −0.24 | 127% |
| Ex27 | BCZ2 | A30 | −0.24 | 128% |
| Ex28 | BCZ2 | A49 | −0.17 | 119% |
| Ex29 | BCZ2 | A63 | −0.14 | 116% |
| Ex30 | BCZ2 | A83 | −0.18 | 121% |
| Ex31 | BCZ2 | A102 | −0.23 | 127% |
| Ex32 | BCZ2 | A105 | −0.22 | 125% |
| Ex33 | BCZ2 | A115 | −0.29 | 134% |
| Ex34 | BCZ2 | A128 | −0.24 | 127% |
| Ex35 | BCZ2 | A132 | −0.24 | 127% |
| Ex36 | BCZ2 | A141 | −0.26 | 130% |

As shown in Tables 1 to 3, in comparison to the OLED in Ref1 to Ref7, the OLED in Ex1 to Ex36, in which the green EML included an example of the organic compound of the present disclosure, has improved driving voltage and emitting efficiency.

In addition, as shown in Examples 15, 16, 18, 33, 34 and 36, when the organic compound of the present disclosure is represented by Formula 1-3 or 1-4 with Ar₂ being Formula 1b-1, L₁ being phenylene and Ar₁ being phenyl, the driving voltage of the OLED is significantly decreased, and the emitting efficiency of the OLED is significantly increased.

### 3. Comparative Examples 8 to 19 (Ref8 to Ref19)

The compounds CF8 to CF19 in Formula 16 were respectively used as the first host and the compound BCZ1 in Formula 4 was used as the second host to form the green EML. (a weight ratio of (the first host):(the second host)=1:1)

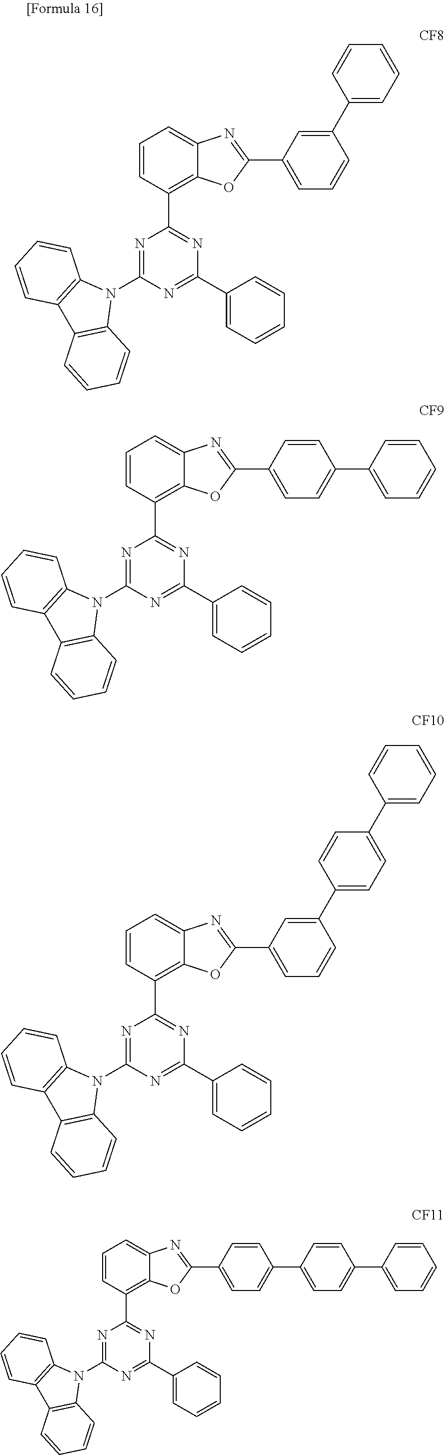 [SMILES: c1ccc(-c2cccc(-c3nc4cccc(-c5nc(-c6ccccc6)nc(-n6c7ccccc7c7ccccc76)n5)c4o3)c2)cc1 ; c1ccc(-c2ccc(-c3nc4cccc(-c5nc(-c6ccccc6)nc(-n6c7ccccc7c7ccccc76)n5)c4o3)cc2)cc1 ; c1ccc(-c2ccc(-c3cccc(-c4nc5cccc(-c6nc(-c7ccccc7)nc(-n7c8ccccc8c8ccccc87)n6)c5o4)c3)cc2)cc1 ; c1ccc(-c2ccc(-c3ccc(-c4nc5cccc(-c6nc(-c7ccccc7)nc(-n7c8ccccc8c8ccccc87)n6)c5o4)cc3)cc2)cc1]

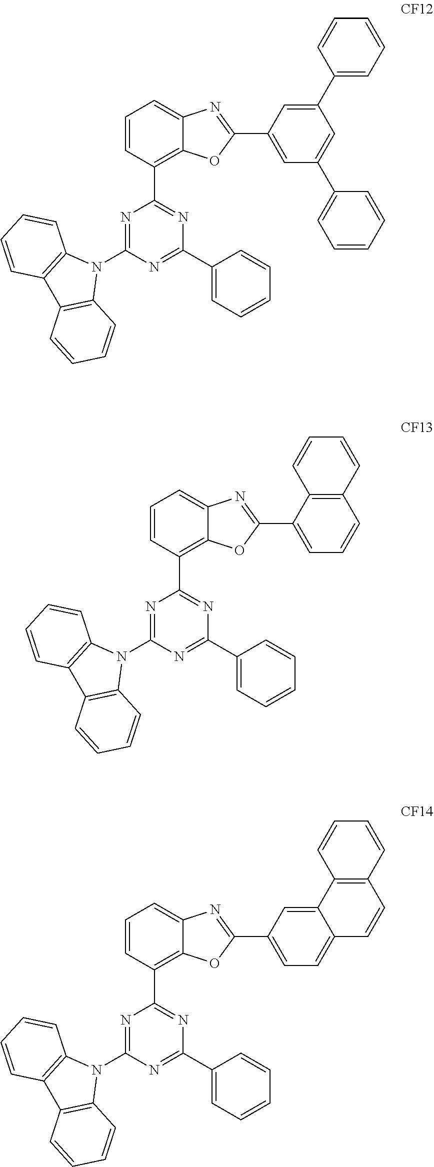 [SMILES: c1ccc(-c2cc(-c3ccccc3)cc(-c3nc4cccc(-c5nc(-c6ccccc6)nc(-n6c7ccccc7c7ccccc76)n5)c4o3)c2)cc1 ; c1ccc(-c2nc(-c3cccc4nc(-c5cccc6ccccc56)oc34)nc(-n3c4ccccc4c4ccccc43)n2)cc1 ; c1ccc(-c2nc(-c3cccc4nc(-c5ccc6ccc7ccccc7c6c5)oc34)nc(-n3c4ccccc4c4ccccc43)n2)cc1]

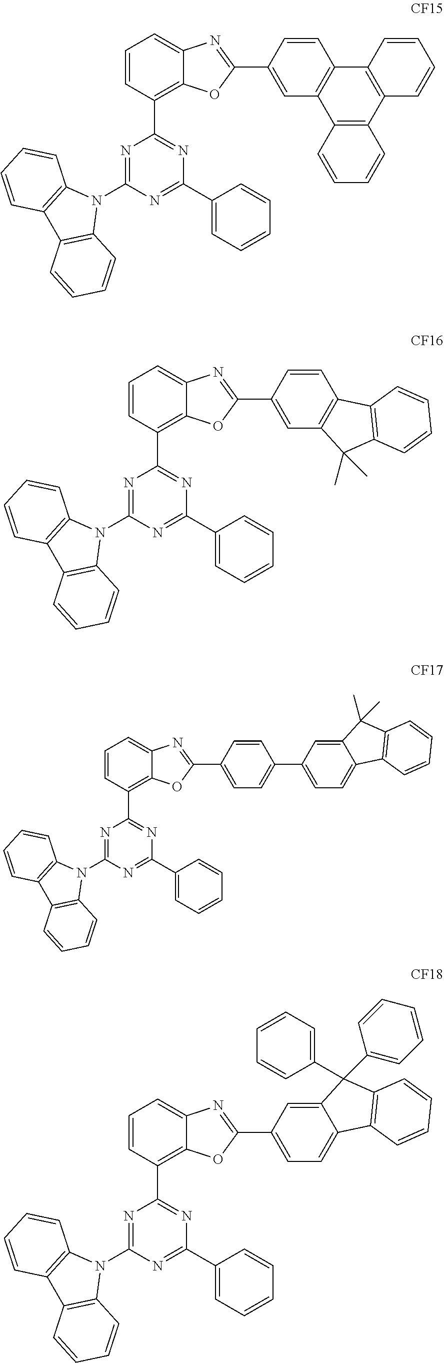 [SMILES: c1ccc(-c2nc(-c3cccc4nc(-c5ccc6c7ccccc7c7ccccc7c6c5)oc34)nc(-n3c4ccccc4c4ccccc43)n2)cc1 ; CC1(C)c2ccccc2-c2ccc(-c3nc4cccc(-c5nc(-c6ccccc6)nc(-n6c7ccccc7c7ccccc76)n5)c4o3)cc21 ; CC1(C)c2ccccc2-c2ccc(-c3ccc(-c4nc5cccc(-c6nc(-c7ccccc7)nc(-n7c8ccccc8c8ccccc87)n6)c5o4)cc3)cc21 ; c1ccc(-c2nc(-c3cccc4nc(-c5ccc6c(c5)C(c5ccccc5)(c5ccccc5)c5ccccc5-6)oc34)nc(-n3c4ccccc4c4ccccc43)n2)cc1]

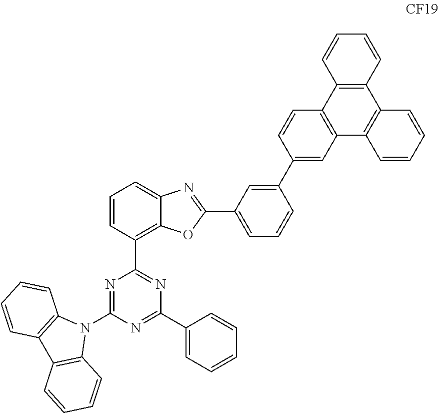 [SMILES: c1ccc(-c2nc(-c3cccc4nc(-c5cccc(-c6ccc7c8ccccc8c8ccccc8c7c6)c5)oc34)nc(-n3c4ccccc4c4ccccc43)n2)cc1]

The properties, e.g., the driving voltage (AV) and the emitting efficiency, of the OLED in Comparative Examples 1, 8 to 19, and Example 1 were measured and listed in Table 4.

TABLE 4

| | Host2 | Host1 | ΔV | Efficiency | Lifespan |
|---|---|---|---|---|---|
| Ref1 | BCZ1 | CF1 | 0.00 | 100% | 100% |
| Ref8 | BCZ1 | CF8 | −0.18 | 121% | 94% |
| Ref9 | BCZ1 | CF9 | −0.21 | 124% | 88% |
| Ref10 | BCZ1 | CF10 | −0.18 | 121% | 83% |
| Ref11 | BCZ1 | CF11 | 0.00 | 100% | 97% |
| Ref12 | BCZ1 | CF12 | −0.20 | 123% | 83% |
| Ref13 | BCZ1 | CF13 | 0.21 | 76% | 89% |
| Ref14 | BCZ1 | CF14 | 0.20 | 77% | 94% |
| Ref15 | BCZ1 | CF15 | 0.01 | 99% | 98% |
| Ref16 | BCZ1 | CF16 | 0.03 | 97% | 97% |
| Ref17 | BCZ1 | CF17 | 0.05 | 94% | 96% |
| Ref18 | BCZ1 | CF18 | 0.02 | 97% | 97% |
| Ref19 | BCZ1 | CF19 | −0.17 | 120% | 83% |
| Ex1 | BCZ1 | A1 | −0.20 | 123% | 104% |

As shown in Table 4, in comparison to the OLED in Ref1 and Ref8 to Ref19, the OLED in Ex1, in which the green EML included an example of the organic compound of the present disclosure, has improved driving voltage, emitting efficiency and lifespan.

For example, each of the compounds CF8 to CF19 used in Ref8 to Ref19 and the compound A1 only have a difference in a substituent at a terminal of a benzoxazole moiety. In the OLED of Ex1 using the compound A1, in which a C6 aryl group, i.e., phenyl, is combined (bonded) to a terminal of the benzoxazole moiety, the emission property is significantly improved.

### 4. Comparative Examples 20 to 31 (Ref20 to Ref3l)

The compounds CF20 to CF31 in Formula 17 was respectively used as the first host and the compound BCZ1 in Formula 4 was used as the second host to form the green EML. (a weight ratio of (the first host):(the second host)=1:1)

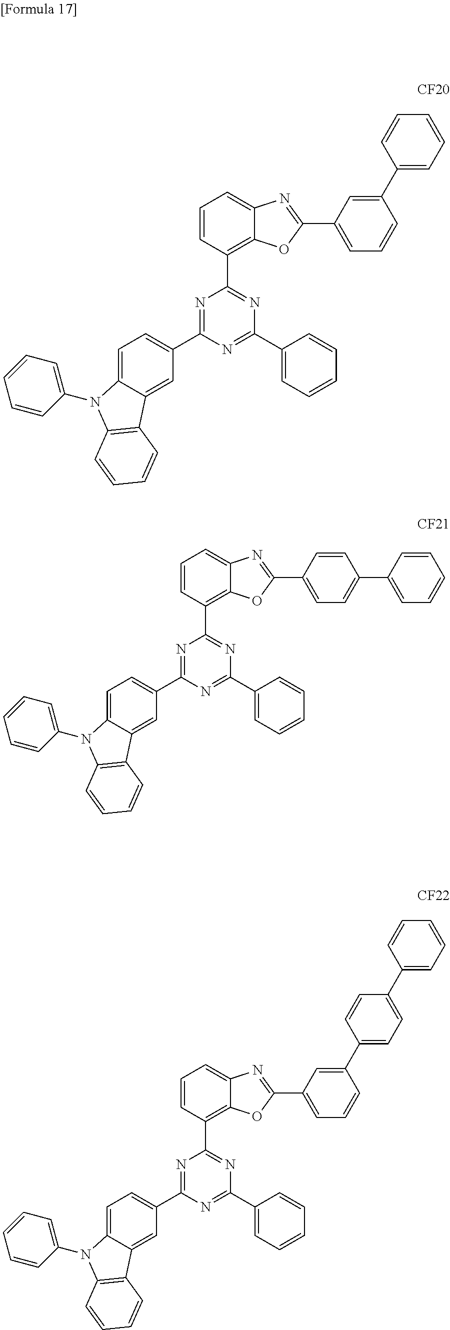 [SMILES: c1ccc(-c2cccc(-c3nc4cccc(-c5nc(-c6ccccc6)nc(-c6ccc7c(c6)c6ccccc6n7-c6ccccc6)n5)c4o3)c2)cc1 ; c1ccc(-c2ccc(-c3nc4cccc(-c5nc(-c6ccccc6)nc(-c6ccc7c(c6)c6ccccc6n7-c6ccccc6)n5)c4o3)cc2)cc1 ; c1ccc(-c2ccc(-c3cccc(-c4nc5cccc(-c6nc(-c7ccccc7)nc(-c7ccc8c(c7)c7ccccc7n8-c7ccccc7)n6)c5o4)c3)cc2)cc1]

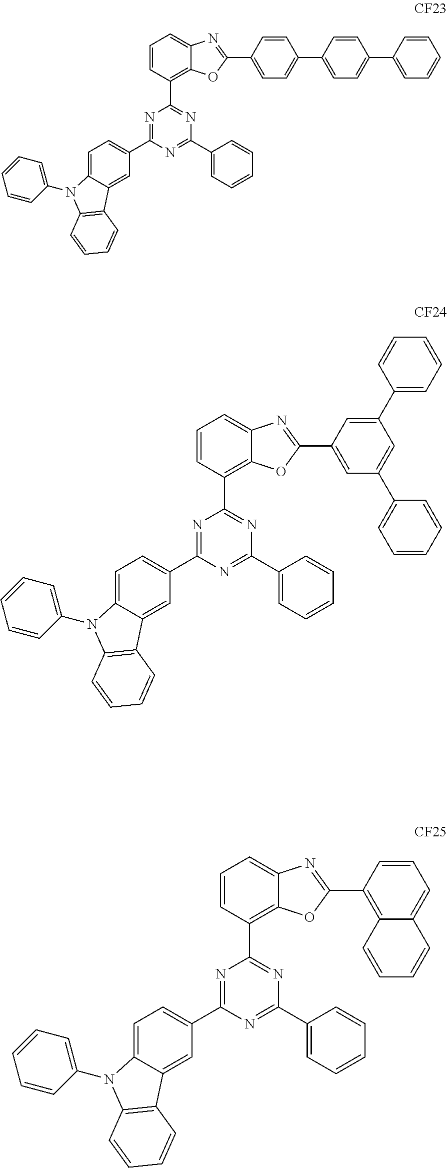 [SMILES: c1ccc(-c2ccc(-c3ccc(-c4nc5cccc(-c6nc(-c7ccccc7)nc(-c7ccc8c(c7)c7ccccc7n8-c7ccccc7)n6)c5o4)cc3)cc2)cc1 ; c1ccc(-c2cc(-c3ccccc3)cc(-c3nc4cccc(-c5nc(-c6ccccc6)nc(-c6ccc7c(c6)c6ccccc6n7-c6ccccc6)n5)c4o3)c2)cc1 ; c1ccc(-c2nc(-c3ccc4c(c3)c3ccccc3n4-c3ccccc3)nc(-c3cccc4nc(-c5cccc6ccccc56)oc34)n2)cc1]

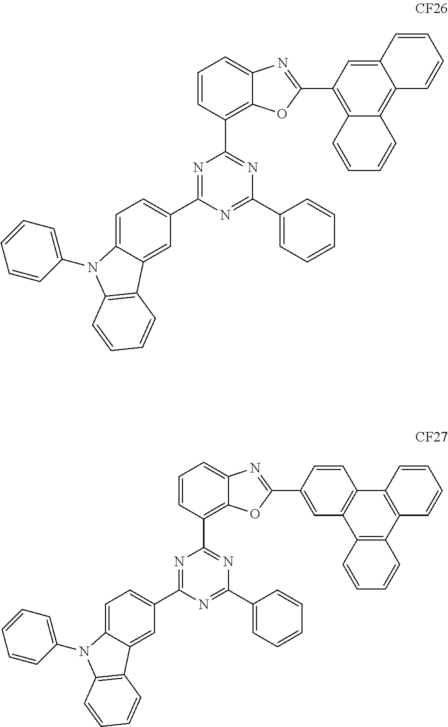 [SMILES: c1ccc(-c2nc(-c3ccc4c(c3)c3ccccc3n4-c3ccccc3)nc(-c3cccc4nc(-c5cc6ccccc6c6ccccc56)oc34)n2)cc1 ; c1ccc(-c2nc(-c3ccc4c(c3)c3ccccc3n4-c3ccccc3)nc(-c3cccc4nc(-c5ccc6c7ccccc7c7ccccc7c6c5)oc34)n2)cc1]

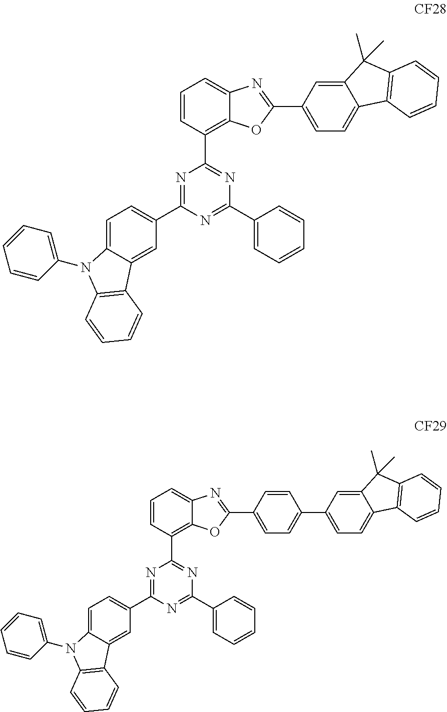 [SMILES: CC1(C)c2ccccc2-c2ccc(-c3nc4cccc(-c5nc(-c6ccccc6)nc(-c6ccc7c(c6)c6ccccc6n7-c6ccccc6)n5)c4o3)cc21 ; CC1(C)c2ccccc2-c2ccc(-c3ccc(-c4nc5cccc(-c6nc(-c7ccccc7)nc(-c7ccc8c(c7)c7ccccc7n8-c7ccccc7)n6)c5o4)cc3)cc21]

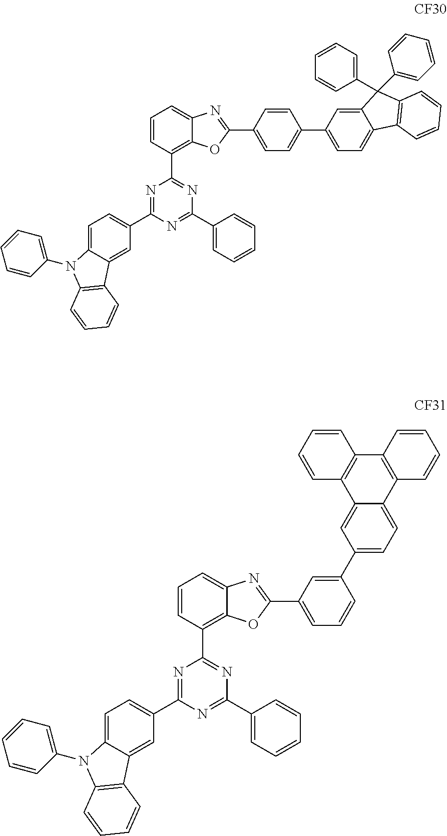 [SMILES: c1ccc(-c2nc(-c3ccc4c(c3)c3ccccc3n4-c3ccccc3)nc(-c3cccc4nc(-c5ccc(-c6ccc7c(c6)C(c6ccccc6)(c6ccccc6)c6ccccc6-7)cc5)oc34)n2)cc1 ; c1ccc(-c2nc(-c3ccc4c(c3)c3ccccc3n4-c3ccccc3)nc(-c3cccc4nc(-c5cccc(-c6ccc7c8ccccc8c8ccccc8c7c6)c5)oc34)n2)cc1]

The properties, e.g., the driving voltage (ΔV) and the emitting efficiency, of the OLED in Comparative Examples 1, 20 to 31, and Example 11 were measured and listed in Table 5.

TABLE 5

| | Host2 | Host1 | ΔV | Efficiency | Lifespan |
|---|---|---|---|---|---|
| Ref1 | BCZ1 | CF1 | 0.00 | 100% | 100% |
| Ref20 | BCZ1 | CF20 | −0.11 | 113% | 94% |
| Ref21 | BCZ1 | CF21 | −0.11 | 112% | 94% |
| Ref22 | BCZ1 | CF22 | −0.11 | 113% | 89% |
| Ref23 | BCZ1 | CF23 | 0.09 | 90% | 92% |
| Ref24 | BCZ1 | CF24 | −0.10 | 112% | 94% |
| Ref25 | BCZ1 | CF25 | 0.24 | 73% | 99% |
| Ref26 | BCZ1 | CF26 | 0.25 | 71% | 94% |
| Ref27 | BCZ1 | CF27 | 0.07 | 92% | 97% |
| Ref28 | BCZ1 | CF28 | 0.11 | 87% | 92% |
| Ref29 | BCZ1 | CF29 | 0.10 | 88% | 97% |
| Ref30 | BCZ1 | CF30 | 0.10 | 88% | 86% |
| Ref31 | BCZ1 | CF31 | −0.12 | 113% | 83% |
| Ex11 | BCZ1 | A63 | −0.15 | 117% | 104% |

As shown in Table 5, in comparison to the OLED in Ref1, Ref20 to Ref31, the OLED in Ex11, in which the green EML included an example of the organic compound of the present disclosure, has improved driving voltage, emitting efficiency and lifespan.

For example, each of the compounds CF20 to CF31 used in Ref20 to Ref31 and the compound A63 only have a difference in a substituent at a terminal of a benzoxazole moiety. In the OLED of Ex11 using the compound A63, in which a C6 aryl group, i.e., phenyl, is combined (bonded) to a terminal of the benzoxazole moiety, the emission property is significantly improved.

### 5. Comparative Examples 32 to 37 (Ref32 to Ref37)

The compounds CF32 to CF37 in Formula 18 was respectively used as the first host and the compound BCZ1 in Formula 4 was used as the second host to form the green EML. (a weight ratio of (the first host):(the second host)=1:1)

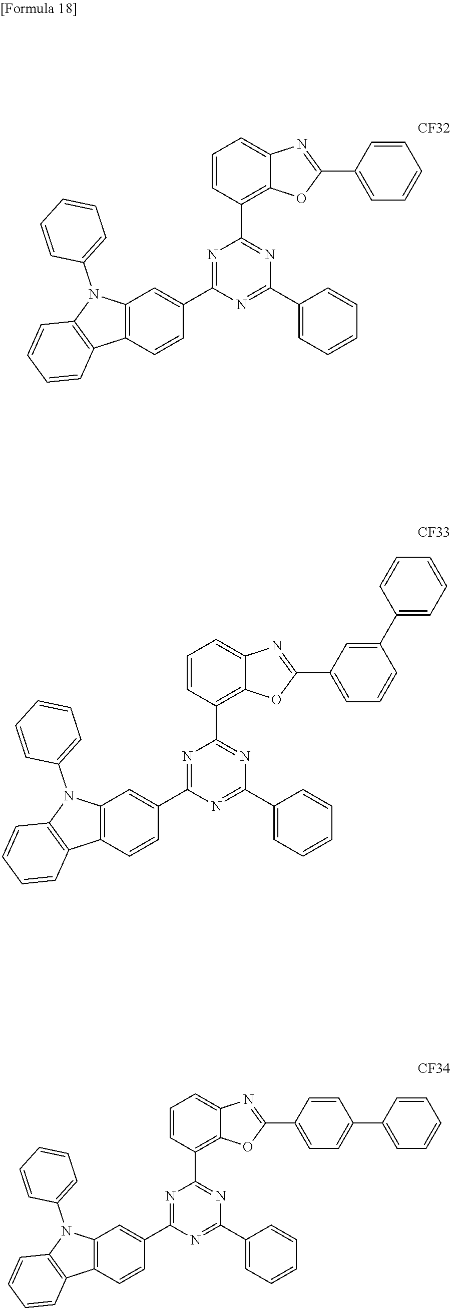 [SMILES: c1ccc(-c2nc(-c3ccc4c5ccccc5n(-c5ccccc5)c4c3)nc(-c3cccc4nc(-c5ccccc5)oc34)n2)cc1 ; c1ccc(-c2cccc(-c3nc4cccc(-c5nc(-c6ccccc6)nc(-c6ccc7c8ccccc8n(-c8ccccc8)c7c6)n5)c4o3)c2)cc1 ; c1ccc(-c2ccc(-c3nc4cccc(-c5nc(-c6ccccc6)nc(-c6ccc7c8ccccc8n(-c8ccccc8)c7c6)n5)c4o3)cc2)cc1]

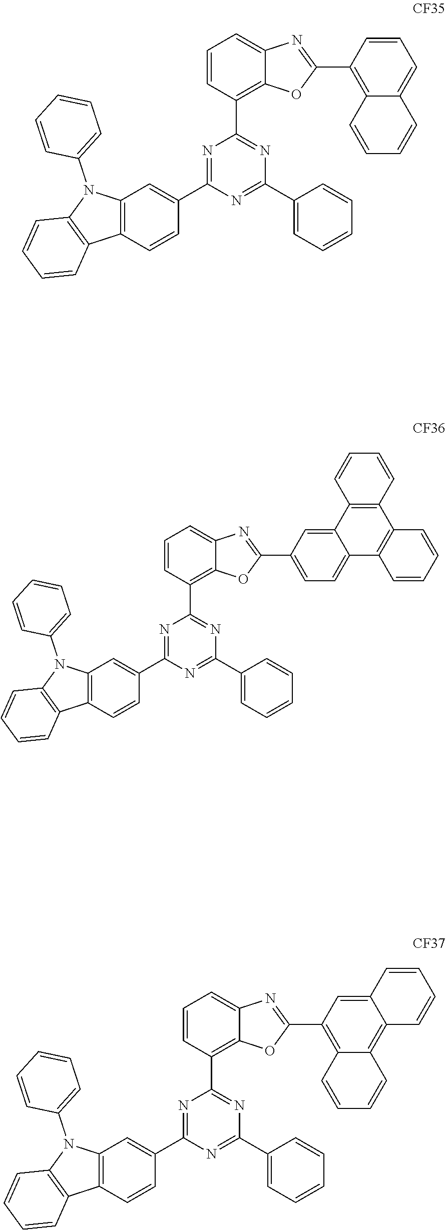 [SMILES: c1ccc(-c2nc(-c3ccc4c5ccccc5n(-c5ccccc5)c4c3)nc(-c3cccc4nc(-c5cccc6ccccc56)oc34)n2)cc1 ; c1ccc(-c2nc(-c3ccc4c5ccccc5n(-c5ccccc5)c4c3)nc(-c3cccc4nc(-c5ccc6c7ccccc7c7ccccc7c6c5)oc34)n2)cc1 ; c1ccc(-c2nc(-c3ccc4c5ccccc5n(-c5ccccc5)c4c3)nc(-c3cccc4nc(-c5cc6ccccc6c6ccccc56)oc34)n2)cc1]

The properties, e.g., the driving voltage (ΔV) and the emitting efficiency, of the OLED in Comparative Examples 1, 32 to 37 and Example 11 were measured and listed in Table 6.

TABLE 6

| | Host2 | Host1 | ΔV | Efficiency | Lifespan |
|---|---|---|---|---|---|
| Ref1 | BCZ1 | CF1 | 0.00 | 100% | 100% |
| Ref32 | BCZ1 | CF32 | −0.03 | 103% | 106% |
| Ref33 | BCZ1 | CF33 | −0.03 | 103% | 90% |
| Ref34 | BCZ1 | CF34 | −0.01 | 101% | 89% |
| Ref35 | BCZ1 | CF35 | 0.33 | 62% | 87% |
| Ref36 | BCZ1 | CF36 | −0.02 | 102% | 95% |
| Ref37 | BCZ1 | CF37 | 0.34 | 61% | 84% |
| Ex11 | BCZ1 | A63 | −0.15 | 117% | 104% |

As shown in Table 6, in comparison to the OLED in Ref1 and Ref32 to Ref37, the OLED in Ex11, in which the green EML included an example of the organic compound of the present disclosure, has improved driving voltage, emitting efficiency and lifespan.

For example, each of the compound CF32 used in Ref32 and the compound A63 only have a difference in a liking position (bonding position) between a carbazole moiety and a triazine moiety. In the OLED of Ex11 using the compound A63, in which the triazine moiety is combined at a 3-position of the carbazole moiety, the driving voltage is significantly decreased and the emitting efficiency is significantly increased.

In addition, in comparison to the OLED in Ref33 to Ref37 respectively using the compounds CF33 to CF37, in which the triazine moiety is combined at a 2-position of the carbazole moiety and a C10 or more aryl group is combined to a terminal of the benzoxazole moiety, the OLED of Ex11 using the compound A63, in which the triazine moiety is combined at a 3-position of the carbazole moiety and a C6 aryl group, i.e., phenyl, is combined (bonded) to a terminal of the benzoxazole moiety, has improved emitting efficiency and lifespan.

FIG. 4 illustrates a schematic cross-sectional view of an OLED according to a third embodiment of the present disclosure.

As illustrated in FIG. 4, an OLED D may include first and second electrodes 160 and 164, which may face each other, and an organic light emitting layer 162 therebetween. The organic light emitting layer 162 may include a first emitting part 310 including a first EML 320 and a second emitting part 330 including a green EML 340. The organic light emitting layer 162 may further include a CGL 350 between the first and second emitting parts 310 and 330.

The first electrode 160 may act as an anode for injecting a hole and may be formed of a conductive material, e.g., ITO or IZO, having a relatively high work function. The second electrode 164 may act a cathode for injecting an electron and may be formed of a conductive material, e.g., A1, Mg or AlMg, having a relatively low work function.

In the top-emission type OLED D, the first electrode 160 may further include a reflection layer to act as a reflective electrode. The second electrode 164 may have a thin profile to act as a transparent (semitransparent) electrode. Alternatively, in the bottom-emission type OLED D, the first electrode 160 may act as a transparent electrode. The second electrode 164 may act as a reflective electrode. However, embodiments of the present disclosure are not limited to such examples.

The CGL 350 may be positioned between the first and second emitting parts 310 and 330. The first emitting part 310, the CGL 350, and the second emitting part 330 may be sequentially stacked on the first electrode 160. For example, the first emitting part 310 may be positioned between the first electrode 160 and the CGL 350. The second emitting part 330 may be positioned between the second electrode 164 and the CGL 350.

The first emitting part 310 may further include a first ETL 316 between the first EML 320 and the CGL 350. For example, the first ETL 316 may be positioned between the first EML 320 and the CGL 350.

In addition, the first emitting part 310 may further include at least one of an HIL 312 between the first EML 320 and the first electrode 160 and an HTL 314 between the first EML 320 and the HIL 312.

Moreover, the first emitting part 310 may further include at least one of a first EBL (not shown) between the first EML 320 and the first HTL 314 and a first HBL (not shown) between the first EML 320 and the first ETL 316.

The second emitting part 330 may further include a second ETL 334 between the second green EML 340 and the second electrode 164.

In addition, the second emitting part 330 may further include at least one of a second HTL 332 under the second green EML 340 and an EIL 336 between the second ETL 334 and the second electrode 164.

Moreover, the second emitting part 330 may further include at least one of a second EBL (not shown) between the second green EML 340 and the second HTL 332 and a second HBL (not shown) between the second green EML 340 and the second ETL 334.

The CGL 350 may be positioned between the first and second emitting parts 310 and 330. For example, the first and second emitting parts 310 and 330 may be connected through the CGL 350. The CGL 350 may be a P-N junction CGL including an N-type CGL 352 and a P-type CGL 354.

The N-type CGL 352 may be positioned between the first ETL 316 and the second HTL 332. The P-type CGL 354 may be positioned between the N-type CGL 352 and the second HTL 332.

The N-type CGL 352 may be an organic layer doped with an alkali metal, e.g., Li, Na, K and Cs, and/or an alkali earth metal, e.g., Mg, Sr, Ba and Ra. For example, the N-type CGL 352 may be formed of an N-type charge generation material including a host being the organic material, e.g., 4,7-dipheny-1,10-phenanthroline (Bphen) and MTDATA, a dopant being an alkali metal and/or an alkali earth metal, and the dopant may be doped with a weight % of 0.01 to 30.

The P-type CGL 354 may be formed of a P-type charge generation material including an inorganic material, e.g., tungsten oxide (WOx), molybdenum oxide (MoOx), beryllium oxide (Be₂O₃) or vanadium oxide (V₂O₅), an organic material, e.g., NPD, HAT-CN, F4TCNQ, TPD, TNB, TCTA, N,N′-dioctyl-3,4,9,10-perylenedicarboximide (PTCDI-C8) or their combination.

The first EML 320 may include a first compound 322, a second compound 324, and a third compound 326. The second EML 340 may include a fourth compound 342, a fifth compound 344, and a sixth compound 346.

At least one of the first and fourth compounds 322 and 342 is an example of the organic compound of the present disclosure represented by Formula 1, and at least one of the second and fifth compounds 324 and 344 is the compound represented by Formula 3.

Each of the first EML 320 and the second EML 340 is a green EML or a yellow-green EML. Namely, the OLED D is positioned in the green pixel region or the yellow-green pixel region.

In the green pixel region, each of the third compound 326 and the sixth compound 346 is selected from the compounds in Formula 5. In the yellow-green pixel region, each of the third compound 326 and the sixth compound 346 is selected from the compounds in Formula 6.

In the first green EML 320, the first compound 322 may act as an n-type host, e.g., a first host, the second compound 324 may act as a p-type host, e.g., a second host, and the third compound 326 may act as an emitter, e.g., a dopant. In the second green EML 340, the fourth compound 342 may act as an n-type host, e.g., a first host, the fifth compound 344 may act as a p-type host, e.g., a second host, and the sixth compound 346 may act as an emitter, e.g., a dopant. Each of the first and second green EMLs 320 and 340 may have a thickness of 50 to 600 Å.

When the first compound 322 and the fourth compound 342 are examples of the organic compound represented by Formula 1, the first compound 322 and the fourth compound 342 may be the same or different. When the second compound 324 and the fifth compound 344 are examples of the organic compound represented by Formula 3, the second compound 324 and the fifth compound 344 may be the same or different. When each of the third compound 326 and the sixth compound 346 is selected from the compounds in Formula 5, the third compound 326 and the sixth compound 346 may be the same or different. When each of the third compound 326 and the sixth compound 346 is selected from the compounds in Formula 6, the third compound 326 and the sixth compound 346 may be the same or different.

In the first EML 320, a weight % of each of the first and second compounds 322 and 324 may be greater than that of the third compound 326. The weight % of the first compound 322 and the weight % of the second compound 324 may be the same or different. In the first EML 320, a weight % ratio of the first compound 322 to the second compound 324 may be 1:9 to 9:1, 2:8 to 8:2 or 7:3 to 3:7. In some embodiments, the weight % of the first compound 322 and the weight % of the second compound 324 may be same. For example, the first compound 322 and the second compound 324 may be present at the same weight %. The third compound 326 may be present in an amount of 5 to 25 weight % in the first green EML 320, based on a total weight of the components in the first EML 320.

In the second EML 340, a weight % of each of the fourth and fifth compounds 342 and 344 may be greater than that of the sixth compound 346. The weight % of the fourth compound 342 and the weight % of the fifth compound 344 may be the same or different. In the second EML 340, a weight % ratio of the fourth compound 342 to the fifth compound 344 may be 1:9 to 9:1, 2:8 to 8:2 or 7:3 to 3:7. In some embodiments, the weight % of the fourth compound 342 and the weight % of the fifth compound 344 may be same. For example, the fourth compound 342 and the fifth compound 344 may be present at the same weight %. The sixth compound 346 may be present in an amount of 5 to 25 weight % in the second green EML 340, based on a total weight of the components in the second EML 340.

Each of the first and second ETLs 316 and 334 may include at least one of a first electron transporting material represented by Formula 7, a second electron transporting material represented by Formula 8 and a third electron transporting material represented by Formula 9.

In the OLED D in at least one of the green pixel region and the yellow-green pixel region, at least one of the first and second EMLs 320 and 340 may include an example of the organic compound of the present disclosure represented by Formula 1. As a result, in the OLED D, the driving voltage may be reduced, and the emitting efficiency and the lifespan may be improved.

In addition, at least one of the first and second EMLs 320 and 340 may further include the compound, which is represented by Formula 3, as a second host with a first host being the organic compound of the present disclosure so that the OLED D may have improvements in aspects such as the driving voltage, the emitting efficiency, and the emitting lifespan.

Moreover, at least one of the first and second EMLs 320 and 340 may further include the compound, which is one of the compounds in Formula 5 or Formula 6, as an emitter with the first host represented by Formula 1 and the second host represented by Formula 3 so that the OLED D may have further improvements in aspects such as the driving voltage, the emitting efficiency, and the emitting lifespan.

Furthermore, in the OLED D in the green pixel region, at least one of the first and second ETLs 316 and 334 may include at least one of a first electron transporting material represented by Formula 7, a second electron transporting material represented by Formula 8 and a third electron transporting material represented by Formula 9 so that the OLED D may have further improvements in aspects such as the driving voltage, the emitting efficiency, and the emitting lifespan.

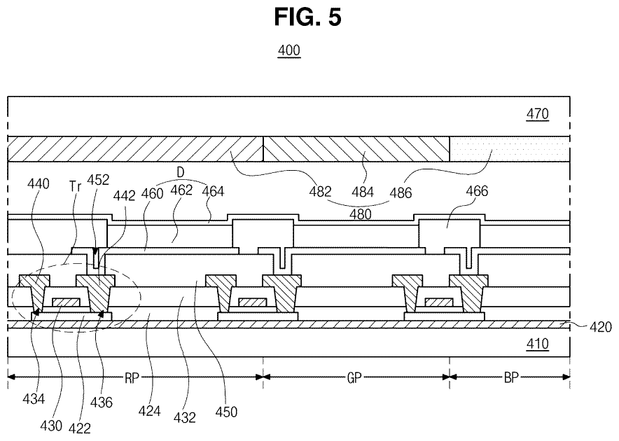

FIG. 5 illustrates a schematic cross-sectional view of an organic light emitting display device according to a fourth embodiment of the present disclosure.

As shown in FIG. 5, an organic light emitting display device 400 may include a first substrate 410, where a red pixel region RP, a green pixel region GP and a blue pixel region BP may be defined, a second substrate 470 facing the first substrate 410, an OLED D, which may be positioned between the first and second substrates 410 and 470 and providing white emission, and a colour filter layer 480 between the OLED D and the second substrate 470.

Each of the first and second substrates 410 and 470 may be a glass substrate or a flexible substrate. For example, each of the first and second substrates 410 and 470 may be a polyimide (PI) substrate, a polyethersulfone (PES) substrate, a polyethylenenaphthalate (PEN) substrate, a polyethylene terephthalate (PET) substrate or a polycarbonate (PC) substrate.

A buffer layer 420 may be formed on the substrate. The TFT Tr corresponding to each of the red, green and blue pixel regions RP, GP and BP may be formed on the buffer layer 420. The buffer layer 420 may be formed of an inorganic insulating material, e.g., silicon oxide or silicon nitride. The buffer layer 420 may have a multi-layered structure including a first layer of silicon oxide and a second layer of silicon nitride. The buffer layer 420 may be omitted, and the TFT Tr may be disposed on the substrate 410.

A semiconductor layer 422 may be formed on the buffer layer 420. The semiconductor layer 422 may include an oxide semiconductor material or polycrystalline silicon.

A gate insulating layer 424 may be formed on the semiconductor layer 422. The gate insulating layer 424 may be formed of an inorganic insulating material such as silicon oxide or silicon nitride.

A gate electrode 430, which may be formed of a conductive material, e.g., metal, may be formed on the gate insulating layer 424 to correspond to a center of the semiconductor layer 422.

An interlayer insulating layer 432, which may be formed of an insulating material, may be formed on the gate electrode 430. The interlayer insulating layer 432 may be formed of an inorganic insulating material, e.g., silicon oxide or silicon nitride, or an organic insulating material, e.g., benzocyclobutene or photo-acryl.

The interlayer insulating layer 432 may include first and second contact holes 434 and 436 exposing both sides of the semiconductor layer 422. The first and second contact holes 434 and 436 may not cover a portion of the surface of the semiconductor layer 422 that is nearer to the opposing ends than to a center of the semiconductor layer 422. The first and second contact holes 434 and 436 may be positioned at both sides of the gate electrode 430 to be spaced apart from the gate electrode 430.

A source electrode 440 and a drain electrode 442, which may be formed of a conductive material, e.g., metal, may be formed on the interlayer insulating layer 432.

The source electrode 440 and the drain electrode 442 may be spaced apart from each other with respect to the gate electrode 430 and contact both sides of the semiconductor layer 422 through the first and second contact holes 434 and 436, respectively.

The semiconductor layer 422, the gate electrode 430, the source electrode 440, and the drain electrode 442 may constitute the TFT Tr. The TFT Tr may serve as a driving element. For example, the TFT Tr may correspond to the driving TFT Td (of FIG. 1).

Although not shown, the gate line and the data line may cross each other to define the pixel region. The switching TFT may be formed to be connected to the gate and data lines. The switching TFT may be connected to the TFT Tr as the driving element.

In addition, the power line, which may be formed to be parallel to and spaced apart from one of the gate and data lines, and the storage capacitor for maintaining the voltage of the gate electrode of the TFT Tr in one frame may be further formed.

A planarization layer 450, which may include a drain contact hole 452 exposing the drain electrode 442 of the TFT Tr, may be formed to cover the TFT Tr. The drain contact hole 452 may not cover the drain electrode 442.

A first electrode 460, which may be connected to the drain electrode 442 of the TFT Tr through the drain contact hole 452, may be separately formed in each pixel region and on the planarization layer 450. The first electrode 460 may be an anode and may include a transparent conductive oxide material layer formed of a conductive material, e.g., a transparent conductive oxide (TCO), having a relatively high work function.

For example, the transparent conductive oxide material layer of the first electrode 460 may include at least one of indium-tin-oxide (ITO) indium-zinc-oxide (IZO), indium-tin-zinc oxide; ITZO), tin oxide (SnO), zinc oxide (ZnO), indium-copper-oxide (ICO) and Al:ZnO (AZO).

The first electrode 460 may further include a reflection layer to have a double-layered structure or a triple-layered structure. For example, the reflection layer may be formed of silver (Ag) or aluminium-palladium-copper (APC) alloy. In the top-emission type organic light emitting display device 400, the first electrode 460 may have a double-layered structure of Ag/ITO or APC/ITO or a triple-layered structure of ITO/Ag/ITO or ITO/APC/ITO. However, embodiments of the present disclosure are not limited to such examples.

A bank layer 466 may be formed on the planarization layer 450 to cover an edge of the first electrode 460. For example, the bank layer 466 may be positioned at a boundary of the pixel region and may expose a center of the first electrode 460 in the pixel region. Since the OLED D may emit white light in the red, green and blue pixel regions RP, GP and BP, the organic light emitting layer 462 may be formed as a common layer in the red, green and blue pixel regions RP, GP and BP without separation. The bank layer 466 may be formed to prevent a current leakage at an edge of the first electrode 460 and may be omitted.

An organic light emitting layer 462 may be formed on the first electrode 460.

In an aspect of the present disclosure, the organic light emitting layer 462 may have a three-stack structure including a first emitting part, which includes at least one of a green EML and a yellow-green EML, and second and third emitting part, each of which includes a blue EML.

In an aspect of the present disclosure, the organic light emitting layer 462 may have a two-stack structure including a first emitting part, which includes at least one of a green EML and a yellow-green EML, and a second emitting part, which includes a blue EML.

At least one of the green EML and the yellow-green EML includes the organic compound of the present disclosure represented by Formula 1.

A second electrode 464 may be formed over the substrate 410 where the organic light emitting layer 462 may be formed.

In the organic light emitting display device 400, since the light emitted from the organic light emitting layer 462 may be incident to the colour filter layer 480 through the second electrode 464, the second electrode 464 may have a thin profile for transmitting the light.

The first electrode 460, the organic light emitting layer 462, and the second electrode 464 may constitute the OLED D.

The color filter layer 480 may be positioned over the OLED D and may include a red color filter pattern 482, a green color filter pattern 484, and a blue color filter pattern 486 corresponding to the red, green, and blue pixel regions RP, GP, and BP, respectively. The red color filter pattern 482 may include at least one of red dye and red pigment. The green color filter pattern 484 may include at least one of green dye and green pigment. The blue color filter pattern 486 may include at least one of blue dye and blue pigment.

Although not shown, the color filter layer 480 may be attached to the OLED D by an adhesive layer. Alternatively, the color filter layer 480 may be formed directly on the OLED D. However, embodiments of the present disclosure are not limited to such examples.

An encapsulation layer (not shown) may be formed to prevent penetration of moisture into the OLED D. For example, the encapsulation layer may include a first inorganic insulating layer, an organic insulating layer, and a second inorganic insulating layer sequentially stacked, but it is not limited thereto. The encapsulation layer may be omitted.

A polarization plate (not shown) for reducing an ambient light reflection may be disposed over the top-emission type OLED D. For example, the polarization plate may be a circular polarization plate.

In the OLED of FIG. 5, the first and second electrodes 460 and 464 may be a reflective electrode and a transparent (or semi-transparent) electrode, respectively. The color filter layer 480 may be disposed over the OLED D. Alternatively, when the first and second electrodes 460 and 464 are a transparent (or semi-transparent) electrode and a reflective electrode, respectively, the color filter layer 480 may be disposed between the OLED D and the first substrate 410. However, embodiments of the present disclosure are not limited to such examples.

A colour conversion layer (not shown) may be formed between the OLED D and the colour filter layer 480. The colour conversion layer may include a red colour conversion layer, a green colour conversion layer, and a blue colour conversion layer corresponding to the red, green, and blue pixel regions RP, GP, and BP, respectively. The white light from the OLED D may be converted into the red light, the green light, and the blue light by the red, green, and blue colour conversion layers, respectively. For example, the colour conversion layer may include a quantum dot. Accordingly, the colour purity of the organic light emitting display device 400 may be further improved.

The colour conversion layer may be included instead of the colour filter layer 480.

As described above, in the organic light emitting display device 400, the OLED D in the red, green, and blue pixel regions RP, GP, and BP may emit the white light. The white light from the organic light emitting diode D may pass through the red colour filter pattern 482, the green colour filter pattern 484, and the blue colour filter pattern 486. As a result, the red light, the green light and the blue light may be provided from the red pixel region RP, the green pixel region GP, and the blue pixel region BP, respectively.

In FIG. 5, the OLED D emitting the white light may be used for a display device. Alternatively, the OLED D may be formed on an entire surface of a substrate without at least one of the driving element and the colour filter layer to be used for a lighting device. The display device and the lighting device each including an example of the OLED D of the present disclosure may be referred to as an organic light emitting device. However, embodiments of the present disclosure are not limited to such examples.

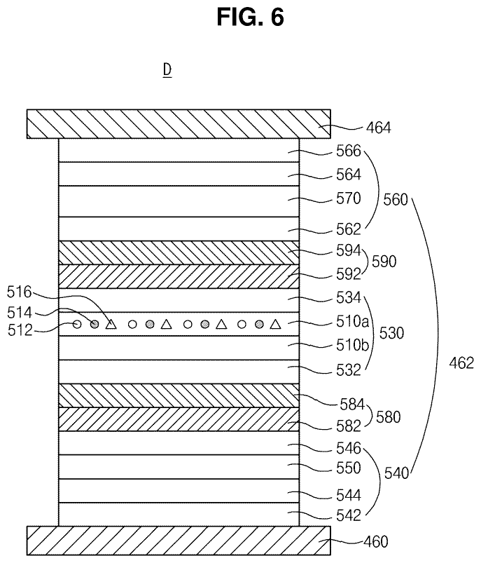

FIG. 6 illustrates a schematic cross-sectional view of an OLED according to a fifth embodiment of the present disclosure.

As illustrated in FIG. 6, the organic light emitting layer 462 may include a first emitting part 530 including a green EML 510a, a second emitting part 540 including a first blue EML 550, and a third emitting part 560 including a second blue EML 570. In addition, the organic light emitting layer 462 may further include a first CGL 580 between the first and second emitting parts 530 and 540 and a second CGL 590 between the first and third emitting parts 530 and 560. In addition, the first emitting part 530 may further include a red EML 510b.

The second emitting part 540 may be positioned between the first electrode 460 and the first emitting part 530. The third emitting part 560 may be positioned between the first emitting part 530 and the second electrode 464. The second emitting part 540 may be positioned between the first electrode 460 and the first CGL 580. The third emitting part 560 may be positioned between the second CGL 590 and the second electrode 464. For example, the second emitting part 540, the first CGL 580, the first emitting part 530, the second CGL 590, and the third emitting part 560 may be sequentially stacked on the first electrode 460.

In the first emitting part 530, the red EML 510b may be disposed under the green EML 510a.

The first emitting part 530 may further include a first ETL 534 disposed on the green EML 510a. In addition, the first emitting part 530 may further include a first HTL 532 disposed under the red EML 510b.

For example, in the first emitting part 530, the red EML 510b may be positioned between the first HTL 532 and the green EML 510a. The green EML 510a may be positioned between the red EML 510b and the first ETL 534.

The second emitting part 540 may further include at least one of a second HTL 544 disposed under the first blue EML 550 and a second ETL 546 disposed on the first blue EML 550. In addition, the second emitting part 540 may further include an HIL 542 between the first electrode 460 and the second HTL 544.

Moreover, the second emitting part 540 may further include a first EBL (not shown) between the second HTL 544 and the first blue EML 550 and a first HBL (not shown) between the second ETL 546 and the first blue EML 550.

The third emitting part 560 may further include at least one of a third HTL 562 disposed under the second blue EML 570 and a third ETL 564 disposed on the second blue EML 570. In addition, the third emitting part 560 may further include an EIL 566 between the second electrode 464 and the third ETL 564.

Moreover, the third emitting part 560 may further include a second EBL (not shown) between the third HTL 562 and the second blue EML 570 and a second HBL (not shown) between the third ETL 564 and the second blue EML 570.

The green EML 510a may include a first compound 512 that is the organic compound of the present disclosure represented by Formula 1. In addition, the green EML 510a may further include a second compound 514 that is the compound represented by Formula 3.

Moreover, the green EML 510a may further include a third compound 516 that is the compound represented by Formula 5.

In the green EML 510a, the first compound 512 may be an n-type host (e.g., a first host), the second compound 514 may be a p-type host (e.g., a second host), and the third compound 516 may be an emitter (e.g., a dopant). The green EML 510a may have a thickness of 50 to 600 Å.

In the green EML 510a, a weight % of each of the first and second compounds 512 and 514 may be greater than that of the third compound 516. The weight % of the first compound 512 and the weight % of the second compound 514 may be the same or different. In the green EML 510a, a weight % ratio of the first compound 512 to the second compound 514 may be 1:9 to 9:1, 2:8 to 8:2 or 7:3 to 3:7. In some embodiments, the weight % of the first compound 512 and the weight % of the second compound 514 may be same. The third compound 516 may be present in an amount of 5 to 25 weight % in the green EML 510a, based on a total weight of the components in the green EML 510a.

Each of the first to third ETLs 534, 546, and 564 may include at least one of the first electron transporting material represented by Formula 7, the second electron transporting material represented by Formula 8 and the third electron transporting material represented by Formula 9.

The red EML 510b may include a red host and a red dopant. The red dopant may include at least one of a red phosphorescent compound, a red fluorescent compound, and a red delayed fluorescent compound. In the red EML 510b, the red host may be present at a weight % greater than the red dopant. In the red EML 510b, the red dopant may be present at a weight % of 1 to 10, or 1 to 5, based on a total weight of the components in the red EML 510b.

For example, the red host may be at least one selected from the group consisting of 9,9′-diphenyl-9H,9′H-3,3′-bicarbazole (BCzPh), CBP, 1,3,5-tris(carbazole-9-yl)benzene (TCP), TCTA, 4,4′-bis(carbazole-9-yl)-2,2′-dimethylbipheyl (CDBP), 2,7-bis(carbazole-9-yl)-9,9-dimethylfluorene (DMFL-CBP), 2,2′,7,7′-tetrakis(carbazole-9-yl)-9,9-spiorofluorene (Spiro-CBP), DPEPO, 4′-(9H-carbazol-9-yl)biphenyl-3,5-dicarbonitrile (PCzB-2CN), 3′-(9H-carbazol-9-yl)biphenyl-3,5-dicarbonitrile (mCzB-2CN), 3,6-bis(carbazole-9-yl)-9-(2-ethyl-hexyl)-9H-carbazole (TCzl), bis(2-hydroxylphenyl)-pyridine)beryllium (Bepp₂), bis(10-hydroxylbenzo[h]quinolinato)beryllium (Bebg₂), and 1,3,5-tris(1-pyrenyl)benzene (TPB₃), but it is not limited thereto.

The red dopant may be at least one selected from the group consisting of [bis(2-(4,6-dimethyl)phenylquinoline)](2,2,6,6-tetramethylheptane-3,5-dionate)iridium(III), bis[2-(4-n-hexylphenyl)quinoline](acetylacetonate)iridium(III) (Hex-Ir(phq)₂(acac)), tris[2-(4-n-hexylphenyl)quinoline]iridium(III), (Hex-Ir(phq)₃), tris[2-phenyl-4-methylquinoline]iridium(III) (Ir(Mphq)₃), bis(2-phenylquinoline)(2,2,6,6-tetramethylheptene-3,5-dionate)iridium(III) (Ir(dpm)PQ₂), bis(phenylisoquinoline)(2,2,6,6-tetramethylheptene-3,5-dionate)iridium(III), (Ir(dpm)(piq)₂), bis[(4-n-hexylphenyl)isoquinoline](acetylacetonate)iridium(III) (Hex-Ir(piq)₂(acac)), tris[2-(4-n-hexylphenyl)quinoline]iridium(III) (Hex-Ir(piq)₃), tris(2-(3-methylphenyl)-7-methyl-quinolato)iridium (Ir(dmpq)₃), bis[2-(2-methylphenyl)-7-methyl-quinoline](acetylacetonate)iridium(III) (Ir(dmpq)₂(acac)), and bis[2-(3,5-dimethylphenyl)-4-methyl-quinoline](acetylacetonate)iridium(III) (Ir(mphmq)₂(acac)), but it is not limited thereto.

The first blue EML 550 in the second emitting part 540 may include a first blue host and a first blue dopant. The second blue EML 570 in the third emitting part 560 may include a second blue host and a second blue dopant.

Each of the first and second blue dopants may include at least one of a blue phosphorescent compound, a blue fluorescent compound, and a blue delayed fluorescent compound. In the first blue EML 550, the first blue host may be present at a weight % greater than the first blue dopant. In the second blue EML 570, the second blue host may be present at a weight % greater than the second blue dopant. In each of the first and second blue EMLs 550 and 570, each of the first and second blue dopants may be present at a weight % of 1 to 10, or 1 to 5, based on a total weight of the components in each of the first and second blue EMLs 550 and 570.

For example, each of the first and second blue hosts may be independently at least one selected from the group consisting of mCP, 9-(3-(9H-carbazol-9-yl)phenyl)-9H-carbazole-3-carbonitrile (mCP-CN), mCBP, CBP-CN, 9-(3-(9H-Carbazol-9-yl)phenyl)-3-(diphenylphosphoryl)-9H-carbazole (mCPPO1) 3,5-Di(9H-carbazol-9-yl)biphenyl (Ph-mCP), TSPO1, 9-(3′-(9H-carbazol-9-yl)-[1,1′-biphenyl]-3-yl)-9H-pyrido[2,3-b]indole (CzBPCb), bis(2-methylphenyl)diphenylsilane (UGH-1), 1,4-bis(triphenylsilyl)benzene (UGH-2), 1,3-bis(triphenylsilyl)benzene (UGH-3), 9,9-spiorobifluoren-2-yl-diphenyl-phosphine oxide (SPPO1), and 9,9′-(5-(triphenylsilyl)-1,3-phenylene)bis(9H-carbazole) (SimCP), but it is not limited thereto.

Each of the first and second blue dopants may be independently at least one selected from the group consisting of 4,4′-bis[4-(di-p-tolylamino)styryl]biphenyl (DPAVBi), 4-(di-p-tolylamino)-4-4′-[(di-p-tolylamino)styryl]stilbene (DPAVB), 4,4′-bis[4-(diphenylamino)styryl]biphenyl (BDAVBi), 2,7-bis(4-diphenylamino)styryl)-9,9-spiorfluorene (spiro-DPVBi), [1,4-bis[2-[4-[N,N-di(p-tolyl)amino]phenyl]vinyl]benzene (DSB), 1-4-di-[4-(N,N-diphenyl)amino]styryl-benzene (DSA), 2,5,8,11-tetra-tert-butylperylene (TBPe), bis(2-hydroxylphenyl)-pyridine)beryllium (Bepp₂), 9-(9-Phenylcarbazole-3-yl)-10-(naphthalene-1-yl)anthracene (PCAN), mer-tris(1-phenyl-3-methylimidazolin-2-ylidene-C,C(2)′iridium(III) (mer-Ir(pmi)₃), fac-Tris(1,3-diphenyl-benzimidazolin-2-ylidene-C,C(2)′iridium(III) (fac-Ir(dpbic)₃), bis(3,4,5-trifluoro-2-(2-pyridyl)phenyl-(2-carboxypyridyl)iridium(III) (Ir(tfpd)₂pic), tris(2-(4,6-difluorophenyl)pyridine))iridium(III) (Ir(Fppy)₃), and bis[2-(4,6-difluorophenyl)pyridinato-C2,N](picolinato)iridium(III) (FIrpic), but it is not limited thereto.

For example, each of the first and second blue EMLs 550 and 570 may include an anthracene derivative as a blue host and a boron derivative as a blue dopant.

The first CGL 580 may be positioned between the first and second emitting parts 530 and 540, and the second CGL 590 is positioned between the first and third emitting parts 530 and 560. For example, the first and second emitting parts 530 and 540 may be connected through the first CGL 580. The first and third emitting parts 530 and 560 may be connected through the second CGL 590. The first CGL 580 may be a P-N junction CGL including a first N-type CGL 582 and a first P-type CGL 584. The second CGL 590 may be a P-N junction CGL including a second N-type CGL 592 and a second P-type CGL 594.

In the first CGL 580, the first N-type CGL 582 may be positioned between the first HTL 532 and the second ETL 546. The first P-type CGL 584 may be positioned between the first N-type CGL 582 and the first HTL 532.

In the second CGL 590, the second N-type CGL 592 may be positioned between the first ETL 534 and the third HTL 562. The second P-type CGL 594 may be positioned between the second N-type CGL 592 and the third HTL 562.

Each of the first and second N-type CGLs 582 and 592 may include the above-mentioned N-type charge generation material, and each of the first and second P-type CGLs 584 and 594 may include the above-mentioned P-type charge generation material.

As illustrated above, the OLED D of the present disclosure may include the first emitting part 530 including the green EML 510a and the red EML 510b, the second emitting part 540 including the first blue EML 550, and the third emitting part 560 including the second blue EML 570 so that the white light may be provided from the OLED D.

The green EML 510a includes the organic compound of the present disclosure represented by Formula 1. As result, in the OLED D, the driving voltage may be reduced, and the emitting efficiency and the lifespan may be improved.

In addition, the green EML 510a may further include the compound, which is represented by Formula 3, as a second host with a first host being the organic compound of the present disclosure so that the OLED D may have improvements in aspects such as the driving voltage, the emitting efficiency, and the emitting lifespan.

Moreover, the green EML 510a may further include the compound, which is represented by Formula 5, as an emitter with the first host represented by Formula 1 and the second host represented by Formula 3 so that the OLED D may have further improvements in aspects such as the driving voltage, the emitting efficiency, and the emitting lifespan.

Furthermore, at least one of the first to third ETLs 534, 546, and 564 may include at least one of a first electron transporting material represented by Formula 7, a second electron transporting material represented by Formula 8 and a third electron transporting material represented by Formula 9 so that the OLED D may have further improvements in aspects such as the driving voltage, the emitting efficiency, and the emitting lifespan.

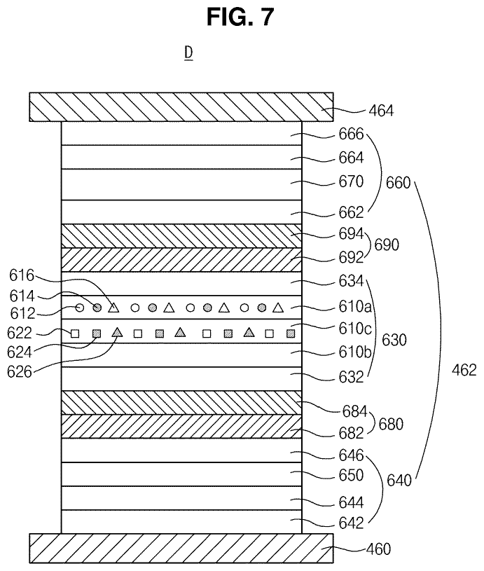

FIG. 7 illustrates a schematic cross-sectional view of an OLED according to a sixth embodiment of the present disclosure.

As illustrated in FIG. 7, the organic light emitting layer 462 may include a first emitting part 630 including a green EML 610a, a red EML 610b, and a yellow-green EML 610c, a second emitting part 640 including a first blue EML 650, and a third emitting part 660 including a second blue EML 670. In addition, the organic light emitting layer 462 may further include a first CGL 680 between the first and second emitting parts 630 and 640 and a second CGL 690 between the first and third emitting parts 630 and 660.

The second emitting part 640 may be positioned between the first electrode 460 and the first emitting part 630. The third emitting part 660 may be positioned between the first emitting part 630 and the second electrode 464. The second emitting part 640 may be positioned between the first electrode 460 and the first CGL 680. The third emitting part 660 may be positioned between the second CGL 690 and the second electrode 464. For example, the second emitting part 640, the first CGL 680, the first emitting part 630, the second CGL 690, and the third emitting part 660 may be sequentially stacked on the first electrode 460.

In the first emitting part 630, the red EML 610b may be disposed under the yellow-green EML 610c. The green EML 610a may be disposed on the yellow-green EML 610c. For example, the first emitting part 530 of the OLED D in FIG. 6 may include an EML having a double-layered structure, which may include EMLs 510a and 510b, while the first emitting part 630 of the OLED D in FIG. 7 may include an EML having a triple-layered structure, which may include EMLs 610a, 610b, and 610c.

The first emitting part 630 may further include a first ETL 634 disposed on the green EML 610a. In addition, the first emitting part 630 may further include a first HTL 632 disposed under the red EML 610b.

For example, in the first emitting part 630, the red EML 610b may be positioned between the first HTL 632 and the yellow-green EML 610c. The green EML 610a may be positioned between the yellow-green EML 610c and the first ETL 634.

The second emitting part 640 may further include at least one of a second HTL 644 disposed under the first blue EML 650 and a second ETL 646 disposed on the first blue EML 650. In addition, the second emitting part 640 may further include an HIL 642 between the first electrode 460 and the second HTL 644.

Moreover, the second emitting part 640 may further include a first EBL (not shown) between the second HTL 644 and the first blue EML 650 and a first HBL (not shown) between the second ETL 646 and the first blue EML 650.

The third emitting part 660 may further include at least one of a third HTL 662 disposed under the second blue EML 670 and a third ETL 664 disposed on the second blue EML 670. In addition, the third emitting part 660 may further include an EIL 666 between the second electrode 464 and the third ETL 664.

Moreover, the third emitting part 660 may further include a second EBL (not shown) between the third HTL 662 and the second blue EML 670 and a second HBL (not shown) between the third ETL 664 and the second blue EML 670.

The green EML 610a may include a first compound 612 that is the organic compound of the present disclosure represented by Formula 1. In addition, the green EML 610a may further include a second compound 614 that is represented by Formula 3. Moreover, the green EML 610a may further include a third compound 616 is the compound represented by Formula 5.

In the green EML 610a, the first compound 612 may be an n-type host (e.g., a first host). The second compound 614 may be a p-type host (e.g., a second host). The third compound 616 may be an emitter (e.g., a dopant). The green EML 610a may have a thickness of 50 to 600 Å.

In the green EML 610a, a weight % of each of the first and second compounds 612 and 614 may be greater than that of the third compound 616, and the weight % of the first compound 612 and the weight % of the second compound 614 may be the same or different. In the green EML 610a, a weight % ratio of the first compound 612 to the second compound 614 may be 1:9 to 9:1, 2:8 to 8:2 or 7:3 to 3:7. In some embodiments, the weight % of the first compound 612 and the weight % of the second compound 614 may be same. For example, the first compound 612 and the second compound 614 may be present at the same weight %, and the third compound 616 may have a weight % of 5 to 25 in the green EML 610a.

The yellow-green EML 610c may include a first compound 622 that is the organic compound of the present disclosure represented by Formula 1. In addition, the yellow-green EML 610c may further include a second compound 624 that is represented by Formula 3. Moreover, the yellow-green EML 610c may further include a third compound 626 is the compound represented by Formula 6.

In the yellow-green EML 610c, the first compound 622 may be an n-type host (e.g., a first host). The second compound 624 may be a p-type host (e.g., a second host). The third compound 626 may be an emitter (e.g., a dopant). The yellow-green EML 610c may have a thickness of 50 to 600 Å.

In the yellow-green EML 610c, a weight % of each of the first and second compounds 622 and 624 may be greater than that of the third compound 626, and the weight % of the first compound 622 and the weight % of the second compound 624 may be the same or different. In the yellow-green EML 610c, a weight % ratio of the first compound 622 to the second compound 624 may be 1:9 to 9:1, 2:8 to 8:2 or 7:3 to 3:7. In some embodiments, the weight % of the first compound 622 and the weight % of the second compound 624 may be same. For example, the first compound 622 and the second compound 624 may be present at the same weight %, and the third compound 626 may have a weight % of 5 to 25 in the yellow-green EML 610c.

Each of the first to third ETLs 634, 646, and 664 may include at least one of the first electron transporting material represented by Formula 7, a second electron transporting material represented by Formula 8 and a third electron transporting material represented by Formula 9.

The red EML 610b may include a red host and a red dopant. The red dopant may include at least one of a red phosphorescent compound, a red fluorescent compound, and a red delayed fluorescent compound. In the red EML 610b, the red host may be present at a weight % greater than the red dopant. In the red EML 610b, the red dopant may be present at a weight % of 1 to 10, e.g., 1 to 5, based on a total weight of the components in the red EML 610b.

The first blue EML 650 in the second emitting part 640 may include a first blue host and a first blue dopant. The second blue EML 670 in the third emitting part 660 may include a second blue host and a second blue dopant.

Each of the first and second blue dopants may include at least one of a blue phosphorescent compound, a blue fluorescent compound, and a blue delayed fluorescent compound. In the first blue EML 650, the first blue host may be present at a weight % greater than the first blue dopant. In the second blue EML 670, the second blue host may be present at a weight % greater than the second blue dopant. In each of the first and second blue EMLs 650 and 670, each of the first and second blue dopants may be present at a weight % of 1 to 10, e.g., 1 to 5, based on a total weight of the components in each of the first and second blue EMLs 650 and 670.

The first CGL 680 may be positioned between the first and second emitting parts 630 and 640. The second CGL 690 may be positioned between the first and third emitting parts 630 and 660. For example, the first and second emitting parts 630 and 640 may be connected through the first CGL 680. The first and third emitting parts 630 and 660 may be connected through the second CGL 690. The first CGL 680 may be a P-N junction CGL including a first N-type CGL 682 and a first P-type CGL 684. The second CGL 690 may be a P-N junction CGL including a second N-type CGL 692 and a second P-type CGL 694.

In the first CGL 680, the first N-type CGL 682 may be positioned between the first HTL 632 and the second ETL 646. The first P-type CGL 684 may be positioned between the first N-type CGL 682 and the first HTL 632.

In the second CGL 690, the second N-type CGL 692 may be positioned between the first ETL 634 and the third HTL 662. The second P-type CGL 694 may be positioned between the second N-type CGL 692 and the third HTL 662.

As illustrated above, the OLED D according to an example embodiment of the present disclosure may include the first emitting part 630 including the green EML 610a, the red EML 610b, and the yellow-green EML 610c, the second emitting part 640 including the first blue EML 650, and the third emitting part 660 including the second blue EML 670 so that the white light may be provided from the OLED D.

At least one of the green EML 610a and the yellow-green EML 610c includes the organic compound of the present disclosure represented by Formula 1. As result, in the OLED D, the driving voltage may be reduced, and the emitting efficiency and the lifespan may be improved.

In addition, at least one of the green EML 610a and the yellow-green EML 610c may further include the compound, which is represented by Formula 3, as a second host with a first host being the organic compound of the present disclosure so that the OLED D may have improvements in aspects such as the driving voltage, the emitting efficiency, and the emitting lifespan.

Moreover, the green EML 610a may further include the compound, which is represented by Formula 5, as an emitter with the first host represented by Formula 1 and the second host represented by Formula 3 so that the OLED D may have further improvements in aspects such as the driving voltage, the emitting efficiency, and the emitting lifespan.

The yellow-green EML 610c may further include the compound, which is represented by Formula 6, as an emitter with the first host represented by Formula 1 and the second host represented by Formula 3 so that the OLED D may have further improvements in aspects such as the driving voltage, the emitting efficiency, and the emitting lifespan.

Furthermore, at least one of the first to third ETLs 634, 646, and 664 may include at least one of a first electron transporting material represented by Formula 7, a second electron transporting material represented by Formula 8 and a third electron transporting material represented by Formula 9 so that the OLED D may have further improvements in aspects such as the driving voltage, the emitting efficiency, and the emitting lifespan.

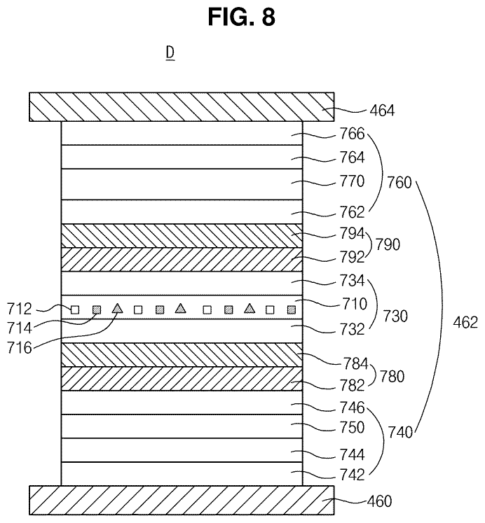

FIG. 8 illustrates a schematic cross-sectional view of an OLED according to a seventh embodiment of the present disclosure.

As illustrated in FIG. 8, the organic light emitting layer 462 may include a first emitting part 730 including a yellow-green EML 710, a second emitting part 740 including a first blue EML 750, and a third emitting part 760 including a second blue EML 770. In addition, the organic light emitting layer 462 may further include a first CGL 780 between the first and second emitting parts 730 and 740 and a second CGL 790 between the first and third emitting parts 730 and 760.

The second emitting part 740 may be positioned between the first electrode 460 and the first emitting part 730. The third emitting part 760 may be positioned between the first emitting part 730 and the second electrode 464. The second emitting part 740 may be positioned between the first electrode 460 and the first CGL 780. The third emitting part 760 may be positioned between the second CGL 790 and the second electrode 464. For example, the second emitting part 740, the first CGL 780, the first emitting part 730, the second CGL 790, and the third emitting part 760 may be sequentially stacked on the first electrode 460.

The first emitting part 530 of the OLED D in FIG. 6 may include an EML having a double-layered structure, which may include EMLs 510a and 510b. The first emitting part 630 of the OLED D in FIG. 7 may include an EML having a triple-layered structure, which may include EMLs 610a, 610b, and 610c. The first emitting part 730 of the OLED D in FIG. 8 may include an EML having a single-layered structure.

The first emitting part 730 may further include at least one of a first ETL 734 disposed on the yellow-green EML 710 and a first HTL 732 disposed under the yellow-green EML 710.

For example, in the first emitting part 730, a lower surface of the yellow-green EML 710 may contact the first HTL 732, and an upper surface of the yellow-green EML 710 may contact the first ETL 734.

The second emitting part 740 may further include at least one of a second HTL 744 disposed under the first blue EML 750 and a second ETL 746 disposed on the first blue EML 750. In addition, the second emitting part 740 may further include an HIL 742 between the first electrode 460 and the second HTL 744.

Moreover, the second emitting part 740 may further include a first EBL (not shown) between the second HTL 744 and the first blue EML 750 and a first HBL (not shown) between the second ETL 746 and the first blue EML 750.

The third emitting part 760 may further include at least one of a third HTL 762 disposed under the second blue EML 770 and a third ETL 764 disposed on the second blue EML 770. In addition, the third emitting part 760 may further include an EIL 766 between the second electrode 464 and the third ETL 764.

Moreover, the third emitting part 760 may further include a second EBL (not shown) between the third HTL 762 and the second blue EML 770 and a second HBL (not shown) between the third ETL 764 and the second blue EML 770.

The yellow-green EML 710 may include a first compound 712 that is the organic compound of the present disclosure represented by Formula 1. In addition, the yellow-green EML 710 may further include a second compound 714 that is represented by Formula 3.

Moreover, the yellow-green EML 710 may further include a third compound 716 is the compound represented by Formula 6.

In the yellow-green EML 710, the first compound 712 may be an n-type host (e.g., a first host). The second compound 714 may be a p-type host (e.g., a second host). The third compound 716 may be an emitter (e.g., a dopant). The yellow-green EML 710 may have a thickness of 50 to 600 Å.

In the yellow-green EML 710, a weight % of each of the first and second compounds 712 and 714 may be greater than that of the third compound 716, and the weight % of the first compound 712 and the weight % of the second compound 714 may be the same or different. In the yellow-green EML 710, a weight % ratio of the first compound 712 to the second compound 714 may be 1:9 to 9:1, 2:8 to 8:2 or 7:3 to 3:7. In some embodiments, the weight % of the first compound 712 and the weight % of the second compound 714 may be same. For example, the first compound 712 and the second compound 714 may be present at the same weight %, and the third compound 716 may have a weight % of 5 to 25 in the yellow-green EML 710.

Each of the first to third ETLs 734, 746, and 764 may include at least one of the first electron transporting material represented by Formula 7, a second electron transporting material represented by Formula 8 and a third electron transporting material represented by Formula 9.

The first blue EML 750 in the second emitting part 740 may include a first blue host and a first blue dopant. The second blue EML 770 in the third emitting part 760 may include a second blue host and a second blue dopant.

Each of the first and second blue dopants may include at least one of a blue phosphorescent compound, a blue fluorescent compound, and a blue delayed fluorescent compound. In the first blue EML 750, the first blue host may be present at a weight % greater than the first blue dopant. In the second blue EML 770, the second blue host may be present at a weight % greater than the second blue dopant. In each of the first and second blue EMLs 750 and 770, each of the first and second blue dopants may be present at a weight % of 1 to 10, e.g., 1 to 5, based on a total weight of the components in each of the first and second blue EMLs 750 and 770.

The first CGL 780 may be positioned between the first and second emitting parts 730 and 740. The second CGL 790 may be positioned between the first and third emitting parts 730 and 760. For example, the first and second emitting parts 730 and 740 may be connected through the first CGL 780. The first and third emitting parts 730 and 760 may be connected through the second CGL 790. The first CGL 780 may be a P-N junction CGL including a first N-type CGL 782 and a first P-type CGL 784. The second CGL 790 may be a P-N junction CGL including a second N-type CGL 792 and a second P-type CGL 794.

In the first CGL 780, the first N-type CGL 782 may be positioned between the first HTL 732 and the second ETL 746. The first P-type CGL 784 may be positioned between the first N-type CGL 782 and the first HTL 732.

In the second CGL 790, the second N-type CGL 792 may be positioned between the first ETL 734 and the third HTL 762. The second P-type CGL 794 may be positioned between the second N-type CGL 792 and the third HTL 762.

As illustrated above, the OLED D according to an example embodiment of the present disclosure may include the first emitting part 730 including the yellow-green EML 710, the second emitting part 740 including the first blue EML 750, and the third emitting part 760 including the second blue EML 770 so that the white light may be provided from the OLED D.

The yellow-green EML 710 includes the organic compound of the present disclosure represented by Formula 1. As result, in the OLED D, the driving voltage may be reduced, and the emitting efficiency and the lifespan may be improved.

In addition, the yellow-green EML 710 may further include the compound, which is represented by Formula 3, as a second host with a first host being the organic compound of the present disclosure so that the OLED D may have improvements in aspects such as the driving voltage, the emitting efficiency, and the emitting lifespan.

The yellow-green EML 710 may further include the compound, which is represented by Formula 6, as an emitter with the first host represented by Formula 1 and the second host represented by Formula 3 so that the OLED D may have further improvements in aspects such as the driving voltage, the emitting efficiency, and the emitting lifespan.

Furthermore, at least one of the first to third ETLs 734, 746 and 764 may include at least one of a first electron transporting material represented by Formula 7, a second electron transporting material represented by Formula 8 and a third electron transporting material represented by Formula 9 so that the OLED D may have further improvements in aspects such as the driving voltage, the emitting efficiency, and the emitting lifespan.

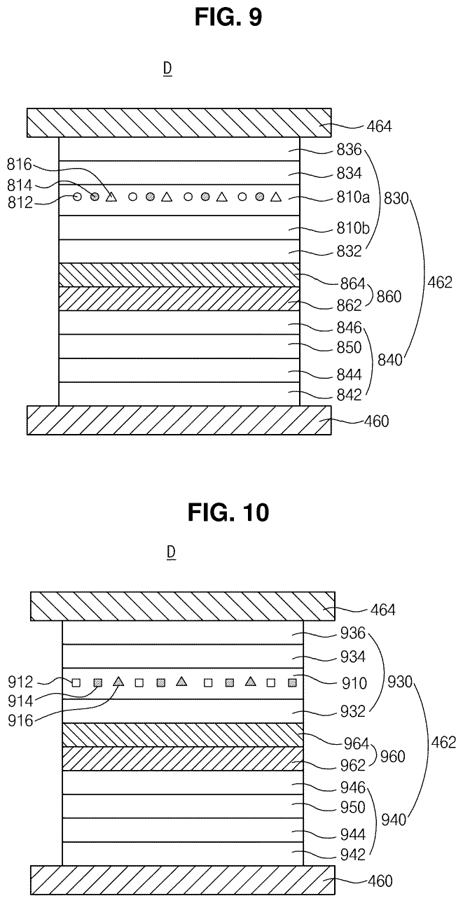

FIG. 9 illustrates a schematic cross-sectional view of an OLED according to a eighth embodiment of the present disclosure.

As illustrated in FIG. 9, the organic light emitting layer 462 may include a first emitting part 830 including a green EML 810a and a second emitting part 840 including a blue EML 850. In addition, the organic light emitting layer 462 may further include a CGL 860 between the first and second emitting parts 830 and 840. In addition, the first emitting part 830 may further include a red EML 810b.

The second emitting part 840 may be positioned between the first electrode 460 and the first emitting part 830. The first emitting part 830 may be positioned between the CGL 860 and the second electrode 464, and the second emitting part 840 may be positioned between the first electrode 460 and the CGL 860. For example, the second emitting part 840, the CGL 860 and the first emitting part 830 may be sequentially stacked on the first electrode 460.

The OLED of FIG. 6 includes the first to third emitting parts 530, 540, and 560, the OLED of FIG. 7 includes the first to third emitting parts 630, 640 and 660, and the OLED of FIG. 8 includes the first to third emitting parts 730, 740 and 760. On the other hand, the OLED of FIG. 9 includes the first and second emitting parts 830 and 840,

In the first emitting part 830, the red EML 810b may be disposed under the green EML 810a.

The first emitting part 830 may further include a first ETL 834 disposed on the green EML 810a. In addition, the first emitting part 830 may further include a first HTL 832 disposed under the red EML 810b.

Moreover, the first emitting part 830 may further include an EIL 836 on or over the first ETL 834.

The first emitting part 830 may further include at least one of a first EBL (not shown) between the first HTL 832 and the red EML 810b and a first HBL (not shown) between the first ETL 834 and the green EML 810a.

The second emitting part 840 may further include at least one of a second HTL 844 disposed under the first blue EML 850 and a second ETL 846 disposed on the first blue EML 850. In addition, the second emitting part 840 may further include an HIL 842 between the first electrode 460 and the second HTL 844.

Moreover, the second emitting part 840 may further include a second EBL (not shown) between the second HTL 844 and the first blue EML 850 and a second HBL (not shown) between the second ETL 846 and the first blue EML 850.

The green EML 810a may include a first compound 812 that is the organic compound of the present disclosure represented by Formula 1. In addition, the green EML 810a may further include a second compound 814 that is the compound represented by Formula 3. Moreover, the green EML 810a may further include a third compound 816 that is the compound represented by Formula 5.

In the green EML 810a, the first compound 812 may be an n-type host (e.g., a first host), the second compound 814 may be a p-type host (e.g., a second host), and the third compound 816 may be an emitter (e.g., a dopant). The green EML 810a may have a thickness of 50 to 600 Å.

In the green EML 810a, a weight % of each of the first and second compounds 812 and 814 may be greater than that of the third compound 816. The weight % of the first compound 812 and the weight % of the second compound 814 may be the same or different. In the green EML 810a, a weight % ratio of the first compound 812 to the second compound 814 may be 1:9 to 9:1, 2:8 to 8:2 or 7:3 to 3:7. In some embodiments, the weight % of the first compound 812 and the weight % of the second compound 814 may be same. The third compound 816 may be present in an amount of 5 to 25 weight % in the green EML 810a, based on a total weight of the components in the green EML 810a.

Each of the first and second ETLs 834 and 846 may include at least one of the first electron transporting material represented by Formula 7, the second electron transporting material represented by Formula 8 and the third electron transporting material represented by Formula 9.

The red EML 810b may include a red host and a red dopant. The red dopant may include at least one of a red phosphorescent compound, a red fluorescent compound, and a red delayed fluorescent compound. In the red EML 810b, the red host may be present at a weight % greater than the red dopant. In the red EML 810b, the red dopant may be present at a weight % of 1 to 10, or 1 to 5, based on a total weight of the components in the red EML 810b.

The blue EML 850 in the second emitting part 840 may include a blue host and a blue dopant. The blue dopant may include at least one of a blue phosphorescent compound, a blue fluorescent compound, and a blue delayed fluorescent compound. The blue host may be the above-mentioned blue host material, and the blue dopant may be the above-mentioned blue dopant.

In the blue EML 850, the blue host may be present at a weight % greater than the blue dopant. In the blue EML 850, the blue dopant may be present at a weight % of 1 to 10, or 1 to 5, based on a total weight of the components in the blue EML 850.

The CGL 860 may be positioned between the first and second emitting parts 830 and 840. For example, the first and second emitting parts 830 and 840 may be connected through the CGL 860. The CGL 860 may be a P-N junction CGL including a N-type CGL 862 and a P-type CGL 864.

In the CGL 860, the N-type CGL 862 may be positioned between the first HTL 832 and the second ETL 846. The P-type CGL 864 may be positioned between the N-type CGL 862 and the first HTL 832.

As illustrated above, the OLED D of the present disclosure may include the first emitting part 830 including the green EML 810a and the red EML 810b and the second emitting part 840 including the blue EML 850 so that the white light may be provided from the OLED D.

The green EML 810a includes the organic compound of the present disclosure represented by Formula 1. As result, in the OLED D, the driving voltage may be reduced, and the emitting efficiency and the lifespan may be improved.

In addition, the green EML 810a may further include the compound, which is represented by Formula 3, as a second host with a first host being the organic compound of the present disclosure so that the OLED D may have improvements in aspects such as the driving voltage, the emitting efficiency, and the emitting lifespan.

Moreover, the green EML 810a may further include the compound, which is represented by Formula 5, as an emitter with the first host represented by Formula 1 and the second host represented by Formula 3 so that the OLED D may have further improvements in aspects such as the driving voltage, the emitting efficiency, and the emitting lifespan.

Furthermore, at least one of the first and second ETLs 834 and 846 may include at least one of a first electron transporting material represented by Formula 7, a second electron transporting material represented by Formula 8 and a third electron transporting material represented by Formula 9 so that the OLED D may have further improvements in aspects such as the driving voltage, the emitting efficiency, and the emitting lifespan.

FIG. 10 illustrates a schematic cross-sectional view of an OLED according to a ninth embodiment of the present disclosure.

As illustrated in FIG. 10, the organic light emitting layer 462 may include a first emitting part 930 including a yellow-green EML 910 and a second emitting part 940 including a blue EML 950. In addition, the organic light emitting layer 462 may further include a CGL 960 between the first and second emitting parts 930 and 940.

The second emitting part 940 may be positioned between the first electrode 460 and the first emitting part 930. The first emitting part 930 may be positioned between the CGL 960 and the second electrode 464, and the second emitting part 940 may be positioned between the first electrode 460 and the CGL 960. For example, the second emitting part 940, the CGL 960 and the first emitting part 930 may be sequentially stacked on the first electrode 460.

The first emitting part 830 in the OLED D of FIG. 9 includes the EML having a double-layered structure, while the first emitting part 930 in the OLED D of FIG. 10 includes the EML having single-layered structure.

The first emitting part 930 may further include a first ETL 934 disposed on the yellow-green EML 910 and a first HTL 932 disposed under the yellow-green EML 910.

Moreover, the first emitting part 930 may further include an EIL 936 on or over the first EML 934.

The first emitting part 930 may further include at least one of a first EBL (not shown) between the first HTL 932 and the yellow-green EML 910 and a first HBL (not shown) between the first ETL 934 and the yellow-green EML 910.

The second emitting part 940 may further include at least one of a second HTL 944 disposed under the first blue EML 950 and a second ETL 946 disposed on the first blue EML 950. In addition, the second emitting part 940 may further include an HIL 942 between the first electrode 460 and the second HTL 944.

Moreover, the second emitting part 940 may further include a second EBL (not shown) between the second HTL 944 and the first blue EML 950 and a second HBL (not shown) between the second ETL 946 and the first blue EML 950.

The yellow-green EML 910 may include a first compound 912 that is the organic compound of the present disclosure represented by Formula 1. In addition, the yellow-green EML 910 may further include a second compound 914 that is the compound represented by Formula 3. Moreover, the yellow-green EML 910 may further include a third compound 916 that is the compound represented by Formula 6.

In the yellow-green EML 910, the first compound 912 may be an n-type host (e.g., a first host), the second compound 914 may be a p-type host (e.g., a second host), and the third compound 916 may be an emitter (e.g., a dopant). The yellow-green EML 910 may have a thickness of 50 to 600 Å.

In the yellow-green EML 910, a weight % of each of the first and second compounds 912 and 914 may be greater than that of the third compound 916. The weight % of the first compound 912 and the weight % of the second compound 914 may be the same or different. In the yellow-green EML 910, a weight % ratio of the first compound 912 to the second compound 914 may be 1:9 to 9:1, 2:8 to 8:2 or 7:3 to 3:7. In some embodiments, the weight % of the first compound 912 and the weight % of the second compound 914 may be same. The third compound 916 may be present in an amount of 5 to 25 weight % in the yellow-green EML 910, based on a total weight of the components in the yellow-green EML 910.

Each of the first and second ETLs 934 and 946 may include at least one of the first electron transporting material represented by Formula 7, the second electron transporting material represented by Formula 8 and the third electron transporting material represented by Formula 9.

The blue EML 950 in the second emitting part 940 may include a blue host and a blue dopant. The blue dopant may include at least one of a blue phosphorescent compound, a blue fluorescent compound, and a blue delayed fluorescent compound. The blue host may be the above-mentioned blue host material, and the blue dopant may be the above-mentioned blue dopant.

In the blue EML 950, the blue host may be present at a weight % greater than the blue dopant. In the blue EML 950, the blue dopant may be present at a weight % of 1 to 10, or 1 to 5, based on a total weight of the components in the blue EML 950.

The CGL 960 may be positioned between the first and second emitting parts 930 and 940. For example, the first and second emitting parts 930 and 940 may be connected through the CGL 960. The CGL 960 may be a P-N junction CGL including a N-type CGL 962 and a P-type CGL 964.

In the CGL 960, the N-type CGL 962 may be positioned between the first HTL 932 and the second ETL 946. The P-type CGL 964 may be positioned between the N-type CGL 962 and the first HTL 932.

As illustrated above, the OLED D of the present disclosure may include the first emitting part 930 including the yellow-green EML 910 and the second emitting part 940 including the blue EML 950 so that the white light may be provided from the OLED D.

The yellow-green EML 910 includes the organic compound of the present disclosure represented by Formula 1. As result, in the OLED D, the driving voltage may be reduced, and the emitting efficiency and the lifespan may be improved.

In addition, the yellow-green EML 910 may further include the compound, which is represented by Formula 3, as a second host with a first host being the organic compound of the present disclosure so that the OLED D may have improvements in aspects such as the driving voltage, the emitting efficiency, and the emitting lifespan.

Moreover, the yellow-green EML 910 may further include the compound, which is represented by Formula 6, as an emitter with the first host represented by Formula 1 and the second host represented by Formula 3 so that the OLED D may have further improvements in aspects such as the driving voltage, the emitting efficiency, and the emitting lifespan.

Furthermore, at least one of the first and second ETLs 934 and 946 may include at least one of a first electron transporting material represented by Formula 7, a second electron transporting material represented by Formula 8 and a third electron transporting material represented by Formula 9 so that the OLED D may have further improvements in aspects such as the driving voltage, the emitting efficiency, and the emitting lifespan.

It will be apparent to those skilled in the art that various modifications and variations can be made in the embodiments of the present disclosure without departing from the spirit or scope of the present disclosure. Thus, it is intended that the modifications and variations cover this disclosure provided they come within the scope of the appended claims and their equivalents.

What is claimed is:

1. An organic compound represented by Formula 1: 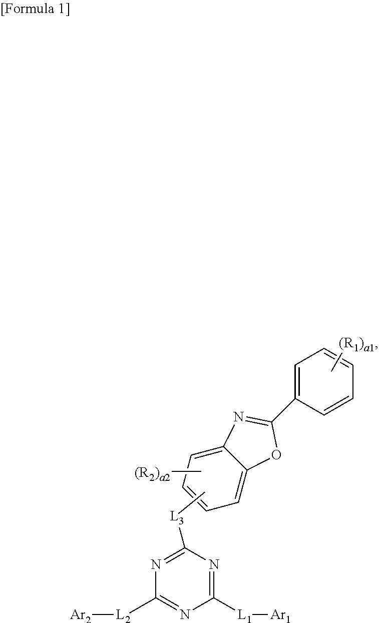
wherein a1 is an integer of 0 to 5, a2 is an integer of 0 to 3,
wherein each R₁ is independently selected from the group consisting of a substituted or unsubstituted C1 to C10 alkyl group,
wherein each R₂ is independently selected from the group consisting of a substituted or unsubstituted C1 to C10 alkyl group, a substituted or unsubstituted C6 to C60 aryl group and a substituted or unsubstituted C3 to C60 heteroaryl group,
wherein when a1 is 2 or more, 2 or more R₁ groups are the same or different,
wherein when a2 is 2 or more, 2 or more R₂ groups are the same or different,
wherein each L₁, L₂ and L₃ is independently selected from the group consisting of a single bond, a substituted or unsubstituted C6 to C60 arylene group and a substituted or unsubstituted C3 to C60 heteroarylene group,
wherein Ar₁ is selected from Formula 1a-1 to Formula 1a-5: 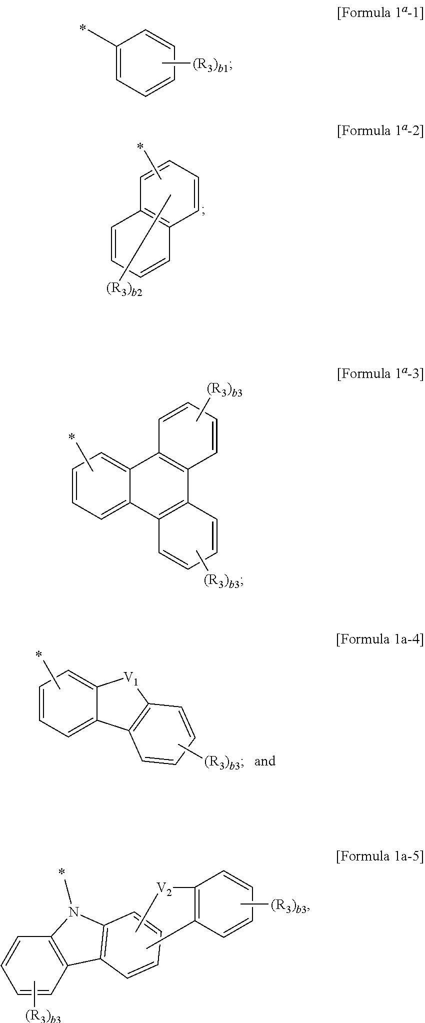
wherein in Formula 1a-1, b1 is an integer of 0 to 5,
wherein in Formula 1a-2, b2 is an integer of 0 to 7,
wherein in each of Formulas 1a-3 to 1a-5, each b3 is independently an integer of 0 to 4,
wherein when each b1, b2, and b3 is 2 or more, 2 or more R₃ groups are the same or different,
wherein in Formula 1a-4, V₁ is selected from O, S, and C(R₃)₂,
wherein in Formula 1a-5, V₂ is selected from O and S,
wherein in each of Formulas 1a-1 to 1a-5, each R₃ is independently selected from the group consisting of deuterium, a substituted or unsubstituted C1 to C10 alkyl group, a substituted or unsubstituted C6 to C60 aryl group and a substituted or unsubstituted C3 to C60 heteroaryl group,
wherein Ar₂ is selected from Formula 1b-1 and Formula 1b-2, 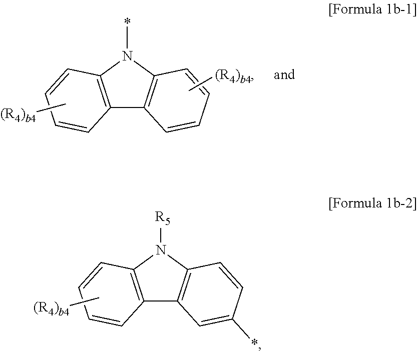
wherein in each of Formulas 1b-1 and 1b-2, b4 is an integer of 0 to 4,
wherein each R₄ is independently selected from the group consisting of deuterium, a substituted or unsubstituted C1 to C10 alkyl group, a substituted or unsubstituted C6 to C60 aryl group and a substituted or unsubstituted C3 to C60 heteroaryl group,
wherein when b4 is 2 or more, 2 or more R₄ groups are the same or different, and
wherein in Formula 1b-2, R₅ is selected from the group consisting of hydrogen, deuterium, a substituted or unsubstituted C1 to C10 alkyl group and a substituted or unsubstituted C6 to C60 aryl group.

2. The organic compound according to claim 1, wherein Ar₁ and Ar₂ are different.

3. The organic compound according to claim 1, wherein Formula 1 is represented by Formula 1-5 or Formula 1-6:

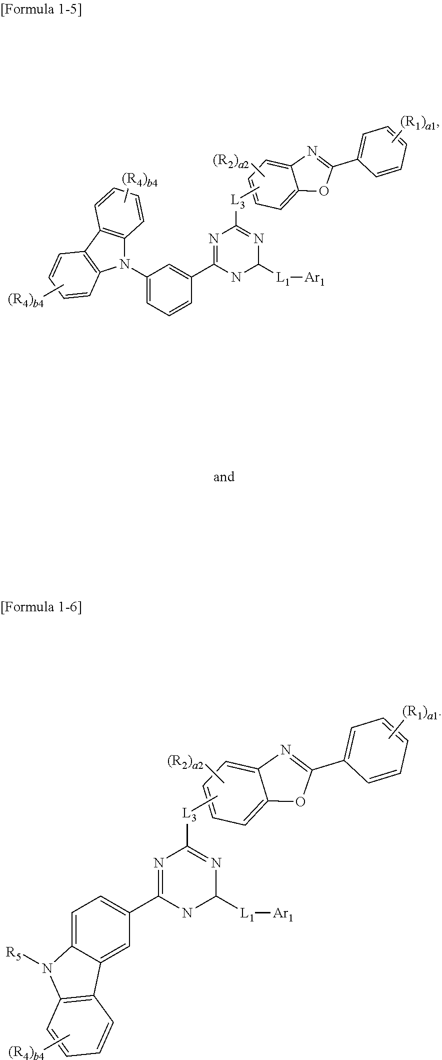

4. The organic compound according to claim 1, wherein the organic compound is one of the compounds in Formula 2:

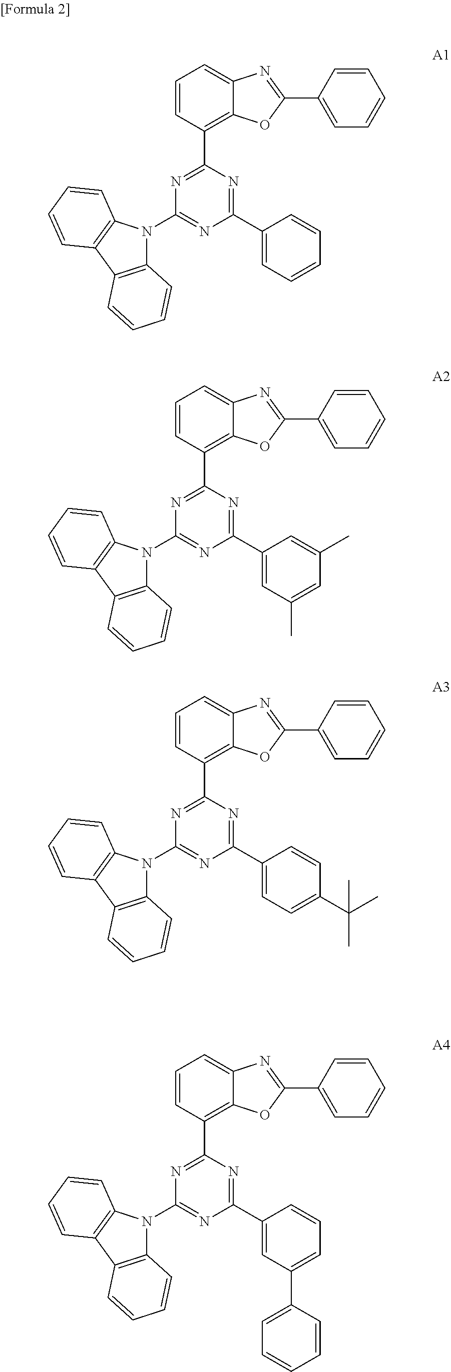 [SMILES: c1ccc(-c2nc(-c3cccc4nc(-c5ccccc5)oc34)nc(-n3c4ccccc4c4ccccc43)n2)cc1 ; Cc1cc(C)cc(-c2nc(-c3cccc4nc(-c5ccccc5)oc34)nc(-n3c4ccccc4c4ccccc43)n2)c1 ; CC(C)(C)c1ccc(-c2nc(-c3cccc4nc(-c5ccccc5)oc34)nc(-n3c4ccccc4c4ccccc43)n2)cc1 ; c1ccc(-c2cccc(-c3nc(-c4cccc5nc(-c6ccccc6)oc45)nc(-n4c5ccccc5c5ccccc54)n3)c2)cc1]

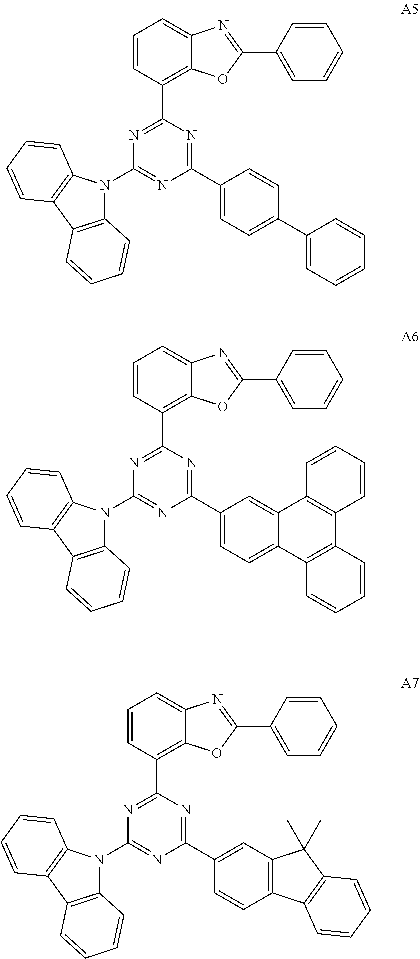 [SMILES: c1ccc(-c2ccc(-c3nc(-c4cccc5nc(-c6ccccc6)oc45)nc(-n4c5ccccc5c5ccccc54)n3)cc2)cc1 ; c1ccc(-c2nc3cccc(-c4nc(-c5ccc6c7ccccc7c7ccccc7c6c5)nc(-n5c6ccccc6c6ccccc65)n4)c3o2)cc1 ; CC1(C)c2ccccc2-c2ccc(-c3nc(-c4cccc5nc(-c6ccccc6)oc45)nc(-n4c5ccccc5c5ccccc54)n3)cc21]

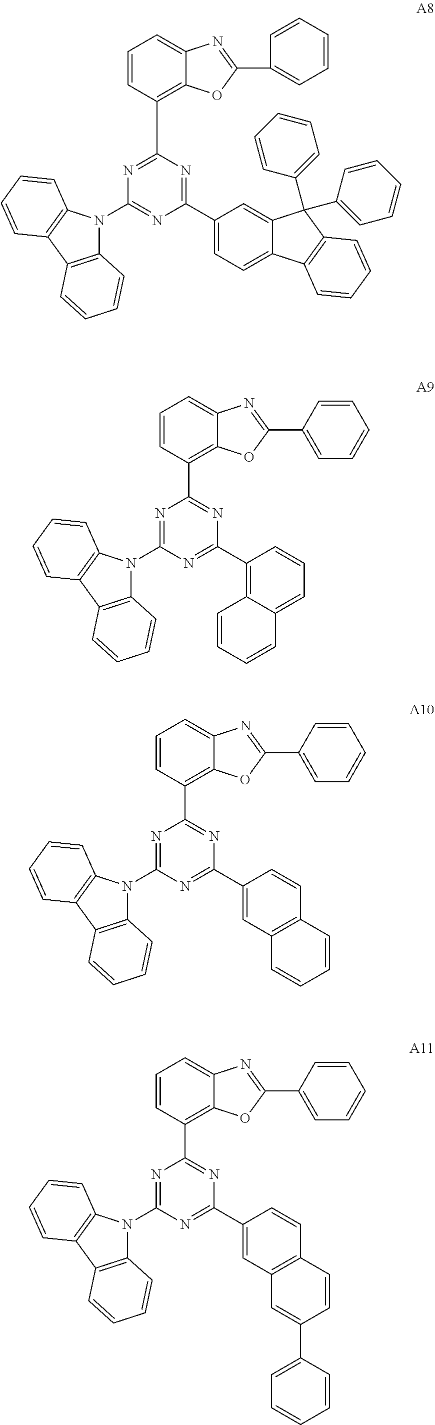 [SMILES: c1ccc(-c2nc3cccc(-c4nc(-c5ccc6c(c5)C(c5ccccc5)(c5ccccc5)c5ccccc5-6)nc(-n5c6ccccc6c6ccccc65)n4)c3o2)cc1 ; c1ccc(-c2nc3cccc(-c4nc(-c5cccc6ccccc56)nc(-n5c6ccccc6c6ccccc65)n4)c3o2)cc1 ; c1ccc(-c2nc3cccc(-c4nc(-c5ccc6ccccc6c5)nc(-n5c6ccccc6c6ccccc65)n4)c3o2)cc1 ; c1ccc(-c2ccc3ccc(-c4nc(-c5cccc6nc(-c7ccccc7)oc56)nc(-n5c6ccccc6c6ccccc65)n4)cc3c2)cc1]

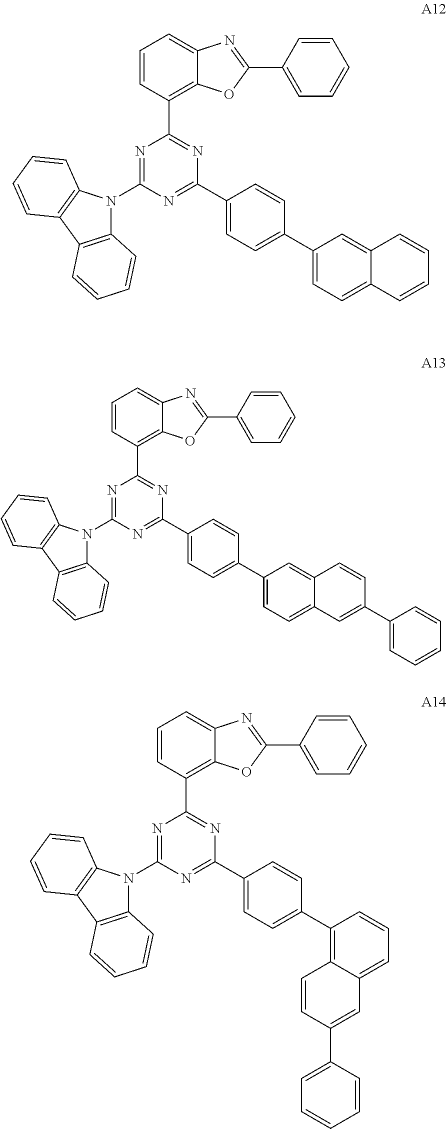 [SMILES: c1ccc(-c2nc3cccc(-c4nc(-c5ccc(-c6ccc7ccccc7c6)cc5)nc(-n5c6ccccc6c6ccccc65)n4)c3o2)cc1 ; c1ccc(-c2ccc3cc(-c4ccc(-c5nc(-c6cccc7nc(-c8ccccc8)oc67)nc(-n6c7ccccc7c7ccccc76)n5)cc4)ccc3c2)cc1 ; c1ccc(-c2ccc3c(-c4ccc(-c5nc(-c6cccc7nc(-c8ccccc8)oc67)nc(-n6c7ccccc7c7ccccc76)n5)cc4)cccc3c2)cc1]

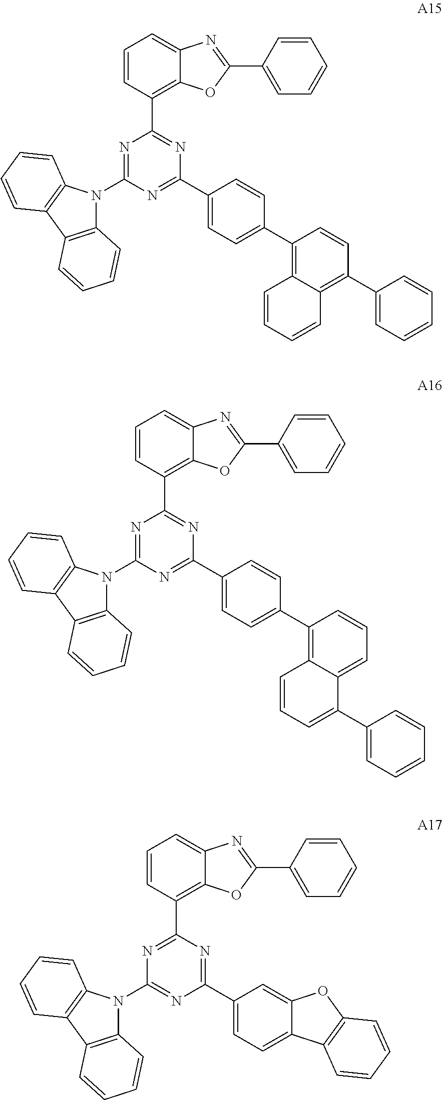

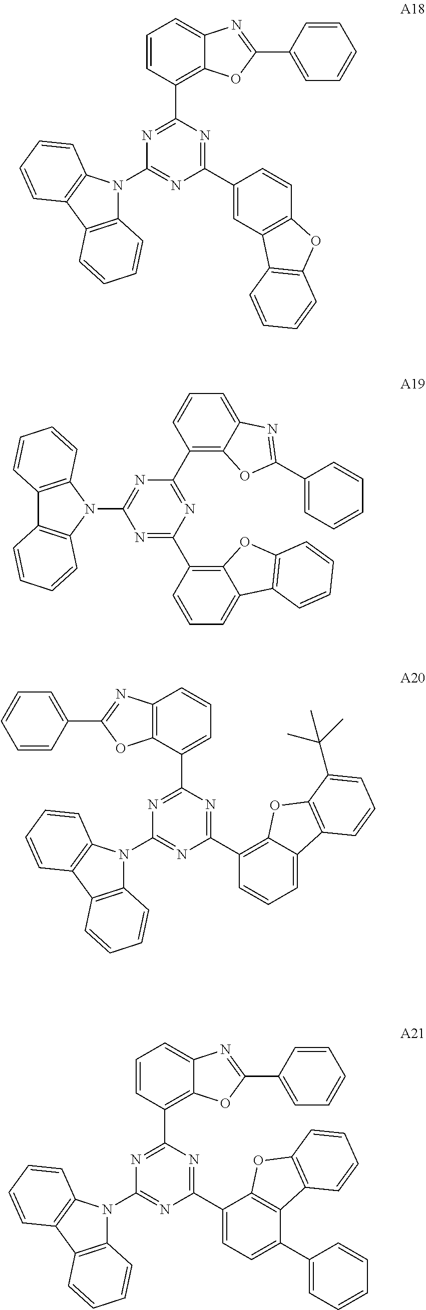 [SMILES: c1ccc(-c2nc3cccc(-c4nc(-c5ccc6oc7ccccc7c6c5)nc(-n5c6ccccc6c6ccccc65)n4)c3o2)cc1 ; c1ccc(-c2nc3cccc(-c4nc(-c5cccc6c5oc5ccccc56)nc(-n5c6ccccc6c6ccccc65)n4)c3o2)cc1 ; CC(C)(C)c1cccc2c1oc1c(-c3nc(-c4cccc5nc(-c6ccccc6)oc45)nc(-n4c5ccccc5c5ccccc54)n3)cccc12 ; c1ccc(-c2nc3cccc(-c4nc(-c5ccc(-c6ccccc6)c6c5oc5ccccc56)nc(-n5c6ccccc6c6ccccc65)n4)c3o2)cc1]

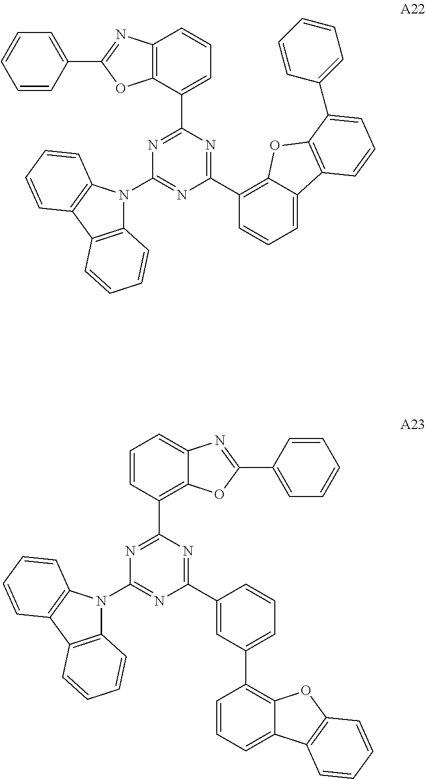 [SMILES: c1ccc(-c2nc3cccc(-c4nc(-c5cccc6c5oc5c(-c7ccccc7)cccc56)nc(-n5c6ccccc6c6ccccc65)n4)c3o2)cc1 ; c1ccc(-c2nc3cccc(-c4nc(-c5cccc(-c6cccc7c6oc6ccccc67)c5)nc(-n5c6ccccc6c6ccccc65)n4)c3o2)cc1]

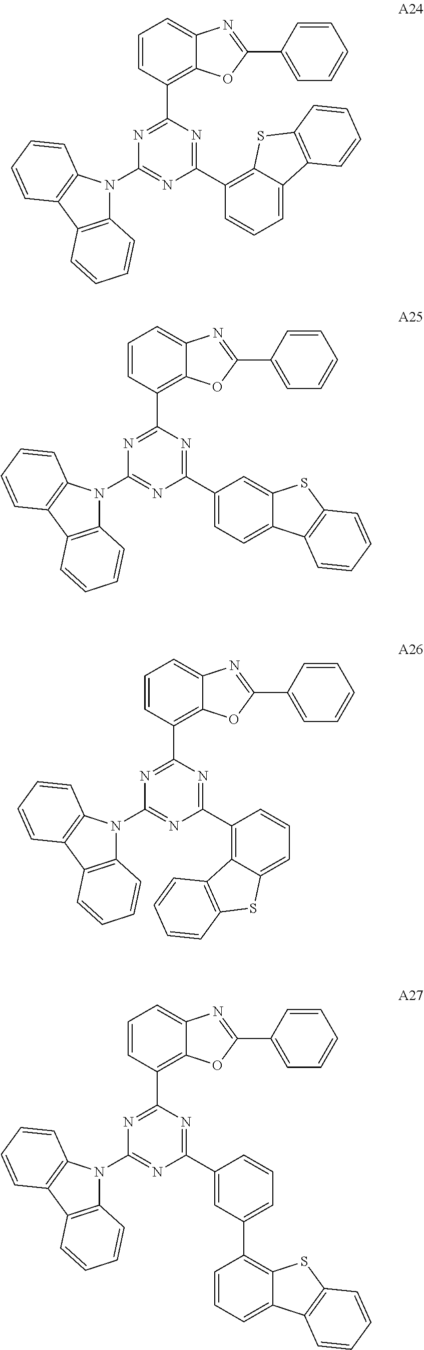 [SMILES: c1ccc(-c2nc3cccc(-c4nc(-c5cccc6c5sc5ccccc56)nc(-n5c6ccccc6c6ccccc65)n4)c3o2)cc1 ; c1ccc(-c2nc3cccc(-c4nc(-c5ccc6c(c5)sc5ccccc56)nc(-n5c6ccccc6c6ccccc65)n4)c3o2)cc1 ; c1ccc(-c2nc3cccc(-c4nc(-c5cccc6sc7ccccc7c56)nc(-n5c6ccccc6c6ccccc65)n4)c3o2)cc1 ; c1ccc(-c2nc3cccc(-c4nc(-c5cccc(-c6cccc7c6sc6ccccc67)c5)nc(-n5c6ccccc6c6ccccc65)n4)c3o2)cc1]

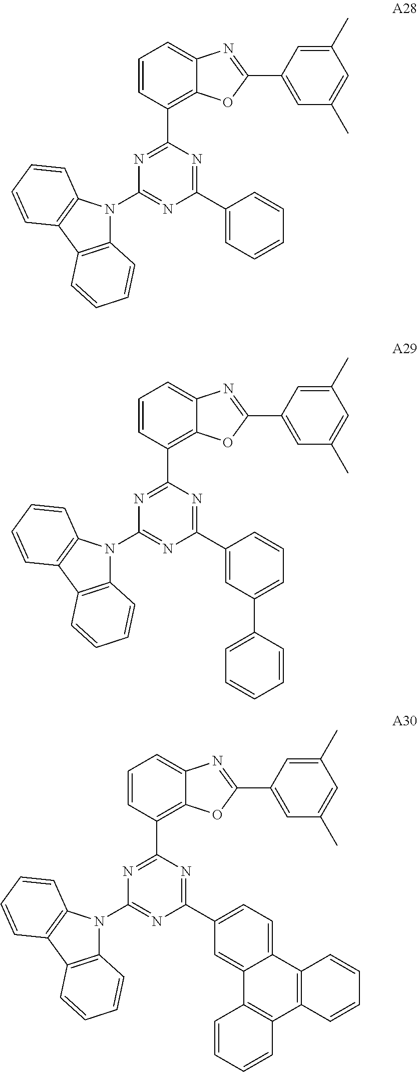 [SMILES: Cc1cc(C)cc(-c2nc3cccc(-c4nc(-c5ccccc5)nc(-n5c6ccccc6c6ccccc65)n4)c3o2)c1 ; Cc1cc(C)cc(-c2nc3cccc(-c4nc(-c5cccc(-c6ccccc6)c5)nc(-n5c6ccccc6c6ccccc65)n4)c3o2)c1 ; Cc1cc(C)cc(-c2nc3cccc(-c4nc(-c5ccc6c7ccccc7c7ccccc7c6c5)nc(-n5c6ccccc6c6ccccc65)n4)c3o2)c1]

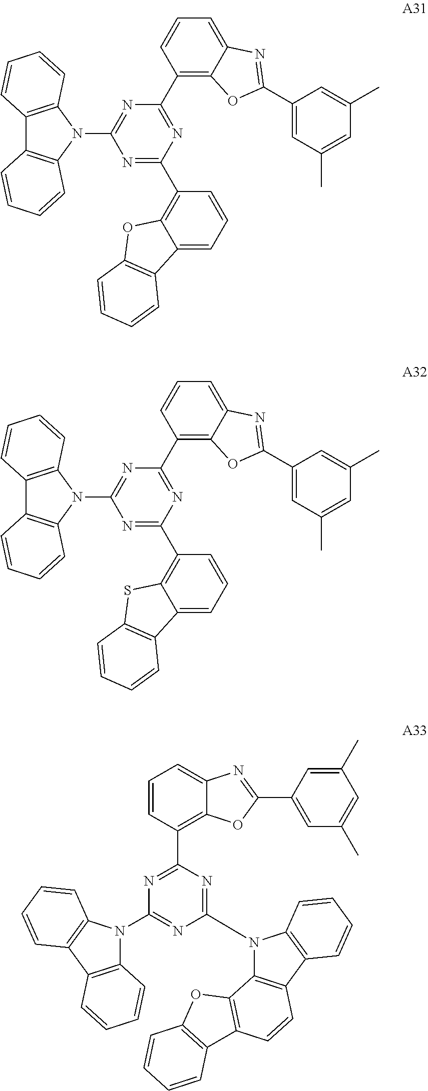 [SMILES: Cc1cc(C)cc(-c2nc3cccc(-c4nc(-c5cccc6c5oc5ccccc56)nc(-n5c6ccccc6c6ccccc65)n4)c3o2)c1 ; Cc1cc(C)cc(-c2nc3cccc(-c4nc(-c5cccc6c5sc5ccccc56)nc(-n5c6ccccc6c6ccccc65)n4)c3o2)c1 ; Cc1cc(C)cc(-c2nc3cccc(-c4nc(-n5c6ccccc6c6ccccc65)nc(-n5c6ccccc6c6ccc7c8ccccc8oc7c65)n4)c3o2)c1]

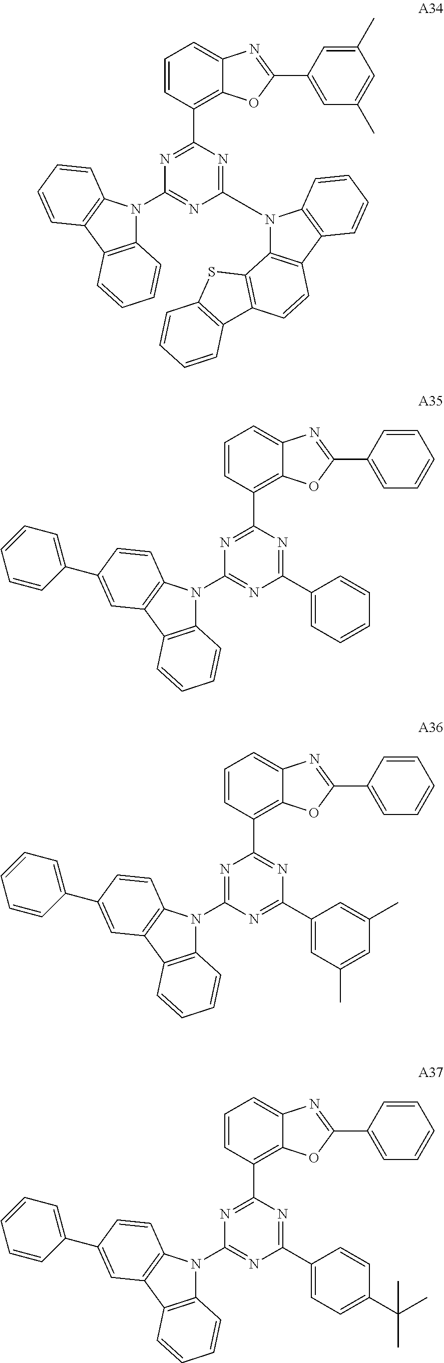 [SMILES: Cc1cc(C)cc(-c2nc3cccc(-c4nc(-n5c6ccccc6c6ccccc65)nc(-n5c6ccccc6c6ccc7c8ccccc8sc7c65)n4)c3o2)c1 ; c1ccc(-c2ccc3c(c2)c2ccccc2n3-c2nc(-c3ccccc3)nc(-c3cccc4nc(-c5ccccc5)oc34)n2)cc1 ; Cc1cc(C)cc(-c2nc(-c3cccc4nc(-c5ccccc5)oc34)nc(-n3c4ccccc4c4cc(-c5ccccc5)ccc43)n2)c1 ; CC(C)(C)c1ccc(-c2nc(-c3cccc4nc(-c5ccccc5)oc34)nc(-n3c4ccccc4c4cc(-c5ccccc5)ccc43)n2)cc1]

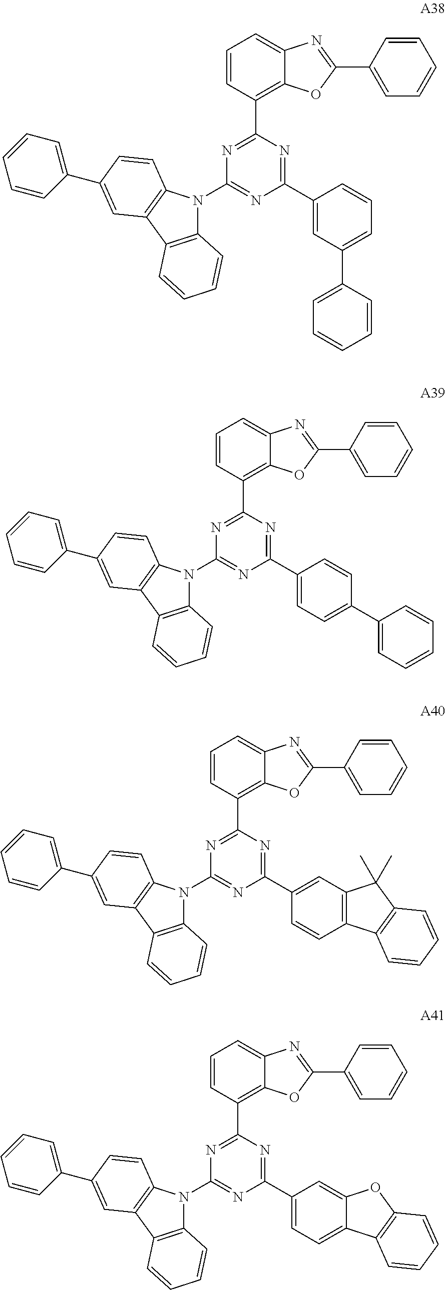 [SMILES: c1ccc(-c2cccc(-c3nc(-c4cccc5nc(-c6ccccc6)oc45)nc(-n4c5ccccc5c5cc(-c6ccccc6)ccc54)n3)c2)cc1 ; c1ccc(-c2ccc(-c3nc(-c4cccc5nc(-c6ccccc6)oc45)nc(-n4c5ccccc5c5cc(-c6ccccc6)ccc54)n3)cc2)cc1 ; CC1(C)c2ccccc2-c2ccc(-c3nc(-c4cccc5nc(-c6ccccc6)oc45)nc(-n4c5ccccc5c5cc(-c6ccccc6)ccc54)n3)cc21 ; c1ccc(-c2ccc3c(c2)c2ccccc2n3-c2nc(-c3ccc4c(c3)oc3ccccc34)nc(-c3cccc4nc(-c5ccccc5)oc34)n2)cc1]

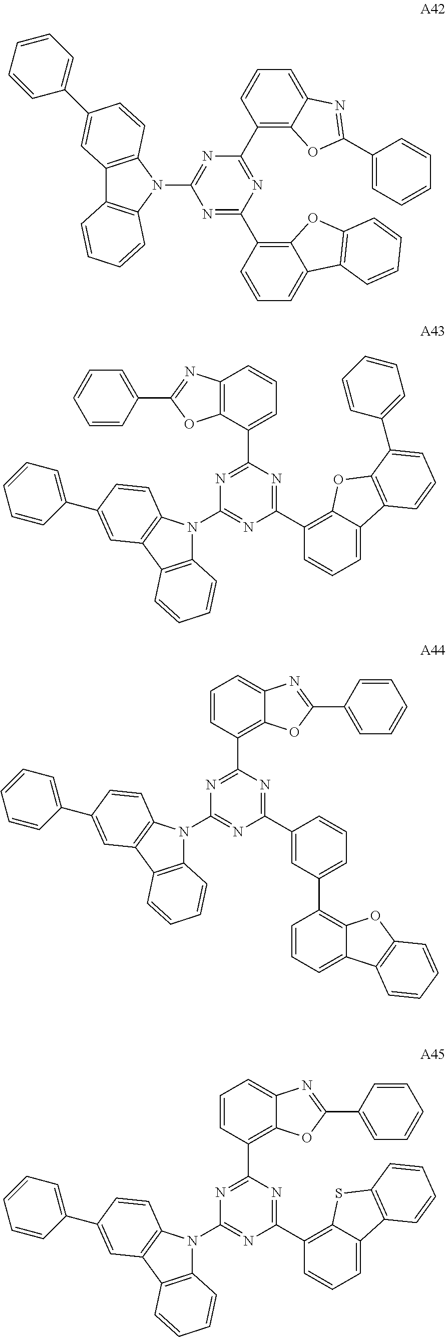 [SMILES: c1ccc(-c2ccc3c(c2)c2ccccc2n3-c2nc(-c3cccc4nc(-c5ccccc5)oc34)nc(-c3cccc4c3oc3ccccc34)n2)cc1 ; c1ccc(-c2ccc3c(c2)c2ccccc2n3-c2nc(-c3cccc4nc(-c5ccccc5)oc34)nc(-c3cccc4c3oc3c(-c5ccccc5)cccc34)n2)cc1 ; c1ccc(-c2ccc3c(c2)c2ccccc2n3-c2nc(-c3cccc(-c4cccc5c4oc4ccccc45)c3)nc(-c3cccc4nc(-c5ccccc5)oc34)n2)cc1 ; c1ccc(-c2ccc3c(c2)c2ccccc2n3-c2nc(-c3cccc4nc(-c5ccccc5)oc34)nc(-c3cccc4c3sc3ccccc34)n2)cc1]

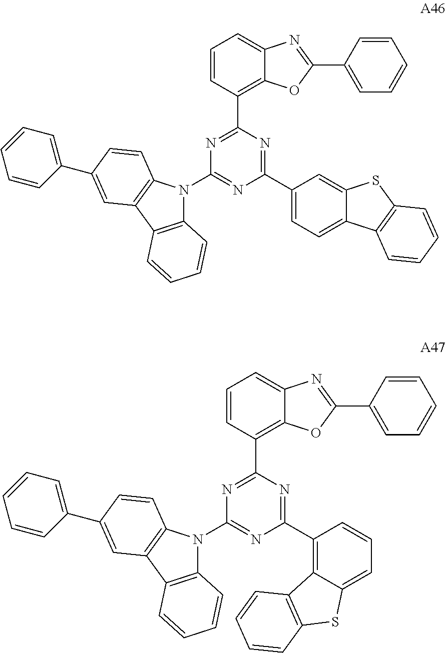 [SMILES: c1ccc(-c2ccc3c(c2)c2ccccc2n3-c2nc(-c3ccc4c(c3)sc3ccccc34)nc(-c3cccc4nc(-c5ccccc5)oc34)n2)cc1 ; c1ccc(-c2ccc3c(c2)c2ccccc2n3-c2nc(-c3cccc4nc(-c5ccccc5)oc34)nc(-c3cccc4sc5ccccc5c34)n2)cc1]

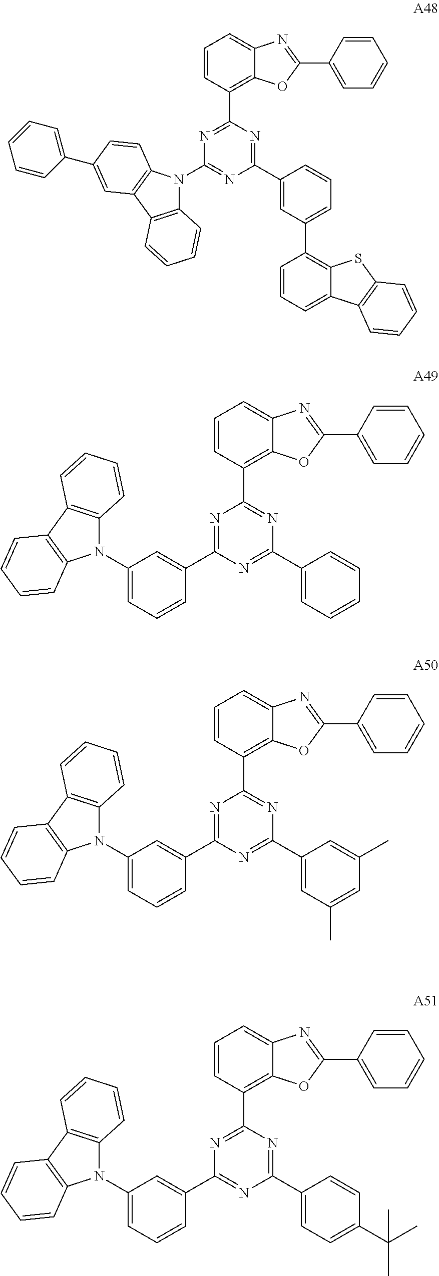 [SMILES: c1ccc(-c2ccc3c(c2)c2ccccc2n3-c2nc(-c3cccc(-c4cccc5c4sc4ccccc45)c3)nc(-c3cccc4nc(-c5ccccc5)oc34)n2)cc1 ; c1ccc(-c2nc(-c3cccc(-n4c5ccccc5c5ccccc54)c3)nc(-c3cccc4nc(-c5ccccc5)oc34)n2)cc1 ; Cc1cc(C)cc(-c2nc(-c3cccc(-n4c5ccccc5c5ccccc54)c3)nc(-c3cccc4nc(-c5ccccc5)oc34)n2)c1 ; CC(C)(C)c1ccc(-c2nc(-c3cccc(-n4c5ccccc5c5ccccc54)c3)nc(-c3cccc4nc(-c5ccccc5)oc34)n2)cc1]

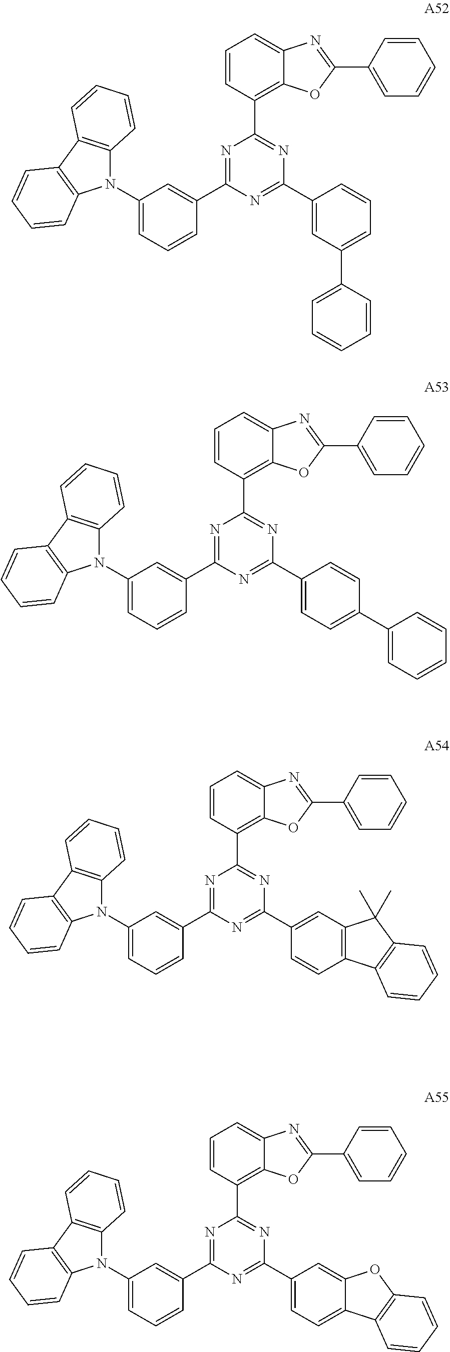 [SMILES: c1ccc(-c2cccc(-c3nc(-c4cccc(-n5c6ccccc6c6ccccc65)c4)nc(-c4cccc5nc(-c6ccccc6)oc45)n3)c2)cc1 ; c1ccc(-c2ccc(-c3nc(-c4cccc(-n5c6ccccc6c6ccccc65)c4)nc(-c4cccc5nc(-c6ccccc6)oc45)n3)cc2)cc1 ; CC1(C)c2ccccc2-c2ccc(-c3nc(-c4cccc(-n5c6ccccc6c6ccccc65)c4)nc(-c4cccc5nc(-c6ccccc6)oc45)n3)cc21 ; c1ccc(-c2nc3cccc(-c4nc(-c5cccc(-n6c7ccccc7c7ccccc76)c5)nc(-c5ccc6c(c5)oc5ccccc56)n4)c3o2)cc1]

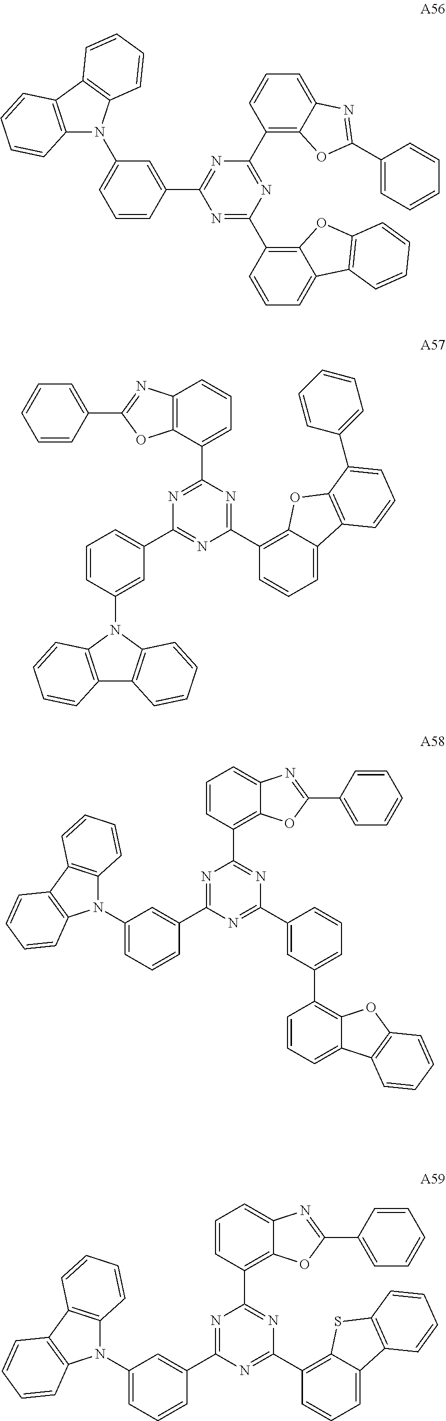 [SMILES: c1ccc(-c2nc3cccc(-c4nc(-c5cccc(-n6c7ccccc7c7ccccc76)c5)nc(-c5cccc6c5oc5ccccc56)n4)c3o2)cc1 ; c1ccc(-c2nc3cccc(-c4nc(-c5cccc(-n6c7ccccc7c7ccccc76)c5)nc(-c5cccc6c5oc5c(-c7ccccc7)cccc56)n4)c3o2)cc1 ; c1ccc(-c2nc3cccc(-c4nc(-c5cccc(-c6cccc7c6oc6ccccc67)c5)nc(-c5cccc(-n6c7ccccc7c7ccccc76)c5)n4)c3o2)cc1 ; c1ccc(-c2nc3cccc(-c4nc(-c5cccc(-n6c7ccccc7c7ccccc76)c5)nc(-c5cccc6c5sc5ccccc56)n4)c3o2)cc1]

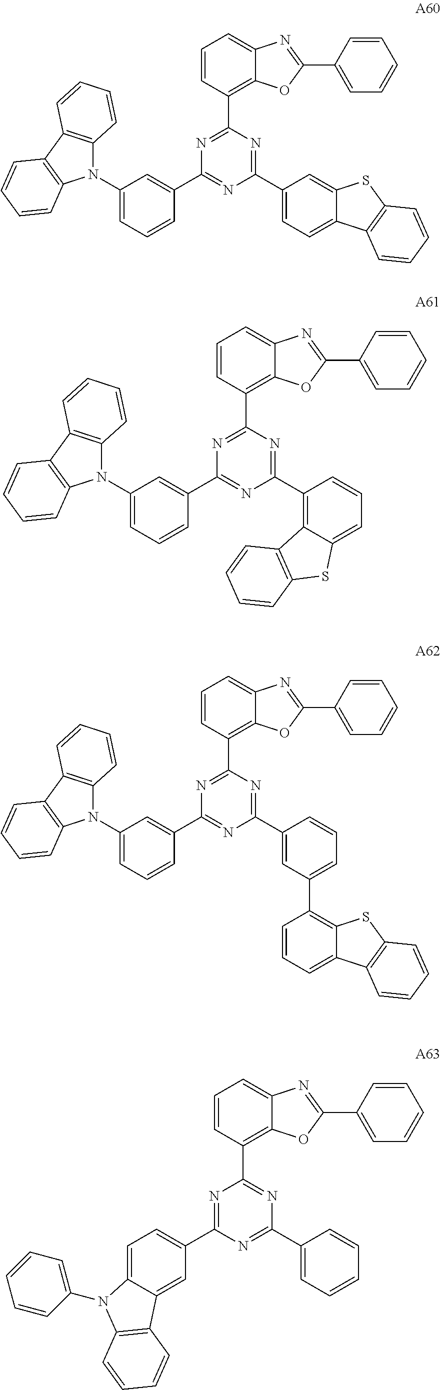 [SMILES: c1ccc(-c2nc3cccc(-c4nc(-c5cccc(-n6c7ccccc7c7ccccc76)c5)nc(-c5ccc6c(c5)sc5ccccc56)n4)c3o2)cc1 ; c1ccc(-c2nc3cccc(-c4nc(-c5cccc(-n6c7ccccc7c7ccccc76)c5)nc(-c5cccc6sc7ccccc7c56)n4)c3o2)cc1 ; c1ccc(-c2nc3cccc(-c4nc(-c5cccc(-c6cccc7c6sc6ccccc67)c5)nc(-c5cccc(-n6c7ccccc7c7ccccc76)c5)n4)c3o2)cc1 ; c1ccc(-c2nc(-c3ccc4c(c3)c3ccccc3n4-c3ccccc3)nc(-c3cccc4nc(-c5ccccc5)oc34)n2)cc1]

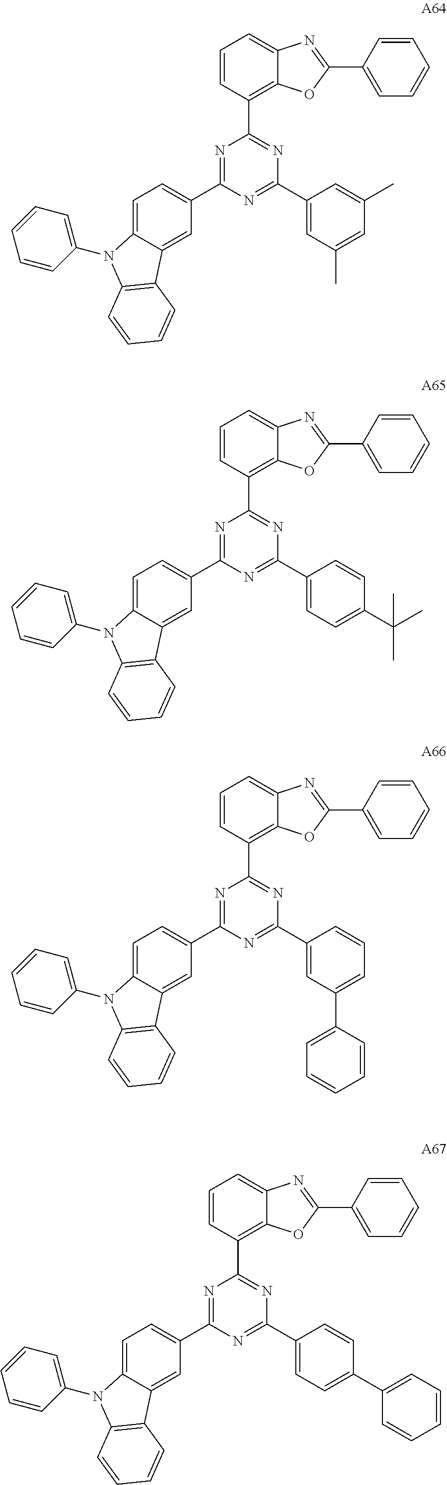 [SMILES: Cc1cc(C)cc(-c2nc(-c3ccc4c(c3)c3ccccc3n4-c3ccccc3)nc(-c3cccc4nc(-c5ccccc5)oc34)n2)c1 ; CC(C)(C)c1ccc(-c2nc(-c3ccc4c(c3)c3ccccc3n4-c3ccccc3)nc(-c3cccc4nc(-c5ccccc5)oc34)n2)cc1 ; c1ccc(-c2cccc(-c3nc(-c4ccc5c(c4)c4ccccc4n5-c4ccccc4)nc(-c4cccc5nc(-c6ccccc6)oc45)n3)c2)cc1 ; c1ccc(-c2ccc(-c3nc(-c4ccc5c(c4)c4ccccc4n5-c4ccccc4)nc(-c4cccc5nc(-c6ccccc6)oc45)n3)cc2)cc1]

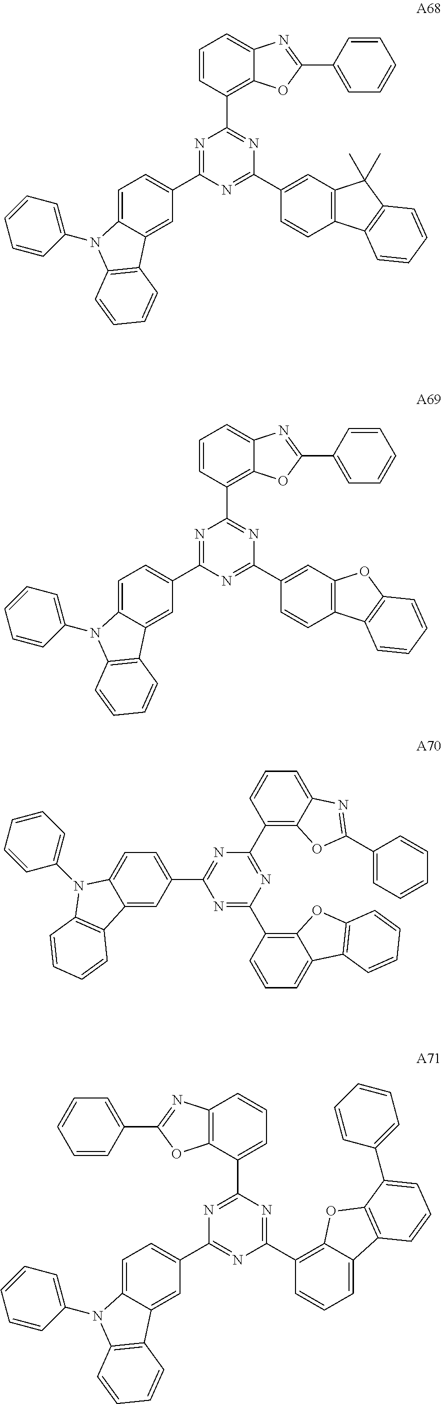 [SMILES: CC1(C)c2ccccc2-c2ccc(-c3nc(-c4ccc5c(c4)c4ccccc4n5-c4ccccc4)nc(-c4cccc5nc(-c6ccccc6)oc45)n3)cc21 ; c1ccc(-c2nc3cccc(-c4nc(-c5ccc6c(c5)oc5ccccc56)nc(-c5ccc6c(c5)c5ccccc5n6-c5ccccc5)n4)c3o2)cc1 ; c1ccc(-c2nc3cccc(-c4nc(-c5ccc6c(c5)c5ccccc5n6-c5ccccc5)nc(-c5cccc6c5oc5ccccc56)n4)c3o2)cc1 ; c1ccc(-c2nc3cccc(-c4nc(-c5ccc6c(c5)c5ccccc5n6-c5ccccc5)nc(-c5cccc6c5oc5c(-c7ccccc7)cccc56)n4)c3o2)cc1]

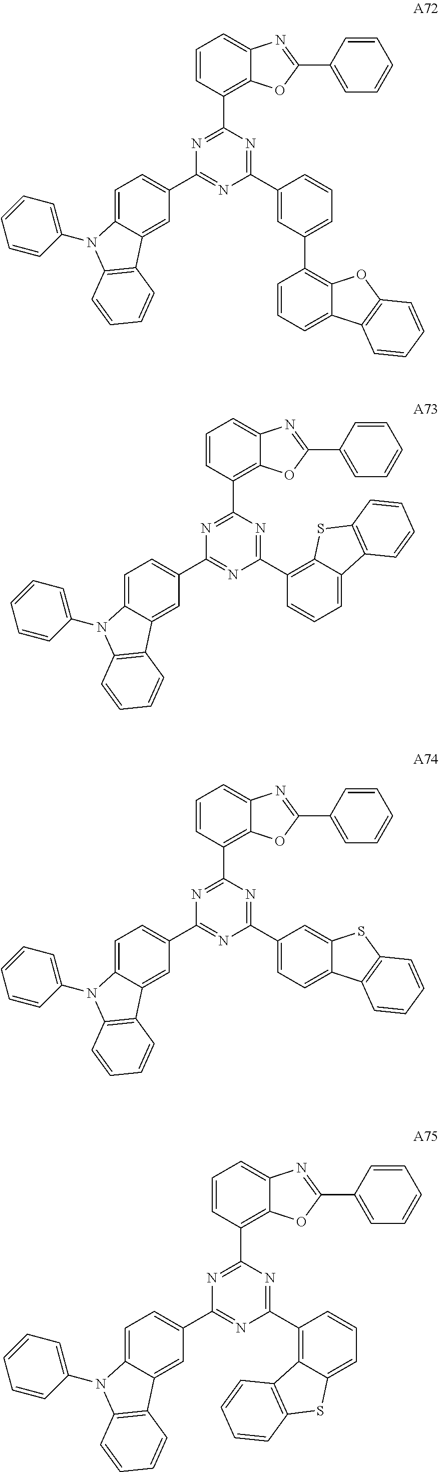 [SMILES: c1ccc(-c2nc3cccc(-c4nc(-c5cccc(-c6cccc7c6oc6ccccc67)c5)nc(-c5ccc6c(c5)c5ccccc5n6-c5ccccc5)n4)c3o2)cc1 ; c1ccc(-c2nc3cccc(-c4nc(-c5ccc6c(c5)c5ccccc5n6-c5ccccc5)nc(-c5cccc6c5sc5ccccc56)n4)c3o2)cc1 ; c1ccc(-c2nc3cccc(-c4nc(-c5ccc6c(c5)sc5ccccc56)nc(-c5ccc6c(c5)c5ccccc5n6-c5ccccc5)n4)c3o2)cc1 ; c1ccc(-c2nc3cccc(-c4nc(-c5ccc6c(c5)c5ccccc5n6-c5ccccc5)nc(-c5cccc6sc7ccccc7c56)n4)c3o2)cc1]

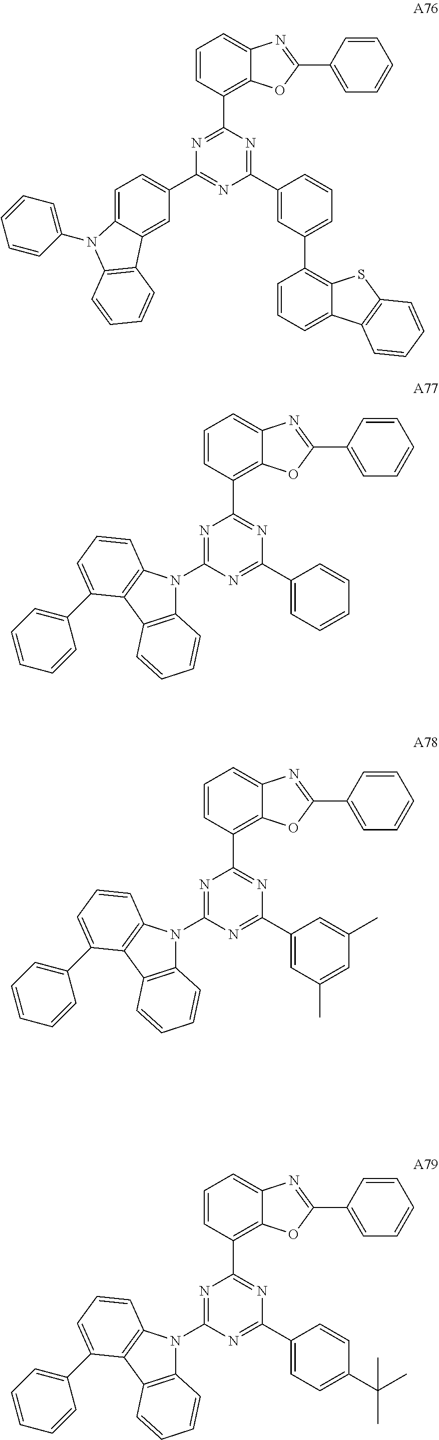 [SMILES: c1ccc(-c2nc3cccc(-c4nc(-c5cccc(-c6cccc7c6sc6ccccc67)c5)nc(-c5ccc6c(c5)c5ccccc5n6-c5ccccc5)n4)c3o2)cc1 ; c1ccc(-c2nc(-c3cccc4nc(-c5ccccc5)oc34)nc(-n3c4ccccc4c4c(-c5ccccc5)cccc43)n2)cc1 ; Cc1cc(C)cc(-c2nc(-c3cccc4nc(-c5ccccc5)oc34)nc(-n3c4ccccc4c4c(-c5ccccc5)cccc43)n2)c1 ; CC(C)(C)c1ccc(-c2nc(-c3cccc4nc(-c5ccccc5)oc34)nc(-n3c4ccccc4c4c(-c5ccccc5)cccc43)n2)cc1]

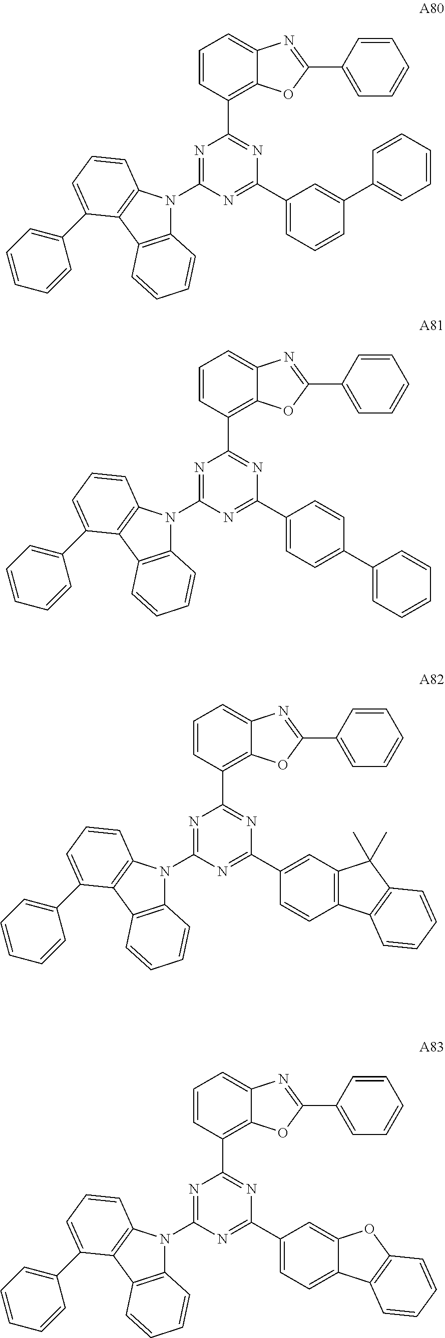 [SMILES: c1ccc(-c2cccc(-c3nc(-c4cccc5nc(-c6ccccc6)oc45)nc(-n4c5ccccc5c5c(-c6ccccc6)cccc54)n3)c2)cc1 ; c1ccc(-c2ccc(-c3nc(-c4cccc5nc(-c6ccccc6)oc45)nc(-n4c5ccccc5c5c(-c6ccccc6)cccc54)n3)cc2)cc1 ; CC1(C)c2ccccc2-c2ccc(-c3nc(-c4cccc5nc(-c6ccccc6)oc45)nc(-n4c5ccccc5c5c(-c6ccccc6)cccc54)n3)cc21 ; c1ccc(-c2nc3cccc(-c4nc(-c5ccc6c(c5)oc5ccccc56)nc(-n5c6ccccc6c6c(-c7ccccc7)cccc65)n4)c3o2)cc1]

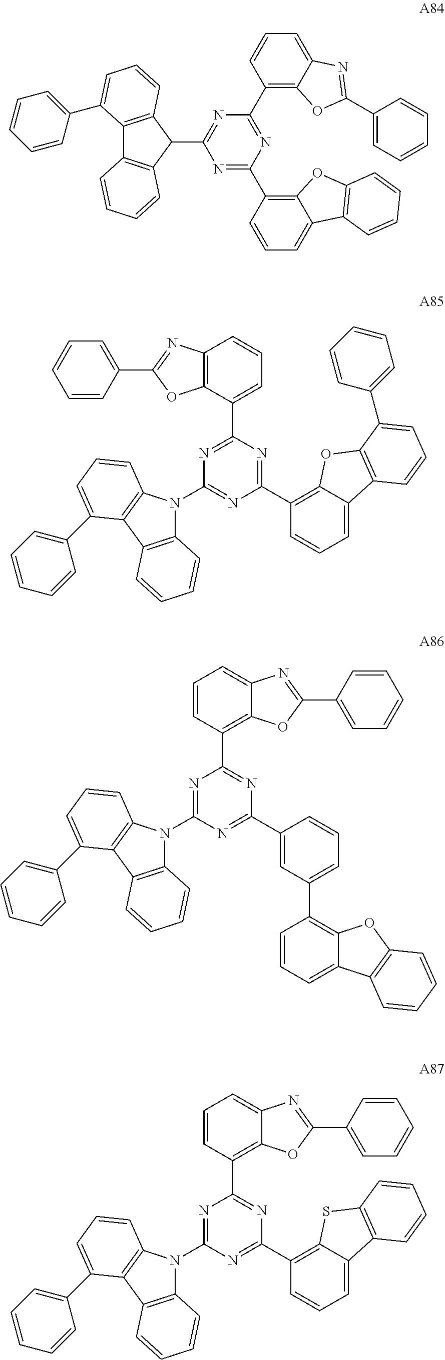 [SMILES: c1ccc(-c2nc3cccc(-c4nc(-c5cccc6c5oc5ccccc56)nc(C5c6ccccc6-c6c(-c7ccccc7)cccc65)n4)c3o2)cc1 ; c1ccc(-c2nc3cccc(-c4nc(-c5cccc6c5oc5c(-c7ccccc7)cccc56)nc(-n5c6ccccc6c6c(-c7ccccc7)cccc65)n4)c3o2)cc1 ; c1ccc(-c2nc3cccc(-c4nc(-c5cccc(-c6cccc7c6oc6ccccc67)c5)nc(-n5c6ccccc6c6c(-c7ccccc7)cccc65)n4)c3o2)cc1 ; c1ccc(-c2nc3cccc(-c4nc(-c5cccc6c5sc5ccccc56)nc(-n5c6ccccc6c6c(-c7ccccc7)cccc65)n4)c3o2)cc1]

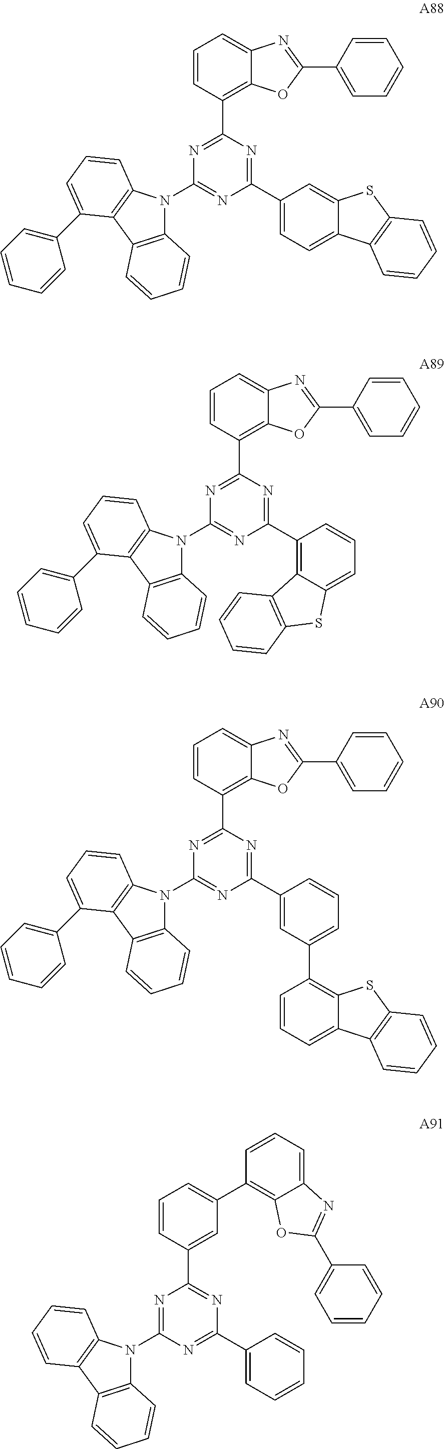 [SMILES: c1ccc(-c2nc3cccc(-c4nc(-c5ccc6c(c5)sc5ccccc56)nc(-n5c6ccccc6c6c(-c7ccccc7)cccc65)n4)c3o2)cc1 ; c1ccc(-c2nc3cccc(-c4nc(-c5cccc6sc7ccccc7c56)nc(-n5c6ccccc6c6c(-c7ccccc7)cccc65)n4)c3o2)cc1 ; c1ccc(-c2nc3cccc(-c4nc(-c5cccc(-c6cccc7c6sc6ccccc67)c5)nc(-n5c6ccccc6c6c(-c7ccccc7)cccc65)n4)c3o2)cc1 ; c1ccc(-c2nc(-c3cccc(-c4cccc5nc(-c6ccccc6)oc45)c3)nc(-n3c4ccccc4c4ccccc43)n2)cc1]

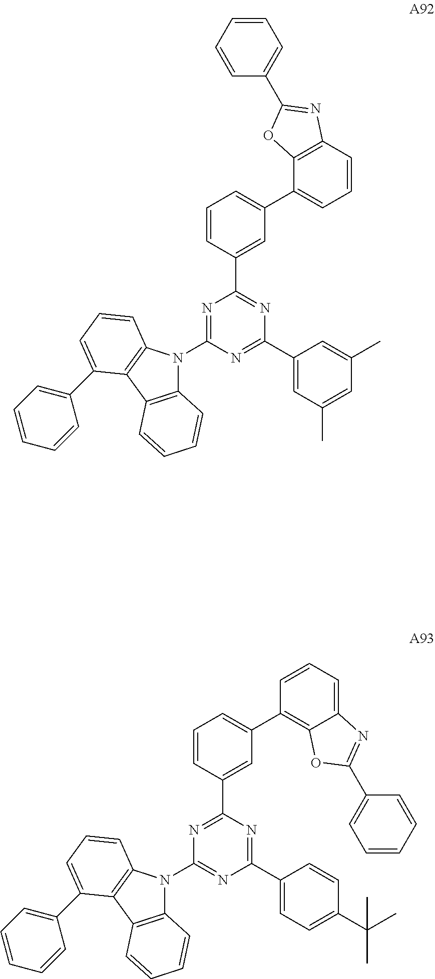 [SMILES: Cc1cc(C)cc(-c2nc(-c3cccc(-c4cccc5nc(-c6ccccc6)oc45)c3)nc(-n3c4ccccc4c4c(-c5ccccc5)cccc43)n2)c1 ; CC(C)(C)c1ccc(-c2nc(-c3cccc(-c4cccc5nc(-c6ccccc6)oc45)c3)nc(-n3c4ccccc4c4c(-c5ccccc5)cccc43)n2)cc1]

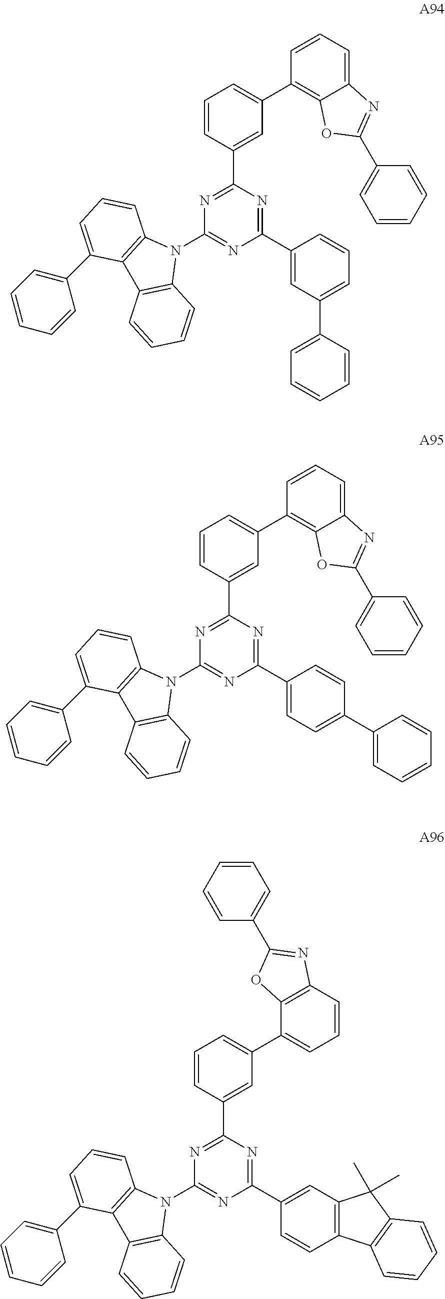 [SMILES: c1ccc(-c2cccc(-c3nc(-c4cccc(-c5cccc6nc(-c7ccccc7)oc56)c4)nc(-n4c5ccccc5c5c(-c6ccccc6)cccc54)n3)c2)cc1 ; c1ccc(-c2ccc(-c3nc(-c4cccc(-c5cccc6nc(-c7ccccc7)oc56)c4)nc(-n4c5ccccc5c5c(-c6ccccc6)cccc54)n3)cc2)cc1 ; CC1(C)c2ccccc2-c2ccc(-c3nc(-c4cccc(-c5cccc6nc(-c7ccccc7)oc56)c4)nc(-n4c5ccccc5c5c(-c6ccccc6)cccc54)n3)cc21]

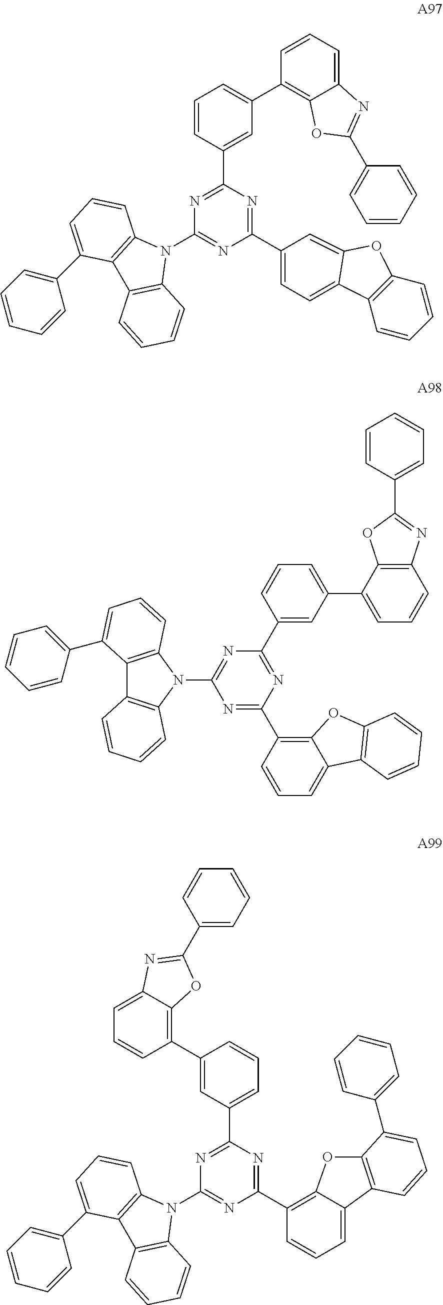 [SMILES: c1ccc(-c2nc3cccc(-c4cccc(-c5nc(-c6ccc7c(c6)oc6ccccc67)nc(-n6c7ccccc7c7c(-c8ccccc8)cccc76)n5)c4)c3o2)cc1 ; c1ccc(-c2nc3cccc(-c4cccc(-c5nc(-c6cccc7c6oc6ccccc67)nc(-n6c7ccccc7c7c(-c8ccccc8)cccc76)n5)c4)c3o2)cc1 ; c1ccc(-c2nc3cccc(-c4cccc(-c5nc(-c6cccc7c6oc6c(-c8ccccc8)cccc67)nc(-n6c7ccccc7c7c(-c8ccccc8)cccc76)n5)c4)c3o2)cc1]

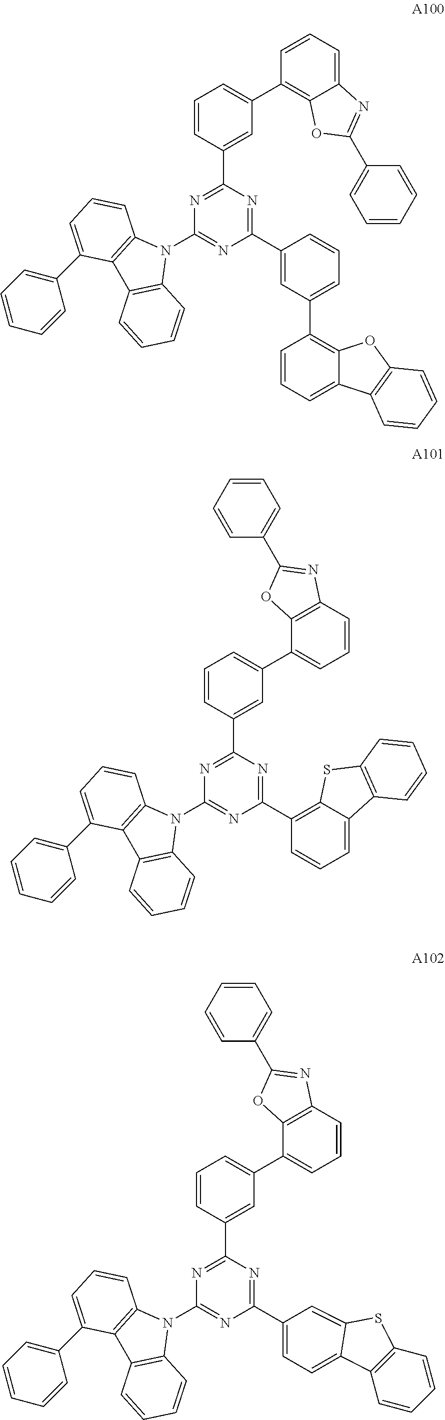 [SMILES: c1ccc(-c2nc3cccc(-c4cccc(-c5nc(-c6cccc(-c7cccc8c7oc7ccccc78)c6)nc(-n6c7ccccc7c7c(-c8ccccc8)cccc76)n5)c4)c3o2)cc1 ; c1ccc(-c2nc3cccc(-c4cccc(-c5nc(-c6cccc7c6sc6ccccc67)nc(-n6c7ccccc7c7c(-c8ccccc8)cccc76)n5)c4)c3o2)cc1 ; c1ccc(-c2nc3cccc(-c4cccc(-c5nc(-c6ccc7c(c6)sc6ccccc67)nc(-n6c7ccccc7c7c(-c8ccccc8)cccc76)n5)c4)c3o2)cc1]

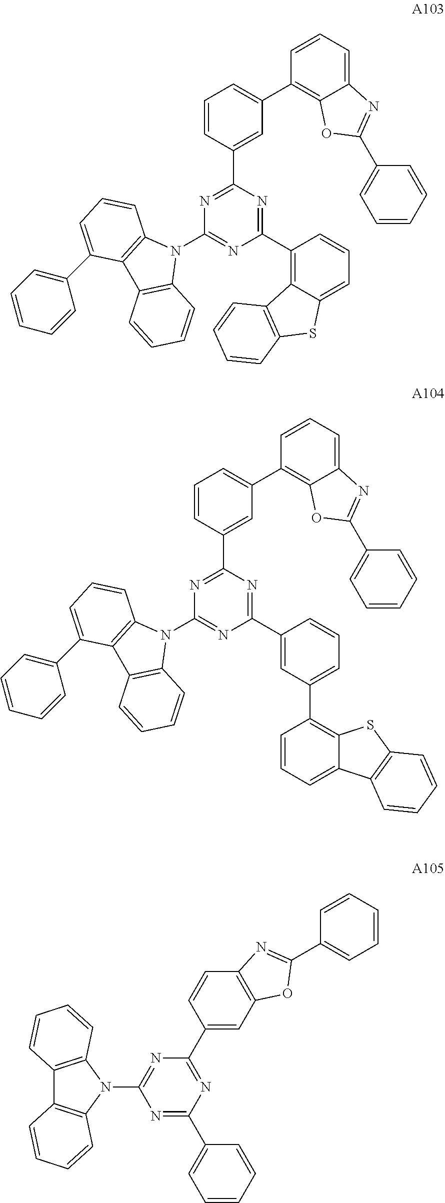 [SMILES: c1ccc(-c2nc3cccc(-c4cccc(-c5nc(-c6cccc7sc8ccccc8c67)nc(-n6c7ccccc7c7c(-c8ccccc8)cccc76)n5)c4)c3o2)cc1 ; c1ccc(-c2nc3cccc(-c4cccc(-c5nc(-c6cccc(-c7cccc8c7sc7ccccc78)c6)nc(-n6c7ccccc7c7c(-c8ccccc8)cccc76)n5)c4)c3o2)cc1 ; c1ccc(-c2nc(-c3ccc4nc(-c5ccccc5)oc4c3)nc(-n3c4ccccc4c4ccccc43)n2)cc1]

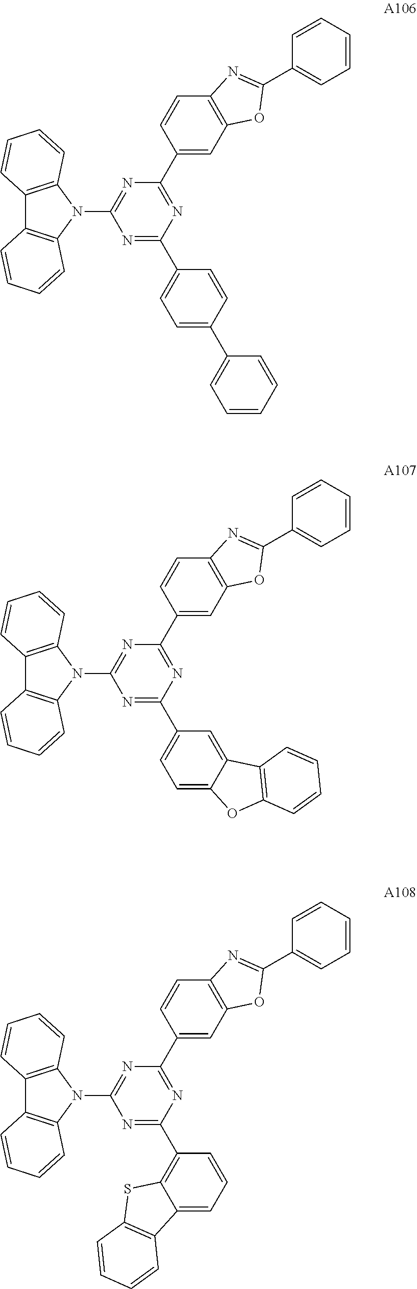 [SMILES: c1ccc(-c2ccc(-c3nc(-c4ccc5nc(-c6ccccc6)oc5c4)nc(-n4c5ccccc5c5ccccc54)n3)cc2)cc1 ; c1ccc(-c2nc3ccc(-c4nc(-c5ccc6oc7ccccc7c6c5)nc(-n5c6ccccc6c6ccccc65)n4)cc3o2)cc1 ; c1ccc(-c2nc3ccc(-c4nc(-c5cccc6c5sc5ccccc56)nc(-n5c6ccccc6c6ccccc65)n4)cc3o2)cc1]

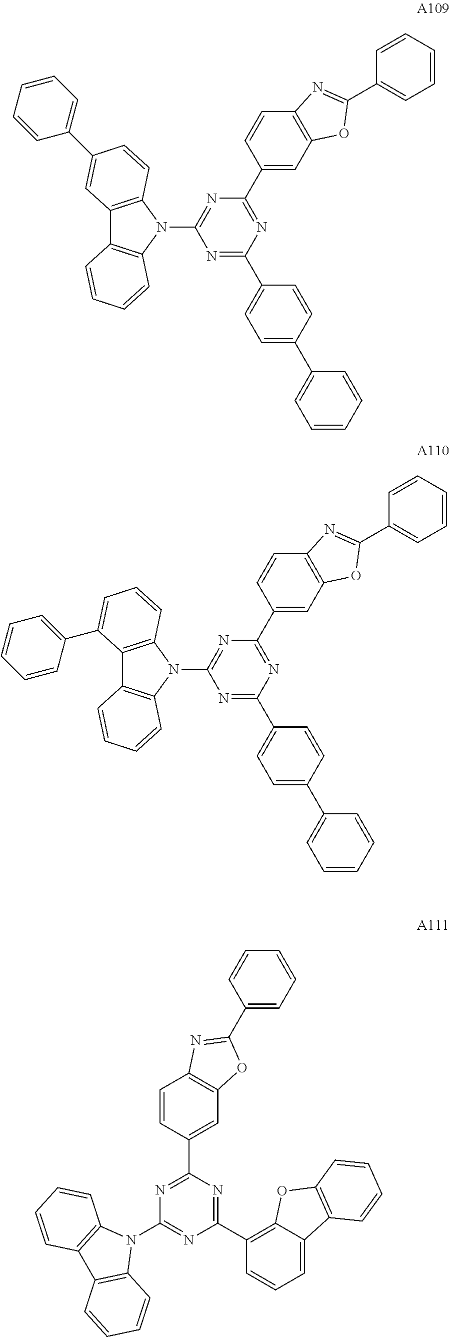 [SMILES: c1ccc(-c2ccc(-c3nc(-c4ccc5nc(-c6ccccc6)oc5c4)nc(-n4c5ccccc5c5cc(-c6ccccc6)ccc54)n3)cc2)cc1 ; c1ccc(-c2ccc(-c3nc(-c4ccc5nc(-c6ccccc6)oc5c4)nc(-n4c5ccccc5c5c(-c6ccccc6)cccc54)n3)cc2)cc1 ; c1ccc(-c2nc3ccc(-c4nc(-c5cccc6c5oc5ccccc56)nc(-n5c6ccccc6c6ccccc65)n4)cc3o2)cc1]

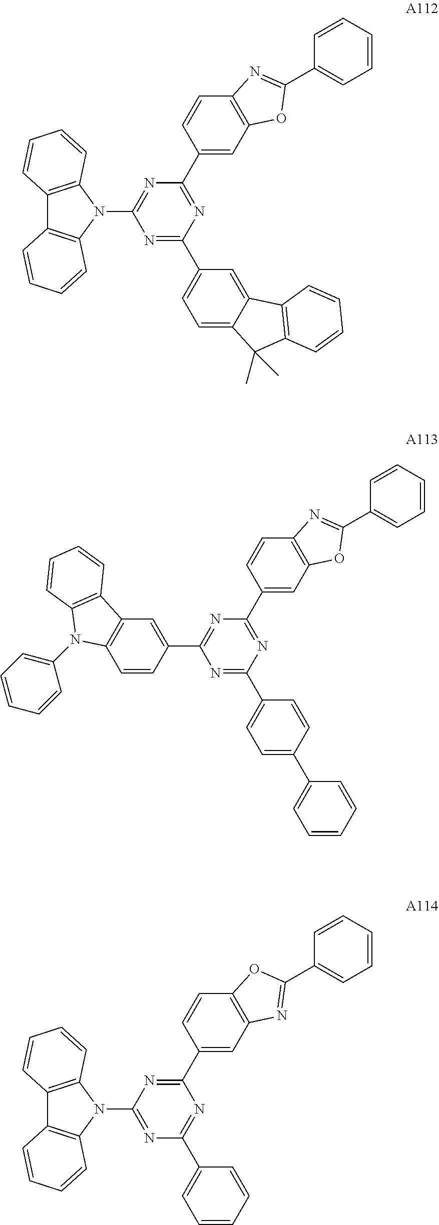 [SMILES: CC1(C)c2ccccc2-c2cc(-c3nc(-c4ccc5nc(-c6ccccc6)oc5c4)nc(-n4c5ccccc5c5ccccc54)n3)ccc21 ; c1ccc(-c2ccc(-c3nc(-c4ccc5nc(-c6ccccc6)oc5c4)nc(-c4ccc5c(c4)c4ccccc4n5-c4ccccc4)n3)cc2)cc1 ; c1ccc(-c2nc(-c3ccc4oc(-c5ccccc5)nc4c3)nc(-n3c4ccccc4c4ccccc43)n2)cc1]

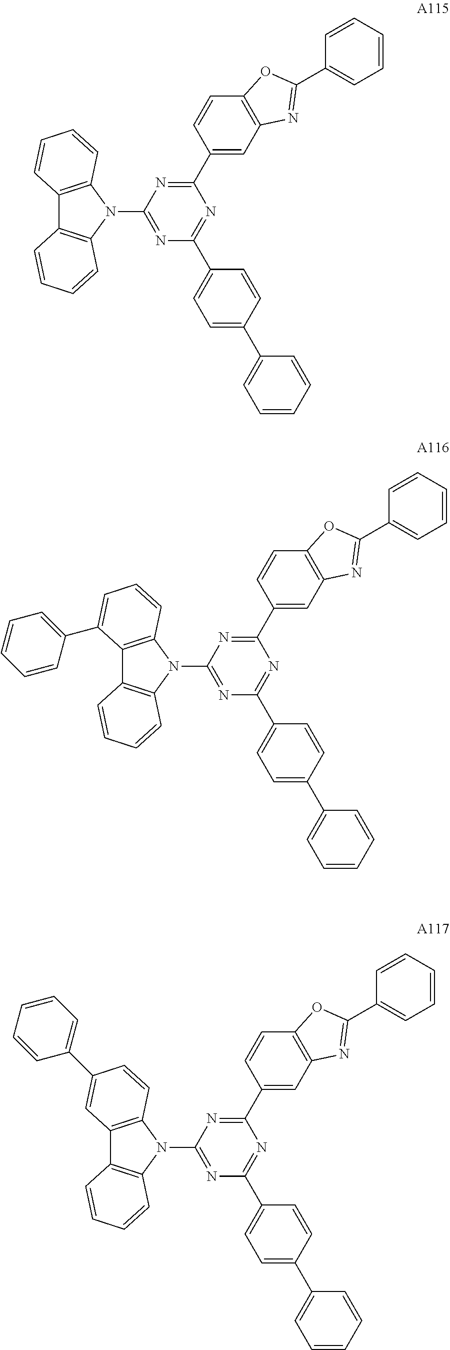 [SMILES: c1ccc(-c2ccc(-c3nc(-c4ccc5oc(-c6ccccc6)nc5c4)nc(-n4c5ccccc5c5ccccc54)n3)cc2)cc1 ; c1ccc(-c2ccc(-c3nc(-c4ccc5oc(-c6ccccc6)nc5c4)nc(-n4c5ccccc5c5c(-c6ccccc6)cccc54)n3)cc2)cc1 ; c1ccc(-c2ccc(-c3nc(-c4ccc5oc(-c6ccccc6)nc5c4)nc(-n4c5ccccc5c5cc(-c6ccccc6)ccc54)n3)cc2)cc1]

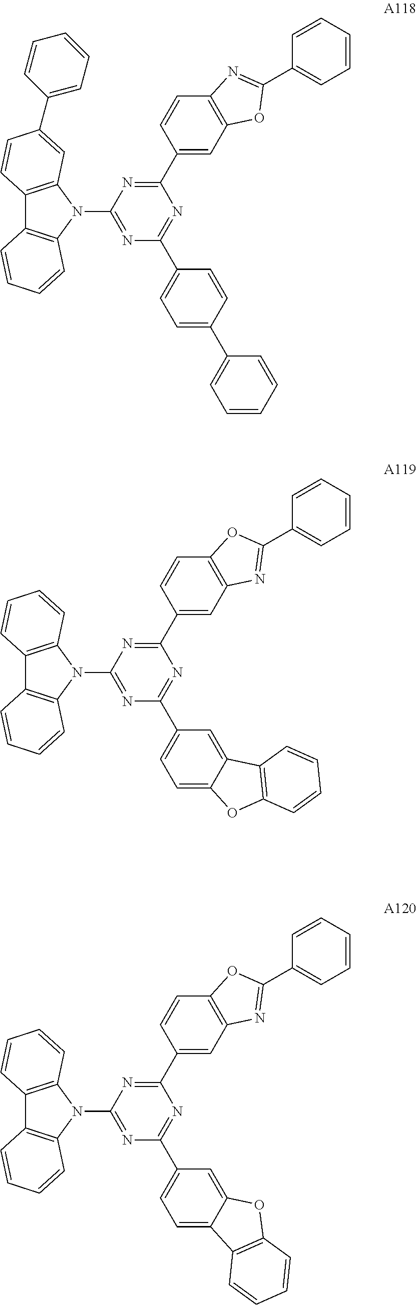 [SMILES: c1ccc(-c2ccc(-c3nc(-c4ccc5nc(-c6ccccc6)oc5c4)nc(-n4c5ccccc5c5ccc(-c6ccccc6)cc54)n3)cc2)cc1 ; c1ccc(-c2nc3cc(-c4nc(-c5ccc6oc7ccccc7c6c5)nc(-n5c6ccccc6c6ccccc65)n4)ccc3o2)cc1 ; c1ccc(-c2nc3cc(-c4nc(-c5ccc6c(c5)oc5ccccc56)nc(-n5c6ccccc6c6ccccc65)n4)ccc3o2)cc1]

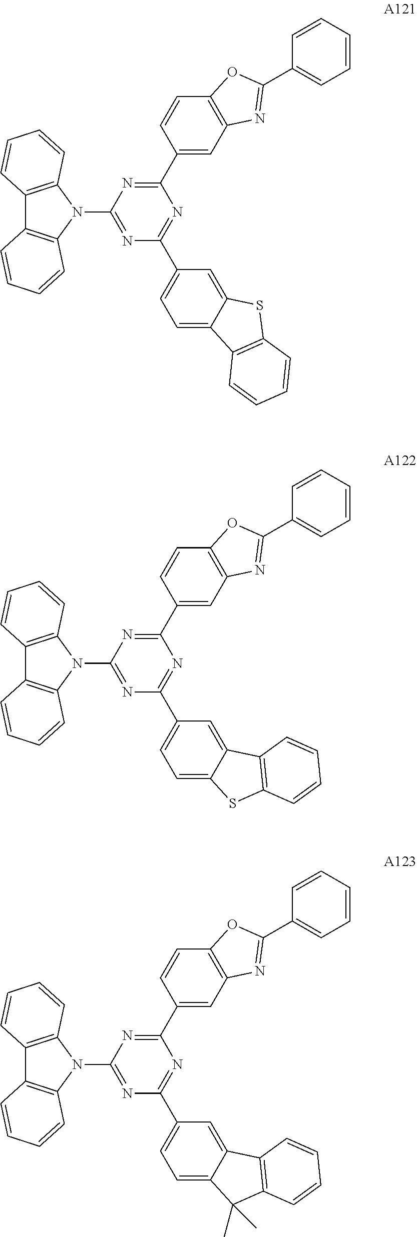 [SMILES: c1ccc(-c2nc3cc(-c4nc(-c5ccc6c(c5)sc5ccccc56)nc(-n5c6ccccc6c6ccccc65)n4)ccc3o2)cc1 ; c1ccc(-c2nc3cc(-c4nc(-c5ccc6sc7ccccc7c6c5)nc(-n5c6ccccc6c6ccccc65)n4)ccc3o2)cc1 ; CC1(C)c2ccccc2-c2cc(-c3nc(-c4ccc5oc(-c6ccccc6)nc5c4)nc(-n4c5ccccc5c5ccccc54)n3)ccc21]

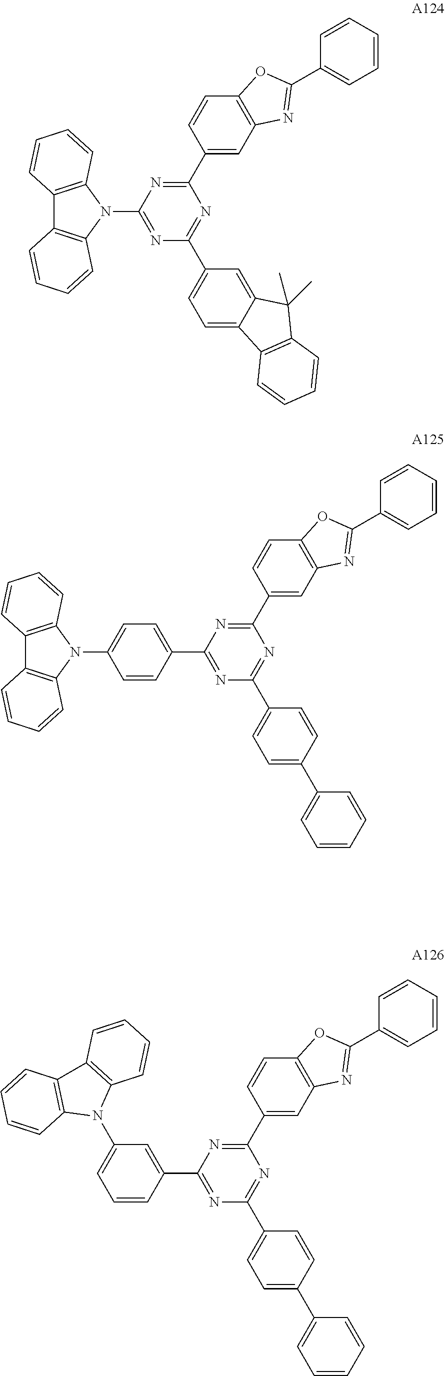 [SMILES: CC1(C)c2ccccc2-c2ccc(-c3nc(-c4ccc5oc(-c6ccccc6)nc5c4)nc(-n4c5ccccc5c5ccccc54)n3)cc21 ; c1ccc(-c2ccc(-c3nc(-c4ccc(-n5c6ccccc6c6ccccc65)cc4)nc(-c4ccc5oc(-c6ccccc6)nc5c4)n3)cc2)cc1 ; c1ccc(-c2ccc(-c3nc(-c4cccc(-n5c6ccccc6c6ccccc65)c4)nc(-c4ccc5oc(-c6ccccc6)nc5c4)n3)cc2)cc1]

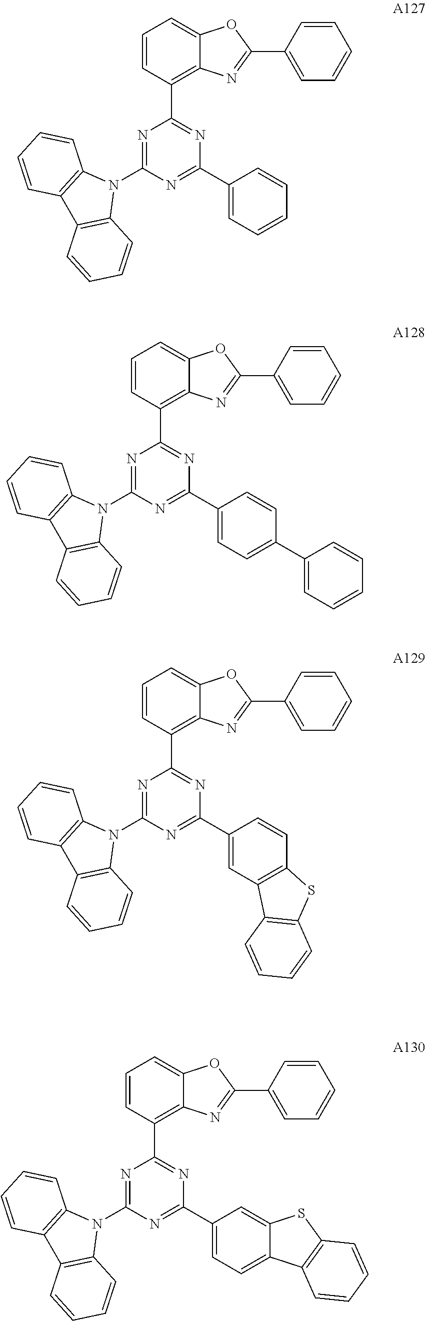 [SMILES: c1ccc(-c2nc(-c3cccc4oc(-c5ccccc5)nc34)nc(-n3c4ccccc4c4ccccc43)n2)cc1 ; c1ccc(-c2ccc(-c3nc(-c4cccc5oc(-c6ccccc6)nc45)nc(-n4c5ccccc5c5ccccc54)n3)cc2)cc1 ; c1ccc(-c2nc3c(-c4nc(-c5ccc6sc7ccccc7c6c5)nc(-n5c6ccccc6c6ccccc65)n4)cccc3o2)cc1 ; c1ccc(-c2nc3c(-c4nc(-c5ccc6c(c5)sc5ccccc56)nc(-n5c6ccccc6c6ccccc65)n4)cccc3o2)cc1]

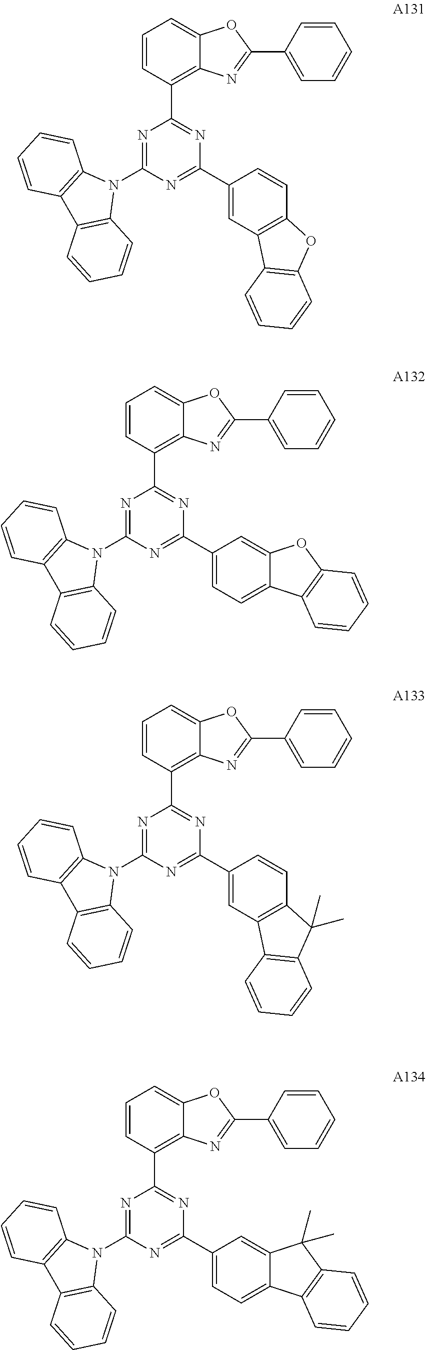 [SMILES: c1ccc(-c2nc3c(-c4nc(-c5ccc6oc7ccccc7c6c5)nc(-n5c6ccccc6c6ccccc65)n4)cccc3o2)cc1 ; c1ccc(-c2nc3c(-c4nc(-c5ccc6c(c5)oc5ccccc56)nc(-n5c6ccccc6c6ccccc65)n4)cccc3o2)cc1 ; CC1(C)c2ccccc2-c2cc(-c3nc(-c4cccc5oc(-c6ccccc6)nc45)nc(-n4c5ccccc5c5ccccc54)n3)ccc21 ; CC1(C)c2ccccc2-c2ccc(-c3nc(-c4cccc5oc(-c6ccccc6)nc45)nc(-n4c5ccccc5c5ccccc54)n3)cc21]

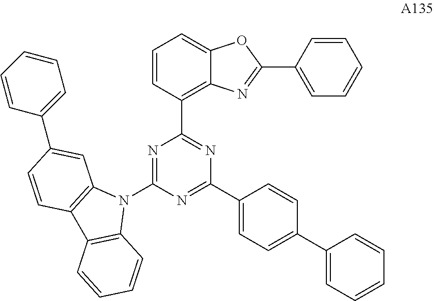 [SMILES: c1ccc(-c2ccc(-c3nc(-c4cccc5oc(-c6ccccc6)nc45)nc(-n4c5ccccc5c5ccc(-c6ccccc6)cc54)n3)cc2)cc1]

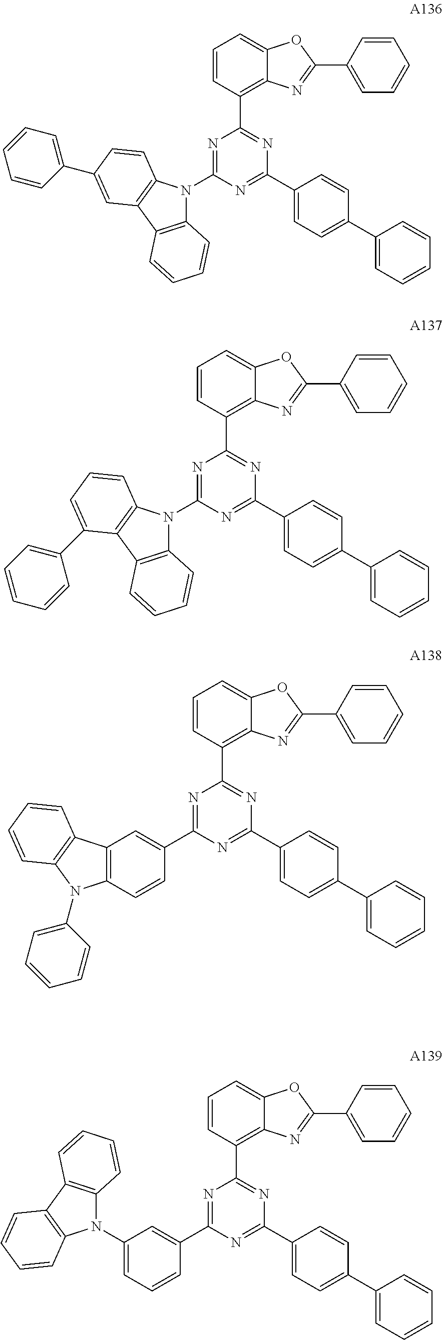 [SMILES: c1ccc(-c2ccc(-c3nc(-c4cccc5oc(-c6ccccc6)nc45)nc(-n4c5ccccc5c5cc(-c6ccccc6)ccc54)n3)cc2)cc1 ; c1ccc(-c2ccc(-c3nc(-c4cccc5oc(-c6ccccc6)nc45)nc(-n4c5ccccc5c5c(-c6ccccc6)cccc54)n3)cc2)cc1 ; c1ccc(-c2ccc(-c3nc(-c4ccc5c(c4)c4ccccc4n5-c4ccccc4)nc(-c4cccc5oc(-c6ccccc6)nc45)n3)cc2)cc1 ; c1ccc(-c2ccc(-c3nc(-c4cccc(-n5c6ccccc6c6ccccc65)c4)nc(-c4cccc5oc(-c6ccccc6)nc45)n3)cc2)cc1]

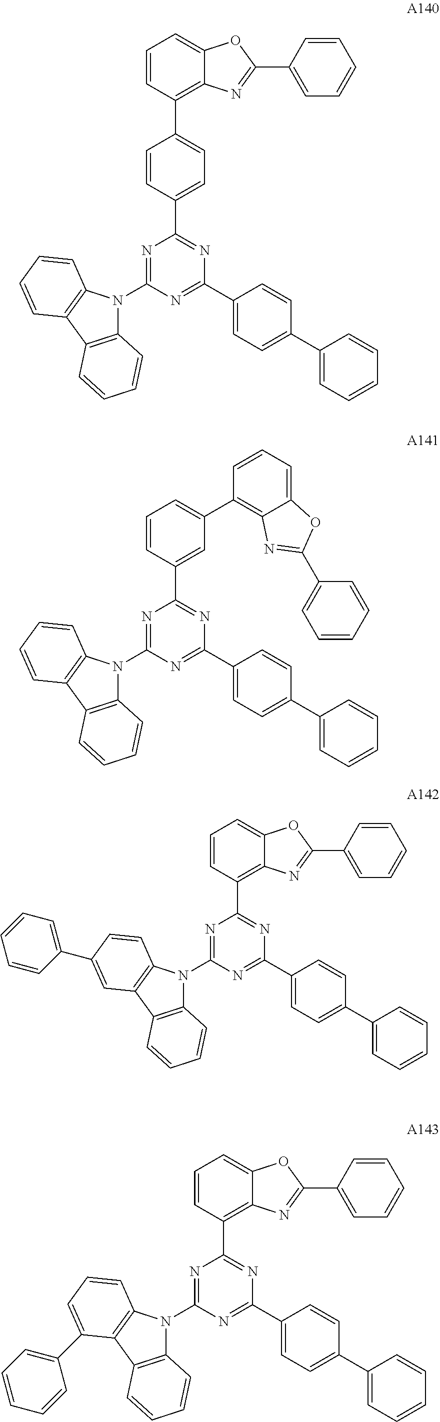 [SMILES: c1ccc(-c2ccc(-c3nc(-c4ccc(-c5cccc6oc(-c7ccccc7)nc56)cc4)nc(-n4c5ccccc5c5ccccc54)n3)cc2)cc1 ; c1ccc(-c2ccc(-c3nc(-c4cccc(-c5cccc6oc(-c7ccccc7)nc56)c4)nc(-n4c5ccccc5c5ccccc54)n3)cc2)cc1 ; c1ccc(-c2ccc(-c3nc(-c4cccc5oc(-c6ccccc6)nc45)nc(-n4c5ccccc5c5cc(-c6ccccc6)ccc54)n3)cc2)cc1 ; c1ccc(-c2ccc(-c3nc(-c4cccc5oc(-c6ccccc6)nc45)nc(-n4c5ccccc5c5c(-c6ccccc6)cccc54)n3)cc2)cc1]

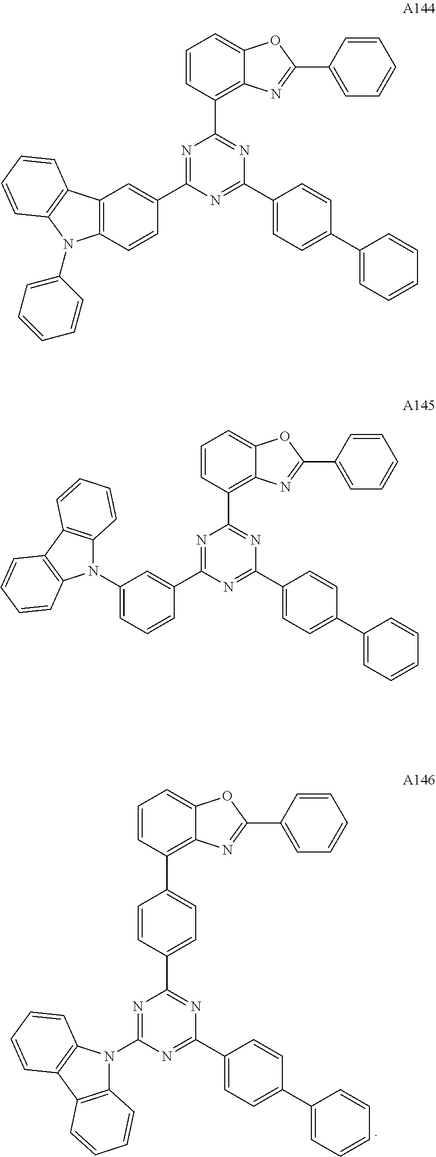 [SMILES: c1ccc(-c2ccc(-c3nc(-c4ccc5c(c4)c4ccccc4n5-c4ccccc4)nc(-c4cccc5oc(-c6ccccc6)nc45)n3)cc2)cc1 ; c1ccc(-c2ccc(-c3nc(-c4cccc(-n5c6ccccc6c6ccccc65)c4)nc(-c4cccc5oc(-c6ccccc6)nc45)n3)cc2)cc1 ; c1ccc(-c2ccc(-c3nc(-c4ccc(-c5cccc6oc(-c7ccccc7)nc56)cc4)nc(-n4c5ccccc5c5ccccc54)n3)cc2)cc1]

5. An organic light emitting device, comprising:
a substrate; and
an organic light emitting diode positioned on the substrate and including:
a first electrode;
a second electrode facing the first electrode; and
a first emitting part between the first electrode and the second electrode, the first emitting part including a first emitting material layer,
wherein the first emitting material layer includes a first compound that is the organic compound of claim 1.

6. The organic light emitting device according to claim 5, wherein the first emitting material layer further includes a second compound represented by Formula 3: 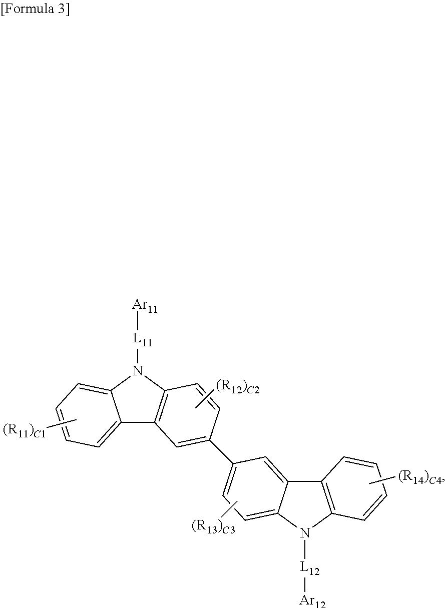
wherein each c1 and c4 is independently an integer of 0 to 4, each c2 and c3 is independently an integer of 0 to 3,
wherein each R₁₁, R₁₂, R₁₃, and R₁₄ is independently selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
wherein each L₁₁ and L₁₂ is independently selected from the group consisting of a single bond, a substituted or unsubstituted C6 to C60 arylene group, and a substituted or unsubstituted C3 to C60 heteroarylene group, and
wherein each Ar₁₁ and Ar₁₂ is independently selected from the group consisting of a substituted or unsubstituted C6 to C60 aryl group, and a substituted or unsubstituted C3 to C60 heteroaryl group.

7. The organic light emitting device according to claim 6, wherein the second compound is one of the compounds in Formula 4:

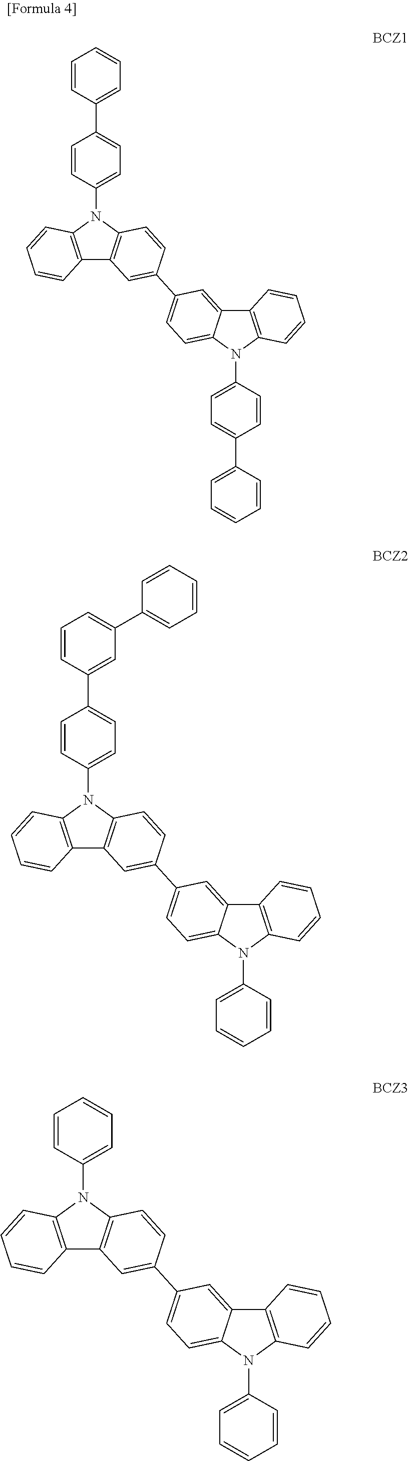 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-c3ccc(-n4c5ccccc5c5cc(-c6ccc7c(c6)c6ccccc6n7-c6ccccc6)ccc54)cc3)c2)cc1 ; c1ccc(-n2c3ccccc3c3cc(-c4ccc5c(c4)c4ccccc4n5-c4ccccc4)ccc32)cc1]

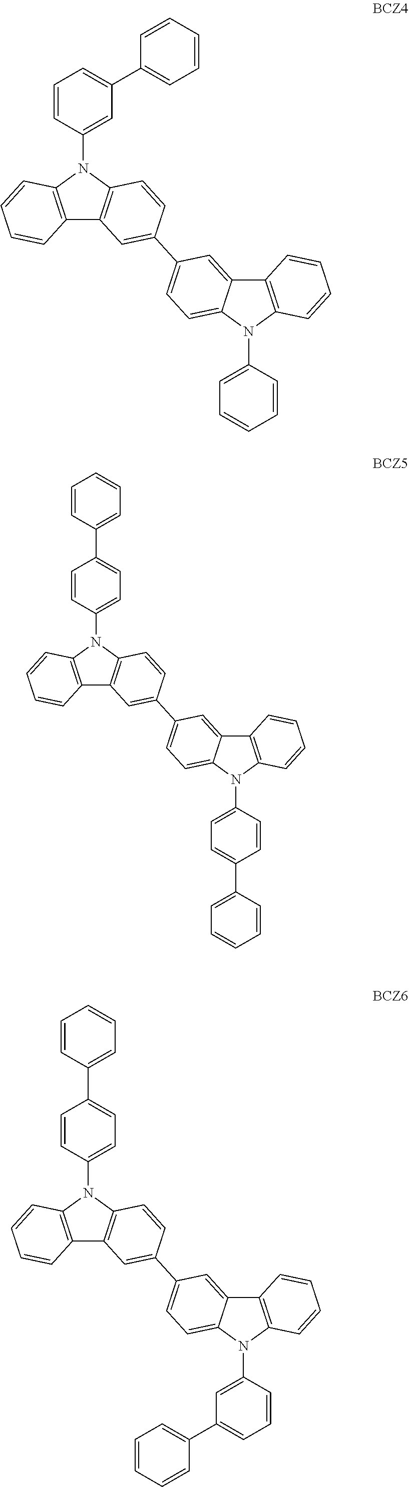 [SMILES: c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

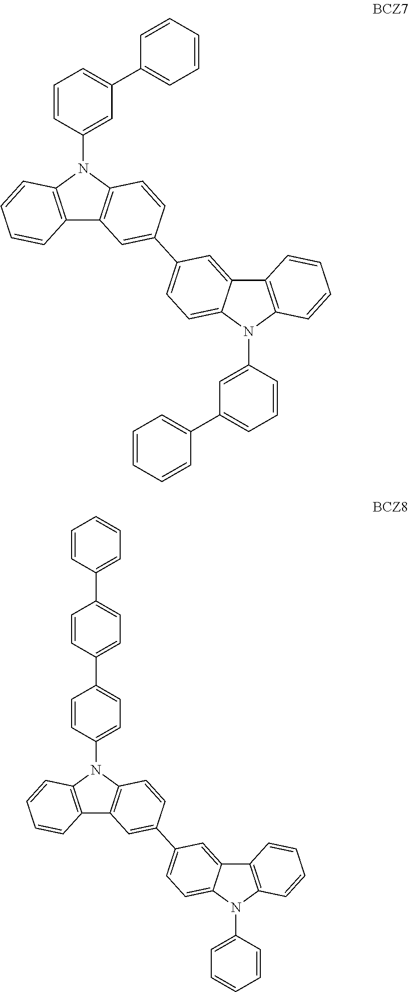 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

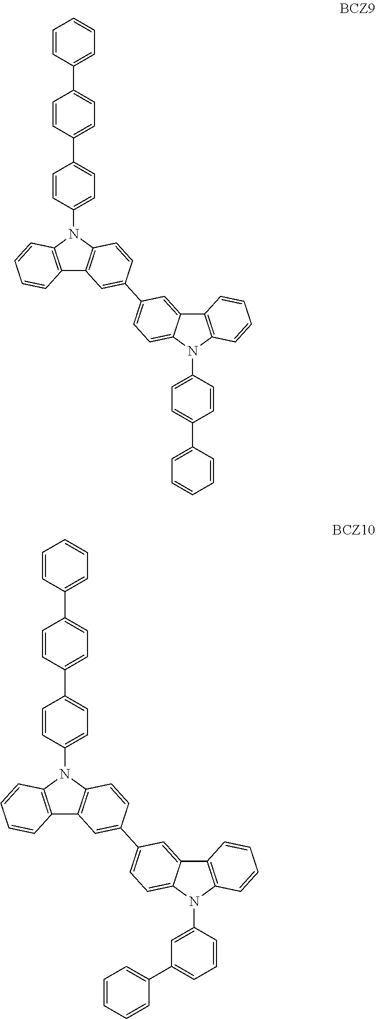 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1]

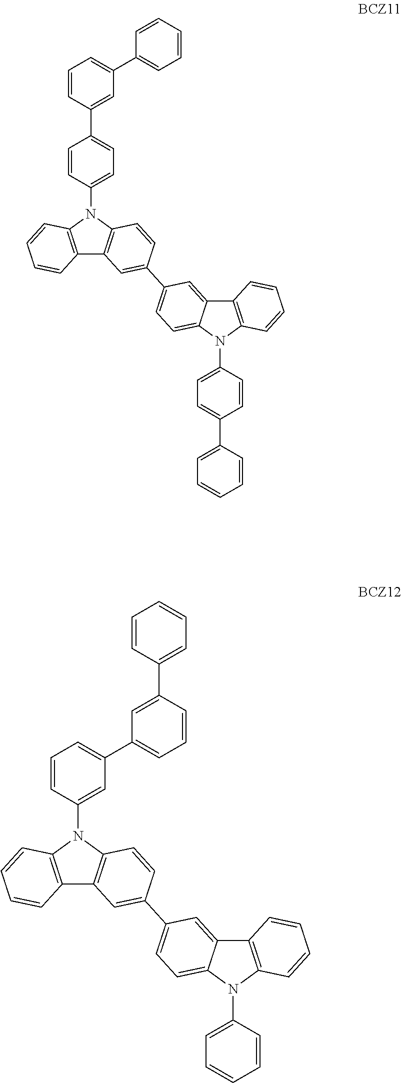 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5cc(-c6ccc7c(c6)c6ccccc6n7-c6ccccc6)ccc54)c3)c2)cc1]

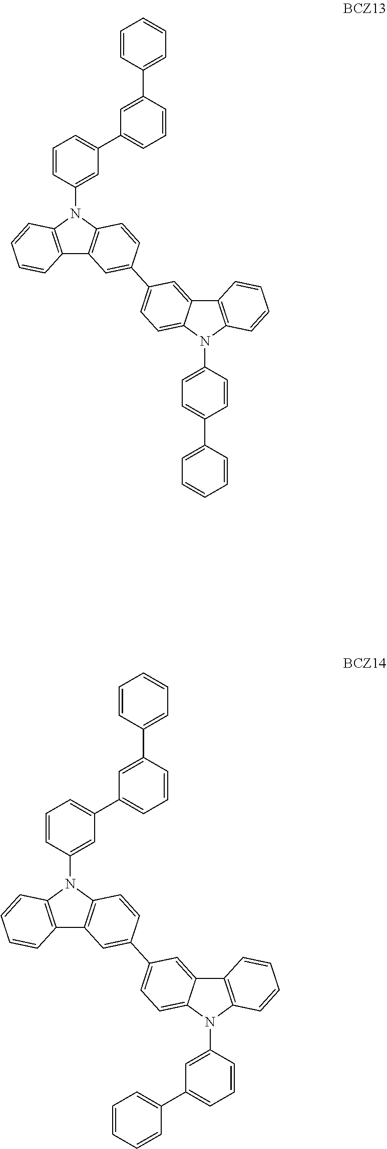 [SMILES: c1ccc(-c2ccc(-n3c4ccccc4c4cc(-c5ccc6c(c5)c5ccccc5n6-c5cccc(-c6cccc(-c7ccccc7)c6)c5)ccc43)cc2)cc1 ; c1ccc(-c2cccc(-c3cccc(-n4c5ccccc5c5cc(-c6ccc7c(c6)c6ccccc6n7-c6cccc(-c7ccccc7)c6)ccc54)c3)c2)cc1]

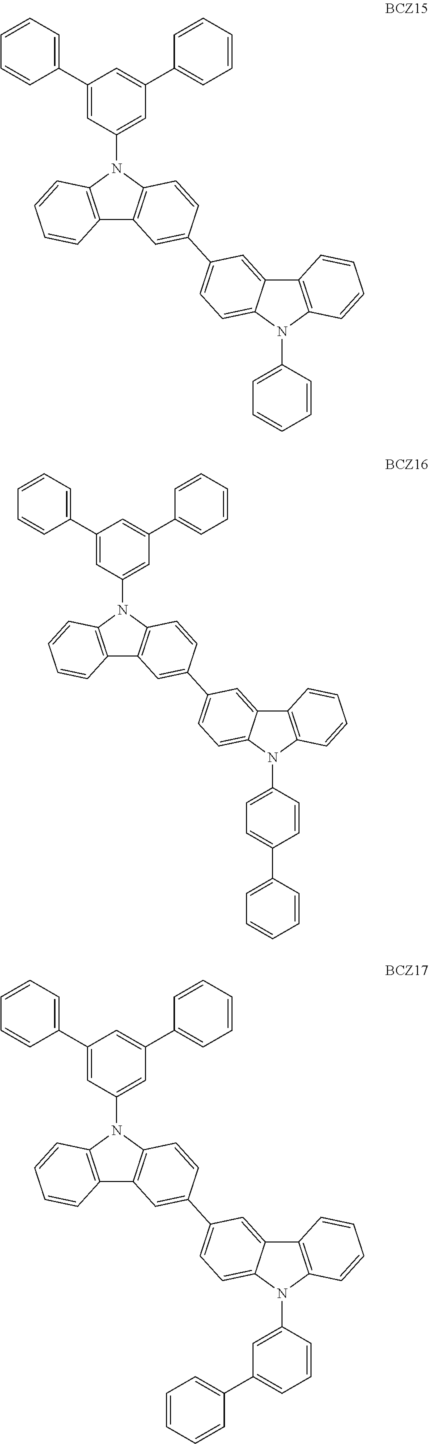 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

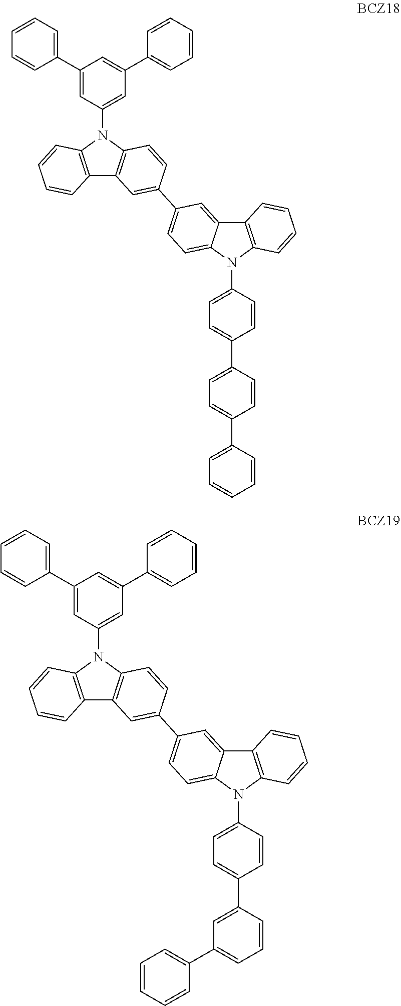

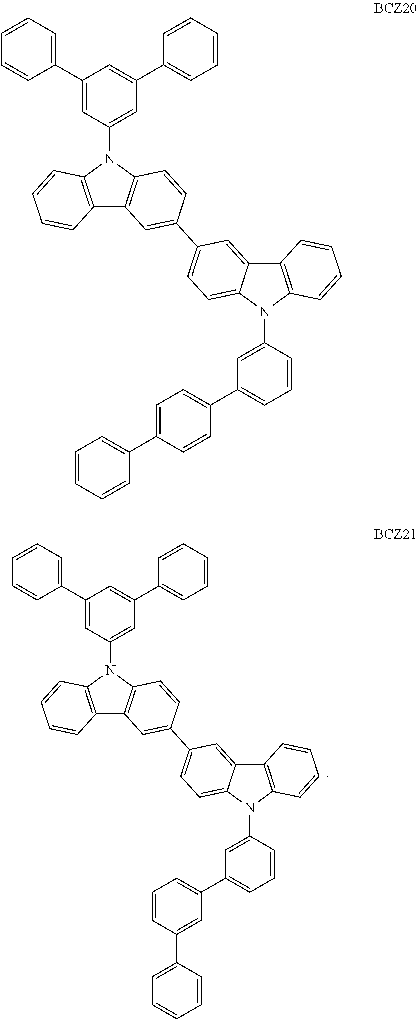

8. The organic light emitting device according to claim 6, wherein the first emitting material layer further includes a third compound being one of the compounds in Formula 5:

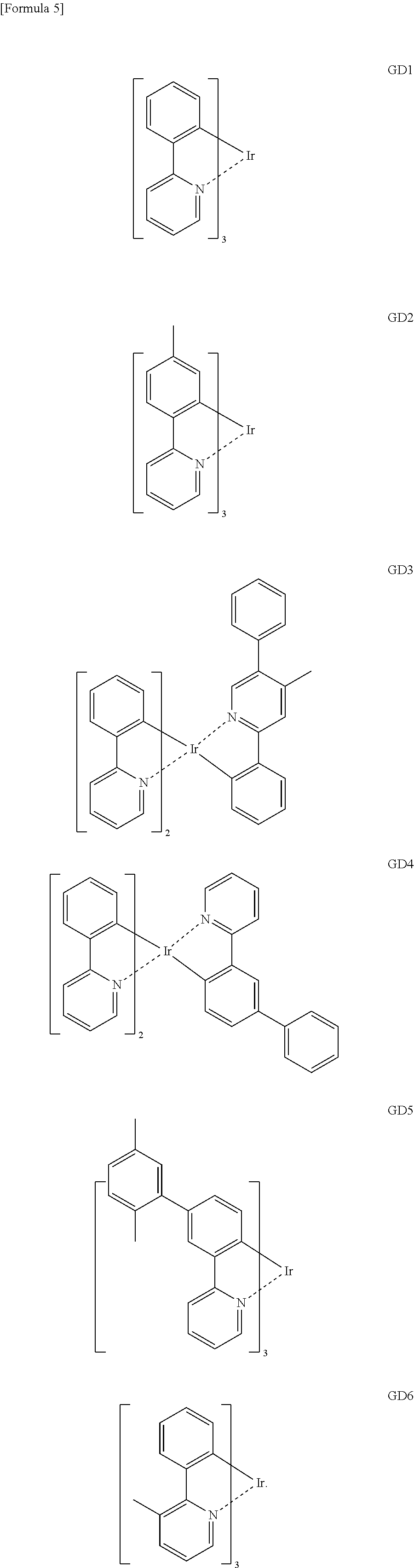

9. The organic light emitting device according to claim 6, wherein the first emitting material layer further includes a third compound being one of the compounds in Formula 6:

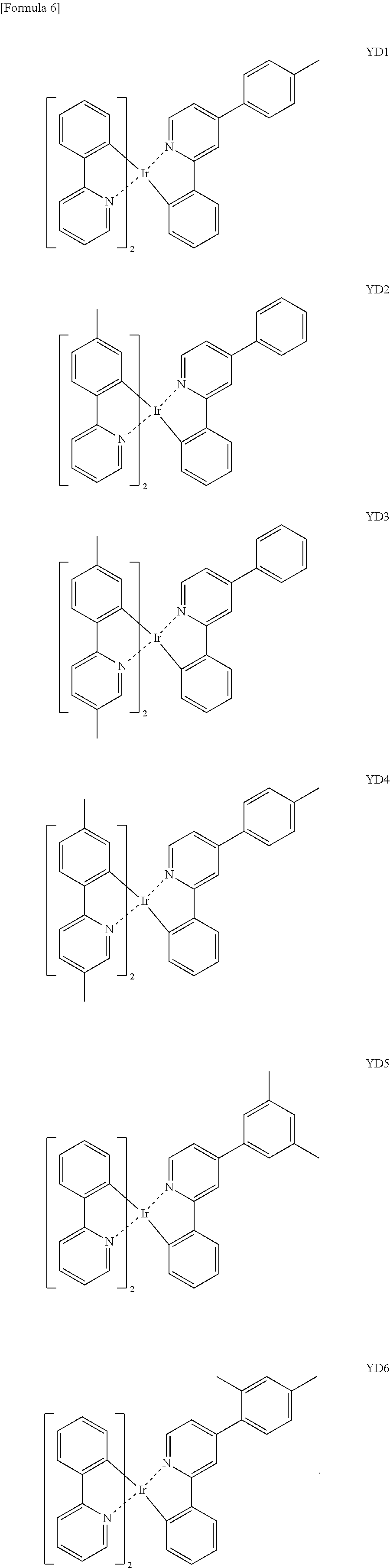

10. The organic light emitting device according to claim 8, wherein a weight % of each of the first compound and the second compound is greater than that of the third compound.

11. The organic light emitting device according to claim 8, wherein the weight % ratio of the first compound to the second compound is 1:9 to 9:1, 2:8 to 8:2, or 7:3 to 3:7.

12. The organic light emitting device according to claim 8, wherein the weight % of the first compound and the weight % of the second compound is the same.

13. The organic light emitting device according to claim 8, wherein the third compound is present in an amount of 5 to 25 weight % in the first emitting material layer, based on a total weight of the components in the first emitting material layer.

14. The organic light emitting device according to claim 5, wherein the first emitting part further includes a first electron transporting layer between the first emitting material layer and the second electrode,
wherein the first electron transporting layer includes at least one of a first electron transporting material represented by Formula 7, a second electron transporting material represented by Formula 8, and a third electron transporting material represented by Formula 9: 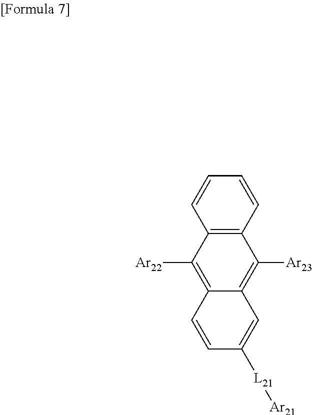
wherein in Formula 7:
L₂₁ is selected from the group consisting of a single bond, a substituted or unsubstituted C6 to C60 arylene group, and a substituted or unsubstituted C3 to C60 heteroarylene group,
Ar₂₁ is represented by Formula 7a or Formula 7b, and
each Ar₂₂ and Ar₂₃ is independently selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group, 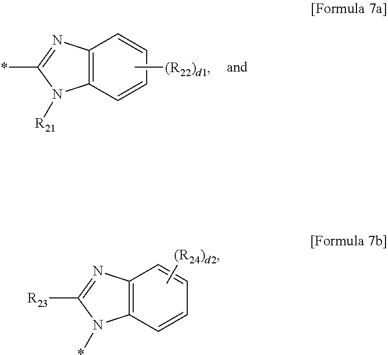
wherein in Formula 7a:
d1 is an integer of 0 to 4,
R₂₁ is selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group, and
each R₂₂ is selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
wherein in Formula 7b:
d2 is an integer of 0 to 4,
R₂₃ is selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group, and
each R₂₄ is selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group, 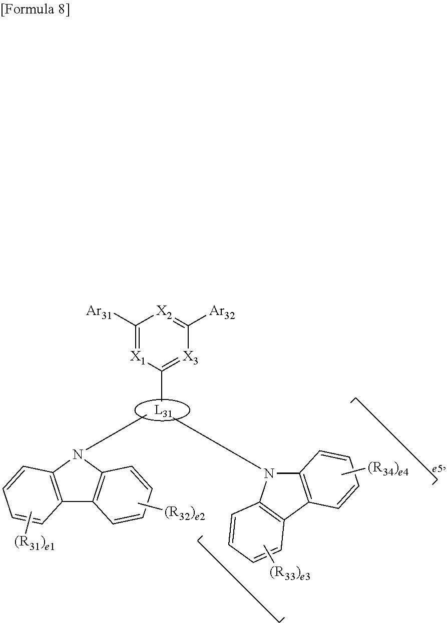
wherein in Formula 8:
each e1, e2, e3, and e4 is independently an integer of 0 to 4, e5 is 0 or 1,
each R₃₁, R₃₂, R₃₃, and R₃₄ is independently selected from the group consisting of deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each X₁, X₂ and X₃ is independently N or CR₃₅, wherein at least two of X₁, X₂ and X₃ are N,
each R₃₅ is selected from the group consisting of hydrogen, deuterium, a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
each Ar₃₁ and Ar₃₂ is independently selected from the group consisting of a substituted or unsubstituted C6 to C60 aryl group, and a substituted or unsubstituted C3 to C60 heteroaryl group, and
L₃₁ is selected from the group consisting of a substituted or unsubstituted C6 to C60 arylene group, and a substituted or unsubstituted C3 to C60 heteroarylene group, 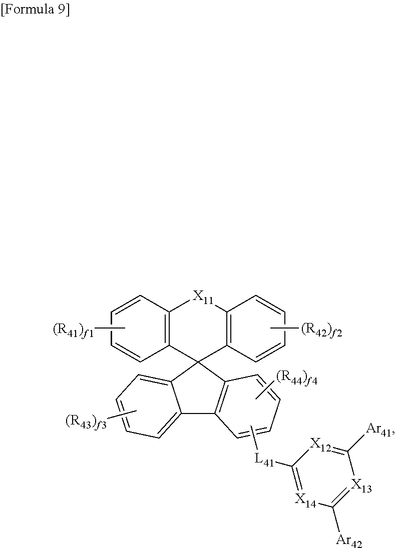
wherein in Formula 9:
each f1, f2, and f3 is independently an integer of 0 to 4,
f4 is an integer of 0 to 3,
each R₄₁, R₄₂, R₄₃, and R₄₄ is independently selected from the group consisting of a substituted or unsubstituted C1 to C10 alkyl group, a substituted or unsubstituted C3 to C30 cycloalkyl group, a substituted or unsubstituted C1 to C10 alkoxy group, a substituted or unsubstituted C6 to C60 aryl group, and a substituted or unsubstituted C3 to C60 heteroaryl group,
X₁₁ is O, S, or NR₄₅,
R₄₅ is a substituted or unsubstituted C6 to C60 aryl group and forms a ring with one of the adjacent benzene rings,
Each X₁₂, X₁₃, and X₁₄ is independently N or CR₄₆, wherein at least two of X₁₂, X₁₃ and X₁₄ are N,
each Ar₄₁, Ar₄₂, and R₄₆ is independently selected from the group consisting of hydrogen, a substituted or unsubstituted C6 to C60 aryl group, and a substituted or unsubstituted C3 to C60 heteroaryl group, and
L₄₁ is selected from the group consisting of a single bond, a substituted or unsubstituted C6 to C60 arylene group, and a substituted or unsubstituted C3 to C60 heteroarylene group.

15. The organic light emitting device according to claim 14, wherein the first electron transporting material is one of compounds in Formula 10:

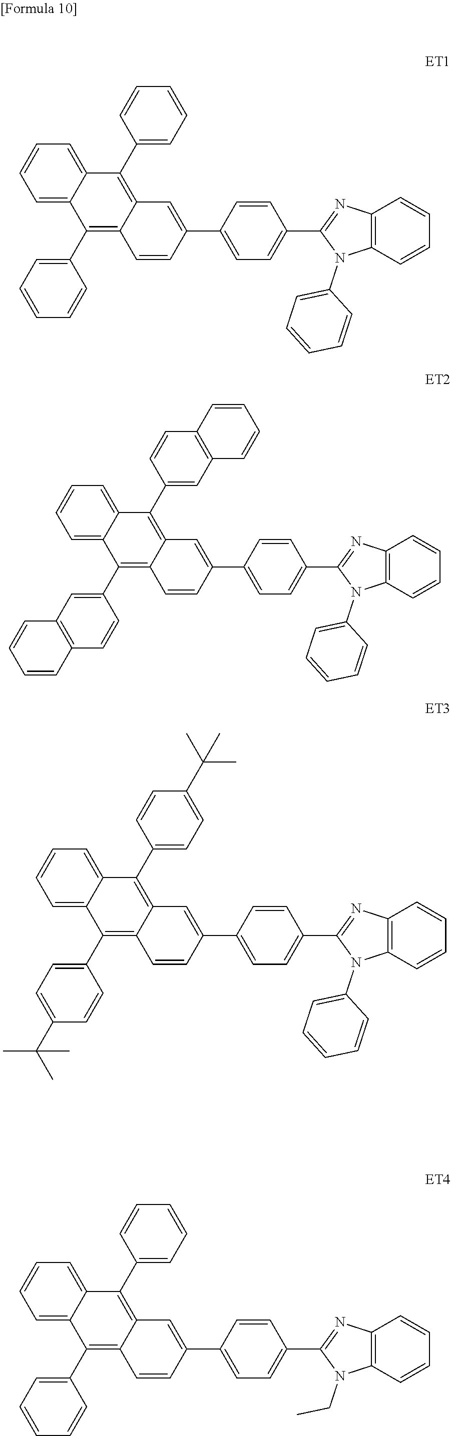

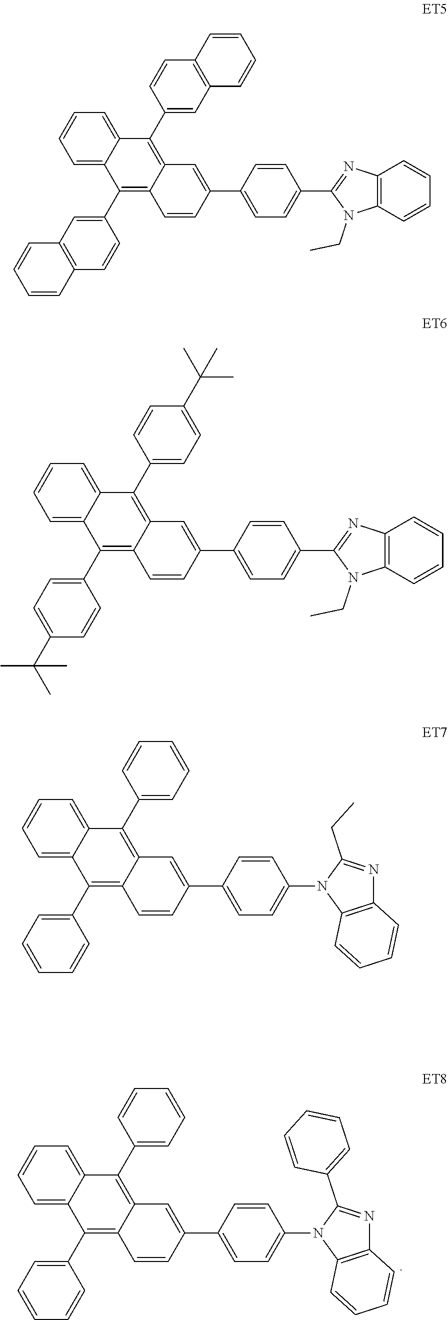

16. The organic light emitting device according to claim 14, wherein the second electron transporting material is one of compounds in Formula 11:

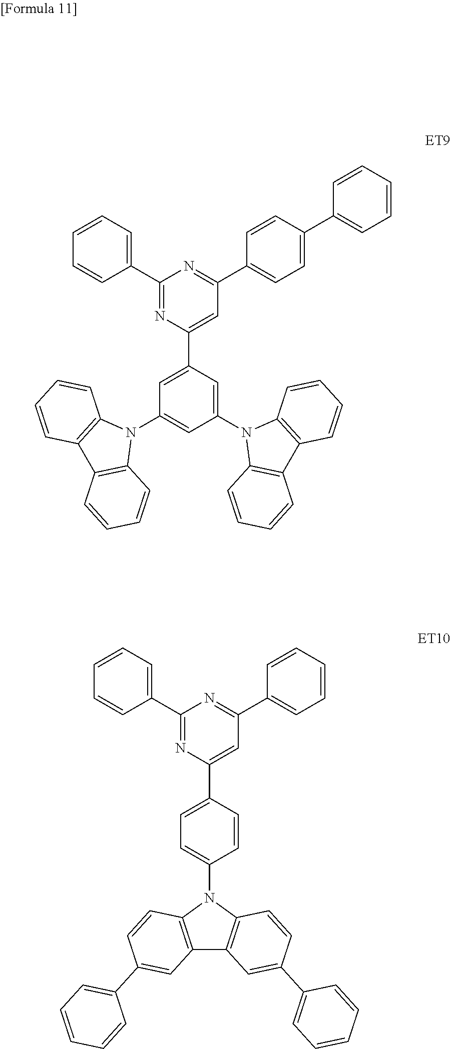 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2ccc3c(c2)c2cc(-c4ccccc4)ccc2n3-c2ccc(-c3cc(-c4ccccc4)nc(-c4ccccc4)n3)cc2)cc1]

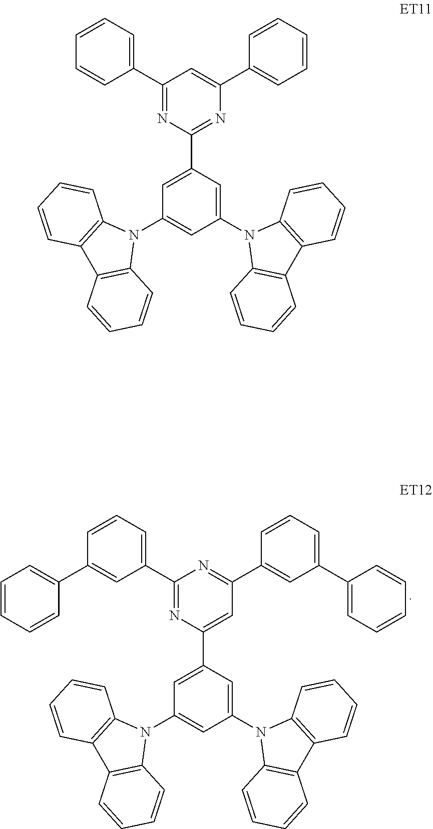 [SMILES: c1ccc(-c2cc(-c3ccccc3)nc(-c3cc(-n4c5ccccc5c5ccccc54)cc(-n4c5ccccc5c5ccccc54)c3)n2)cc1 ; c1ccc(-c2cccc(-c3cc(-c4cc(-n5c6ccccc6c6ccccc65)cc(-n5c6ccccc6c6ccccc65)c4)nc(-c4cccc(-c5ccccc5)c4)n3)c2)cc1]

17. The organic light emitting device according to claim 14, wherein the third electron transporting material is one of compounds in Formula 12:

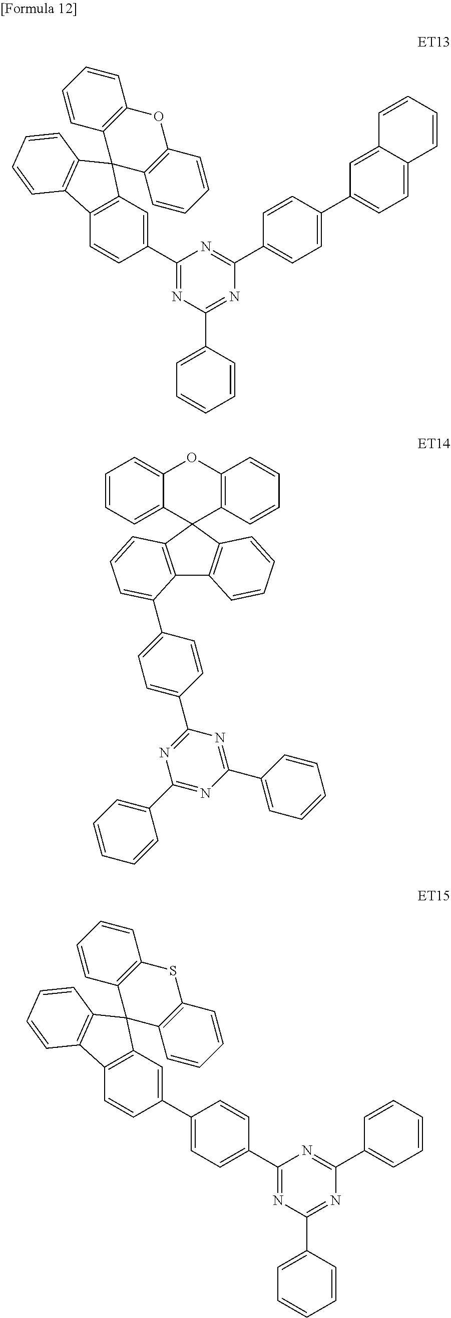

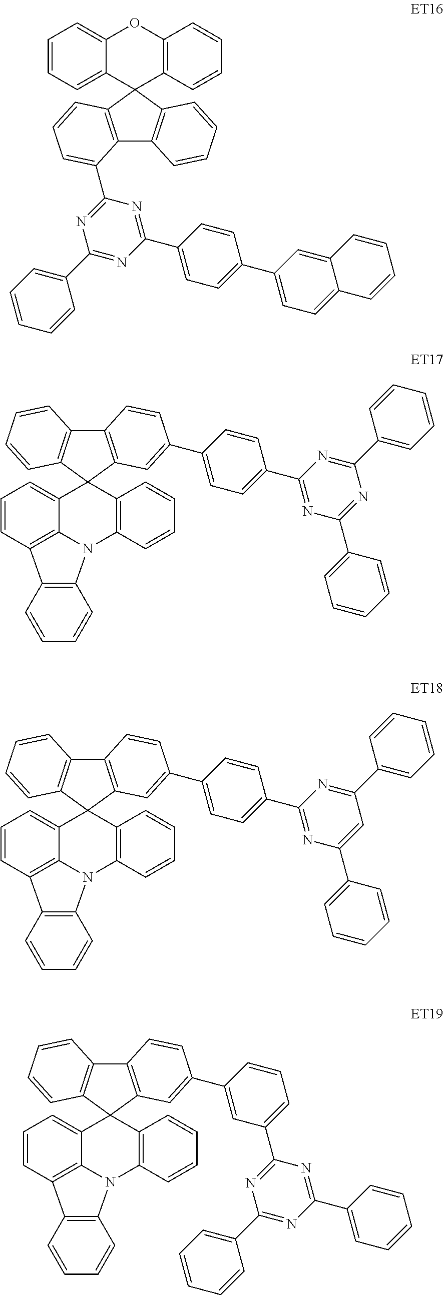 [SMILES: c1ccc(-c2nc(-c3ccc(-c4ccc5ccccc5c4)cc3)nc(-c3cccc4c3-c3ccccc3C43c4ccccc4Oc4ccccc43)n2)cc1 ; c1ccc(-c2nc(-c3ccccc3)nc(-c3ccc(-c4ccc5c(c4)C4(c6ccccc6-5)c5ccccc5-n5c6ccccc6c6cccc4c65)cc3)n2)cc1 ; c1ccc(-c2cc(-c3ccccc3)nc(-c3ccc(-c4ccc5c(c4)C4(c6ccccc6-5)c5ccccc5-n5c6ccccc6c6cccc4c65)cc3)n2)cc1 ; c1ccc(-c2nc(-c3ccccc3)nc(-c3cccc(-c4ccc5c(c4)C4(c6ccccc6-5)c5ccccc5-n5c6ccccc6c6cccc4c65)c3)n2)cc1]

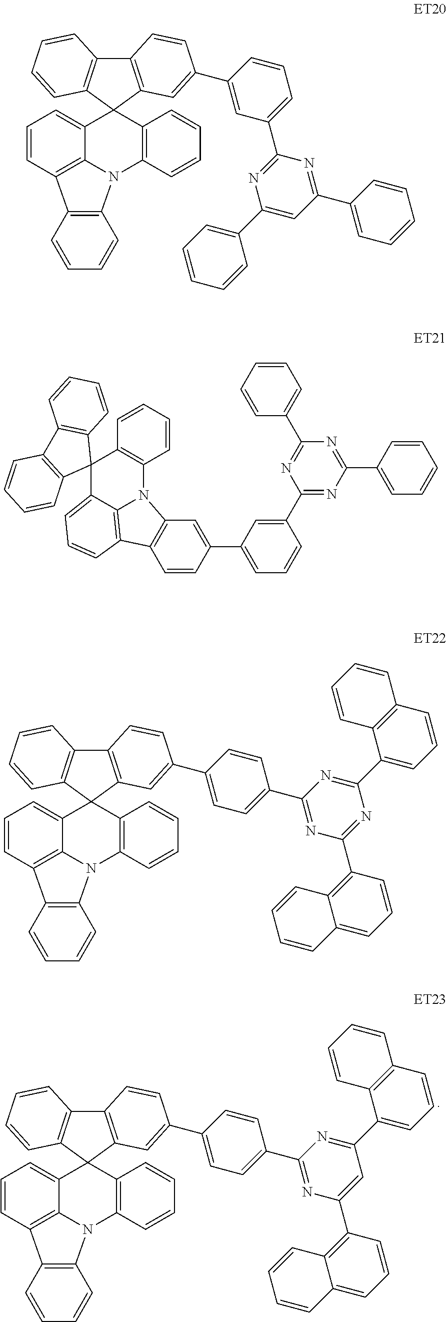 [SMILES: c1ccc(-c2cc(-c3ccccc3)nc(-c3cccc(-c4ccc5c(c4)C4(c6ccccc6-5)c5ccccc5-n5c6ccccc6c6cccc4c65)c3)n2)cc1 ; c1ccc(-c2nc(-c3ccccc3)nc(-c3cccc(-c4ccc5c6cccc7c6n(c5c4)-c4ccccc4C74c5ccccc5-c5ccccc54)c3)n2)cc1 ; c1ccc2c(c1)-c1ccc(-c3ccc(-c4nc(-c5cccc6ccccc56)nc(-c5cccc6ccccc56)n4)cc3)cc1C21c2ccccc2-n2c3ccccc3c3cccc1c32 ; c1ccc2c(c1)-c1ccc(-c3ccc(-c4nc(-c5cccc6ccccc56)cc(-c5cccc6ccccc56)n4)cc3)cc1C21c2ccccc2-n2c3ccccc3c3cccc1c32 ; "."]

18. The organic light emitting device according to claim 5, wherein the organic light emitting diode further includes:
a second emitting part including a second emitting material layer and positioned between the first emitting part and the second electrode,
wherein the second emitting material layer includes the first compound.

19. The organic light emitting device according to claim 5, wherein the organic light emitting diode further includes:
a second emitting part including a first blue emitting material layer and positioned between the first electrode and the first emitting part.

20. The organic light emitting device according to claim 19, wherein the first emitting part further includes a red emitting material layer between the second emitting part and the first emitting material layer.

21. The organic light emitting device according to claim 20, wherein the first emitting part further includes a yellow-green emitting material layer between the first emitting material layer and the red emitting material layer.

22. The organic light emitting device according to claim 15, wherein the organic light emitting diode further includes:
a third emitting part including a second blue emitting material layer and positioned between the first emitting part and the second electrode.

23. The organic light emitting device according to claim 22, wherein the first emitting part further includes a red emitting material layer between the second emitting part and the first emitting material layer.

24. The organic light emitting device according to claim 23, wherein the first emitting part further includes a yellow-green emitting material layer between the first emitting material layer and the red emitting material layer.

25. The organic light emitting device according to claim 5, further comprising:
a color filter layer corresponding to a red pixel region, a green pixel region, and a blue pixel region.
